SEQUENCE LISTING (203 sequences)

SEQ ID NO: 1 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 4 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 5 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 220 aa, Homo sapiens
DGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 8 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 9 - protein, 227 aa, Mus musculus
NGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 10 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 11 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 13 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 21 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 22 - protein, 9 aa, synthetic construct
GGGGGGGGG

SEQ ID NO: 23 - protein, 11 aa, synthetic construct
  VARIANT 1..10: note = This region may encompass 1-10 residues
GGGGGGGGGGS

SEQ ID NO: 24 - protein, 11 aa, synthetic construct
  VARIANT 2..11: note = This region may encompass 1-10 residues
SGGGGGGGGGG

SEQ ID NO: 25 - protein, 5 aa, synthetic construct
GGGGS

SEQ ID NO: 26 - protein, 5 aa, synthetic construct
GGGGS

SEQ ID NO: 27 - protein, 4 aa, synthetic construct
GGGG

SEQ ID NO: 28 - protein, 5 aa, synthetic construct
GGGGG

SEQ ID NO: 29 - protein, 6 aa, synthetic construct
GGGGGG

SEQ ID NO: 30 - protein, 7 aa, synthetic construct
GGGGGGG

SEQ ID NO: 31 - protein, 8 aa, synthetic construct
GGGGGGGG

SEQ ID NO: 32 - protein, 4 aa, synthetic construct
CPPC

SEQ ID NO: 33 - protein, 4 aa, Homo sapiens
CPSC

SEQ ID NO: 34 - protein, 20 aa, synthetic construct
EPKSCDKTHTCPPCPAPPVA

SEQ ID NO: 35 - protein, 17 aa, synthetic construct
ESKYGPPCPPCPAPPVA

SEQ ID NO: 36 - protein, 15 aa, synthetic construct
CPPCPAPGGGGPSVF

SEQ ID NO: 37 - protein, 14 aa, synthetic construct
CPPCPAPGGGPSVF

SEQ ID NO: 38 - protein, 13 aa, synthetic construct
CPPCPAPGGPSVF

SEQ ID NO: 39 - protein, 12 aa, synthetic construct
CPPCPAPGPSVF

SEQ ID NO: 40 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFEKTKIKLCILLHAFRIRAVTIDRVMSYLNAS

SEQ ID NO: 41 - protein, 228 aa, synthetic construct
ESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 235 aa, synthetic construct; identical to SEQ ID NO: 17

SEQ ID NO: 43 - protein, 235 aa, synthetic construct
DKKVEPKSCDKTHTCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 5 aa, synthetic construct
  VARIANT 3: note = Any amino acid
WSXWS

SEQ ID NO: 45 - protein, 6 aa, Homo sapiens
WSEWAS

SEQ ID NO: 46 - protein, 124 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 47 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 48 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 49 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 50 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 51 - protein, 15 aa, synthetic construct
GGGGSGGGGSGGGGS

SEQ ID NO: 52 - protein, 30 aa, synthetic construct
  VARIANT 1..30: note = This sequence may encompass 2-6 GGGGS repeating units
GGGGSGGGGSGGGGSGGGGSGGGGSGGGGS

SEQ ID NO: 53 - protein, 121 aa, synthetic construct
QVQLVESGGGVVQPGRSLRLSCAASGFTFTSYGMSWVRQAPGKGLEWVAGISYSGSDTEYADSVKGRFTISRDNSKNTLYLQMNSLRAEDTAVYYCARSPDYIIDYGFDYWGRGTLVTVSS

SEQ ID NO: 54 - protein, 109 aa, synthetic construct
AIQMTQSPSSLSASVGDRVTITCRASQGISSDLAWYQQKPGKAPKLLIYDASSLQSGVPSRFSGSGSGTDFTLTISSLQPEDFATYYCQQYWIYPFTFGQGTKVEIKRT

SEQ ID NO: 55 - protein, 121 aa, synthetic construct
QVQLVESGGGVVQPGRSLRLSCAASGFTFTSYGMSWVRQAPGKGLEWVAGISYDASDTEYADSVKGRFTISRDNSKNTLYLQMNSLRAEDTAVYYCARSPDYIIDYGFDYWGRGTLVTVSS

SEQ ID NO: 56 - protein, 109 aa, synthetic construct
AIQMTQSPSSLSASVGDRVTITCRASQGISSDLAWYQQKPGKAPKLLIYDASSLQSGVPSRFSGSGSGTDFTLTISSLQPEDFATYYCQQYWWYPFTFGQGTKVEIKRT

SEQ ID NO: 57 - protein, 121 aa, synthetic construct
QVQLVQSGAEVKKPGASVKVSCKASGYTFTGYYMHWVRQAPGQGLEWMGMIGPQHGEAIYAQKFQGRVTMTRDTSISTAYMELSRLRSEDTAVYYCARESTDSDESPFDYWGQGTLVTVSS

SEQ ID NO: 58 - protein, 110 aa, synthetic construct
DIELTQPPSVSVSPGQTASITCSGDNIRSYYVSWYQQKPGQAPVLVIYDDSDRPSGIPERFSGSNSGNTATLTISGTQAEDEADYYCQSYGSHSNFVVFGGGTKLTVLGQ

SEQ ID NO: 59 - protein, 121 aa, synthetic construct
QVQLVQSGAEVKKPGASVKVSCKASGYTFTGYYMHWVRQAPGQGLEWMGMIGPQHGEAIYAQKFQGRVTMTRDTSISTAYMELSRLRSDDTAVYYCARESTDSDESPFDYWGQGTLVTVSS

SEQ ID NO: 60 - protein, 110 aa, synthetic construct
SYELTQPLSVSVALGQTARITCSGDNIRSYYVSWYQQKPGQAPVLVIYDDSDRPSGIPERFSGSNSGNTATLTISRAQAGDEADYYCQSYGSHSNFVVFGGGTKLTVLGQ

SEQ ID NO: 61 - protein, 120 aa, synthetic construct
QVQLKESGPALVKPTQTLTLTCTFSGFSLSTSTMGVSWIRQPPGKALEWLAWIYWDDDKDYSTSLKSRLTISKDTSKNQVVLTMTNMDPVDTATYYCARANPDLGYFDYWGQGTLVTVSS

SEQ ID NO: 62 - protein, 121 aa, synthetic construct
QVQLKESGPALVKPTQTLTLTCTFSGFSLSTSGMGVSWIRQPPGKALEWLALIDWTDDKYYSTSLKTRLTISKDTSKNQVVLTMTNMDPVDTATYYCARTVGKGLYRVDNWGQGTLVTVSS

SEQ ID NO: 63 - protein, 122 aa, synthetic construct
QVQLVESGGGLVQPGGSLRLSCAASGFTFSSYGMSWVRQAPGKGLEWVSYIEPKLFWYATFYAASVKGRFTISRDNSKNTLYLQMNSLRAEDTAVYYCARNDFMEPAYFALWGQGTLVTVSS

SEQ ID NO: 64 - protein, 126 aa, synthetic construct
QVQLKESGPALVKPTQTLTLTCTFSGFSLSTRGVGVSWIRQPPGKALEWLALIYWDEDKYYSTSLKTRLTISKDTSKNQVVLTMTNMDPVDTATYYCARYQSGYYYNNDGWGVDIWGQGTLVTVSS

SEQ ID NO: 65 - protein, 111 aa, synthetic construct
DIALTQPASVSGSPGQSITISCTGTSSDLGESNYVSWYQQHPGKAPKVMIYDVNKRPSGVSNRFSGSKSGNTASLTISGLQAEDEADYYCGSYDEEDNVFGGGTKLTVLGQ

SEQ ID NO: 66 - protein, 110 aa, synthetic construct
DIELTQPPSVSVAPGQTARISCSGDNLGSKFAYWYQQKPGQAPVLVIYDDSKRPSGIPERFSGSNSGNTATLTISGTQAEDEADYYCQSWDSSSGNDVFGGGTKLTVLGQ

SEQ ID NO: 67 - protein, 109 aa, synthetic construct
DIELTQPPSVSVAPGQTARISCSGDNLGSYYAYWYQQKPGQAPVGVIYDDSERPSGIPERFSGSNSGNTATLTISGTQAEDEADYYCSSYTYSKNNVFGGGTKLTVLGQ

SEQ ID NO: 68 - protein, 109 aa, synthetic construct
DIQMTQSPSSLSASVGDRVTITCRASQSISSWLNWYQQKPGKAPKLLIYAASSLQSGVPSRFSGSGSGTDFTLTISSLQPEDFATYYCQQYYAFPHTFGQGTKVEIKRT

SEQ ID NO: 69 - protein, 126 aa, synthetic construct
QVQLQESGGGSVQAGGSLRLSCVASGYGYCGYDMSWYRQAPGKEREFVALITSDRSISYEDSVKARFIISRDNAANTGYLDMTRLTPDDTAIYYCKTSAAARESSWCRSRYRVASWGQGTQVTVSS

SEQ ID NO: 70 - protein, 127 aa, synthetic construct
QVQLQESGGGSVQAGGSLRLSCTASGYTYSSAFMAWFRQAPGKEREGVAAIYTRDGGTVYADSVKGRFTISQDNAKNILYLQMNSLKAEDTAMYYCAAKIPQPGRASLLDSQTYDYWGQGTQVTVSS

SEQ ID NO: 71 - protein, 126 aa, synthetic construct
QVQLQESGGGSVQAGGSLRLSCVASGYSYCGYDMMWYRQAPGKEREFVALITSDYSIRYEDSVEGRFSISRDNAKNTGYLLMSNLTPADTAIYYCKTSTAARESSWCRSRYRVASWGQGTQVTVSS

SEQ ID NO: 72 - protein, 126 aa, synthetic construct
QVQLQESGGGSVQAGGSLRLSCVASGYGYCGYDMSWYRQTPGKEREFVALITSDRIASYEDSVKGRFIISRDNAKNTGYLDMTRVTPDDTAIYYCKTSAAARENSWCRSRYRVASWGQGTQVTVSS

SEQ ID NO: 73 - protein, 126 aa, synthetic construct
QVQLQESGGGSVQAGGFLRLSCVASGYGYCGYDMSWYRQVPGKEREFVALITSDRSVSYEDSVKGRFSISRDNAKNTAYLEMNRLTPDDTAVYYCKTSTAARENNWCRSRYRIAYWGQGTQVTVSS

SEQ ID NO: 74 - protein, 127 aa, synthetic construct
QVQLQESGGGSVQAGGSLRLSCAASRYTYTNNFMAWFRQAPGKEREGVAAIYTGDGYAYYFYSVKGRFTISQDNDENMLYLQMNSLKPEDTAMYYCAAMERRIGTRRMTENAEYKYWGQGTQVTVSS

SEQ ID NO: 75 - protein, 126 aa, synthetic construct
QVQLQESGGGSVQAGGSLRLSCAVSGYDYCGYDVRWYRRAPGKEREFVSGIDSDGSTSYADSVKGRFTISQDNAENTSYLHMFSLKPEDTAMYYCKTESPAGESAWCRNFRGMDYWGKGTQVTVSS

SEQ ID NO: 76 - protein, 126 aa, synthetic construct
QVQLQESGGGSVQAGGSLRLSCVASGYSYCGYDMMWYRQAPGKEREFVALITSDYSIRYEDSVEGRFSISRDNAKNTGYLLMSNLTPADTAIYYCKTSTAARESGWCRSRYRVASWGQGTQVTVSS

SEQ ID NO: 77 - protein, 126 aa, synthetic construct
QVQLQESGGGSVQAGGSLRLSCAVSGYDYCGYDVRWYRQAPGKEREFVSGIDSDGSTSYADSVKGRFTISQDNAENTSYLHMFSLKPEDTAMYYCKTESPAGESAWCRNFRGMDYWGKGTQVTVSS

SEQ ID NO: 78 - protein, 127 aa, synthetic construct
QVQLQESGGGSVQAGGSLRLSCTASGYTYSSAFMAWFRQAPGKEREGVAAIYTRDGGTVYADSVKGRFTISQDNAKNTLYLQMNSLKPEDTAMYYCAAKMPQPGRASLLDSQTYDYWGQGTQVTVSS

SEQ ID NO: 79 - protein, 126 aa, synthetic construct
QVQLQESGGGSVQAGGFLRLSCVASGYGYCGYDMSWYRQAPGKEREFVALITSERVISYEDSVKGRFSISRDNAENTGYLEMNRLTPDDTAIYYCKTSAAARESSWCRSRYRVASWGQGTQVTVSS

SEQ ID NO: 80 - protein, 126 aa, synthetic construct
QVQLQESGGGSVQAGGSLRLSCAVSGYDYCGYDVRWYRQAPGKEREFVSGINSDGSTSYADSVKGRFTISQDNAENTSYLHMFSLKPEDTAMYYCKTESPAGESAWCRNFRGMDYWGKGTQVTVSS

SEQ ID NO: 81 - protein, 127 aa, synthetic construct
QVQLQESGGGSVQAGGSLRLSCTASGYTYSSAFMAWFRQAPGKEREGVAAMYTRDGGTVYADSVKGRFTISQDNAKNTLYLQIHTLKAEDTAMYYCAAKIPQPGRASLLDSQTYDYWGQGTQVTVSS

SEQ ID NO: 82 - protein, 126 aa, synthetic construct
QVQLQESGGGSVQAGGFLRLSCVASGYGYCGYDMSWYRQVPGKEREFVALITSDRSVSYEDSVKGRFSISRDNAKNTAYLEMNRLTPDDTAIYYCKTSTAARENNWCRSRYRIASWGQGTQVTVSS

SEQ ID NO: 83 - protein, 127 aa, synthetic construct
QVQLQESGGGSVQAGGSLRLSCAASRYTYTNNFMAWFRQAPGKEREGVAAIYTGDGYAYYFDSVKGRFTISQDNDKNMLYLQMNSLKPEDTAMYYCAAMERRSGRRRMTENAEYKYWGQGTQVTVSS

SEQ ID NO: 84 - protein, 126 aa, synthetic construct
QVQLQESGGGSVQAGGSLRLSCAVSGYDYCGYDVRWYRQAPGKEREFVSGINSDGSTSYADSVKGRFTISQDNAENTSYLHMFSLKPEDTAMYYCKTEGPAGESAWCRNFRGMDYWGKGTQVTVSS

SEQ ID NO: 85 - protein, 127 aa, synthetic construct
QVQLQESGGGSVQAGGSLRLSCTASGYTYSSAFMAWFRQAPGKEREGVAAIYTRDGSPVYADSLKGRFTISQDNAKNTLHLQMNSLKPEDTAMYYCAAKIPEPGRISLLDSQTYDYWGHGTQVTVSS

SEQ ID NO: 86 - protein, 127 aa, synthetic construct
QVQLQESGGGSVQAGGSLRLSCTASGYTYSSAFMAWFRQAPGKEREGVAAMYTRDGGTVYADSVKGRFTISQDNAKNTLYLQMNSLKTEDTAMYYCAAKIPQPGRASLLDSQTYDYWGQGTQVTVSS

SEQ ID NO: 87 - protein, 127 aa, synthetic construct
QVQLQESGGGSVQAGGSLRLSCTASGYTYSSAFMAWFRQAPGKEREGVAAIYTRDGGTVYADSVKGRFTISQDNAKNTLYLQMNSLKAEDTAMYYCAAKIPQPGRASLLDSQTYDYWGQGTQVTVSS

SEQ ID NO: 88 - protein, 121 aa, synthetic construct
QVQLQESGGGSVQAGETLRLSCTVSGFTIDDSEMGWYRQAPGHECELVASGSSDDDTYYVDSVKGRFTISLDNAKNMVYLQMNSLKPEDTAVYYCATGPTYPPKDGDCAHWGQGTQVTVSS

SEQ ID NO: 89 - protein, 118 aa, synthetic construct
EVQLVESGGGLVQAGGSLRLSCAVSGIAFRYNSVAWSRQAPGSQRELVARITNSARTNYADSVKGRFTISRDNDKNMVYLQMNSLKPEDTAVYYCGAGRSMTGDVAYWGQGTQVTVSS

SEQ ID NO: 90 - protein, 124 aa, synthetic construct
EVQLVESGGGLVQAGGSLRLSCAASGITFDDDYAIGWFRQAPGKEREGVSLISSSDGSTYYADSVKGRFTITSDNAKNTVYLQMNSLKPEDTAVYYCAADPTSGLPSDDEYDYWGQGTQVTVSS

SEQ ID NO: 91 - protein, 118 aa, synthetic construct
EVQLVESGGGLVQAGGSLRLSCAASGIAFRYNSVAWSRQAPGSQREVVAGITNSARTNYADSVKGRFTISRDNDKKMVYLQMNSLKPEDTAVYYCGAGRSMAGDVAYWGQGTQVTVSS

SEQ ID NO: 92 - protein, 118 aa, synthetic construct
EVQLVESGGGLVQAGGSLRLSCAASGIAFRYNSVAWSRQAPGSQRELVASISNSARTKYADSVKGRFTISRDNEKKMLYLQMDSLKPEDTAVYYCGAGRSMAGDVAYWGQGTQVTVSS

SEQ ID NO: 93 - protein, 117 aa, synthetic construct
EVQLVESGGGLVQPGGSLRLSCVASGRPLTGSTMAWFRQAPGKECEFVARISGSGTINYADSLRGRFTISRDVPKNTVWLQMDSLKPDDTAVYYCAAVKVAGSYEYWGQGTQVTVSS

SEQ ID NO: 94 - protein, 123 aa, synthetic construct
EVQLVESGGGSVQTGGSLTLSCTASGRTGSTDGVGWFRQAPGKEREFVSAIKWIGGSKYYSNSAEGRFTIAVDNAKNTVYLQMNSLNPEDTAVYYCAAGAIMFPSRPRDFDFWGQGTQVTVSS

SEQ ID NO: 95 - protein, 123 aa, synthetic construct
EVQLVESGGGLVQAGGSLRLSCAASGRTFSSYAMGWFRQAPGKEREFVAHINWNGGNTYYADSVRGRFIISRDNAKNTLYLQMNRLKPEDTAVYYCAARPDRVIVKWDEYDYWGQGTQVTVSS

SEQ ID NO: 96 - protein, 117 aa, synthetic construct
EVQLVESGGGLVQPGESLTLSCAASGFTFSSYWMYWVRQAPGRELEWVARIQPGSSYTAYADSVKGRFTISRDLAKNTLYLQMNRLKSDDTAVYYCAKDWEMAAPSLGQGTQVTVSS

SEQ ID NO: 97 - protein, 120 aa, synthetic construct
EVQLVESGGGLVQAGGSLRLSCAASGTFLSINRMGWYRQAPGKERELVAVIISGGSTNYADSVKGRFTISRENAELTVYLQMNSLKPEDTAVYYCNVWINTDGIYTYEYWGQGTQVTVSS

SEQ ID NO: 98 - protein, 113 aa, synthetic construct
EVQLVESGGGSVQPGGSLRLSCAASGLIVGRPAMGWYRQAPGKQRELVAIIGSGGNTNYPESVKGRFTIARENANNTVYLQMNSLKPEDTAVYHCNLHGTRFWGQGTQVTVSS

SEQ ID NO: 99 - protein, 122 aa, synthetic construct
EVQLVESGGGLVQAGGSLRLSCAASGLTFSSPAMAWFRQVPGKEREFVATIRRSSAWTDYADSVKGRFTISRDRPTNTAYLQMSSLKPEDTAVYYCAADKISRGIDPNWTYWGRGTQVTVSS

SEQ ID NO: 100 - protein, 111 aa, synthetic construct
EVQLVESGGGLVQPGGSLRLSCAVSGISVRSSVVNWYRQAPGKQRELVALIMGGAIRKYADDVKGRFTISSDSAKNTVYLQMNSLRPEDTAVYYCSALEYWGQGTQVTVSS

SEQ ID NO: 101 - protein, 111 aa, synthetic construct
EVQLVESGGGLVQPGGSLRLSCAVSGISVRSSVVNWYRQAPGKQRELVALIMGGAITKYADDVKGRFTISSDSARNTVYLQMNSLRPEDTAVYSCSALEYWGQGTQVTVSS

SEQ ID NO: 102 - protein, 125 aa, synthetic construct
EVQLVESGGGLAQEGGSLRLSCAASGRPLTTYGMAWFRQAPGKEREFVARIGAEPGATVYGDSVKGRFTISRDNAKNTVYLQMNTLKPEDTAVYYCAADAPPFGPYYRESVYDYWGQGTQVTVSS

SEQ ID NO: 103 - protein, 121 aa, synthetic construct
EVQLVESGGGLVTAGGSLRLSCAASGFRFSVYDMGWFRQAPGKEREFVAVIVGSRTTDYADSVKGRFIIFRDNAKNTLYLQMNGLKPDDTAVYYCARRVGTYETVLGYDKWGQGTQVTVSS

SEQ ID NO: 104 - protein, 134 aa, synthetic construct
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCAAPLLRGGCPITYYSGSYPHVYHAMDYWGKGTLVTVSS

SEQ ID NO: 105 - protein, 117 aa, synthetic construct
EVQLVESGGGLVQPGGSLRLSCAASGFTFSRAWMYWVRQAPGETLEWVSRIQPGGGSTSYADSVKGRFTISRDNAKNTLYLQMNSLKSEDTAVYYCAKDWEMAAPSLGQGTLVTVSS

SEQ ID NO: 106 - protein, 115 aa, synthetic construct
EVQLVESGGGLVQPGGSLRLSCKASRSIFSINTMDWHRQVPGKQRELVAAIVNGVWKNYADSVKGRFTISRDNAENTVYLQMNNLKPEDTAVYYCHAKRGVSDYWGQGTQVTVSS

SEQ ID NO: 107 - protein, 125 aa, synthetic construct
  VARIANT 3: note = Any amino acid
EVXLVESGGGLVETGGSLRLSCAARGRIKSIADMGWYRQAPGKQRELVATITFGGTTTYADSAKGRFTISRDNAENTVYLQMNSLKPEDTAVYFCNADRILYVSEGLYRTEVDSWGQGTQVTVSS

SEQ ID NO: 108 - protein, 117 aa, synthetic construct
EVQLVESGGGLVQPGGSLRLSCAASGFTFSRAWMYWVRQAPGETLEWVSRIQPGGGSTSYADSVKGRFTISRDNAKNTLYLQMNSLKSEDTAVYYCAKDWEMAAPSLGQGTQVTVSS

SEQ ID NO: 109 - protein, 123 aa, synthetic construct
EVQLMESGGGVVQVGGSLRLSCAASGGTLYSYIVGWFSQAPGQDREFVGAIEYSGGITDYKDSVKGRFTISKDNPKNTVFLQMDSLKPEDTAVYYCGLTRVVGARNPGDYAYWGQGTQVTVSS

SEQ ID NO: 110 - protein, 127 aa, synthetic construct
EVQLVESGGGLAQTGGSLTLSCAASGRTPSIVAMGWFRQIPGKDREPVGEIILSKGFTYYADSVKGRFTISRANAKNTITMSLQMHSLKSEDTAVYYCAARQNWSGNPTRPNEYEYWGQGTQVTVSS

SEQ ID NO: 111 - protein, 127 aa, synthetic construct
EVQLVESGGGLVQTGGSLRLSCAASGRTSRLVAMGWFRQTPGKEREFVGEIILSKDFTYYADSVKGRFTISRANAKNTITMYLQMSSLKSEDTAVYYCAARQNWSGHPARTNEYEYWGQGTQVTVSS

SEQ ID NO: 112 - protein, 127 aa, synthetic construct
EVQLVESGGGLVQTGGSLRLSCAASGRTPSIVAMGWFRQTPGKEREFVGEIILSKGFTYYADSVKGRFTISRANAKNTITMYLQMHSLKSEDSAVYYCAARQNWSGGPTRTNEYEYWGQGTQVTVSS

SEQ ID NO: 113 - protein, 127 aa, synthetic construct
EVQLVESGGGLVQTGGSLRLSCAASGRTPSIVAMGWFRQTPGKERESVGEIILSKGFTYYADSVKGRFTISRANAKNTITMYLQMNSLKSEDTAVYYCAARQNWSGNPTRTNEYEYWGQGTQVTVSS

SEQ ID NO: 114 - protein, 127 aa, synthetic construct
  VARIANT 19: note = Any amino acid
EVQLVESGGGLVQTGGSLXLSCAASGRTPRLVAMGWFRQTPGKEREFVGEIILSKGFTYYADSVKGRFTISRVNAKNTITMYLQMNSLKSEDTAVYYCAGRQNWSGSPARTNEYEYWGQGTQVTVSS

SEQ ID NO: 115 - protein, 127 aa, synthetic construct
EVQLVESGGGLVQTGGSLRLSCAASGRTPSIIAMGWFRQTPGKEREFVGEIILSKGFTYYADSVKGRFTISRANAKNTITMYLQMNSLKSEDTAVYYCAARQNWSGNPTRTNEYEYWGQGTQVTVSS

SEQ ID NO: 116 - protein, 119 aa, synthetic construct
QVQLQESGGGSVQAGGSLRLSCAASGFTVTRYCMGWLRQAPGKQREGVAIIERDGRTGYADSVKGRFTISKDNAKNTLYLQMNSLKPEDTAMYYCGAIEGSCRPDFGYRGQGTQVTVSS

SEQ ID NO: 117 - protein, 119 aa, synthetic construct
QVQLQESGGGSVQAGGSLRLSCAASGFTISRYCMGWLRQAPGKQREGVAIIERDGRTGYADSVKGRFTISKDNAKNTLYLQMNSLKPEDTAMYYCGAIEGSCRPDFGYRGQGTQVTVSS

SEQ ID NO: 118 - protein, 121 aa, synthetic construct
QVQLQESGGGSVQAGGSLRLSCTASGLTFDDVEMAWYRQGPGDDYDLVSSINTDSRVYYVDSVKDRFTISRDNAKNTLYLQMNNLKPEDTAVYYCAADPWGGDLRGYPNYWGQGTQVTVSS

SEQ ID NO: 119 - protein, 119 aa, synthetic construct
QVQLQESGGGSVQAGGSLRLSCVASGFTISRYCMGWLRQAPGKQREGVAIIERDGRTGYADSVKGRFTISKDNAKNTLYLQMNSLKPGDTAMYYCGAIEGSCRPDFGYRGQGTQVTVSS

SEQ ID NO: 120 - protein, 121 aa, synthetic construct
QVQLQESGGGLVQPGGSLKLSCAASGFTFSTYAMSWVRQAPGKEPEWISRISSGGGNTYYADAVKGRFAISRDNAKNTLYLQLNSLKTEDTAIYVCTMDDYYGGSWHPISRGHGTQVTVSS

SEQ ID NO: 121 - protein, 124 aa, synthetic construct
QVQLQESGGGLVQAGGSLRLSCQASGYTYGLFCMGWFRQVSGKKREGVAVVDSPGGRHVADSLKGRFTISKDNANNILYLDMTNLKSEDTATYYCAADPEKYCFLFSDAGYQYWGQGTQVTVSS

SEQ ID NO: 122 - protein, 128 aa, synthetic construct
QVQLQESGGGSVQAGGSLRLSCAASGVTYSRYCMGWFRQAPGLERERVATIYSRGIITYYTDSVKGRFTISQDSAKKTVYLQMNSLKPEDTAMYYCAATRETYGGSGDCDYESVYNYWAQGTQVTVSS

SEQ ID NO: 123 - protein, 119 aa, synthetic construct
QVQLQESGGGSVQAGGSLRLSCAASGFTVSRYCMGWLRQAPGKQREGVAIIEREGRTGYADSVKGRFTISKDNAKNTLYLQMNSLKPEDTAMYYCGAIEGSCRPDFGYRGQGTQVTVSS

SEQ ID NO: 124 - protein, 119 aa, synthetic construct
QVQLQESGGGSVQAGGSLRLSCAASGFTISRYCMGWLRQAPGKQREGVAIIERDGRTGYADSVKGRFTISKDNAKNTLYLQMNSLKPEDTAMYFCGAIEGSCRPDFGYRGQGTQVTVSS

SEQ ID NO: 125 - protein, 119 aa, synthetic construct
QVQLQESGGGSVQAGGSLRLSCAASGFTVSRYCMGWLRQAPGKQREGVAIIERDGRTGYADSVKGRFTISKDDAKNTLYLQMNSLKPEDTAMYYCGAIEGSCRPDFGYRGQGTQVTVSS

SEQ ID NO: 126 - protein, 128 aa, synthetic construct
QVQLQESGGGSVQAGGSLRLSCAASGVTYSRYCMGWFRQAPGLERERVATIYSRGIITYYTDSVKGRFTISQDSAKKTVYLQMNMLKPEDTAMYYCAATRETYGGSGDCDYESVYNYWAQGTQVTVSS

SEQ ID NO: 127 - protein, 119 aa, synthetic construct
QVQLQESGGGSVQAGGSLRLSCAASGFTISKYCMGWLRQAPGKQREGVAIIERDGRTGYADSVKGRFTISKDNAKNTLYLQMNSLKPEDTAMYYCGAIEGSCRPDFGYRGQGTQVTVSS

SEQ ID NO: 128 - protein, 128 aa, synthetic construct
QVQLQESGGGSVQAGGSLRLSCAASGVTYSRYCMGWFRQAPGLERERVAHIYSRGIITYYTDSVKGRFTISQDSAKKTVYLQMNSLKPEDTAMYYCAATRETYGGSGDCGYESVYNYWAQGTQVTVSS

SEQ ID NO: 129 - protein, 119 aa, synthetic construct
QVQLQESGGGSVQAGGSLRLSCAASGFTISRYCMGWLRQAPGKQREGVAIIERDGRTGYADSVKGRFTISKDNAKNTLYLQMNSLKPEDTAMYYCGAIEGSCRPDLGYRGQGTQVTVSS

SEQ ID NO: 130 - protein, 128 aa, synthetic construct
QVQLQESGGGSVQAGGSLRLSCAASGVTYSRYCMGWFRQAPGLERERVAHIYSRGIITYYTDSVKGRFTISQDSAKKTVYLQMNSLKPEDTAMYYCAATRETYGGSGDCSYESVYNHWAQGTQVTVSS

SEQ ID NO: 131 - protein, 119 aa, synthetic construct
QVQLQESGGGSVQAGGSLRLSCAASGLTISRYCMGWLRQAPGKQREGVAIIERDGRTGYADSVKGRFTISKDNAKNTLYLQMNSLKPEDTAMYYCGAIEGSCRPDFGYRGQGTQVTVSS

SEQ ID NO: 132 - protein, 117 aa, synthetic construct
QVQLQESGGGSVQAGGSLRLSCSASGFTVDDFAMGWYRQAPGNECELVSTISSGGSTYYADSVKGRFTISQDSAKNTVYLQMNSLKPEDTAVYYCAPSSVGCPLGYWGQGTQVTVSS

SEQ ID NO: 133 - protein, 126 aa, synthetic construct
QVQLQESGGGSVQAGGSLRLSCAASGYTYSNRHMGWFRQAPGKEREGVAAIYTGGGSTYYADSVKDRFTISQDNAKNTLYLQMNSLTPEDTAMYYCAADLTRWYSGGWRDPRGYKYWGQGTQVTVS

SEQ ID NO: 134 - protein, 128 aa, synthetic construct
QVQLQESGGGSVQAGGSLRLSCAASGVTYGSYYMAAWFRQAPGKEREGVASIYGGSDSTYYADSVLGRFTISQDNGKNTLYLQMNSLKPDDTAMYYCAAAPPGKWFLKRLEGHNYSYWGQGTQVTVSS

SEQ ID NO: 135 - protein, 131 aa, synthetic construct
QVQLQESGGGSVQVGGSLRLSCAASGFTYSSSCLGWFRQAPGKEREGVATIYPAGGNIFYADSVKGRFTISQDNAKNTVYLQMDSLKPEDTAMYYCAARGGQTWGSGGNRCSLWLPAYNYWGQGTQVTVSS

SEQ ID NO: 136 - protein, 123 aa, synthetic construct
QVQLQESGGGSVQVGGSLRLSCAVSGKLYGGAWFRQAQGKGREGVAAIWIGTGTTFYADSVKGRFTISRDNAKNTVYLQMDGLKPEDTALYYCAADDRPGYRDPLAPVSYNHWGQGTQVTVSS

SEQ ID NO: 137 - protein, 126 aa, synthetic construct
QVQLQESGGGSVQAGGSLRLSCAASGITYRGVWMGWFRQAPGKEREGVATIYTGSGHTYYADSVKGRFTISQDNAKNTVYLQMNSLKPEDTAMYYCAARTVGGTFYTLAADSFNTWGQGTQVTVSS

SEQ ID NO: 138 - protein, 123 aa, synthetic construct
QVQLQESGGGSVQAGGSLRLSCAVSGKAYGGAWFRQAQGKGREGVAAIWIGTGTTFYADSVKGRFTISRDNAKNTVYLQMDGLKPEDTAVYYCAADDRPGYRDPLAPVSYNHWGQGTQVTVSS

SEQ ID NO: 139 - protein, 123 aa, synthetic construct
QVQLQESGGGSVQAGGSLKLSCAVSGNPYGGAWFRQAQGKSREGVAAIWLGTGTTFYADSVKGRFTISRDNAKNTVYVQIDGLKPEDTAMYYCAADDRPGYRDPLAPVSYNHWGQGTQVTVSS

SEQ ID NO: 140 - protein, 123 aa, synthetic construct
QVQLQESGGGSVQAGGSLRLSCVVSGKAYGGAWFRQAQGKSREGVAAIWIGTGTTFYADSVKGRFTISRDNAKNTVYLQMDGLKPEDTAMYYCAADDRPGYRDPLAPVSYNHWGQGTQVTVSS

SEQ ID NO: 141 - protein, 123 aa, synthetic construct
QVQLQESGGGSVQAGGSLTLSCVVSGKAFGGAWFRQAQGKGREGVAAIWIGTGTTFYADSVKGRFTISRDNAKNTVYLQMDGLKPDDTAMYYCAADDRPGYRDPLAPVSYNHWGQGTQVTVSS

SEQ ID NO: 142 - protein, 127 aa, synthetic construct
QVQLQESGGGSVQAGGSLRLSCAASGYTFSNHHMGWFRQAPGKEREGVAAIYTGAGNIYYADSVKDRFTISKDTAKNTLYLQMNSLTPEDTGMYYCAADLTRWYSGGWRDPRGYKYWGQGTQVTVSS

SEQ ID NO: 143 - protein, 127 aa, synthetic construct
QVQLQESGGGPVQAGGSLRLSCAASGYTFSNHHMGWFRQAPGKEREGVAAIYTGAGNIYYADSVKDRFTISKDTAKNTLYLQMNSLTPEDTGMYYCAADLTRWYSGGWRDPRGYKYWGQGTQVTVSS

SEQ ID NO: 144 - protein, 127 aa, synthetic construct
QVQLQESGGGVVQPGGSLRLSCAASGYTFSNHHMGWFRQAPGKEREGVAAIYTGAGNIYYADSVKDRFTISKDTAKNTLYLQMNSLTPEDTGMYYCAADLTRWYSGGWRDPRGYKYWGQGTQVTVSS

SEQ ID NO: 145 - protein, 127 aa, synthetic construct
QVQLQESGGGSVQAGGSLRLSCAVSGYTFSNHHMGWFRQAPGKEREGVAAIYTGAGNIYYADSVKDRFTISKDTAKNTLYLQMNSLTPEDTGMYYCAADLTRWYSGGWRDPRGYKYWGQGTQVTVSS

SEQ ID NO: 146 - protein, 130 aa, synthetic construct
QVQLQESGGGSVQAGGSLRLSCAASGATNSNRHMGWFRQAPGKEREGVAAIYTGYTGGGNTYYADSVRDRFTISQDNAKNTLYLQMNSLTPEDTAMYYCAADLTRWYSGGWRDPRGYKYWGQGTQVTVSS

SEQ ID NO: 147 - protein, 126 aa, synthetic construct
QVQLQESGGGSVQDGGSLRLSCAASGDIYARNCMGWFRQAPGKEREKIAVADTGGRSPYYADSVKGRFTISRDNAKNTVDLQMNSLKPEDTAVYYCAAGPLVPVVNTAARCVYEYWGQGTQVTVSS

SEQ ID NO: 148 - protein, 130 aa, synthetic construct
QVQLQESGGGSVQAGGSLRLSCAASGATNSNRHMGWFRQAPGKEREGVAAIYTGYTGGGNTYYADSVKDRFTISQDNAKNTLYLQMNSLTPEDTAMYYCAADLTRWYSGGWRDPRGYKYWGQGTQVTVSS

SEQ ID NO: 149 - protein, 124 aa, synthetic construct
EVQLVESGGGLVQTGGSLRLSCTHSGRPLSMFAVGWFRQAPGEQRVLVAAISRSGGATYYPDSVKGRFAISRDNAKNTVNLQMNSLTPEDTAVYYCAAALGGVYNAIANDYKYWGQGTQVTVSS

SEQ ID NO: 150 - protein, 127 aa, synthetic construct
EVQLVESGGGSVQAGGSLRLSCAASGRTFSDYAMGWFRQAPGKEREFVAGISRSGSTTGYAGSVKGRFSISRDNAKNAVYLQMNSLKPEDTAVYYCASKRSPYTGIYYSDLSQSEDWGQGTQVTVSS

SEQ ID NO: 151 - protein, 125 aa, synthetic construct
EVQLVESGGGLVQAGGSLRLSCAASGFAFDDYEIGWFRQAPGKEREGVACINNSDGSTYYIDSVKGRFTISKDSATNTVYLQMSSLKPEDTAVYYCAADSWCTVVAGKIHPFDSWGQGTQVTVSS

SEQ ID NO: 152 - protein, 124 aa, synthetic construct
EVQLVESGGGLVQAGGSLRLSCAASGRTSSTYVMGWFRQAPGKEREFVAGISWSAGNTRYADSVKGRFTISRDIAKNTFDLQMNSLKPEDTAVYYCAADRRYGGSLDPSAWDFWGQGTQVTVSS

SEQ ID NO: 153 - protein, 124 aa, synthetic construct
EVQLVESGGGSVQAGGSLRLSCAASGRTFSSYVMGWFRQAPGKEREFVAAVSWSAGNTRYANSVKGRFTISRDIAKNTFYLQMNSLKPEDTAVYYCVADRRYGGSLDPSAWDFWGQGTQVTVSS

SEQ ID NO: 154 - protein, 125 aa, synthetic construct
EVQLVESGGGLVQPGGSLRLSCAVSGFTSDYYVIAWFRQTPGHEREGVSSIRKGDGATYYADSVKGRFSISRDNAKNMVYLQMNSLKPEDTAVYTCAARYDTLLGGGRPREFPYWGQGTQVTVSS

SEQ ID NO: 155 - protein, 125 aa, synthetic construct
EVQLVESGGGLVQPGGSLRLSCAVSGFTSDYYVVAWFRQTPGHEREGVSSIRIGDYATYYADSVKGRFSISRDNAKNMVYLQMNSLKPEDTAVYTCAARYDSHLGGGRPREFPYWGQGTQVTVSS

SEQ ID NO: 156 - protein, 125 aa, synthetic construct
EVQLVESGGGLVQPGGSLRLSCAVSGFTSDYYVIAWFRQTPGHEREGVSSIRITDNATYYADSVKGRFSISRDNAKNMVFLQMNSLKPEDTAVYTCAARYETLLGGGRPREFPYWGQGTQVTVSS

SEQ ID NO: 157 - protein, 125 aa, synthetic construct
EVQLVESGGGLVQPGGSLILSCAVSGFTSDYYVIAWFRQTPGHEREGVSSIRIGDGATYYADSVKGRFSISRDNAKNMVYLQMNSLKPEDTAVYTCAARYDTLLGGGRPREFPYWGQGTQVTVSS

SEQ ID NO: 158 - protein, 124 aa, synthetic construct
EVQLVESGGGLVQPGGSLRLSCAASGFSFSRNWMYWVRQAPGKGLEWVGDISMEGTNTYYRDSVQGRFTISRDNAKNILYLQMNSLKSEDTAVYYCARAKNEGFVPGGYDFDYRGQGTQVTVSS

SEQ ID NO: 159 - protein, 125 aa, synthetic construct
EVQLVESGGGLVQPGGSLRLSCAVSGFTSDYYVIAWFRQTPGHEREGLSSIRIGDSATFYADSVKGRFSISRDNAKNMVYLQMNSLKPEDTAVYTCAGRYDTLLGGGRPREFPYWGQGTQVTVSS

SEQ ID NO: 160 - protein, 125 aa, synthetic construct
EVQLVESGGGLVQPGGSLRLSCAVSGFTSDFYVIAWFRQTPGHEREGVSSIRIGDNAAYYADSVKGRFSISRDYAKNMVYLQMNSLKAEDTAVYTCAGRYDSNLGGGRPREFPYWGRGTQVTVSS

SEQ ID NO: 161 - protein, 118 aa, synthetic construct
EVQLVESGGGLVQPGGSLRLSCAASGRTIAAYKMAWFRQAPDKARERVALITSFGLTVYADSVKGRFTISRDNAYNTVYLQMNHLKFEDTAVYYCAAGQQDSSNYNSWGQGTQVTVSS

SEQ ID NO: 162 - protein, 125 aa, synthetic construct
EVQLVESRGGLVQPGGSLRLSCAVSGFTSDYYVIAWFRQTPGHEREGVSSIRKGDGATYYADSVKGRFSISRDNAKNMVYLQMNSLKPEDTAVYTCAARYDTLLGGGRPREFPYWGQGTQVTVSS

SEQ ID NO: 163 - protein, 127 aa, synthetic construct
EVQLVESGGRLVQAGDSLRLSCAASGRTFTSYRMGWFRQAPGKEREFVSALRWSSGNIDYTYYADSVKGRFSISGDYAKNTVYLQMNSLKAEDTAVYYCAASTRWGVMESDTEYTSWGQGTQVTVSS

SEQ ID NO: 164 - protein, 126 aa, synthetic construct
EVQLVESGGGLVQAGGSLTLSCAASGRTFSSYHMGWFRQAPGKEREYVAAISWSGHMTYYKDSAKGRFTISRDNAKNTVYLQMNNLKPEDTAVYYCAARNRDYWSDFDVPGRYAYWGQGTQVTVSS

SEQ ID NO: 165 - protein, 127 aa, synthetic construct
EVQLVESGGRLVQAGDSLRLSCAASGRTFTNYRVGWFRQAPGKEREFVSALRWSSSNIDYTYYADSVKGRFSISGDYAKNTVYLQMNSLKAEDTAVYYCAASTSWGVLESDTEYTSWGQGTQVTVSS

SEQ ID NO: 166 - protein, 125 aa, synthetic construct
  VARIANT 123: note = Any amino acid
  VARIANT 125: note = Any amino acid
EVQLVESGGGLVQPGGSLRLSCAVSGFTSEYYVIAWFRQTPGHEREGVSSIRIRDNAAYYADSVKGRFSISRDNHKNMVYLQMNSLKPEDTAVYTCAGRYDTLLGGGRPREFPYWGQGTQVTXSX

SEQ ID NO: 167 - protein, 122 aa, synthetic construct
  VARIANT 3: note = Any amino acid
EVXLVESGGALVQDGGSLRLSCAASGLTLSNYVAAWFRQAPGKEREYVGSIRWSSEQTYYANSVKGRFSISRDNAKNAVYLEMNTLKPEDTAVYYCALGTSFSALPKLYNYWGQGTQVTVSS

SEQ ID NO: 168 - protein, 127 aa, synthetic construct
  VARIANT 3: note = Any amino acid
EVXLVESGGRLVQAGDSLRLSCAASGRTFISYRMGWFRQAPGKEREFVAALRWSSSNIDYTYYADSVKGRFSISGDYAKNTVYLQMNSLKAEDTAVYYCAASTRWGVMESDTEYTSWGQGTQVTVSS

SEQ ID NO: 169 - protein, 121 aa, synthetic construct
  VARIANT 98: note = Any amino acid
  VARIANT 108: note = Any amino acid
EVQLVESGGGLVQSGGSLRLSCAASEGTFTIYPLGWFRQAPGKDRKFVAALPWSAGIPQYSDSVKGRFTISRDNAKNTVYLQMNNLKPEDTAVYYCAXKGRDDSYQPXNYWGQGTQVTVSS

SEQ ID NO: 170 - protein, 122 aa, synthetic construct
HVQLVESGGGLVQPGRSLRLSCAASGFTFSSYWMYWVRQTPGKGLEWVSTINRDGSATWYADSVKGRFTISRDNAKNTGYLQMNSLEPDDTAVYYCVSDPDNYSSDEMVPYWGQGTQVTVSS

SEQ ID NO: 171 - protein, 122 aa, synthetic construct
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 172 - protein, 122 aa, synthetic construct
  VARIANT 1: note = Any amino acid
  VARIANT 16: note = Any amino acid
  VARIANT 87: note = Any amino acid
XVQLVESGGGLVQPGXSLRLSCAASGFTFSSYWMYWVRQTPGKGLEWVSTINRDGSATWYADSVKGRFTISRDNAKNTGYLQMNSLXPDDTAVYYCVSDPDNYSSDEMVPYWGQGTQVTVSS

SEQ ID NO: 173 - protein, 130 aa, synthetic construct
QVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSS

SEQ ID NO: 174 - protein, 123 aa, synthetic construct [sequence omitted]

SEQ ID NO: 175 - protein, 129 aa, synthetic construct
DVQLVESGGGSVQSGGSLRLSCAASGYTYRRYYMGWFRQAPGEQREGVAVINNDGRTNYADSVKGRFRISRDNAENTLHLEMNSLKPEDTAMYYCAATGNILPPMTAVPPLGRQWYPYWGRGTLVTVSS

SEQ ID NO: 176 - protein, 130 aa, synthetic construct
HVQLVESGGGSVQSGGSLRLSCAASGYAVKNCMGWFRQAPGKEREGVAVINRNGITTYADSVKGRFTISQDKDKNTLDLQMNSLKPEDTAMYYCAATPTLLTIPARFLCDVRNPSGFTDWGQGTLVTVSS

SEQ ID NO: 177 - protein, 128 aa, synthetic construct
QVQLVESGGGSVQAGGSLRLSCVVSAYSAYTYKTMCMGWFRQAPGKEREGVAAIYRGGLNTYYADSVKGRFIISRDNAESTMYLQMNSLKPEDTAMYYCAADWLRGDDCNIGANFDYWGQGTQVTVSS

SEQ ID NO: 178 - protein, 132 aa, synthetic construct
QVQLVESGGGSVQAGGSLRLSCVATGFTISRKCMGWFREAPGKKREVIATINTGSSSPYYADGVKGRFTISQDNAKNTVYLQMNSLKPEDTAMYYCAATKGVVVGTGYCGGPYVERPNSAYWGQGTQVTVSS

SEQ ID NO: 179 - protein, 126 aa, synthetic construct
DVQLVESGGGSVQAGRSLRLSCELSDYTWSTVCMGWFRQAPGKEREGVAVIYTRSGGTTYADSAKGRFTISRDNAKDTLYLQMDSLKPEDTAMYYCAAGPLYDGRCTYRSPAFHYWGQGTQVTVSS

SEQ ID NO: 180 - protein, 122 aa, synthetic construct
DVQLVESGGGSAQAGGSLRLSCAASGPTSSLRTMGWFRQASGKERERVAVIWDGRTTDYDDSVQDRFTISQDNAKSTVYLQMNTLKPEDTAMYYCAASPRIVPFASTYFQHWGQGTQVTVSS

SEQ ID NO: 181 - protein, 127 aa, synthetic construct
HVQLVESGGGSVQAGGSLKLSCAASGSIFSGSIFSRCGMRWYRQAPGKERELVSSTSKDGFTSYTDSVKGRFTISQDNANNTLYLQMSSLKTEDTAVYSCAAICAVGGYSLSTYTYWGQGTQVTVSS

SEQ ID NO: 182 - protein, 124 aa, synthetic construct
EVQLVESGGDSVQAGGSLRLSCAASGYSPGSYCMGWFRQAPGKERERVAIIESRGTVTYVDSVKGRFTISKDNAKNTLYLQMNSLKPEDTAMYYCAASRPWSGVRCLHDKYDYWGQGTQVTVSS

SEQ ID NO: 183 - protein, 129 aa, synthetic construct
HVQLVESGGGSVQSGGSLRLSCAVSGYAYSSLAWFRQAPGKEREGVAALLTAIGGPTRTTYADSVKGRLAISQDHAKNTLYLQMSSLKPEDTAMYYCAAGRPAGTPRWLLLAPRDYNYWGQGTQVTVSS

SEQ ID NO: 184 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 185 - protein, 117 aa, synthetic construct
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSS

SEQ ID NO: 186 - protein, 127 aa, synthetic construct
QVQLVQSGGGLVQAGGSLSLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSS

SEQ ID NO: 187 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 188 - protein, 117 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 189 - protein, 120 aa, synthetic construct
QVQLQESGGGLVQAGGSLRLSCAASGRTVSSLFMGWFRQAPGKERELVAAISRYSLYTYYADSVKGRFTISADNAKNAVYLQMNSLKPEDTAVYYCASKLEYTSNDYDSWGQGTQVTVSS

SEQ ID NO: 190 - protein, 120 aa, synthetic construct
QVQLQESGGGLVQAGDSLRLSCAASGRAVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 191 - protein, 120 aa, synthetic construct
QVQLQESGGGLVQAGDSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 192 - protein, 116 aa, synthetic construct
QVQLQESGGGLVQPGDSMRLSCAAEGDSLDGYVVGWFRQAPGKERQGVSSISGDGSMRYVADSVKGRFTISRDNAKNTVYLQMIDLKPEDTGVYYCAADPPTWDYWGQGTQVTVSS

SEQ ID NO: 193 - protein, 120 aa, synthetic construct
QVQLQESGGGLVQPGGSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 194 - protein, 120 aa, synthetic construct
QVQLQESGGGLVQAGESLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 195 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 196 - protein, 125 aa, synthetic construct
QLQLVESGGGLVHAGGSLRLSCAASGSTFSINAIGWYRQAPGKQRELVAALSSGGSKNYADSVKGRFTISRDNAKNTVYLQMNRLKPEDTAVYYCNAEIYYSDGVDDGYRGMDYWGKGTQVTVSS

SEQ ID NO: 197 - protein, 129 aa, synthetic construct
EVQVVESGGGLVQTGGSLRLSCAASGPPLSSYAVAWFRQTPGKEREFVAAISWSGSNTYYADSVKGRFTISKDNAKNTVLVYLQMNSLKPEDTAVYYCAADRRGGPLSDYEWEDEYADWGQGTQVTVSS

SEQ ID NO: 198 - protein, 450 aa, synthetic construct
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 199 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 761 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFEKTKIKLCILLHAFRIRAVTIDRVMSYLNASGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 201 - protein, 443 aa, synthetic construct
EVQLVESGGGLVQSGGSLRLSCEASGFTFSNYWMTWIRQGPGKGLEWVANIKPDGTENYYVDSVKGRFTISRDNARNSLYLQMTSLKAEDTAVYYCGRMIQFVINIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 675 aa, synthetic construct
DGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 203 - protein, 443 aa, synthetic construct
EFQVVESGGGLVKPGGSLRLSCVVSGFTFSNYNMNWVRQAPAKGLEWVSSISTGSSDIYYADSVKGRFTISRDNAKNSLYLQMNSLRVEDTAVYYCARDIIGTTRDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0361309 A1
(43) Pub. Date: Nov. 27, 2025

(54) TUMOR-TARGETED SPLIT IL12 RECEPTOR AGONISTS

(71) Applicant: Regeneron Pharmaceuticals, Inc., Tarrytown, NY (US)

(72) Inventors: Jiaxi WU, Pleasantville, NY (US); Aaron CHANG, Stamford, CT (US); Amanda D'ORVILLIERS, Long Island City, NY (US); Tong ZHANG, New Rochelle, NY (US); Erica ULLMAN, Yorktown Heights, NY (US); Chia-Yang LIN, Scarsdale, NY (US); Andrew J. MURPHY, Croton-on-Hudson, NY (US); George D. YANCOPOULOS, Yorktown Heights, NY (US)

(73) Assignee: Regeneron Pharmaceuticals, Inc., Tarrytown, NY (US)

(21) Appl. No.: 19/218,015

(22) Filed: May 23, 2025

Related U.S. Application Data
(60) Provisional application No. 63/651,855, filed on May 24, 2024.
(60) Provisional application No. 63/730,129, filed on Dec. 10, 2024.

(51) Int. Cl. C07K 16/28; A61K 40/33; A61K 40/42; C07K 16/30

(52) U.S. Cl. CPC C07K 16/2866; A61K 40/33; A61K 40/421; C07K 16/3069; C07K 16/3092; C07K 2317/31; C07K 2317/51; C07K 2317/515; C07K 2317/52; C07K 2317/53; C07K 2317/55; C07K 2317/569; C07K 2317/71

(57) ABSTRACT

The present disclosure relates to tumor-targeted split IL12 receptor agonists with improved therapeutic profiles.

### 1. CROSS-REFERENCE TO RELATED APPLICATIONS

This application claims the priority benefit of U.S. provisional application No. 63/651,855, filed May 24, 2024 and U.S. provisional application No. 63/730,129, filed Dec. 10, 2024, the contents of each of which are incorporated herein in their entireties by reference thereto.

### 2. SEQUENCE LISTING

The instant application contains a Sequence Listing which has been submitted electronically and is hereby incorporated by reference in its entirety. Said copy, created on May 20, 2025, is named RGN-052WO_SL.xml and is 208,693 bytes in size.

### 3. BACKGROUND

Interleukin 12 (IL-12 or IL12) is a pro-inflammatory cytokine having an important role in both innate and adaptive immunity. Hamza et al., 2010, Int. J. Mol. Sci., 11(3):789-806. IL12 functions primarily as a 70 kDa heterodimer consisting of disulfide-linked p35 and p40 subunits. Id. A variety of different immune cells, including B cells, dendritic cells, macrophages, monocytes, and neutrophils express IL12 when stimulated (Tugues et al., 2015, Cell Death Differ., 22:237-246), with the active heterodimer forming following protein synthesis. Binding of IL12 to the IL12 receptor complex on T and natural killer (NK) cells leads to signaling via signal transducer and activator of transcription 4 (STAT4) and signal transducer and activator of transcription 3 (STAT3), and subsequent interferon gamma (IFN-γ) production and secretion. Ullrich et al., 2020, EXCLI J., 19:1563-1589. Signaling downstream of IFN-γ includes activation of T-box transcription factor TBX21 (Tbet) and induces pro-inflammatory functions of T helper 1 (T_{H}1) cells. Id.

Due to its ability to activate NK cells and cytotoxic T-cells, IL12 has been studied as an anti-cancer therapeutic since the early 1990's. Lasek et al., 2014, Cancer Immunol. Immunother. 63(5):419-435. However, in most patients, repeated administration of IL12 led to adaptive response and a progressive decline of IL12-induced IFN-γ blood levels. Id. Further, severe toxicity resulted from the concomitant induction of IFN-γ along with other cytokines (e.g., TNF-α) and/or chemokines (IP-10 or MIG). Id. Different dosing and timing protocols were developed in an attempt to minimize IFN-γ toxicity and improve IL12 efficacy. Id. These approaches had minimal effect and have not significantly improved patient survival. Id.

Despite the general acceptance in the field of IL12 therapies being developed for immunotherapy, including anticancer therapy, IL12 molecules have generally displayed poor therapeutic indices, with high, toxic doses required to confer modest anti-cancer effects.

Thus, there is a need in the art for novel IL12 therapies with improved therapeutic efficacy and safety profiles.

### 4. SUMMARY

The present disclosure provides tumor-targeted split IL12 receptor agonists.

In certain aspects, the tumor-targeted split IL12 receptor agonists address the drawbacks of IL12 therapy and are characterized by improved therapeutic profiles by virtue of efficacy and/or improved safety profiles. The tumor-targeted split IL12 receptor agonists of the disclosure typically comprise two components, or a “combination”, formulated in a single formulation or separate formulations, comprising a tumor-targeted IL12Rβ1 agonist and a tumor-targeted IL12Rβ2 agonist. Exemplary tumor-targeted split IL12 receptor agonists are disclosed in Section 6.2 and numbered embodiments 1 to 9 and 19 to 136. Exemplary tumor-targeted IL12Rβ1 agonists are disclosed in Section 6.3 and numbered embodiments 19 to 44, 60 to 97, 106 to 107, and 110 to 118. Exemplary tumor-targeted IL12Rβ2 agonists are disclosed in Section 6.4 and numbered embodiments 19 to 29, 45 to 79, 98 to 105, 108 to 112, and 125 to 136.

The disclosure further provides nucleic acids encoding the tumor-targeted split IL12 receptor agonists of the disclosure and their components. The nucleic acids can be in the form of a single nucleic acid (e.g., a vector encoding all components of the tumor-targeted split IL12 receptor agonists) or a plurality of nucleic acids (e.g., two or more vectors encoding the different components and/or their individual polypeptide chains). The disclosure further provides host cells and cell lines engineered to express the nucleic acids and the tumor-targeted split IL12 receptor agonists of the disclosure. The disclosure further provides methods of producing a tumor-targeted split IL12 receptor agonist of the disclosure. Exemplary nucleic acids, host cells, cell lines, and methods of producing tumor-targeted split IL12 receptor agonists are described in Section 6.10.

The disclosure further provides pharmaceutical compositions comprising the tumor-targeted split IL12 receptor agonists of the disclosure. Exemplary pharmaceutical compositions are described in Section 6.11.

Further provided herein are methods of using the tumor-targeted split IL12 receptor agonists, e.g., for eliciting anti-tumor cytotoxicity and treating cancerous conditions. Exemplary methods are described in Section 6.12 and numbered embodiments 10 to 142, infra.

Also described are combination methods using the tumor-targeted split IL12 receptor agonists in combination with one or more additional therapeutic agents, such as a multispecific T-cell engager as described in Section 6.6. Exemplary combination therapy methods are described in Section 6.13 and numbered embodiments 137 to 142, infra.

Also provided are tumor-targeted IL12Rβ1 agonists for use in a method (e.g., a method described in Section 6.12 or 6.13) comprising administrating to a subject the tumor-targeted IL12Rβ1 agonist (e.g., as described in Section 6.3) and a tumor-targeted IL12Rβ2 agonist (e.g., as described in Section 6.4). Exemplary tumor-targeted IL12Rβ1 agonists for use in such methods are described in numbered embodiments 153 to 160.

Further provided are tumor-targeted IL12Rβ2 agonists for use in a method (e.g., a method described in Section 6.12 or 6.13) comprising administrating to a subject the tumor-targeted IL12Rβ2 agonist (e.g., as described in Section 6.4) and a tumor-targeted IL12Rβ1 agonist (e.g., as described in Section 6.3). Exemplary tumor-targeted IL12Rβ2 agonists for use in such methods are described in numbered embodiments 161 to 168.

### 5. BRIEF DESCRIPTION OF THE DRAWINGS

FIGS. 1A-1L show exemplary tumor-targeted IL12Rβ1 agonist structures. FIG. 1A shows a tumor-targeted IL12Rβ1 agonist comprising: (a) a tumor targeting moiety in Fab format (e.g., a Fab derived from an antibody against a tumor-associated antigen) connected to the N-terminus of a first Fc domain via a first linker (1), and (b) an IL12Rβ1 binding moiety comprising an IL12 moiety connected to the N-terminus of a second Fc domain via a second linker (2). The p35 and p40 moieties of the IL12 moiety are connected to one another via a third linker (3). Mutation(s) in the p35 moiety are represented by a star. FIG. 1B shows a tumor-targeted IL12Rβ1 agonist comprising (a) a tumor targeting moiety in Fab format (e.g., a Fab derived from an antibody against a tumor-associated antigen) connected to the N-terminus of a first Fc domain via a first linker (1), and (b) an IL12Rβ1 binding moiety in Fab format (e.g., a Fab derived from an antibody against IL12Rβ1) connected to the N-terminus of a second Fc domain via a second linker (2). FIG. 1C shows a tumor-targeted IL12Rβ1 agonist comprising (a) a tumor targeting moiety in Fab format (e.g., a Fab derived from an antibody against a tumor-associated antigen) connected to the N-terminus of a first Fc domain via a first linker (1), and (b) an IL12Rβ1 binding moiety in single domain antibody (sdAb) format (e.g., an sdAb against IL12Rβ1) connected to the N-terminus of a second Fc domain via a second linker (2). FIG. 1D shows a tumor-targeted IL12Rβ1 agonist comprising (a) a tumor targeting moiety in sdAb format (e.g., an sdAb against a tumor-associated antigen) connected to the N-terminus of a first Fc domain via a first linker (1), and (b) an IL12Rβ1 binding moiety comprising an IL12 moiety connected to the N-terminus of a second Fc domain via a second linker (2). FIG. 1E shows a tumor-targeted IL12Rβ1 agonist comprising (a) a tumor targeting moiety in a sdAb format (e.g., an sdAb against a tumor-associated antigen) connected to the N-terminus of a first Fc domain via a first linker (1), and (b) an IL12Rβ1 binding moiety in Fab format (e.g., a Fab derived from an antibody against IL12Rβ1) connected to the N-terminus of a second Fc domain via a second linker (2). FIG. 1F shows a tumor-targeted IL12Rβ1 agonist comprising (a) a tumor targeting moiety in sdAb format (e.g., an sdAb against a tumor-associated antigen) connected to the N-terminus of a first Fc domain via a first linker (1) and (b) an IL12Rβ1 binding moiety in sdAb format (e.g., an sdAb against IL12Rβ1) connected to the N-terminus of a second Fc domain via a second linker (2). FIG. 1G shows a tumor-targeted IL12Rβ1 agonist comprising (a) a tumor targeting moiety in scFv format (e.g., an scFv against a tumor-associated antigen) connected to the N-terminus of a first Fc domain via a first linker (1) and an IL12Rβ1 binding moiety in sdAb format (e.g., an sdAb against IL12Rβ1) connected to the N-terminus of a second Fc domain via a second linker (2). FIG. 1H shows a tumor-targeted IL12Rβ1 agonist comprising (a) a tumor targeting moiety in scFv format (e.g., an scFv derived from an antibody against a tumor-associated antigen) connected to the N-terminus of a first Fc domain via a first linker (1) and an IL12Rβ1 binding moiety in scFv format (e.g., an scFv derived from an antibody against IL12Rβ1) connected to the N-terminus of a second Fc domain via a second linker (2). FIG. 1I shows a tumor-targeted IL12Rβ1 agonist comprising (a) a tumor targeting moiety in scFv format (e.g., an scFv derived from an antibody against a tumor-associated antigen) connected to the N-terminus of a first Fc domain via a first linker (1) and (b) an IL12Rβ1 binding moiety comprising an IL12 moiety connected to the N-terminus of a second Fc domain via a second linker (2). The p35 and p40 moieties of the IL12 moiety are connected to one another via a third linker (3). Mutation(s) in the p35 moiety are represented by a star. FIG. 1J shows a tumor-targeted IL12Rβ1 agonist comprising (a) a tumor targeting moiety in sdAb format (e.g., an sdAb against a tumor-associated antigen) connected to the N-terminus of a first Fc domain via a first linker (1) and an IL12Rβ1 binding moiety in scFv format (e.g., an scFv derived from an antibody against IL12Rβ1) connected to the N-terminus of a second Fc domain via a second linker (2). FIG. 1K shows a tumor-targeted IL12Rβ1 agonist comprising (a) a tumor targeting moiety in Fab format (e.g., a Fab derived from an antibody against a tumor-associated antigen) connected to the N-terminus of a first Fc domain via a first linker (1) and an IL12Rβ1 binding moiety in scFv format (e.g., an scFv derived from an antibody against IL12Rβ1) connected to the N-terminus of a second Fc domain via a second linker (2). FIG. 1L shows a tumor-targeted IL12Rβ1 agonist comprising (a) a tumor targeting moiety in scFv format (e.g., an scFv derived from an antibody against a tumor-associated antigen) connected to the N-terminus of a first Fc domain via a first linker (1) and an IL12Rβ1 binding moiety in Fab format (e.g., a Fab derived from an antibody against IL12Rβ1) connected to the N-terminus of a second Fc domain via a second linker (2).

FIGS. 2A-2L show exemplary tumor-targeted IL12Rβ2 agonist structures. FIG. 2A shows a tumor-targeted IL12Rβ2 agonist comprising: (a) a tumor targeting moiety in Fab format (e.g., a Fab derived from an antibody against a tumor-associated antigen) connected to the N-terminus of a first Fc domain via a first linker (1), and (b) an IL12Rβ2 binding moiety comprising an IL12 moiety, connected to the N-terminus of a second Fc domain via a second linker (2). The p35 and p40 moieties of the IL12 moiety are connected to one another via a third linker (3). Mutation(s) in the p40 moiety are represented by a star. FIG. 2B shows a tumor-targeted IL12Rβ2 agonist comprising (a) a tumor targeting moiety in Fab format (e.g., a Fab derived from an antibody against a tumor-associated antigen) connected to the N-terminus of a first Fc domain via a first linker (1), and (b) an IL12Rβ2 binding moiety in Fab format (e.g., a Fab derived from an antibody against IL12Rβ2) connected to the N-terminus of a second Fc domain via a second linker (2). FIG. 2C shows a tumor-targeted IL12Rβ2 agonist comprising (a) a tumor targeting moiety in Fab format (e.g., a Fab derived from an antibody against a tumor-associated antigen) connected to the N-terminus of a first Fc domain via a first linker (1), and (b) an IL12Rβ2 binding moiety in single domain antibody (sdAb) format (e.g., an sdAb against IL12Rβ2) connected to the N-terminus of a second Fc domain via a second linker (2). FIG. 2D shows a tumor-targeted IL12Rβ2 agonist comprising (a) a tumor targeting moiety in sdAb format (e.g., an sdAb against a tumor-associated antigen) connected to the N-terminus of a first Fc domain via a first linker (1), and (b) an IL12Rβ2 binding moiety comprising an IL12 moiety connected to the N-terminus of a second Fc domain via a second linker (2). FIG. 2E shows a tumor-targeted IL12Rβ2 agonist comprising (a) a tumor targeting moiety in a sdAb format (e.g., an sdAb against a tumor-associated antigen) connected to the N-terminus of a first Fc domain via a first linker (1), and (b) an IL12Rβ2 binding moiety in Fab format (e.g., a Fab derived from an antibody against IL12Rβ2) connected to the N-terminus of a second Fc domain via a second linker (2). FIG. 2F shows a tumor-targeted IL12Rβ2 agonist comprising (a) a tumor targeting moiety in sdAb format (e.g., an sdAb against a tumor-associated antigen) connected to the N-terminus of a first Fc domain via a first linker (1) and (b) an IL12Rβ2 binding moiety in sdAb format (e.g., an sdAb against IL12Rβ2) connected to the N-terminus of a second Fc domain via a second linker (2). FIG. 2G shows a tumor-targeted IL12Rβ2 agonist comprising (a) a tumor targeting moiety in scFv format (e.g., an scFv against a tumor-associated antigen) connected to the N-terminus of a first Fc domain via a first linker (1) and an IL12Rβ2 binding moiety in sdAb format (e.g., an sdAb against IL12Rβ2) connected to the N-terminus of a second Fc domain via a second linker (2). FIG. 2H shows a tumor-targeted IL12Rβ2 agonist comprising (a) a tumor targeting moiety in scFv format (e.g., an scFv derived from an antibody against a tumor-associated antigen) connected to the N-terminus of a first Fc domain via a first linker (1) and an IL12Rβ2 binding moiety in scFv format (e.g., an scFv derived from an antibody against IL12Rβ2) connected to the N-terminus of a second Fc domain via a second linker (2). FIG. 2I shows a tumor-targeted IL12Rβ2 agonist comprising (a) a tumor targeting moiety in scFv format (e.g., an scFv derived from an antibody against a tumor-associated antigen) connected to the N-terminus of a first Fc domain via a first linker (1) and (b) an IL12Rβ2 binding moiety comprising an IL12 moiety connected to the N-terminus of a second Fc domain via a second linker (2). The p35 and p40 moieties of the IL12 moiety are connected to one another via a third linker (3). Mutation(s) in the p40 moiety are represented by a star. FIG. 2J shows a tumor-targeted IL12Rβ2 agonist comprising (a) a tumor targeting moiety in sdAb format (e.g., an sdAb against a tumor-associated antigen) connected to the N-terminus of a first Fc domain via a first linker (1) and an IL12Rβ2 binding moiety in scFv format (e.g., an scFv derived from an antibody against IL12Rβ2) connected to the N-terminus of a second Fc domain via a second linker (2). FIG. 2K shows a tumor-targeted IL12Rβ2 agonist comprising (a) a tumor targeting moiety in Fab format (e.g., a Fab derived from an antibody against a tumor-associated antigen) connected to the N-terminus of a first Fc domain via a first linker (1) and an IL12Rβ2 binding moiety in scFv format (e.g., an scFv derived from an antibody against IL12Rβ2) connected to the N-terminus of a second Fc domain via a second linker (2). FIG. 2L shows a tumor-targeted IL12Rβ2 agonist comprising (a) a tumor targeting moiety in scFv format (e.g., an scFv derived from an antibody against a tumor-associated antigen) connected to the N-terminus of a first Fc domain via a first linker (1) and an IL12Rβ2 binding moiety in Fab format (e.g., a Fab derived from an antibody against IL12Rβ2) connected to the N-terminus of a second Fc domain via a second linker (2).

FIGS. 3A-3B are cartoon illustrations depicting the cell-cell linkage between a tumor cell and a T-cell facilitated by combinations of tumor-targeted IL12Rβ1 and IL12Rβ2 agonists. FIG. 3A illustrates a combination of a tumor-targeted IL12Rβ1 and IL12Rβ2 agonists, each binding to a tumor-associated antigen via a Fab domain. The tumor-targeted IL12Rβ1 and IL12Rβ2 agonists bind to IL12Rβ1 or to IL12Rβ2 via their IL12 moieties, respectively. 1: Tumor-associated antigen (TAA); 2: IL12Rβ1; 3: IL12Rβ2. FIG. 3B illustrates a combination of a tumor-targeted IL12Rβ1 and IL12Rβ2 agonists, each binding to a tumor-associated antigen via a Fab domain. The tumor-targeted IL12Rβ1 and IL12Rβ2 agonists bind to IL12Rβ1 or to IL12Rβ2 via anti-IL12Rβ1 Fab or anti-IL12Rβ2 Fab domains, respectively. 1: Tumor-associated antigen (TAA)); 2: IL12Rβ1; 3: IL12Rβ2. FIG. 3C illustrates a combination of a tumor-targeted IL12Rβ1 and IL12Rβ2 agonists, each binding to a different tumor-associated antigen via a Fab domain. The tumor-targeted IL12Rβ1 and IL12Rβ2 agonists bind to IL12Rβ1 or to IL12Rβ2 via their IL12 moieties, respectively. 1a: First tumor-associated antigen (TAA); 1b: Second tumor-associated antigen (TAA); 2: IL12Rβ1; 3: IL12Rβ2. FIG. 3D illustrates a combination of a tumor-targeted IL12Rβ1 and IL12Rβ2 agonists, each binding to a different tumor-associated antigen via a Fab domain. The tumor-targeted IL12Rβ1 and IL12Rβ2 agonists bind to IL12Rβ1 or to IL12Rβ2 via anti-IL12Rβ1 Fab or anti-IL12Rβ2 Fab domains, respectively. 1a: First tumor-associated antigen (TAA); 1b: Second tumor-associated antigen (TAA); 2: IL12Rβ1; 3: IL12Rβ2.

FIGS. 4A-4D are graphs that show STAT3-Luc activity associated with IL12 moiety-comprising tumor-targeted IL12Rβ1 and IL12Rβ2 agonists in combinations. FIG. 4A is a graph that shows the STAT3-Luc activity of tumor-targeted IL12Rβ1 and IL12Rβ2 agonists in NK92/STAT3-Luc cells that were not co-cultured with other cells. FIG. 4B is a close-up of the graph displayed in FIG. 4A, showing an enhanced view of the non-hIL12 data points. FIG. 4C is a graph that shows the STAT3-Luc activity of tumor-targeted IL12Rβ1 and IL12Rβ2 agonists in NK92/STAT3-Luc cells co-cultured with Raji/hPSMA cells. FIG. 4D is a close-up of the graph displayed in FIG. 4C, showing a closer view of the non-hIL12 data points.

FIGS. 5A-5B are graphs that show STAT3-Luc activity associated with tumor-targeted IL12Rβ1 and IL12Rβ2 agonists in combinations in NK92/STAT3-Luc cells co-cultured with Raji cells that do not express PSMA or PSMA-expressing Raji cells. FIG. 5A shows STAT3-Luc activity associated with tumor-targeted IL12Rβ1 and IL12Rβ2 agonists alone or in combinations in NK92/STAT3-Luc cells co-cultured with Raji cells that do not express PSMA. FIG. 5B shows STAT3-Luc activity associated with tumor-targeted IL12Rβ1 and IL12Rβ2 agonists alone or in combinations in NK92/STAT3-Luc cells co-cultured with PSMA-expressing Raji cells.

FIG. 6A-6D are graphs that show the release of IFNγ from primary human T-cells upon treatment with tumor-targeted IL12Rβ1 and IL12Rβ2 agonists in combination or control treatments, in the presence of a tumor-targeting CD3 bispecific Ab. FIGS. 6A and 6C show the release of IFNγ from primary human T-cells in the presence of Raji cells that do not express PSMA. FIGS. 6B and 6D show the release of IFNγ from primary human T-cells in the presence of PSMA-expressing Raji cells.

FIGS. 7A-7B are graphs that show the activation of phospho-STAT4 (pSTAT4) signaling in primary human T-cells upon treatment with tumor-targeted IL12Rβ1 and IL12Rβ2 agonists. FIG. 7A shows percent pSTAT4 activation in primary human T-cells in the absence of tumor cells. FIG. 7B shows percent pSTAT4 activation in primary human T-cells in the presence of PSMA-expressing C4-2 tumor cells.

FIGS. 8A-8D show target cell killing and IFNγ release from primary human T-cells upon treatment with tumor-targeted IL12Rβ1 and IL12Rβ2 agonists in combination or control treatments, in the presence of a hMUC16-targeting CD3 bispecific Ab. FIG. 8A is a graph that shows the extent of killing of PSMA-expressing HEK293/hMUC16 cells. FIG. 8B is a graph that shows the extent of killing of HEK293/hMUC16 cells that do not express PSMA. FIG. 8C is a graph that shows IFNγ release from primary human T-cells in the presence of PSMA-expressing HEK293/hMUC16 cells. FIG. 8D is a graph that shows IFNγ release from primary human T-cells in the presence of HEK293/hMUC16 cells that do not express PSMA.

FIGS. 9A-9D are graphs that show STAT3-Luc activity associated with tumor-targeted IL12Rβ1 and IL12Rβ2 agonists in combination. FIG. 9A shows STAT3-Luc activity associated with a first set of PSMA-targeted IL12Rβ1 and IL12Rβ2 agonists alone or in combination in NK92/STAT3-Luc cells co-cultured with Jurkat cells that do not express PSMA. FIG. 9B shows STAT3-Luc activity associated with the first set of PSMA-targeted IL12Rβ1 and IL12Rβ2 agonists alone or in combination in NK92/STAT3-Luc cells co-cultured with PSMA-expressing Jurkat cells. FIG. 9C shows STAT3-Luc activity associated with a second set of PSMA-targeted IL12Rβ1 and IL12Rβ2 agonists alone or in combination in NK92/STAT3-Luc cells co-cultured with Jurkat cells that do not express PSMA. FIG. 9D shows STAT3-Luc activity associated with the second set of PSMA-targeted IL12Rβ1 and IL12Rβ2 agonists alone or in combination in NK92/STAT3-Luc cells co-cultured with PSMA-expressing Jurkat cells.

FIGS. 10A-10D are graphs that show target cell killing and IFNγ release from primary human T-cells upon treatment with PSMA-targeted IL12Rβ1 and IL12Rβ2 agonists, alone or in combination, in the presence of a hSTEAP1-targeting CD3 bispecific antibody. FIG. 10A shows the extent of killing of PSMA-expressing LNCaP cells associated with a first set of PSMA-targeted IL12Rβ1 and IL12Rβ2 agonists alone or in combination. FIG. 10B shows IFNγ release from PSMA-expressing LNCaP cells associated with the first set of PSMA-targeted IL12Rβ1 and IL12Rβ2 agonists alone or in combination. FIG. 10C shows the extent of killing of PSMA-expressing LNCaP cells associated with a second set of PSMA-targeted IL12Rβ1 and IL12Rβ2 agonists alone or in combination. FIG. 10D shows IFNγ release from PSMA-expressing LNCaP cells associated with the second set of PSMA-targeted IL12Rβ1 and IL12Rβ2 agonists alone or in combination.

FIGS. 11A-11B are graphs that show the activation of phospho-STAT4 (pSTAT4) signaling in primary human T-cells upon treatment with PSMA-targeted IL12Rβ1 and IL12Rβ2 agonists. FIG. 11A shows percent pSTAT4 activation in primary human T-cells in the presence of PSMA-expressing C4-2 tumor cells associated with a first set of PSMA-targeted IL12Rβ1 and IL12Rβ2 agonists alone or in combination. FIG. 11B shows percent pSTAT4 activation in primary human T-cells in the presence of PSMA-expressing C4-2 tumor cells associated with a second set of PSMA-targeted IL12Rβ1 and IL12Rβ2 agonists alone or in combination.

FIG. 12A shows activation of phospho-STAT4 (pSTAT4) signaling in primary human T-cells in the presence OVCAR3 tumor cells upon treatment with MUC16-targeted IL12Rβ1 and IL12Rβ2 agonists alone or in combination. FIG. 12B shows target cell killing upon treatment with MUC16-targeted IL12Rβ1 and IL12Rβ2 agonists, alone or in combination, in the presence of a hMSLN-targeting CD3 bispecific antibody.

FIG. 13A shows activation of phospho-STAT4 (pSTAT4) signaling in primary human T-cells in the presence of OVCAR3 tumor cells upon treatment with MSLN-targeted IL12Rβ1 and IL12Rβ2 agonists alone or in combination. FIG. 13B shows target cell killing upon treatment with MSLN-targeted IL12Rβ1 and IL12Rβ2 agonists, alone or in combination, in the presence of a hMUC16-targeting CD3 bispecific antibody.

FIG. 14A-14B are graphs that show STAT3-Luc activity associated with tumor-targeted IL12Rβ1 and IL12Rβ2 agonists in combination. FIG. 14A shows STAT3-Luc activity associated with sets of PSMA-targeted IL12Rβ1 and IL12Rβ2 agonists in combination in NK92/STAT3-Luc cells co-cultured with Raji cells that do not express PSMA. FIG. 14B shows STAT3-Luc activity associated with sets of PSMA-targeted IL12Rβ1 and IL12Rβ2 agonists in combination in NK92/STAT3-Luc cells co-cultured with PSMA-expressing Raji cells.

FIG. 15A-15B show activation of pSTAT4 signaling in primary human T-cells upon treatment with tumor-targeted IL12Rβ1 and IL12Rβ2 agonists in combination. FIG. 15A shows activation of pSTAT4 signaling in primary human T-cells in the absence of tumor cells upon treatment with PSMA-targeted IL12Rβ1 and IL12Rβ2 agonists in combination. FIG. 15B shows activation of pSTAT4 signaling in primary human T-cells in the presence of C4-2 tumor cells upon treatment with PSMA-targeted IL12Rβ1 and IL12Rβ2 agonists in combination.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antigen Binding Domain or ABD: The term “antigen binding domain” or “ABD” as used herein refers to the portion of a targeting moiety that is capable of specific, non-covalent, and reversible binding to a target molecule.

Associated: The term “associated” in the context of a protein or protein component (e.g., a tumor-targeted IL12Rβ1 agonist; a tumor-targeted IL12Rβ2 agonist; a targeting moiety such as a Fab) refers to a functional relationship between two amino acid sequences on one or more polypeptide chains. In particular, the term “associated” means that two or more sequences or polypeptide chains are associated with one another, e.g., non-covalently through molecular interactions or covalently through one or more disulfide bridges or chemical cross-linkages, so as to produce a functional protein or protein component. Examples of associations that might be present in a tumor-targeted split IL12 receptor agonist of the disclosure include (but are not limited to) associations between p40 and p35 moieties, associations between homodimeric or heterodimeric Fc domains in an Fc region, associations between VH and VL regions in a Fab or scFv, associations between CH1 and CL in a Fab, and associations between CH3 and CH3 in a domain substituted Fab.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Though most naturally occurring antibodies are composed of heavy chains and light chains, camelids (e.g., camels, dromedaries, llamas, and alpacas) and some sharks produce antibodies that consist only of heavy chains. These antibodies bind antigenic epitopes using a single variable domain known as VHH and contain only heavy chain CDRs (CDR-H1, CDR-H2, CDR-H3). Exemplary conventions that can be used to identify the boundaries of CDRs include, e.g., the Kabat definition, the Chothia definition, the ABM definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABM numbering scheme); and Lefranc et al., 2003, Dev. Comp. Immunol. 27:55-77 (IMGT numbering scheme). Public databases are also available for identifying CDR sequences within an antibody.

EC50: The term “EC50” refers to the half maximal effective concentration of a molecule or combination of molecules (such as a tumor-targeted split IL12 receptor agonist) which induces a response halfway between the baseline and maximum after a specified exposure time. The EC50 essentially represents the concentration of an antibody or Tumor-targeted split IL12 receptor agonist where 50% of its maximal effect is observed. In certain embodiments, the EC50 value equals the concentration of a tumor-targeted split IL12 receptor agonist that gives half-maximal STAT3 activation in an assay as described in Section 8.1.2.

Epitope: An epitope, or antigenic determinant, is a portion of an antigen (e.g., target molecule) recognized by an antibody or other antigen-binding moiety as described herein. An epitope can be linear or conformational.

Fab: The term “Fab” in the context of a targeting moiety of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

IL12 Moiety: The term “IL12 moiety” refers to an amino acid sequence comprising a p35 moiety and a p40 moiety, which may be on a single polypeptide chain. The p35 moiety may be N- or C-terminal to the p40 moiety. In some embodiments, the p35 moiety and the p40 moiety are configured to associate with one another. In some embodiments, the p35 moiety and the p40 moiety are connected via a linker. The related term “IL12 moiety linker” refers to a linker connecting a p35 moiety and a p40 moiety.

IL12 p35 moiety or p35 moiety: An IL12 p35 moiety or a p35 moiety is an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ2 binding portion of a mammalian, e.g., human or murine, p35 (sometimes referred to as the alpha subunit of IL12 or IL12a), optionally with one or amino acid substitutions as defined in Section 6.3.2.2 or Section 6.4.2.2 below.

In eukaryotic cells, the human IL12 p35 subunit is synthesized as a precursor polypeptide of 219 amino acids, from which 22 amino acids are removed to generate mature IL12 p35. In some embodiments, the mammalian p35 is full-length human p35. In other embodiments, the mammalian p40 is mature human p35. The sequence of human p35 has the Uniprot identifier P29459 (uniprot.org/uniprot/P29459). In some embodiments, the mammalian p35 is full-length murine p35. In some embodiments, the mammalian p35 is mature murine p40. The sequence of murine p40 has the Uniprot identifier P43431 (uniprot.org/uniprot/P43431).

Full-length human IL12 p35 has the following amino acid sequence (signal sequence=underlined):

(SEQ ID NO: 1)
MCPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS.

p35 comprises a signal sequence (at amino acids 1-22 of human p35). Thus, amino acid 23 of full-length human p35 is amino acid 1 of mature human p35.

In native IL12, p35 has four conserved cysteine residues that form two inter-strand disulfide bonds, which bridge C64 and C96 as well as C85 and C123 of human p35. p35 also includes a cysteine (C74 of human p35) that forms an inter-chain bond with p40 (at amino acid C177 of human p40)).

The p35 moiety preferably comprises an amino acid sequence comprising at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a mature a mammalian p35, e.g., human or murine p35 (corresponding to amino acids 23-219 of human p35), optionally with one or amino acid substitutions as defined in Section 6.3.2 below.

IL12 p40 moiety or p40 moiety: An IL12 p40 moiety or a p40 moiety is an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ1 binding portion of a mammalian, e.g., human or murine, p40 (sometimes referred to as the beta subunit of IL12 or IL12P), optionally with one or amino acid substitutions as defined in Section 6.3.2.2 or Section 6.4.2.2 below.

In eukaryotic cells, the human IL12 p40 subunit is synthesized as a precursor polypeptide of 328 amino acids, from which 22 amino acids are removed to generate mature IL12 p40. The sequence of human p40 has the Uniprot identifier P29460 (uniprot.org/uniprot/P29460). In some embodiments, the mammalian p40 is full-length murine p40. In some embodiments, the mammalian p40 is mature murine p40. The sequence of murine p40 has the Uniprot identifier P43432 (uniprot.org/uniprot/P43432).

In some embodiments, the p40 moiety comprises p40 D2 and D3 domains, to the exclusion of the p40 D1 domain. In other embodiments, the p40 moiety comprises p40 D1, D2, and D3 domains.

Full-length human IL12 p40 has the following amino acid sequence (signal sequence=underlined; D1 domain=italicized; D2 domain=bold; D3 domain=bold and underlined):

(SEQ ID NO: 5)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS.

p40 comprises a signal sequence at amino acids 1-22 of human p40. Thus, amino acid 23 of full-length human p40 is amino acid 1 of mature human p40.

The sequence of human p40 has the Uniprot identifier P29460 (uniprot.org/uniprot/P29460). The sequence of murine p40 has the Uniprot identifier P43432 (uniprot.org/uniprot/P43432). p40 comprises an Ig-like C2-type domain referred to as D1 (at amino acids 23 to 106 of human p40), a first fibronectin type-III domain referred to as D2 (at amino acids 107 to 236 of human p40) and a second fibronectin type-III domain referred to as D3 (at amino acids 237 to 328 of human p40). In native IL12, the D2 domain of p40 has four conserved cysteine residues which form two inter-strand disulfide bonds, which bridge C109 and C120 and C148 and C171 in human p40 and the D3 domain also contains an inter-strain disulfide bond, which bridges C278 and C305 in human p40. D2 also includes a cysteine (C177 in human p40) that forms an inter-chain bond with p35 (at amino acid C74 of human p35). D3 also contains the highly conserved WSXWS motif (SEQ ID NO: 44) (WSEWAS (SEQ ID NO: 45) in human p40).

The p40 moiety preferably includes a D2 domain and a D3 domain (or an amino acid sequence comprising at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the D2 and D3 domains) of a mammalian, e.g., human or murine, p40, optionally with one or amino acid substitutions as defined in Section 6.3.2.2 or Section 6.4.2.2 below.

The p40 moiety can also include a D1 domain or an amino acid sequence comprising at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the D1 domain of a mammalian, e.g., human or murine, p40, optionally with one or amino acid substitutions as defined in Section 6.3.2.2 or Section 6.4.2.2 below.

In various embodiments, the p40 moiety of an IL12 moiety of the disclosure retains any combination of (a) none, any one, any two or all three inter-strand disulfide bonds and/or (b) the cysteine that forms an inter-chain bond with p35 and/or (c) the conserved WSXWS motif (SEQ ID NO: 44).

IL12Rβ1: IL12Rβ1 is the IL12 receptor subunit beta-1 (IL12Rβ1), which binds to IL12 p40. The sequence of human IL12Rβ1 has the Uniprot identifier P42701 (uniprot.org/uniprot/P42701), with amino acids 24 to 545 making up the extracellular domain. The sequence of murine IL12Rβ1 has the Uniprot identifier Q60837 (uniprot.org/uniprot/Q60837), with amino acids 20 to 565 making up the extracellular domain. IL12Rβ1 comprises a signal sequence (at amino acids 1-23 of human IL12Rβ1), an extracellular p40-binding domain (at amino acids 24 to 545 of human IL12Rβ1), a helical transmembrane domain (at amino acids 546 to 570 of human IL12Rβ1) and a cytoplasmic domain (at amino acids 571 to 662 of human IL12Rβ1).

IL12Rβ2: IL12Rβ2 is the IL12 receptor subunit beta-2 (IL12Rβ2), which binds to IL12 p30. The sequence of human IL12Rβ has the Uniprot identifier Q99665 (uniprot.org/uniprot/Q99665), with amino acids 24 to 622 making up the extracellular domain. The sequence of murine IL12Rβ2 has the Uniprot identifier P97378 (uniprot.org/uniprot/Q60837), with amino acids 24 to 637 making up the extracellular domain. IL12Rβ2 comprises a signal sequence (at amino acids 1-23 of human IL12Rβ2), an extracellular p40-binding domain (at amino acids 24 to 622 of human IL12Rβ2), a helical transmembrane domain (at amino acids 623 to 643 of human IL12Rβ2) and a cytoplasmic domain (at amino acids 644 to 862 of human IL12Rβ2).

IL12 Variant or Variant IL12: An “IL12 variant” or “variant IL12” is an IL12 moiety composed or one or more polypeptide chains comprising an IL12 p35 (referred to as “p35”) moiety and an IL12 p40 (“p40”) moiety in association with one another and which varies from native IL12 by the primary amino acid sequence of its p35 moiety (a “variant p35 moiety” or “variant p35”) and/or p40 moiety (a “variant p40 moiety” or “variant p40”) a relative to wild type p35 and/or p40, respectively.

In some embodiments, the variant IL12 has increased relative affinity to the IL12Rβ1 receptor vs. the IL12Rβ2 receptor as compared to wild-type IL12, for example through one or more mutations in p40 that increase binding to IL12Rβ1 and/or through one or more mutations in p35 that reduce binding to IL12Rβ2. Such variants are sometimes referred to herein as “IL12Rβ1 ligands,” “IL12Rβ1-skewed IL12 variants,” and the like.

In some embodiments, the variant IL12 has increased relative affinity to the IL12Rβ2 receptor vs. the IL12Rβ1 receptor as compared to wild-type IL12, for example through one or more mutations in p35 that increase binding to IL12Rβ2 and/or through one or more mutations in p40 that reduce binding to IL12Rβ1. Such variants are sometimes referred to herein as “IL12Rβ2 ligands,” “IL12Rβ2-skewed IL12 variants,” and the like.

Binding affinity of p40 to IL12Rβ1 and of p35 to IL12Rβ2 can be assayed by surface plasmon resonance (SPR) techniques (analyzed on a Biacore instrument) (Liljeblad et al., 2000, Glyco J 17:323-329).

The variant IL12 can thus comprise a p35 and/or p40 moiety with one or more amino acid substitutions, deletions and/or insertions compared to wild type p35 and/or p40. Exemplary mutations, e.g., substitutions, are disclosed, inter alia, in Sections 6.3.2.2 and 6.4.2.2.

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Single Chain Fv or scFv: The term “single chain Fv” or “scFv” as used herein refers to a polypeptide chain comprising the VH and VL domains of antibody, where these domains are present in a single polypeptide chain.

Single Domain Antibody or sdAb: The term “single domain antibody” or “sdAb” as used herein refers to an antibody or antigen binding fragment thereof comprising a single binding domain (e.g., heavy chain variable region) capable of binding a target molecule without pairing with a corresponding CDR-containing polypeptide (e.g., a light chain). An sdAb or sdAb fragment can be derived from a VH, a VHH, or from a non-antibody scaffold protein, for example a designed ankyrin repeat protein (darpin), an avimer, an anticalin/lipocalin, a centyrin or a fynomer. A sdAb typically lacks a CH1 domain and thus cannot associate with a light chain.

Single Domain VH Antibody or sdVH: The term “single domain VH” or “sdVH” as used herein refers to a variable region of an sdAb that is not of camelid or cartilaginous fish origin. An sdVH can be, for example, of human or non-human mammalian origin. A basic sdVH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

Specifically (or selectively) binds: The term “specifically (or selectively) binds” as used herein means that a targeting moiety, e.g., an antibody, or antigen binding domain (“ABD”) thereof, forms a complex with a target molecule that is relatively stable under physiologic conditions. Specific binding can be characterized by a Ko of about 5×10⁻²M or less (e.g., less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment, e.g., a tumor-targeted split IL12 receptor agonist or a component targeting moiety, to a target molecule are well known in the art and include, for example, equilibrium dialysis, surface plasmon resonance (e.g., Biacore assays), fluorescent-activated cell sorting (FACS) binding assays and the like. A tumor-targeted split IL12 receptor agonist of the disclosure comprising a targeting moiety or an ABD thereof that specifically binds a target molecule from one species can, however, have cross-reactivity to the target molecule from one or more other species.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in a tumor-targeted split IL12 receptor agonist of the disclosure. In various embodiments of the tumor-targeted split IL12 receptor agonists of the disclosure, a target molecule can be tumor-associated antigen, IL12Rβ1 or IL12Rβ2.

Targeting Moiety: The term “targeting moiety” as used herein refers to any molecule or binding portion (e.g., an immunoglobulin or an antigen binding fragment) thereof that can bind to a cell surface molecule, e.g., at a site to which a tumor-targeted split IL12 receptor agonist of the disclosure is to be localized. In some embodiments, a targeting moiety binds to a cell surface molecule on tumor cells or on lymphocytes in the tumor microenvironment. The targeting moiety can also have a functional activity in addition to localizing a molecule to a particular site. For example, a targeting moiety in a tumor-targeted split IL12 receptor that is an anti-IL12Rβ1 or anti-IL12Rβ2 antibody or an antigen binding portion thereof can modulate (e.g., agonize) IL12 signaling in T-lymphocytes.

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more Tumor-targeted split IL12 receptor agonists of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment, and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment, and not synthesized or expressed on the surface of a normal cell. Accordingly, the term “TAA” encompasses antigens that are specific to cancer cells, sometimes known in the art as tumor-specific antigens (“TSAs”).

Tumor-targeted IL12Rβ1 agonist: The term “tumor-targeted IL12Rβ1 agonist” as used herein refers to a molecule comprising a tumor-associated antigen (“TAA”) targeting moiety and an IL12Rβ1 binding moiety. The word “agonist” is used for convenience only and is not intended to imply that the molecule need possess IL12 signaling or other activity, whether alone or in combination with another molecule (e.g., a tumor-targeted IL12Rβ2 agonist). In some embodiments, the combination of a tumor-targeted IL12Rβ1 agonist and a tumor-targeted IL12Rβ2 agonist results in signaling via the IL12 receptor and/or clustering of IL12Rβ1 and IL12Rβ2 receptor subunits.

Tumor-targeted IL12Rβ2 agonist: The term “tumor-targeted IL12Rβ2 agonist” as used herein refers to a molecule comprising a tumor-associated antigen (“TAA”) targeting moiety and an IL12Rβ2 binding moiety. The word “agonist” is used for convenience only and is not intended to imply that the molecule need possess IL12 signaling or other activity, whether alone or in combination with another molecule (e.g., a tumor-targeted IL12Rβ1 agonist). In some embodiments, the combination of a tumor-targeted IL12Rβ1 agonist and a tumor-targeted IL12Rβ2 agonist results in signaling via the IL12 receptor and/or clustering of IL12Rβ1 and IL12Rβ2 receptor subunits.

Universal Light Chain: The term “universal light chain” as used herein in the context of a targeting moiety refers to a light chain polypeptide capable of pairing with the heavy chain region of an antibody or antibody fragment, e.g., a targeting moiety, and also capable of pairing with other heavy chain regions. Universal light chains are also known as “common light chains.”

VHH: The term “VHH” refers to a variable region of an antibody consisting of only a heavy chain, e.g., an antibody of camelid or cartilaginous fish origin. A VHH variable region can bind to a target molecule in the absence of a light chain. A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 6.2. Tumor-Targeted Split L12 Receptor Agonists

The present disclosure provides tumor-targeted split IL12 receptor agonists. Tumor-targeted split IL12 receptor agonists comprise two components, a tumor-targeted IL12Rβ1 agonist and a tumor-targeted IL12Rβ2 agonist. Thus, a tumor-targeted split IL12 receptor agonist of the disclosure is sometimes referred to herein as a “combination.”

The tumor-targeted IL12Rβ1 agonist and a tumor-targeted IL12Rβ2 agonists each comprises a tumor-associated antigen (“TAA”) targeting moiety, and the TAA targeting moiety of the tumor-targeted IL12Rβ1 agonist and the TAA targeting moiety of the tumor-targeted IL12Rβ2 agonist are typically capable of binding to the same cell, e.g., a tumor cell.

The tumor-targeted IL12Rβ1 agonist further comprises an IL12Rβ1 binding moiety, and the tumor-targeted IL12Rβ2 agonist further comprises an IL12Rβ2 binding moiety. The IL12Rβ1 and IL12Rβ2 binding moieties can each be a targeting moiety (e.g., an antigen binding fragment of an anti-IL12Rβ1 or anti-IL12Rβ2 antibody, respectively), or it can be a ligand (e.g., an IL12 moiety with preferential binding to the IL12Rβ1 or IL12Rβ2 receptor, respectively).

When the tumor-targeted IL12Rβ1 agonist and tumor-targeted IL12Rβ2 agonist are in proximity of a tumor cell recognized by the TAA targeting moiety and a cell harboring the IL12 receptor such as a cytotoxic T-lymphocyte, the tumor-targeted split IL12 receptor agonist can cross-link the tumor cell and cytotoxic T-lymphocyte, thereby triggering a cytotoxic immune response against the tumor cell. Accordingly, in some embodiments, disclosed are tumor-targeted IL12Rβ1 agonists for use in a method of triggering a cytotoxic immune response against a tumor cell, the method comprising administration of the tumor-targeted IL12Rβ1 agonist and a tumor-targeted IL12Rβ2 agonist. Also disclosed, in some embodiments, are tumor-targeted IL12Rβ2 agonists for use in a method of triggering a cytotoxic immune response against a tumor cell, the method comprising administration of the tumor-targeted IL12Rβ2 agonist and a tumor-targeted IL12Rβ1 agonist. The tumor-targeted IL12Rβ1 agonist and the tumor-targeted IL12Rβ2 agonist may be administered simultaneously or sequentially, and may be administered in the same composition or different compositions.

The use of singular terms, such as “tumor-targeted split IL12 receptor agonist” and “combination” is for convenience only and does not necessitate that the tumor-targeted IL12Rβ1 agonist and tumor-targeted IL12Rβ2 agonist be present in the same composition, but merely that the two components be capable of being used with one another, e.g., to trigger a cytotoxic immune response against a tumor cell.

Examples of tumor-targeted IL12Rβ1 agonists and their components are described in Section 6.3 and subsections thereof.

Examples of tumor-targeted IL12Rβ2 agonists and their components are described in Section 6.4 and subsections thereof.

Suitable TAA targeting moieties for inclusion in the tumor-targeted IL12Rβ1 agonists and tumor-targeted IL12Rβ2 agonists in a tumor-targeted split IL12 receptor agonist are exemplified in Section 6.5.

Suitable formats of the targeting moieties in a tumor-targeted split IL12 receptor agonist (e.g., a TAA targeting moiety, an IL12Rβ1 targeting moiety, or an IL12Rβ2 targeting moiety) are disclosed in Section 6.6.

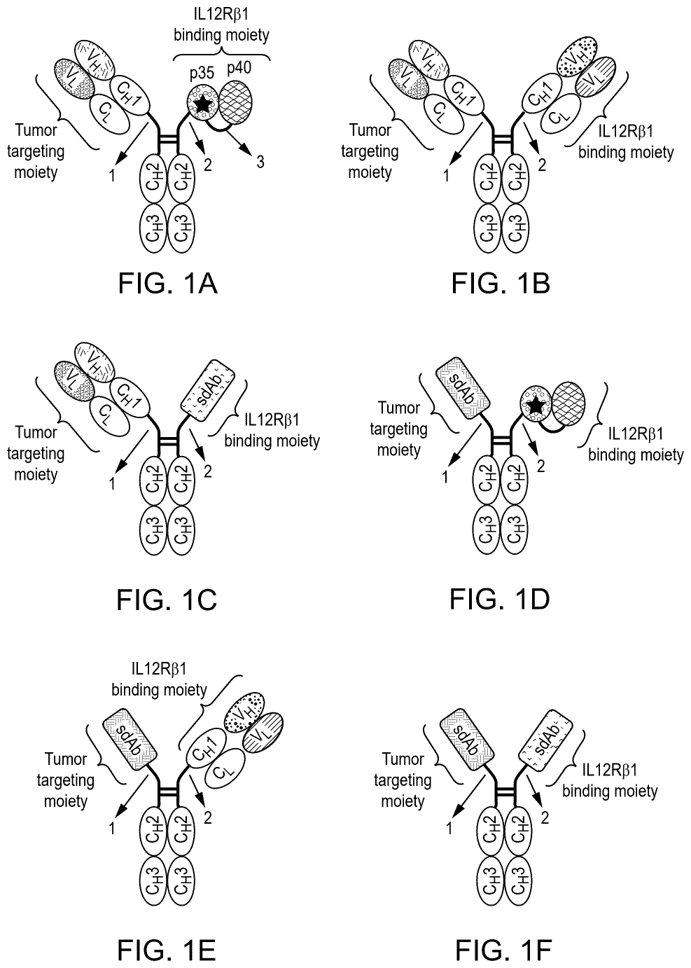

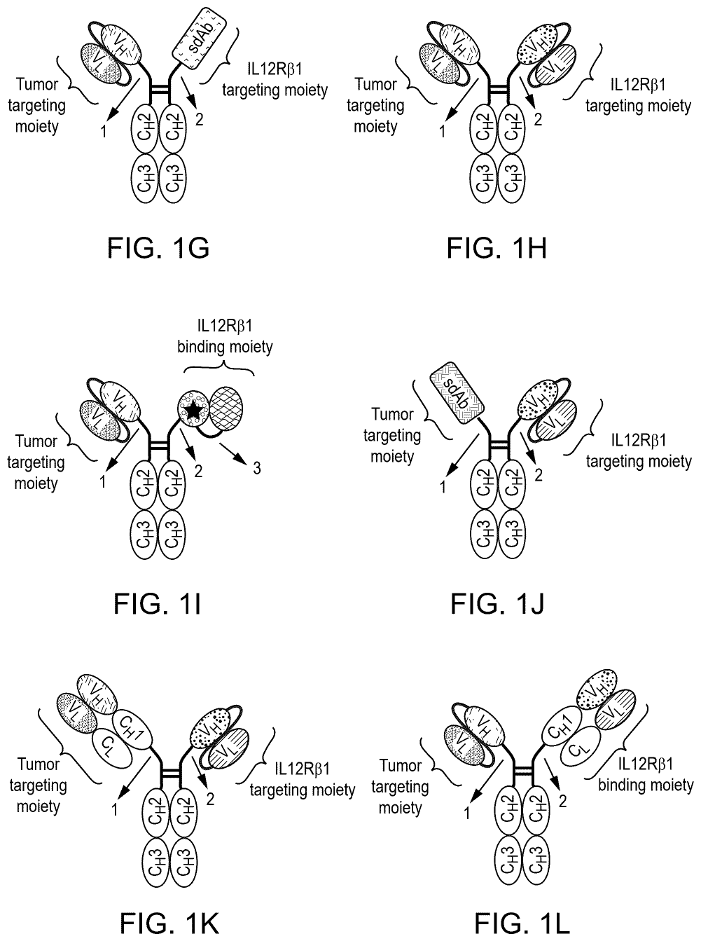

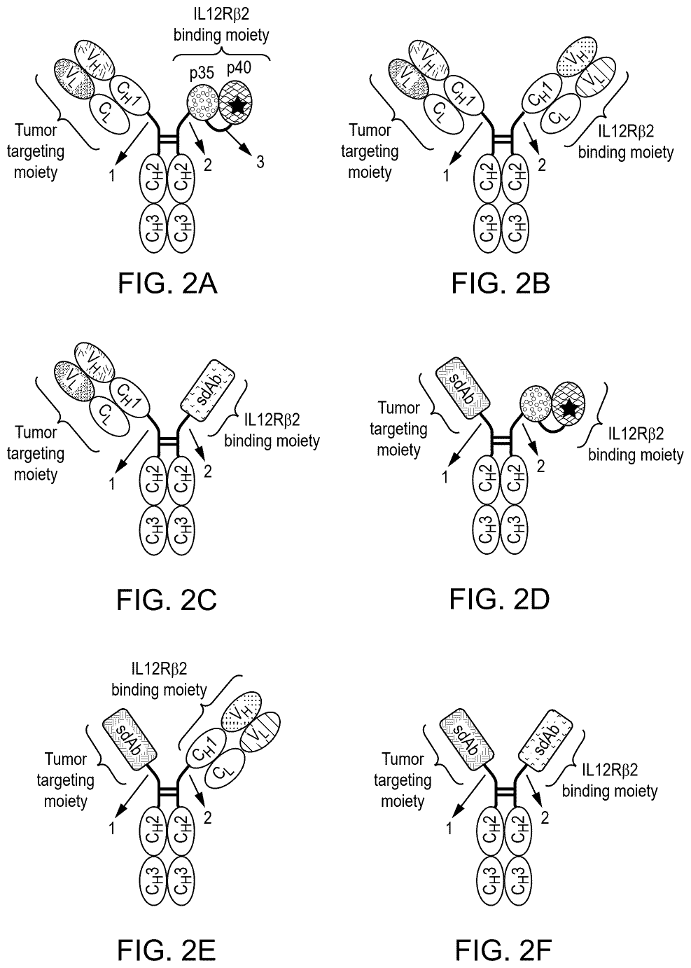

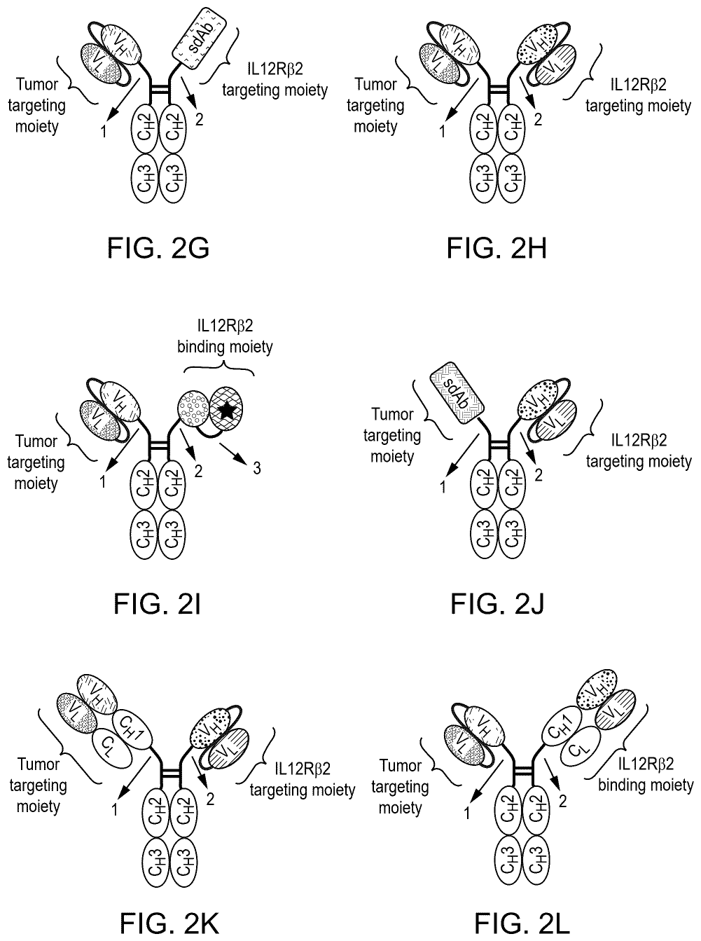

The tumor-targeted IL12Rβ1 agonist and a tumor-targeted IL12Rβ2 agonist typically contain Fc domains to which the TAA targeting moieties and the IL12Rβ1 or IL12Rβ1 binding moieties are operably linked. Suitable Fc domains are disclosed in Section 6.8. Suitable arrangements of Fc domain, TAA targeting moiety and IL12Rβ1 binding moiety in a tumor-targeted IL12Rβ1 agonist are disclosed in FIG. 1 (FIGS. 1A-1L). Suitable arrangements of Fc domain, TAA targeting moiety and IL12Rβ2 binding moiety in a tumor-targeted IL12Rβ2 agonist are disclosed in FIG. 2 (FIGS. 2A-2L).

One or more domains in a tumor-targeted IL12Rβ1 agonist and/or a tumor-targeted IL12Rβ2 agonist may be connected to one another via one or more linkers. Suitable linkers are disclosed in Section 6.9.

Nucleic acids encoding and host cells capable of expressing a tumor-targeted IL12Rβ1 agonist and/or a tumor-targeted IL12Rβ2 agonist are disclosed in Section 6.10.

Pharmaceutical compositions comprising the tumor-targeted IL12Rβ1 agonists, tumor-targeted IL12Rβ2 agonists and tumor-targeted split IL12 receptor agonists are disclosed in Section 6.11.

Methods of using the tumor-targeted split IL12 receptor agonists, e.g., to treat cancer or elicit anti-cancer immunity, are disclosed in Section 6.12.

Combination methods using the targeted split IL12 receptor agonists in combination with an additional therapeutic agent (e.g., a multispecific T-cell engager as described in Section 6.6), e.g., to treat cancer or elicit anti-cancer immunity, are disclosed in Section 6.13.

### 6.3. Tumor-Targeted IL12Rβ1 Agonist

### 6.3.1. Tumor-Associated Antigen Targeting Moieties

The tumor-targeted IL12Rβ1 agonist of the tumor-targeted split IL12 receptor agonists of the disclosure comprise a tumor-associated antigen (“TAA”) targeting moiety. Typically, the TAA recognized by the TAA targeting moiety of the tumor-targeted IL12Rβ1 agonist is expressed on the same cancer cell as the TAA recognized by the TAA targeting moiety of the tumor-targeted IL12Rβ2 agonist.

In some embodiments, both TAA targeting moieties recognize the same TAA, whether on the same epitope or on different epitopes. If the TAA targeting moieties recognize different epitopes, they preferably can bind to the cancer cell simultaneously and/or in a non-competing manner. When the TAA(s) recognized by both TAA targeting moieties are expressed on the same cancer cell, the TAA(s) may be the same TAA or different TAAs.

In some embodiments, both TAA targeting moieties recognize different TAAs expressed on the same cancer cell.

Suitable TAA targeting moieties are described in Section 6.5.

### 6.3.2. IL12Rβ1 Binding Moieties

### 6.3.2.1. IL12Rβ1 Targeting Moieties

In some embodiments, the tumor-targeted IL12Rβ1 agonist of the tumor-targeted split IL12 receptor agonists of the disclosure comprises an IL12Rβ1 targeting moiety as an IL12Rβ1 binding moiety.

The IL12Rβ1 targeting moiety typically is or comprises an antigen binding domain of an antibody. The IL12Rβ1 targeting moiety can be any format, e.g., as disclosed in Section 6.6 or subsections thereof. In some embodiments, the IL12Rβ1 targeting moiety is a Fab. In some embodiments, the IL12Rβ1 targeting moiety is an scFv. In some embodiments, the IL12Rβ1 targeting moiety is a sdAb (e.g., a VHH or an sdVH).

In some embodiments, the IL12Rβ1 targeting moiety is based on an antibody comprising both heavy and light chain variable regions. Exemplary anti-IL12Rβ1 antibodies comprising both heavy and light chain variable regions are set forth in Table R1 below.

TABLE R1
Exemplary Anti-IL12Rβ1 Variable Heavy (VH) and Light (VL) Chain Amino Acid
Sequences

| Target or | | | SEQ |
|---|---|---|---|
| Description | Reference | Sequence | ID NO |
| α-IL12Rβ1 | SEQ ID NO: 49 of | QVQLVESGGGVVQPGRSLRLSCAASGFTFTSYGMS | 53 |
| antibody VH | U.S. Publication | WVRQAPGKGLEWVAGISYSGSDTEYADSVKGRFTI | |
| sequence | No. US | SRDNSKNTLYLQMNSLRAEDTAVYYCARSPDYIID | |
| | 2023/0279127 A1 | YGFDYWGRGTLVTVSS | |
| α-IL12Rβ1 | SEQ ID NO: 50 of | AIQMTQSPSSLSASVGDRVTITCRASQGISSDLAW | 54 |
| antibody | U.S. Publication | YQQKPGKAPKLLIYDASSLQSGVPSRFSGSGSGTD | |
| VL | No. US | FTLTISSLQPEDFATYYCQQYWIYPFTFGQGTKVE | |
| sequence | 2023/0279127 A1 | IKRT | |
| α-IL12Rβ1 | SEQ ID NO: 51 of | QVQLVESGGGVVQPGRSLRLSCAASGFTFTSYGMS | 55 |
| antibody VH | U.S. Publication | WVRQAPGKGLEWVAGISYDASDTEYADSVKGRFTI | |
| sequence | No. US | SRDNSKNTLYLQMNSLRAEDTAVYYCARSPDYIID | |
| | 2023/0279127 A1 | YGFDYWGRGTLVTVSS | |
| α-IL12Rβ1 | SEQ ID NO: 52 of | AIQMTQSPSSLSASVGDRVTITCRASQGISSDLAW | 56 |
| antibody | U.S. Publication | YQQKPGKAPKLLIYDASSLQSGVPSRFSGSGSGTD | |
| VL | No. US | FTLTISSLQPEDFATYYCQQYWWYPFTFGQGTKVE | |
| sequence | 2023/0279127 A1 | IKRT | |
| α-IL12Rβ1 | SEQ ID NO: 53 of | QVQLVQSGAEVKKPGASVKVSCKASGYTFTGYYMH | 57 |
| antibody VH | U.S. Publication | WVRQAPGQGLEWMGMIGPQHGEAIYAQKFQGRVTM | |
| sequence | No. US | TRDTSISTAYMELSRLRSEDTAVYYCARESTDSDE | |
| | 2023/0279127 A1 | SPFDYWGQGTLVTVSS | |
| α-IL12Rβ1 | SEQ ID NO: 54 of | DIELTQPPSVSVSPGQTASITCSGDNIRSYYVSWY | 58 |
| antibody | U.S. Publication | QQKPGQAPVLVIYDDSDRPSGIPERFSGSNSGNTA | |
| VL | No. US | TLTISGTQAEDEADYYCQSYGSHSNFVVFGGGTKL | |
| sequence | 2023/0279127 A1 | TVLGQ | |
| α-IL12Rβ1 | SEQ ID NO: 55 of | QVQLVQSGAEVKKPGASVKVSCKASGYTFTGYYMH | 59 |
| antibody VH | U.S. Publication | WVRQAPGQGLEWMGMIGPQHGEAIYAQKFQGRVTM | |
| sequence | No. US | TRDTSISTAYMELSRLRSDDTAVYYCARESTDSDE | |
| | 2023/0279127 A1 | SPFDYWGQGTLVTVSS | |
| α-IL12Rβ1 | SEQ ID NO: 56 of | SYELTQPLSVSVALGQTARITCSGDNIRSYYVSWY | 60 |
| antibody | U.S. Publication | QQKPGQAPVLVIYDDSDRPSGIPERFSGSNSGNTA | |
| VL | No. US | TLTISRAQAGDEADYYCQSYGSHSNFVVFGGGTKL | |
| sequence | 2023/0279127 A1 | TVLGQ | |
| α-IL12Rβ1 | SEQ ID NO: 29 of | QVQLKESGPALVKPTQTLTLTCTFSGFSLSTSTMG | 61 |
| antibody VH | PCT Publication | VSWIRQPPGKALEWLAWIYWDDDKDYSTSLKSRLT | |
| sequence | No. WO | ISKDTSKNQVVLTMTNMDPVDTATYYCARANPDLG | |
| | 2010/112458 A1 | YFDYWGQGTLVTVSS | |
| α-IL12Rβ1 | SEQ ID NO: 30 of | QVOLKESGPALVKPTQTLTLTCTFSGFSLSTSGMG | 62 |
| antibody VH | PCT Publication | VSWIRQPPGKALEWLALIDWTDDKYYSTSLKTRLT | |
| sequence | No. WO | ISKDTSKNQVVLTMTNMDPVDTATYYCARTVGKGL | |
| | 2010/112458 A1 | YRVDNWGQGTLVTVSS | |
| α-IL12Rβ1 | SEQ ID NO: 31 of | QVQLVESGGGLVQPGGSLRLSCAASGFTFSSYGMS | 63 |
| antibody VH | PCT Publication | WVRQAPGKGLEWVSYIEPKLFWYATFYAASVKGRF | |
| sequence | No. WO | TISRDNSKNTLYLQMNSLRAEDTAVYYCARNDFME | |
| | 2010/112458 A1 | PAYFALWGQGTLVTVSS | |
| α-IL12Rβ1 | SEQ ID NO: 32 of | QVQLKESGPALVKPTQTLTLTCTFSGFSLSTRGVG | 64 |
| antibody VH | PCT Publication | VSWIRQPPGKALEWLALIYWDEDKYYSTSLKTRLT | |
| sequence | No. WO | ISKDTSKNQVVLTMTNMDPVDTATYYCARYQSGYY | |
| | 2010/112458 A1 | YNNDGWGVDIWGQGTLVTVSS | |
| α-IL12Rβ1 | SEQ ID NO: 25 of | DIALTQPASVSGSPGQSITISCTGTSSDLGESNYV | 65 |
| antibody | PCT Publication | SWYQQHPGKAPKVMIYDVNKRPSGVSNRFSGSKSG | |
| VL | No. WO | NTASLTISGLQAEDEADYYCGSYDEEDNVFGGGTK | |
| sequence | 2010/112458 A1 | LTVLGQ | |
| α-IL12Rβ1 | SEQ ID NO: 26 of | DIELTQPPSVSVAPGQTARISCSGDNLGSKFAYWY | 66 |
| antibody | PCT Publication | QQKPGQAPVLVIYDDSKRPSGIPERFSGSNSGNTA | |
| VL | No. WO | TLTISGTQAEDEADYYCQSWDSSSGNDVFGGGTKL | |
| sequence | 2010/112458 A1 | TVLGQ | |
| α-IL12Rβ1 | SEQ ID NO: 27 of | DIELTQPPSVSVAPGQTARISCSGDNLGSYYAYWY | 67 |
| antibody | PCT Publication | QQKPGQAPVGVIYDDSERPSGIPERFSGSNSGNTA | |
| VL | No. WO | TLTISGTQAEDEADYYCSSYTYSKNNVFGGGTKLT | |
| sequence | 2010/112458 A1 | VLGQ | |
| α-IL12Rβ1 | SEQ ID NO: 28 of | DIQMTQSPSSLSASVGDRVTITCRASQSISSWLNW | |
| antibody | PCT Publication | YQQKPGKAPKLLIYAASSLQSGVPSRFSGSGSGTD | |
| VL | No. WO | FTLTISSLQPEDFATYYCQQYYAFPHTFGQGTKVE | |
| sequence | 2010/112458 A1 | IKRT | |

In some aspects, the IL12Rβ1 targeting moiety competes with an antibody set forth in Table R1 for binding to IL12Rβ1. In further aspects, the IL12Rβ1 targeting moiety comprises CDRs having CDR sequences of an antibody set forth in Table R1. In some embodiments, the targeting moiety comprises all 6 CDR sequences of an antibody set forth in Table R1. In other embodiments, the IL12Rβ1 targeting moiety comprises at least the heavy chain CDR sequences (CDR-H1, CDR-H2, CDR-H3) of such antibody and the light chain CDR sequences of a universal light chain. In further aspects, an IL12Rβ1 targeting moiety comprises a VH comprising the amino acid sequence of the VH of an antibody set forth in Table R1. In some embodiments, the IL12Rβ1 targeting moiety further comprises a VL comprising the amino acid sequence of the VL of the antibody set forth above in Table R1. In other embodiments, the IL12Rβ1 targeting moiety further comprises a universal light chain VL sequence.

In some embodiments, the IL12Rβ1 targeting moiety is based on a single domain antibody. In some embodiments, the IL12Rβ1 targeting moieties are based on the exemplary anti-IL12Rβ1 single domain antibodies or antibody sequences set forth in Table R2 below.

TABLE R2
Exemplary Anti-IL12Rβ1 Single Domain Antibody (sdAb) Amino Acid Sequences

| Target or | | | SEQ |
|---|---|---|---|
| Description | Reference | Sequence | ID NO |
| hIL12Rb1_ | SEQ ID NO: 262 of | QVQLQESGGGSVQAGGSLRLSCVASGYGYCGYDMS | 69 |
| VHH1 | U.S. Publication | WYRQAPGKEREFVALITSDRSISYEDSVKARFIIS | |
| | No. US | RDNAANTGYLDMTRLTPDDTAIYYCKTSAAARESS | |
| | 2023/0279127 A1 | WCRSRYRVASWGQGTQVTVSS | |
| hIL12Rb1_ | SEQ ID NO: 263 of | QVQLQESGGGSVQAGGSLRLSCTASGYTYSSAFMA | 70 |
| VHH2 | U.S. Publication | WFRQAPGKEREGVAAIYTRDGGTVYADSVKGRFTI | |
| | No. US | SQDNAKNILYLQMNSLKAEDTAMYYCAAKIPQPGR | |
| | 2023/0279127 A1 | ASLLDSQTYDYWGQGTQVTVSS | |
| hIL12Rb1_ | SEQ ID NO: 264 of | QVQLQESGGGSVQAGGSLRLSCVASGYSYCGYDMM | 71 |
| VHH3 | U.S. Publication | WYRQAPGKEREFVALITSDYSIRYEDSVEGRFSIS | |
| | No. US | RDNAKNTGYLLMSNLTPADTAIYYCKTSTAARESS | |
| | 2023/0279127 A1 | WCRSRYRVASWGQGTQVTVSS | |
| hIL12Rb1_ | SEQ ID NO: 265 of | QVQLQESGGGSVQAGGSLRLSCVASGYGYCGYDMS | 72 |
| VHH4 | U.S. Publication | WYRQTPGKEREFVALITSDRIASYEDSVKGRFIIS | |
| | No. US | RDNAKNTGYLDMTRVTPDDTAIYYCKTSAAARENS | |
| | 2023/0279127 A1 | WCRSRYRVASWGQGTQVTVSS | |
| hIL12Rb1_ | SEQ ID NO: 266 of | QVQLQESGGGSVQAGGFLRLSCVASGYGYCGYDMS | 73 |
| VHH5 | U.S. Publication | WYRQVPGKEREFVALITSDRSVSYEDSVKGRFSIS | |
| | No. US | RDNAKNTAYLEMNRLTPDDTAVYYCKTSTAARENN | |
| | 2023/0279127 A1 | WCRSRYRIAYWGQGTQVTVSS | |
| hIL12Rb1_ | SEQ ID NO: 267 of | QVQLQESGGGSVQAGGSLRLSCAASRYTYTNNFMA | 74 |
| VHH6 | U.S. Publication | WFRQAPGKEREGVAAIYTGDGYAYYFYSVKGRFTI | |
| | No. US | SQDNDENMLYLQMNSLKPEDTAMYYCAAMERRIGT | |
| | 2023/0279127 A1 | RRMTENAEYKYWGQGTQVTVSS | |
| hIL12Rb1_ | SEQ ID NO: 268 of | QVQLQESGGGSVQAGGSLRLSCAVSGYDYCGYDVR | 75 |
| VHH7 | U.S. Publication | WYRRAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| hIL12Rb1_ | SEQ ID NO: 269 of | QVQLQESGGGSVQAGGSLRLSCVASGYSYCGYDMM | 71 |
| VHH8 | U.S. Publication | WYRQAPGKEREFVALITSDYSIRYEDSVEGRFSIS | |
| | No. US | RDNAKNTGYLLMSNLTPADTAIYYCKTSTAARESS | |
| | 2023/0279127 A1 | WCRSRYRVASWGQGTQVTVSS | |
| hIL12Rb1_ | SEQ ID NO: 270 of | QVQLQESGGGSVQAGGSLRLSCVASGYSYCGYDMM | 76 |
| VHH9 | U.S. Publication | WYRQAPGKEREFVALITSDYSIRYEDSVEGRFSIS | |
| | No. US | RDNAKNTGYLLMSNLTPADTAIYYCKTSTAARESG | |
| | 2023/0279127 A1 | WCRSRYRVASWGQGTQVTVSS | |
| hIL12Rb1_ | SEQ ID NO: 271 of | QVQLQESGGGSVQAGGSLRLSCAVSGYDYCGYDVR | 77 |
| VHH10 | U.S. Publication | WYRQAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| hIL12Rb1_ | SEQ ID NO: 272 of | QVQLQESGGGSVQAGGSLRLSCAVSGYDYCGYDVR | 77 |
| VHH11 | U.S. Publication | WYRQAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| hIL12Rb1_ | SEQ ID NO: 273 of | QVQLQESGGGSVQAGGSLRLSCTASGYTYSSAFMA | 78 |
| VHH12 | U.S. Publication | WFRQAPGKEREGVAAIYTRDGGTVYADSVKGRFTI | |
| | No. US | SQDNAKNTLYLQMNSLKPEDTAMYYCAAKMPQPGR | |
| | 2023/0279127 A1 | ASLLDSQTYDYWGQGTQVTVSS | |
| hIL12Rb1_ | SEQ ID NO: 274 of | QVQLQESGGGSVQAGGFLRLSCVASGYGYCGYDMS | 79 |
| VHH13 | U.S. Publication | WYRQAPGKEREFVALITSERVISYEDSVKGRFSIS | |
| | No. US | RDNAENTGYLEMNRLTPDDTAIYYCKTSAAARESS | |
| | 2023/0279127 A1 | WCRSRYRVASWGQGTQVTVSS | |
| hIL12Rb1_ | SEQ ID NO: 275 of | QVQLQESGGGSVQAGGSLRLSCAVSGYDYCGYDVR | 75 |
| VHH14 | U.S. Publication | WYRRAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| hIL12Rb1_ | SEQ ID NO: 276 of | QVQLQESGGGSVQAGGSLRLSCAVSGYDYCGYDVR | 80 |
| VHH15 | U.S. Publication | WYRQAPGKEREFVSGINSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| hIL12Rb1_ | SEQ ID NO: 277 of | QVQLQESGGGSVQAGGSLRLSCTASGYTYSSAFMA | 81 |
| VHH16 | U.S. Publication | WFRQAPGKEREGVAAMYTRDGGTVYADSVKGRFTI | |
| | No. US | SQDNAKNTLYLQIHTLKAEDTAMYYCAAKIPQPGR | |
| | 2023/0279127 A1 | ASLLDSQTYDYWGQGTQVTVSS | |
| hIL12Rb1_ | SEQ ID NO: 278 of | QVQLQESGGGSVQAGGFLRLSCVASGYGYCGYDMS | 82 |
| VHH17 | U.S. Publication | WYRQVPGKEREFVALITSDRSVSYEDSVKGRFSIS | |
| | No. US | RDNAKNTAYLEMNRLTPDDTAIYYCKTSTAARENN | |
| | 2023/0279127 A1 | WCRSRYRIASWGQGTQVTVSS | |
| hIL12Rb1_ | SEQ ID NO: 279 of | QVQLQESGGGSVQAGGSLRLSCAASRYTYTNNFMA | 83 |
| VHH18 | U.S. Publication | WFRQAPGKEREGVAAIYTGDGYAYYFDSVKGRFTI | |
| | No. US | SQDNDKNMLYLQMNSLKPEDTAMYYCAAMERRSGR | |
| | 2023/0279127 A1 | RRMTENAEYKYWGQGTQVTVSS | |
| hIL12Rb1_ | SEQ ID NO: 280 of | QVQLQESGGGSVQAGGSLRLSCAVSGYDYCGYDVR | 84 |
| VHH19 | U.S. Publication | WYRQAPGKEREFVSGINSDGSTSYADSVKGRFTIS | |
| | No. US | QDNAENTSYLHMFSLKPEDTAMYYCKTEGPAGESA | |
| | 2023/0279127 A1 | WCRNFRGMDYWGKGTQVTVSS | |
| hIL12Rb1_ | SEQ ID NO: 281 of | QVQLQESGGGSVQAGGSLRLSCTASGYTYSSAFMA | 85 |
| VHH20 | U.S. Publication | WFRQAPGKEREGVAAIYTRDGSPVYADSLKGRFTI | |
| | No. US | SQDNAKNTLHLQMNSLKPEDTAMYYCAAKIPEPGR | |
| | 2023/0279127 A1 | ISLLDSQTYDYWGHGTQVTVSS | |
| hIL12Rb1_ | SEQ ID NO: 282 of | QVQLQESGGGSVQAGGSLRLSCTASGYTYSSAFMA | 86 |
| VHH21 | U.S. Publication | WFRQAPGKEREGVAAMYTRDGGTVYADSVKGRFTI | |
| | No. US | SQDNAKNTLYLQMNSLKTEDTAMYYCAAKIPQPGR | |
| | 2023/0279127 A1 | ASLLDSQTYDYWGQGTQVTVSS | |
| hIL12Rb1_ | SEQ ID NO: 283 of | QVQLQESGGGSVQAGGSLRLSCTASGYTYSSAFMA | 87 |
| VHH22 | U.S. Publication | WFRQAPGKEREGVAAIYTRDGGTVYADSVKGRFTI | |
| | No. US | SQDNAKNTLYLQMNSLKAEDTAMYYCAAKIPQPGR | |
| | 2023/0279127 A1 | ASLLDSQTYDYWGQGTQVTVSS | |
| mIL12Rb1_ | SEQ ID NO: 284 of | QVQLQESGGGSVQAGGSLRLSCTASGYTYSSAFMA | 87 |
| VHH1 | U.S. Publication | WFRQAPGKEREGVAAIYTRDGGTVYADSVKGRFTI | |
| | No. US | SQDNAKNTLYLQMNSLKAEDTAMYYCAAKIPQPGR | |
| | 2023/0279127 A1 | ASLLDSQTYDYWGQGTQVTVSS | |
| mIL12Rb1_ | SEQ ID NO: 285 of | QVQLQESGGGSVQAGGSLRLSCAVSGYDYCGYDVR | 77 |
| VHH2 | U.S. Publication | WYRQAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| mIL12b1_ | SEQ ID NO: 286 of | QVQLQESGGGSVQAGGSLRLSCVASGYSYCGYDMM | 71 |
| VHH3 | U.S. Publication | WYRQAPGKEREFVALITSDYSIRYEDSVEGRFSIS | |
| | No. US | RDNAKNTGYLLMSNLTPADTAIYYCKTSTAARESS | |
| | 2023/0279127 A1 | WCRSRYRVASWGQGTQVTVSS | |
| mIL12Rb1_ | SEQ ID NO: 287 of | QVQLQESGGGSVQAGGSLRLSCAASRYTYTNNFMA | 83 |
| VHH4 | U.S. Publication | WFRQAPGKEREGVAAIYTGDGYAYYFDSVKGRFTI | |
| | No. US | SQDNDKNMLYLQMNSLKPEDTAMYYCAAMERRSGR | |
| | 2023/0279127 A1 | RRMTENAEYKYWGQGTQVTVSS | |
| mIL12Rb1_ | SEQ ID NO: 288 of | QVQLQESGGGSVQAGETLRLSCTVSGFTIDDSEMG | 88 |
| VHH5 | U.S. Publication | WYRQAPGHECELVASGSSDDDTYYVDSVKGRFTIS | |
| | No. US | LDNAKNMVYLQMNSLKPEDTAVYYCATGPTYPPKD | |
| | 2023/0279127 A1 | GDCAHWGQGTQVTVSS | |
| mIL12Rb1_ | SEQ ID NO: 289 of | QVQLQESGGGSVQAGGSLRLSCTASGYTYSSAFMA | 85 |
| VHH6 | U.S. Publication | WFRQAPGKEREGVAAIYTRDGSPVYADSLKGRFTI | |
| | No. US | SQDNAKNTLHLQMNSLKPEDTAMYYCAAKIPEPGR | |
| | 2023/0279127 A1 | ISLLDSQTYDYWGHGTQVTVSS | |
| mIL12Rb1_ | SEQ ID NO: 290 of | QVQLQESGGGSVQAGGSLRLSCAVSGYDYCGYDVR | 75 |
| VHH7 | U.S. Publication | WYRRAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| mIL12Rb1_ | SEQ ID NO: 291 of | QVQLQESGGGSVQAGGFLRLSCVASGYGYCGYDMS | 79 |
| VHH8 | U.S. Publication | WYRQAPGKEREFVALITSERVISYEDSVKGRFSIS | |
| | No. US | RDNAENTGYLEMNRLTPDDTAIYYCKTSAAARESS | |
| | 2023/0279127 A1 | WCRSRYRVASWGQGTQVTVSS | |
| mIL12Rb1_ | SEQ ID NO: 292 of | QVQLQESGGGSVQAGGSLRLSCVASGYGYCGYDMS | 69 |
| VHH9 | U.S. Publication | WYRQAPGKEREFVALITSDRSISYEDSVKARFIIS | |
| | No. US | RDNAANTGYLDMTRLTPDDTAIYYCKTSAAARESS | |
| | 2023/0279127 A1 | WCRSRYRVASWGQGTQVTVSS | |
| mIL12Rb1_ | SEQ ID NO: 293 of | QVQLQESGGGSVQAGGSLRLSCAVSGYDYCGYDVR | 77 |
| VHH10 | U.S. Publication | WYRQAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| mIL12Rb1_ | SEQ ID NO: 294 of | QVQLQESGGGSVQAGGSLRLSCVASGYSYCGYDMM | 71 |
| VHH11 | U.S. Publication | WYRQAPGKEREFVALITSDYSIRYEDSVEGRFSIS | |
| | No. US | RDNAKNTGYLLMSNLTPADTAIYYCKTSTAARESS | |
| | 2023/0279127 A1 | WCRSRYRVASWGQGTQVTVSS | |
| mIL12Rb1_ | SEQ ID NO: 295 of | QVQLQESGGGSVQAGGSLRLSCAASRYTYTNNFMA | 74 |
| VHH12 | U.S. Publication | WFRQAPGKEREGVAAIYTGDGYAYYFYSVKGRFTI | |
| | No. US | SQDNDENMLYLQMNSLKPEDTAMYYCAAMERRIGT | |
| | 2023/0279127 A1 | RRMTENAEYKYWGQGTQVTVSS | |
| mIL12Rb1_ | SEQ ID NO: 296 of | QVQLQESGGGSVQAGGSLRLSCVASGYSYCGYDMM | 76 |
| VHH13 | U.S. Publication | WYRQAPGKEREFVALITSDYSIRYEDSVEGRFSIS | |
| | No. US | RDNAKNTGYLLMSNLTPADTAIYYCKTSTAARESG | |
| | 2023/0279127 A1 | WCRSRYRVASWGQGTQVTVSS | |
| mIL12Rb1_ | SEQ ID NO: 297 of | QVQLQESGGGSVQAGGSLRLSCAVSGYDYCGYDVR | 80 |
| VHH14 | U.S. Publication | WYRQAPGKEREFVSGINSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| mIL12Rb1_ | SEQ ID NO: 298 of | QVQLQESGGGSVQAGGFLRLSCVASGYGYCGYDMS | 73 |
| VHH15 | U.S. Publication | WYRQVPGKEREFVALITSDRSVSYEDSVKGRFSIS | |
| | No. US | RDNAKNTAYLEMNRLTPDDTAVYYCKTSTAARENN | |
| | 2023/0279127 A1 | WCRSRYRIAYWGQGTQVTVSS | |
| mIL12Rb1_ | SEQ ID NO: 299 of | QVQLQESGGGSVQAGGSLRLSCAVSGYDYCGYDVR | 84 |
| VHH16 | U.S. Publication | WYRQAPGKEREFVSGINSDGSTSYADSVKGRFTIS | |
| | No. US | QDNAENTSYLHMFSLKPEDTAMYYCKTEGPAGESA | |
| | 2023/0279127 A1 | WCRNFRGMDYWGKGTQVTVSS | |
| mIL12Rb1_ | SEQ ID NO: 300 of | QVQLQESGGGSVQAGGSLRLSCTASGYTYSSAFMA | 81 |
| VHH17 | U.S. Publication | WFRQAPGKEREGVAAMYTRDGGTVYADSVKGRFTI | |
| | No. US | SQDNAKNTLYLQIHTLKAEDTAMYYCAAKIPQPGR | |
| | 2023/0279127 A1 | ASLLDSQTYDYWGQGTQVTVSS | |
| mIL12Rb1_ | SEQ ID NO: 301 of | QVQLQESGGGSVQAGGSLRLSCTASGYTYSSAFMA | 86 |
| VHH18 | U.S. Publication | WFRQAPGKEREGVAAMYTRDGGTVYADSVKGRFTI | |
| | No. US | SQDNAKNTLYLQMNSLKTEDTAMYYCAAKIPQPGR | |
| | 2023/0279127 A1 | ASLLDSQTYDYWGQGTQVTVSS | |
| mIL12Rb1_ | SEQ ID NO: 302 of | QVQLQESGGGSVQAGGFLRLSCVASGYGYCGYDMS | 82 |
| VHH19 | U.S. Publication | WYRQVPGKEREFVALITSDRSVSYEDSVKGRFSIS | |
| | No. US | RDNAKNTAYLEMNRLTPDDTAIYYCKTSTAARENN | |
| | 2023/0279127 A1 | WCRSRYRIASWGQGTQVTVSS | |
| mIL12Rb1_ | SEQ ID NO: 303 of | QVQLQESGGGSVQAGGSLRLSCVASGYGYCGYDMS | 72 |
| VHH20 | U.S. Publication | WYRQTPGKEREFVALITSDRIASYEDSVKGRFIIS | |
| | No. US | RDNAKNTGYLDMTRVTPDDTAIYYCKTSAAARENS | |
| | 2023/0279127 A1 | WCRSRYRVASWGQGTQVTVSS | |
| mIL12Rb1_ | SEQ ID NO: 304 of | QVQLQESGGGSVQAGGSLRLSCAVSGYDYCGYDVR | 75 |
| VHH21 | U.S. Publication | WYRRAPGKEREFVSGIDSDGSTSYADSVKGRFTIS | |
| | No. US | QDNAENTSYLHMFSLKPEDTAMYYCKTESPAGESA | |
| | 2023/0279127 A1 | WCRNFRGMDYWGKGTQVTVSS | |
| mIL12Rb1_ | SEQ ID NO: 305 of | QVQLQESGGGSVQAGGSLRLSCTASGYTYSSAFMA | 70 |
| VHH22 | U.S. Publication | WFRQAPGKEREGVAAIYTRDGGTVYADSVKGRFTI | |
| | No. US | SQDNAKNILYLQMNSLKAEDTAMYYCAAKIPQPGR | |
| | 2023/0279127 A1 | ASLLDSQTYDYWGQGTQVTVSS | |
| mIL12Rb1_ | SEQ ID NO: 306 of | QVQLQESGGGSVQAGGSLRLSCTASGYTYSSAFMA | 78 |
| VHH23 | U.S. Publication | WFRQAPGKEREGVAAIYTRDGGTVYADSVKGRFTI | |
| | No. US | SQDNAKNTLYLQMNSLKPEDTAMYYCAAKMPQPGR | |
| | 2023/0279127 A1 | ASLLDSQTYDYWGQGTQVTVSS | |
| PMPIL12RB1- | SEQ ID NO: 2077 | EVOLVESGGGLVQAGGSLRLSCAVSGIAFRYNSVA | 89 |
| 134-5A | of PCT Publication | WSRQAPGSQRELVARITNSARTNYADSVKGRFTIS | |
| | No. WO | RDNDKNMVYLQMNSLKPEDTAVYYCGAGRSMTGDV | |
| | 2009/068627 A2 | AYWGQGTQVTVSS | |
| PMPIL12RB1- | SEQ ID NO: 2078 | EVOLVESGGGLVQAGGSLRLSCAASGITFDDDYAI | 90 |
| 134-5B | of PCT Publication | GWFRQAPGKEREGVSLISSSDGSTYYADSVKGRFT | |
| | No. WO | ITSDNAKNTVYLQMNSLKPEDTAVYYCAADPTSGL | |
| | 2009/068627 A2 | PSDDEYDYWGQGTQVTVSS | |
| PMPIL12RB1- | SEQ ID NO: 2079 | EVOLVESGGGLVQAGGSLRLSCAASGIAFRYNSVA | 91 |
| 134-5C | of PCT Publication | WSRQAPGSQREVVAGITNSARTNYADSVKGRFTIS | |
| | No. WO | RDNDKKMVYLQMNSLKPEDTAVYYCGAGRSMAGDV | |
| | 2009/068627 A2 | AYWGQGTQVTVSS | |
| PMPIL12RB1- | SEQ ID NO: 2080 | EVOLVESGGGLVQAGGSLRLSCAASGIAFRYNSVA | 92 |
| 134-5E | of PCT Publication | WSRQAPGSQRELVASISNSARTKYADSVKGRFTIS | |
| | No. WO | RDNEKKMLYLQMDSLKPEDTAVYYCGAGRSMAGDV | |
| | 2009/068627 A2 | AYWGQGTQVTVSS | |
| PMPIL12RB1- | SEQ ID NO: 2081 | EVOLVESGGGLVQPGGSLRLSCVASGRPLTGSTMA | 93 |
| 134-5F | of PCT Publication | WFRQAPGKECEFVARISGSGTINYADSLRGRFTIS | |
| | No. WO | RDVPKNTVWLQMDSLKPDDTAVYYCAAVKVAGSYE | |
| | 2009/068627 A2 | YWGQGTQVTVSS | |
| PMPIL12RB | SEQ ID NO: 2082 | EVOLVESGGGSVQTGGSLTLSCTASGRTGSTDGVG | 94 |
| 1-134-5G | of PCT Publication | WFRQAPGKEREFVSAIKWIGGSKYYSNSAEGRFTI | |
| | No. WO | AVDNAKNTVYLQMNSLNPEDTAVYYCAAGAIMFPS | |
| | 2009/068627 A2 | RPRDFDFWGQGTQVTVSS | |
| PMPIL12RB1- | SEQ ID NO: 2083 | EVOLVESGGGLVQAGGSLRLSCAASGRTFSSYAMG | 95 |
| 134-8B | of PCT Publication | WFRQAPGKEREFVAHINWNGGNTYYADSVRGRFII | |
| | No. WO | SRDNAKNTLYLQMNRLKPEDTAVYYCAARPDRVIV | |
| | 2009/068627 A2 | KWDEYDYWGQGTQVTVSS | |
| PMPIL12RB1- | SEQ ID NO: 2084 | EVOLVESGGGLVQPGESLTLSCAASGFTFSSYWMY | 96 |
| 134-58D | of PCT Publication | WVRQAPGRELEWVARIQPGSSYTAYADSVKGRFTI | |
| | No. WO | SRDLAKNTLYLQMNRLKSDDTAVYYCAKDWEMAAP | |
| | 2009/068627 A2 | SLGQGTQVTVSS | |
| PMPIL12RB1- | SEQ ID NO: 2085 | EVOLVESGGGLVQAGGSLRLSCAASGTFLSINRMG | 97 |
| 134-9B | of PCT Publication | WYRQAPGKERELVAVIISGGSTNYADSVKGRFTIS | |
| | No. WO | RENAELTVYLQMNSLKPEDTAVYYCNVWINTDGIY | |
| | 2009/068627 A2 | TYEYWGQGTQVTVSS | |
| PMPIL12RB1- | SEQ ID NO: 2086 | EVOLVESGGGSVQPGGSLRLSCAASGLIVGRPAMG | 98 |
| 134-9C | of PCT Publication | WYRQAPGKQRELVAIIGSGGNTNYPESVKGRFTIA | |
| | No. WO | RENANNTVYLQMNSLKPEDTAVYHCNLHGTRFWGQ | |
| | 2009/068627 A2 | GTQVTVSS | |
| PMPIL12RB1- | SEQ ID NO: 2087 | EVOLVESGGGLVQAGGSLRLSCAASGLTFSSPAMA | 99 |
| 148-2D | of PCT Publication | WFRQVPGKEREFVATIRRSSAWTDYADSVKGRFTI | |
| | No. WO | SRDRPTNTAYLQMSSLKPEDTAVYYCAADKISRGI | |
| | 2009/068627 A2 | DPNWTYWGRGTQVTVSS | |
| PMPIL12RB1- | SEQ ID NO: 2088 | EVOLVESGGGLVQPGGSLRLSCAVSGISVRSSVVN | 100 |
| 148-2F | of PCT Publication | WYRQAPGKQRELVALIMGGAIRKYADDVKGRFTIS | |
| | No. WO | SDSAKNTVYLQMNSLRPEDTAVYYCSALEYWGQGT | |
| | 2009/068627 A2 | QVTVSS | |
| PMPIL12RB1- | SEQ ID NO: 2089 | EVOLVESGGGLVQPGGSLRLSCAVSGISVRSSVVN | 10 |
| 148-2G | of PCT Publication | WYRQAPGKQRELVALIMGGAITKYADDVKGRFTIS | |
| | No. WO | SDSARNTVYLQMNSLRPEDTAVYSCSALEYWGQGT | |
| | 2009/068627 A2 | QVTVSS | |
| PMPIL12RB1- | SEQ ID NO: 2090 | EVOLVESGGGLAQEGGSLRLSCAASGRPLTTYGMA | 102 |
| 148-3C | of PCT Publication | WFRQAPGKEREFVARIGAEPGATVYGDSVKGRFTI | |
| | No. WO | SRDNAKNTVYLQMNTLKPEDTAVYYCAADAPPFGP | |
| | 2009/068627 A2 | YYRESVYDYWGQGTQVTVSS | |
| PMPIL12RB | SEQ ID NO: 2091 | EVOLVESGGGLVTAGGSLRLSCAASGFRFSVYDMG | 103 |
| 1-148-3F | of PCT Publication | WFRQAPGKEREFVAVIVGSRTTDYADSVKGRFIIF | |
| | No. WO | RDNAKNTLYLQMNGLKPDDTAVYYCARRVGTYETV | |
| | 2009/068627 A2 | LGYDKWGQGTQVTVSS | |
| PMPIL12RB | SEQ ID NO: 2092 | EVOLVESGGGLVQAGGSLRLSCAASGFTEDDYAIG | 104 |
| 1-148-5D | of PCT Publication | WFRQAPGKEREGVSCISSSDGSTYYADSVKGRFTI | |
| | No. WO | SSDNAKNTVYLQMNSLKPEDTAVYYCAAPLLRGGC | |
| | 2009/068627 A2 | PITYYSGSYPHVYHAMDYWGKGTLVTVSS | |
| PMPIL12RB | SEQ ID NO: 2093 | EVOLVESGGGLVQPGGSLRLSCAASGFTFSRAWMY | 105 |
| 1-148-7A | of PCT Publication | WVRQAPGETLEWVSRIQPGGGSTSYADSVKGRFTI | |
| | No. WO | SRDNAKNTLYLQMNSLKSEDTAVYYCAKDWEMAAP | |
| | 2009/068627 A2 | SLGQGTLVTVSS | |
| PMPIL12RB1- | SEQ ID NO: 2094 | EVOLVESGGGLVQPGGSLRLSCKASRSIFSINTMD | 106 |
| 148-7B | of PCT Publication | WHRQVPGKQRELVAAIVNGVWKNYADSVKGRFTIS | |
| | No. WO | RDNAENTVYLQMNNLKPEDTAVYYCHAKRGVSDYW | |
| | 2009/068627 A2 | GQGTQVTVSS | |
| PMPIL12RB1- | SEQ ID NO: 2095 | EVXLVESGGGLVETGGSLRLSCAARGRIKSIADMG | 107 |
| 134-11G | of PCT Publication | WYRQAPGKQRELVATITFGGTTTYADSAKGRFTIS | |
| | No. WO | RDNAENTVYLQMNSLKPEDTAVYFCNADRILYVSE | |
| | 2009/068627 A2 | GLYRTEVDSWGQGTQVTVSS | |
| PMPIL12RB1- | SEQ ID NO: 2096 | EVOLVESGGGLVQPGGSLRLSCAASGFTFSRAWMY | 108 |
| 134-8A | of PCT Publication | WVRQAPGETLEWVSRIQPGGGSTSYADSVKGRFTI | |
| | No. WO | SRDNAKNTLYLQMNSLKSEDTAVYYCAKDWEMAAP | |
| | 2009/068627 A2 | SLGQGTQVTVSS | |
| PMPIL12RB1- | SEQ ID NO: 2097 | EVQLMESGGGVVQVGGSLRLSCAASGGTLYSYIVG | 109 |
| 134-4C | of PCT Publication | WFSQAPGQDREFVGAIEYSGGITDYKDSVKGRFTI | |
| | No. WO | SKDNPKNTVFLQMDSLKPEDTAVYYCGLTRVVGAR | |
| | 2009/068627 A2 | NPGDYAYWGQGTQVTVSS | |
| PMPIL12RB1- | SEQ ID NO: 2098 | EVOLVESGGGLAQTGGSLTLSCAASGRTPSIVAMG | 110 |
| 134-8C | of PCT Publication | WFRQIPGKDREPVGEIILSKGFTYYADSVKGRFTI | |
| | No. WO | SRANAKNTITMSLQMHSLKSEDTAVYYCAARQNWS | |
| | 2009/068627 A2 | GNPTRPNEYEYWGQGTQVTVSS | |
| PMPIL12RB1- | SEQ ID NO: 2099 | EVOLVESGGGLVQTGGSLRLSCAASGRTSRLVAMG | 111 |
| 148-10E | of PCT Publication | WFRQTPGKEREFVGEIILSKDFTYYADSVKGRFTI | |
| | No. WO | SRANAKNTITMYLQMSSLKSEDTAVYYCAARQNWS | |
| | 2009/068627 A2 | GHPARTNEYEYWGQGTQVTVSS | |
| PMPIL12RB1- | SEQ ID NO: 2100 | EVOLVESGGGLVQTGGSLRLSCAASGRTPSIVAMG | 112 |
| 148-11F | of PCT Publication | WFRQTPGKEREFVGEIILSKGFTYYADSVKGRFTI | |
| | No. WO | SRANAKNTITMYLQMHSLKSEDSAVYYCAARONWS | |
| | 2009/068627 A2 | GGPTRTNEYEYWGQGTQVTVSS | |
| PMPIL12RB1- | SEQ ID NO: 2101 | EVOLVESGGGLVQTGGSLRLSCAASGRTPSIVAMG | 113 |
| 148-12D | of PCT Publication | WFRQTPGKERESVGEIILSKGFTYYADSVKGRFTI | |
| | No. WO | SRANAKNTITMYLQMNSLKSEDTAVYYCAARQNWS | |
| | 2009/068627 A2 | GNPTRTNEYEYWGQGTQVTVSS | |
| PMPIL12RB1- | SEQ ID NO: 2102 | EVOLVESGGGLVQTGGSLXLSCAASGRTPRLVAMG | 114 |
| 148-9C | of PCT Publication | WFRQTPGKEREFVGEIILSKGFTYYADSVKGRFTI | |
| | No. WO | SRVNAKNTITMYLQMNSLKSEDTAVYYCAGRONWS | |
| | 2009/068627 A2 | GSPARTNEYEYWGQGTQVTVSS | |
| PMPIL12RB1- | SEQ ID NO: 2103 | EVOLVESGGGLVQTGGSLRLSCAASGRTPSIIAMG | 115 |
| 148-9F | of PCT Publication | WFRQTPGKEREFVGEIILSKGFTYYADSVKGRFTI | |
| | No. WO | SRANAKNTITMYLQMNSLKSEDTAVYYCAARQNWS | |
| | 2009/068627 A2 | GNPTRTNEYEYWGQGTQVTVSS | |

In some aspects, the IL12Rβ1 targeting moiety competes with an antibody set forth above in Table R2 for binding to IL12Rβ1. In further aspects, the IL12Rβ1 targeting moiety comprises CDRs having CDR sequences of an antibody set forth in Table R2. In some embodiments, the IL12Rβ1 targeting moiety comprises all 3 CDR sequences of an antibody set forth in Table R2. In further aspects, an IL12Rβ2 targeting moiety comprises a VH (e.g., a VHH) comprising the amino acid sequence of the VH of an antibody set forth in Table R2.

In some embodiments, the IL12Rβ1 targeting moiety binds an epitope at similar proximity to cell membrane as the IL12Rβ2 targeting of the tumor-targeted split IL12 receptor agonist. In some embodiments, if the IL12Rβ2 targeting moiety binds to the D1 domain of IL12Rβ2, then the IL12Rβ1 targeting moiety binds to D2 domain of IL12Rβ1.

### 6.3.2.2. IL12Rβ1 Ligands

In some embodiments, the IL12Rβ1 binding domain of a tumor-targeted split IL12 receptor agonist is an IL12Rβ1 ligand. An IL12Rβ1 ligand refers to a variant IL12 moiety which has preferential binding to IL12Rβ1 vs. IL12Rβ2 as compared to wild-type IL12.

In some embodiments, the preferential binding to IL12Rβ1 is achieved through mutations in the p35 moiety that reduce its binding to IL12Rβ2, while maintaining or increasing the binding of the p40 moiety to IL12Rβ1.

In some embodiments, the IL12Rβ1 ligand comprises a p35 moiety whose amino acid sequence has at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ2 binding portion of a mammalian, e.g., human or murine, p35 (sometimes referred to as the alpha subunit of IL12 or IL12a). For example, the p35 moiety can comprise an amino acid having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO:3 or SEQ ID NO:5. In some embodiments, the p35 moiety is a variant p35 moiety having an amino acid comprising one or more mutations that reduce IL12Rβ32 binding as compared to the amino acid sequence of SEQ ID NO:3 or SEQ ID NO:5. For example, in some embodiments, the variant p35 moiety can have up to 1,000-fold attenuated binding to human IL12Rβ32 as compared to wild type human p35. In some embodiments, the variant p35 moiety can have up to 100-fold, up to 50-fold, up to 25-fold, up to 20-fold, up to 15-fold, up to 10-fold, or up to 5-fold attenuated binding to human L12Rβ2 as compared to wild type human IL12 p35.

Other characteristics of useful IL12 p35 variants may include the ability to destabilize dimerization with IL12 p40.

Exemplary amino acid substitutions include, but are not limited to substitutions at N21, Q35, E38, E45, D55, N71, L75, N76, E79, N85, L89, F96, M97, L124, M125, Q130, Q135, N136, E143, Q146, Y167, I171, and R189, wherein amino acid positions are relative to the mature human IL12 p35 amino acid sequence, excluding the 22-amino acid signal sequence. Corresponding amino acid positions in the full-length human sequence, full-length murine sequence, and mature murine sequence are provided in Table B1. Table B1 also provides exemplary substitutions at each noted positions.

TABLE B1
IL12 p35 moiety Amino Acid Substitutions

| Amino Acid | Amino Acid | Amino Acid | Amino Acid | |
|---|---|---|---|---|
| (Human - | (Human - | (Murine - | (Murine - | Exemplary |
| Full Length) | Mature) | Full Length) | Mature) | substitutions |
| N43 | N21 | N39 | N17 | D |
| Q57 | Q35 | E53 | E31 | D |
| E60 | E38 | K56 | K34 | Q |
| E67 | E45 | E63 | E41 | Q |
| D77 | D55 | D73 | D51 | Q, K |
| N93 | N71 | N89 | N67 | D |
| L97 | L75 | L93 | L71 | A |
| N98 | N76 | A94 | A72 | D |
| E101 | E79 | E97 | E75 | Q |
| N107 | N85 | R103 | R81 | D, Q |
| L111 | L89 | L107 | L85 | A |
| F118 | F96 | L114 | L92 | A |
| M119 | M97 | M115 | M93 | A |
| L146 | L124 | Q142 | Q120 | A |
| M147 | M125 | N143 | N121 | A |
| Q152 | Q130 | Q156 | Q126 | E |
| Q157 | Q135 | K153 | K131 | E |
| N158 | N136 | G154 | G132 | D |
| E165 | E143 | E161 | E139 | Q |
| Q168 | Q146 | Q164 | Q142 | E |
| Y189 | Y167 | Y185 | Y163 | A, V, R, E |
| I193 | I171 | M189 | M167 | A, V, E |
| R211 | R189 | R207 | R185 | A, K |

An exemplary amino acid substitution at mature human N21 is N21D.

An exemplary amino acid substitution at mature human Q35 is Q35D.

An exemplary amino acid substitution at mature human E38 is E38Q.

An exemplary amino acid substitution at mature human E45 is E45Q.

Exemplary amino acid substitutions at mature human D55 include D55Q and D55K.

An exemplary amino acid substitution at mature human N71 is N71D.

An exemplary amino acid substitution at mature human L75 is L75A.

An exemplary amino acid substitution at mature human N76 is N76D.

An exemplary amino acid substitution at mature human E79 is E79Q.

Exemplary amino acid substitutions at mature human N85 include N85D and N85Q.

An exemplary amino acid substitution at mature human L89 is L89A.

An exemplary amino acid substitution at mature human F96 is F96A.

An exemplary amino acid substitution at mature human M97 is M97A.

An exemplary amino acid substitution at mature human L124 is L124A.

An exemplary amino acid substitution at mature human M125 is M125A.

An exemplary amino acid substitution at mature human Q130 is Q130E.

An exemplary amino acid substitution at mature human Q135 is Q135E.

An exemplary amino acid substitution at mature human N136 is N136D.

An exemplary amino acid substitution at mature human E143 is E143Q.

An exemplary amino acid substitution at mature human Q146 is Q146E.

Exemplary amino acid substitutions at mature human Y167 include Y167A, Y167V, Y167R, and Y167E. In some embodiments, the substitution is Y167E (corresponding to Y189E in the context of full length p35). An exemplary variant p35 moiety with the substitution at Y167E in the context of mature human p35 or Y189E in the context of full length p35 is provided herein as SEQ ID NO:40. For convenience, this variant is sometimes referred to as a “Y189E” variant, wherein the position of the substitution is referred to in the context of the full length, rather than the mature, human p35 sequence.

Exemplary amino acid substitutions at mature human 1171 include 1171A, 1171V, and 1171E.

In certain embodiments, an amino acid substitution at mature human R189 destabilizes the p40/p35 heterodimer by preventing formation of a disulfide bond between the two subunits. Exemplary amino acid substitutions at mature human R189 include R189A and R189K.

In some embodiments, the IL12Rβ1 ligand comprises a p40 moiety whose amino acid sequence has at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ1 binding portion of a mammalian, e.g., human or murine, p40 (sometimes referred to as the beta subunit of IL12 or IL12P). For example, the p40 moiety can comprise an amino acid having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of any one of SEQ ID NO:6, SEQ ID NO:7, SEQ ID NO:9 and SEQ ID NO:10. In some embodiments, the p40 moiety comprises an amino acid having the amino acid sequence of any one of SEQ ID NO:6, SEQ ID NO:7, SEQ ID NO:9 or SEQ ID NO:10 and/or a variant of SEQ ID NO:6, SEQ ID NO:7, SEQ ID NO:9 or SEQ ID NO:10 that does not have reduced IL12Rβ1 binding as compared to the amino acid sequence of SEQ ID NO:6, SEQ ID NO:7, SEQ ID NO:9 or SEQ ID NO:10, respectively.

### 6.4. Tumor-Targeted IL12Rβ2 Agonist

### 6.4.1. Tumor-Associated Antigen Targeting Moieties

The tumor-targeted IL12Rβ2 agonist of the tumor-targeted split IL12 receptor agonists of the disclosure comprise a tumor-associated antigen (“TAA”) targeting moiety. Typically, the TAA recognized by the TAA targeting moiety of the tumor-targeted IL12Rβ2 agonist is expressed on the same cancer cell as the TAA recognized by the TAA targeting moiety of the tumor-targeted IL12Rβ1 agonist.

In some embodiments, both TAA targeting moieties recognize the same TAA, whether on the same epitope or on different epitopes. If the TAA targeting moieties recognize different epitopes, they preferably can bind to the cancer cell simultaneous and/or in a non-competing manner. TAA targeting moieties both expressed on the same cancer cell and may be the same TAA or different TAAs.

In some embodiments, both TAA targeting moieties recognize different TAAs expressed on the same cancer cell.

Suitable TAA targeting moieties are described in Section 6.5.

### 6.4.2. IL12Rβ2 Binding Moieties

### 6.4.2.1. IL12Rβ2 Targeting Moieties

In some embodiments, the tumor-targeted IL12Rβ2 agonist of the tumor-targeted split IL12 receptor agonists of the disclosure comprise an IL12Rβ2 targeting moiety as an IL12Rβ2 binding moiety.

The IL12Rβ2 targeting moiety typically is or comprises an antigen binding domain of an antibody. The IL12Rβ2 targeting moiety can be any format, e.g., as disclosed in Section 6.6 or subsections thereof. In some embodiments, the IL12Rβ2 targeting moiety is a Fab. In some embodiments, the IL12Rβ2 targeting moiety is an scFv. In some embodiments, the IL12Rβ2 targeting moiety is a sdAb.

In some embodiments, the IL12Rβ2 targeting moiety is based on an antibody comprising both heavy and light chain variable regions. Exemplary anti-IL12Rβ2 antibodies are set forth in Table R3 below.

TABLE R3
Exemplary Anti-IL12Rβ2 Antibody Variable Heavy
(VH) and Light (VL) Chain Amino Acid Sequences

| Target | Antibody Name and/or Binding Sequences |
|---|---|
| IL12Rβ2 | IL12Rβ2 Monoclonal Antibody (2H6) (ThermoFisher; Cat. No. H00003595- |
| | M02) |
| IL12Rβ2 | Mouse anti-human IL12Rβ2 monoclonal antibody (RayBiotech; Cat. No. 101- |
| | 10794) |
| IL12Rβ2 | Mouse anti-human IL12Rβ2 monoclonal antibody (US Biological; Cat. No. |
| | 128388) |
| IL12Rβ2 | PE anti-human mouse monoclonal anti-IL12Rβ2 antibody (BioLegend; Cat. No. |
| | 394205) |
| IL12Rβ2 | Mouse anti-IL12Rβ2 recombinant antibody (clone 26A9) (Creative Biolabs; |
| | Cat. No. MOB-2643z) |
| IL12Rβ2 | Anti-human monoclonal anti-IL12Rβ2 antibody (Miltenyi; Cat. No. 130-125- |
| | 974) |
| IL12Rβ2 | Monoclonal anti-IL12Rβ2 antibody (R&D Systems; Cat. No. MAB19591) |

In some aspects, the IL12Rβ2 targeting moiety competes with an antibody set forth in Table R3 for binding to IL12Rβ2. In further aspects, the IL12Rβ2 targeting moiety comprises CDRs having CDR sequences of an antibody set forth in Table R3. In some embodiments, the targeting moiety comprises all 6 CDR sequences of an antibody set forth in Table R3. In other embodiments, the targeting moiety comprises at least the heavy chain CDR sequences (CDR-H1, CDR-H2, CDR-H3) of such antibody and the light chain CDR sequences of a universal light chain. In further aspects, an IL12Rβ2 targeting moiety comprises a VH comprising the amino acid sequence of the VH of an antibody set forth in Table R3. In some embodiments, the IL12Rβ2 targeting moiety further comprises a VL comprising the amino acid sequence of the VL of an antibody set forth above in Table R3. In other embodiments, the IL12Rβ2 targeting moiety further comprises a universal light chain VL sequence.

In some embodiments, the IL12Rβ2 targeting moieties are based on the exemplary anti-IL12Rβ2 single domain antibodies or antibody sequences set forth in Table R4 below.

TABLE R4
Exemplary Anti-IL12R32 Single Domain Antibody (sdAb)
Amino Acid Sequences

| Target or | | | SEQ |
|---|---|---|---|
| Description | Reference | Sequence | ID NO |
| hIL12Rb2 | SEQ ID NO: 307 of | QVQLQESGGGSVQAGGSLRLSCAASGFTVTRYCMG | 116 |
| VHH1 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 308 of | QVQLQESGGGSVQAGGSLRLSCAASGFTISRYCMG | 117 |
| VHH2 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 309 of | QVQLQESGGGSVQAGGSLRLSCTASGLTFDDVEMA | 118 |
| VHH3 | U.S. Publication | WYRQGPGDDYDLVSSINTDSRVYYVDSVKDRFTIS | |
| | No. US | RDNAKNTLYLQMNNLKPEDTAVYYCAADPWGGDLR | |
| | 2023/0279127 A1 | GYPNYWGQGTQVTVSS | |
| hIL12Rb2 | SEQ ID NO: 310 of | QVQLQESGGGSVQAGGSLRLSCVASGFTISRYCMG | 119 |
| VHH4 | U.S. Publication | WLRQAPGKQREGVAIIERDGRTGYADSVKGRFTIS | |
| | No. US | KDNAKNTLYLQMNSLKPGDTAMYYCGAIEGSCRPD | |
| | 2023/0279127 A1 | FGYRGQGTQVTVSS | |
| hIL12Rb2 | SEQ ID NO: 311 of | QVQLQESGGGLVQPGGSLKLSCAASGFTFSTYAMS | 120 |
| VHH5 | U.S. Publication | WVRQAPGKEPEWISRISSGGGNTYYADAVKGRFAI | |
| | No. US | SRDNAKNTLYLQLNSLKTEDTAIYVCTMDDYYGGS | |
| | 2023/0279127 A1 | WHPISRGHGTQVTVSS | |
| hIL12Rb2 | SEQ ID NO: 312 of | QVQLQESGGGLVQAGGSLRLSCQASGYTYGLFCMG | 121 |
| VHH6 | U.S. Publication | WFRQVSGKKREGVAVVDSPGGRHVADSLKGRFTIS | |
| | No. US | KDNANNILYLDMTNLKSEDTATYYCAADPEKYCFL | |
| | 2023/0279127 A1 | FSDAGYQYWGQGTQVTVSS | |
| hIL12Rb2 | SEQ ID NO: 313 of | QVQLQESGGGSVQAGGSLRLSCAASGVTYSRYCMG | 122 |
| VHH7 | U.S. Publication | WFRQAPGLERERVATIYSRGIITYYTDSVKGRFTI | |
| | No. US | SQDSAKKTVYLQMNSLKPEDTAMYYCAATRETYGG | |
| | 2023/0279127 A1 | SGDCDYESVYNYWAQGTQVTVSS | |
| hIL12Rb2 | SEQ ID NO: 314 of | QVQLQESGGGSVQAGGSLRLSCAASGFTVSRYCMG | 123 |
| VHH8 | U.S. Publication | WLRQAPGKQREGVAIIEREGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 315 of | QVQLQESGGGSVQAGGSLRLSCAASGFTISRYCMG | 124 |
| VHH9 | U.S. Publication | WLRQAPGKQREGVAIIERDGRTGYADSVKGRFTIS | |
| | No. US | KDNAKNTLYLQMNSLKPEDTAMYFCGAIEGSCRPD | |
| | 2023/0279127 A1 | FGYRGQGTQVTVSS | |
| hIL12Rb2 | SEQ ID NO: 316 of | QVQLQESGGGSVQAGGSLRLSCAASGFTVTRYCMG | 116 |
| VHH10 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 317 of | QVQLQESGGGSVQAGGSLRLSCAASGFTVSRYCMG | 125 |
| VHH11 | U.S. Publication | WLRQAPGKQREGVAIIERDGRTGYADSVKGRFTIS | |
| | No. US | KDDAKNTLYLQMNSLKPEDTAMYYCGAIEGSCRPD | |
| | 2023/0279127 A1 | FGYRGQGTQVTVSS | |
| hIL12Rb2 | SEQ ID NO: 318 of | QVQLQESGGGSVQAGGSLRLSCAASGVTYSRYCMG | 126 |
| VHH12 | U.S. Publication | WFRQAPGLERERVATIYSRGIITYYTDSVKGRFTI | |
| | No. US | SQDSAKKTVYLQMNMLKPEDTAMYYCAATRETYGG | |
| | 2023/0279127 A1 | SGDCDYESVYNYWAQGTQVTVSS | |
| hIL12Rb2 | SEQ ID NO: 319 of | QVQLQESGGGSVQAGGSLRLSCAASGFTISKYCMG | 127 |
| VHH13 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 320 of | QVQLQESGGGSVQAGGSLRLSCAASGVTYSRYCMG | 128 |
| VHH14 | U.S. Publication | WFRQAPGLERERVAHIYSRGIITYYTDSVKGRFTI | |
| | No. US | SQDSAKKTVYLQMNSLKPEDTAMYYCAATRETYGG | |
| | 2023/0279127 A1 | SGDCGYESVYNYWAQGTQVTVSS | |
| hIL12Rb2 | SEQ ID NO: 321 of | QVQLQESGGGSVQAGGSLRLSCAASGFTISRYCMG | 129 |
| VHH15 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | LGYRGQGTQVTVSS | |
| hIL12Rb2 | SEQ ID NO: 322 of | QVQLQESGGGSVQAGGSLRLSCAASGVTYSRYCMG | 130 |
| VHH16 | U.S. Publication | WFRQAPGLERERVAHIYSRGIITYYTDSVKGRFTI | |
| | No. US | SQDSAKKTVYLQMNSLKPEDTAMYYCAATRETYGG | |
| | 2023/0279127 A1 | SGDCSYESVYNHWAQGTQVTVSS | |
| hIL12Rb2 | SEQ ID NO: 323 of | QVQLQESGGGSVQAGGSLRLSCAASGLTISRYCMG | 131 |
| VHH17 | U.S. Publication | WLRQAPGKQREGVAIIERDGRTGYADSVKGRFTIS | |
| | No. US | KDNAKNTLYLQMNSLKPEDTAMYYCGAIEGSCRPD | |
| | 2023/0279127 A1 | FGYRGQGTQVTVSS | |
| hIL12Rb2 | SEQ ID NO: 324 of | QVQLQESGGGSVQAGGSLRLSCSASGFTVDDFAMG | 132 |
| VHH18 | U.S. Publication | WYRQAPGNECELVSTISSGGSTYYADSVKGRFTIS | |
| | No. US | QDSAKNTVYLQMNSLKPEDTAVYYCAPSSVGCPLG | |
| | 2023/0279127 A1 | YWGQGTQVTVSS | |
| mIL12Rb2 | SEQ ID NO: 325 of | QVQLQESGGGSVQAGGSLRLSCAASGYTYSNRHMG | 133 |
| VHH1 | U.S. Publication | WFRQAPGKEREGVAAIYTGGGSTYYADSVKDRFTI | |
| | No. US | SQDNAKNTLYLQMNSLTPEDTAMYYCAADLTRWYS | |
| | 2023/0279127 A1 | GGWRDPRGYKYWGQGTQVTVS | |
| mIL12Rb2 | SEQ ID NO: 326 of | QVQLQESGGGSVQAGGSLRLSCAASGVTYGSYYMA | 134 |
| VHH2 | U.S. Publication | AWFRQAPGKEREGVASIYGGSDSTYYADSVLGRFT | |
| | No. US | ISQDNGKNTLYLQMNSLKPDDTAMYYCAAAPPGKW | |
| | 2023/0279127 A1 | FLKRLEGHNYSYWGQGTQVTVSS | |
| mIL12Rb2 | SEQ ID NO: 327 of | QVQLQESGGGSVQVGGSLRLSCAASGFTYSSSCLG | 135 |
| VHH3 | U.S. Publication | WFRQAPGKEREGVATIYPAGGNIFYADSVKGRFTI | |
| | No. US | SQDNAKNTVYLQMDSLKPEDTAMYYCAARGGQTWG | |
| | 2023/0279127 A1 | SGGNRCSLWLPAYNYWGQGTQVTVSS | |
| mIL12Rb2 | SEQ ID NO: 328 of | QVQLQESGGGSVQVGGSLRLSCAVSGKLYGGAWFR | 136 |
| VHH4 | U.S. Publication | QAQGKGREGVAAIWIGTGTTFYADSVKGRFTISRD | |
| | No. US | NAKNTVYLQMDGLKPEDTALYYCAADDRPGYRDPL | |
| | 2023/0279127 A1 | APVSYNHWGQGTQVTVSS | |
| mIL12Rb2 | SEQ ID NO: 329 of | QVQLQESGGGSVQAGGSLRLSCAASGITYRGVWMG | 137 |
| VHH5 | U.S. Publication | WFRQAPGKEREGVATIYTGSGHTYYADSVKGRFTI | |
| | No. US | SQDNAKNTVYLQMNSLKPEDTAMYYCAARTVGGTF | |
| | 2023/0279127 A1 | YTLAADSFNTWGQGTQVTVSS | |
| mIL12Rb2 | SEQ ID NO: 330 of | QVQLQESGGGSVQAGGSLRLSCAVSGKAYGGAWFR | 138 |
| VHH6 | U.S. Publication | QAQGKGREGVAAIWIGTGTTFYADSVKGRFTISRD | |
| | No. US | NAKNTVYLQMDGLKPEDTAVYYCAADDRPGYRDPL | |
| | 2023/0279127 A1 | APVSYNHWGQGTQVTVSS | |
| mIL12Rb2 | SEQ ID NO: 331 of | QVQLQESGGGSVQAGGSLKLSCAVSGNPYGGAWFR | 139 |
| VHH7 | U.S. Publication | QAQGKSREGVAAIWLGTGTTFYADSVKGRFTISRD | |
| | No. US | NAKNTVYVQIDGLKPEDTAMYYCAADDRPGYRDPL | |
| | 2023/0279127 A1 | APVSYNHWGQGTQVTVSS | |
| mIL12Rb2 | SEQ ID NO: 332 of | QVQLQESGGGSVQAGGSLRLSCVVSGKAYGGAWFR | 140 |
| VHH8 | U.S. Publication | QAQGKSREGVAAIWIGTGTTFYADSVKGRFTISRD | |
| | No. US | NAKNTVYLQMDGLKPEDTAMYYCAADDRPGYRDPL | |
| | 2023/0279127 A1 | APVSYNHWGQGTQVTVSS | |
| mIL12Rb2 | SEQ ID NO: 333 of | QVQLQESGGGSVQAGGSLTLSCVVSGKAFGGAWFR | 141 |
| VHH9 | U.S. Publication | QAQGKGREGVAAIWIGTGTTFYADSVKGRFTISRD | |
| | No. US | NAKNTVYLQMDGLKPDDTAMYYCAADDRPGYRDPL | |
| | 2023/0279127 A1 | APVSYNHWGQGTQVTVSS | |
| mIL12Rb2 | SEQ ID NO: 334 of | QVQLQESGGGSVQAGGSLRLSCAASGYTFSNHHMG | 142 |
| VHH10 | U.S. Publication | WFRQAPGKEREGVAAIYTGAGNIYYADSVKDRFTI | |
| | No. US | SKDTAKNTLYLQMNSLTPEDTGMYYCAADLTRWYS | |
| | 2023/0279127 A1 | GGWRDPRGYKYWGQGTQVTVSS | |
| mIL12Rb2 | SEQ ID NO: 335 of | QVQLQESGGGPVQAGGSLRLSCAASGYTFSNHHMG | 143 |
| VHH11 | U.S. Publication | WFRQAPGKEREGVAAIYTGAGNIYYADSVKDRFTI | |
| | No. US | SKDTAKNTLYLQMNSLTPEDTGMYYCAADLTRWYS | |
| | 2023/0279127 A1 | GGWRDPRGYKYWGQGTQVTVSS | |
| mIL12Rb2 | SEQ ID NO: 336 of | QVQLQESGGGVVQPGGSLRLSCAASGYTFSNHHMG | 144 |
| VHH12 | U.S. Publication | WFRQAPGKEREGVAAIYTGAGNIYYADSVKDRFTI | |
| | No. US | SKDTAKNTLYLQMNSLTPEDTGMYYCAADLTRWYS | |
| | 2023/0279127 A1 | GGWRDPRGYKYWGQGTQVTVSS | |
| mIL12Rb2 | SEQ ID NO: 337 of | QVQLQESGGGSVQAGGSLRLSCAVSGYTFSNHHMG | 145 |
| VHH13 | U.S. Publication | WFRQAPGKEREGVAAIYTGAGNIYYADSVKDRFTI | |
| | No. US | SKDTAKNTLYLQMNSLTPEDTGMYYCAADLTRWYS | |
| | 2023/0279127 A1 | GGWRDPRGYKYWGQGTQVTVSS | |
| mIL12Rb2 | SEQ ID NO: 338 of | QVQLQESGGGSVQAGGSLRLSCAASGATNSNRHMG | 146 |
| VHH14 | U.S. Publication | WFRQAPGKEREGVAAIYTGYTGGGNTYYADSVRDR | |
| | No. US | FTISQDNAKNTLYLQMNSLTPEDTAMYYCAADLTR | |
| | 2023/0279127 A1 | WYSGGWRDPRGYKYWGQGTQVTVSS | |
| mIL12Rb2 | SEQ ID NO: 339 of | QVQLQESGGGSVQDGGSLRLSCAASGDIYARNCMG | 147 |
| VHH15 | U.S. Publication | WFRQAPGKEREKIAVADTGGRSPYYADSVKGRFTI | |
| | No. US | SRDNAKNTVDLQMNSLKPEDTAVYYCAAGPLVPVV | |
| | 2023/0279127 A1 | NTAARCVYEYWGQGTQVTVSS | |
| mIL12Rb2 | SEQ ID NO: 340 of | QVQLQESGGGSVQAGGSLRLSCAASGATNSNRHMG | 148 |
| VHH16 | U.S. Publication | WFRQAPGKEREGVAAIYTGYTGGGNTYYADSVKDR | |
| | No. US | FTISQDNAKNTLYLQMNSLTPEDTAMYYCAADLTR | |
| | 2023/0279127 A1 | WYSGGWRDPRGYKYWGQGTQVTVSS | |
| PMPIL12RB | SEQ ID NO: 2104 | EVQLVESGGGLVQTGGSLRLSCTHSGRPLSMFAVG | 149 |
| 2-135-10B | of PCT Publication | WFRQAPGEQRVLVAAISRSGGATYYPDSVKGRFAI | |
| | No. WO | SRDNAKNTVNLQMNSLTPEDTAVYYCAAALGGVYN | |
| | 2009/068627 A2 | AIANDYKYWGQGTQVTVSS | |
| PMPIL12RB | SEQ ID NO: 2105 | EVQLVESGGGSVQAGGSLRLSCAASGRTFSDYAMG | 150 |
| 2-135-10C | of PCT Publication | WFRQAPGKEREFVAGISRSGSTTGYAGSVKGRFSI | |
| | No. WO | SRDNAKNAVYLQMNSLKPEDTAVYYCASKRSPYTG | |
| | 2009/068627 A2 | IYYSDLSQSEDWGQGTQVTVSS | |
| PMPIL12RB | SEQ ID NO: 2106 | EVQLVESGGGLVQAGGSLRLSCAASGFAFDDYEIG | 151 |
| 2-135-12A | of PCT Publication | WFRQAPGKEREGVACINNSDGSTYYIDSVKGRFTI | |
| | No. WO | SKDSATNTVYLQMSSLKPEDTAVYYCAADSWCTVV | |
| | 2009/068627 A2 | AGKIHPFDSWGQGTQVTVSS | |
| PMPIL12RB | SEQ ID NO: 2107 | EVQLVESGGGLVQAGGSLRLSCAASGRTSSTYVMG | 152 |
| 2-135-12B | of PCT Publication | WFRQAPGKEREFVAGISWSAGNTRYADSVKGRFTI | |
| | No. WO | SRDIAKNTFDLQMNSLKPEDTAVYYCAADRRYGGS | |
| | 2009/068627 A2 | LDPSAWDFWGQGTQVTVSS | |
| PMPIL12RB | SEQ ID NO: 2108 | EVQLVESGGGSVQAGGSLRLSCAASGRTFSSYVMG | 153 |
| 2-135-12C | of PCT Publication | WFRQAPGKEREFVAAVSWSAGNTRYANSVKGRFTI | |
| | No. WO | SRDIAKNTFYLQMNSLKPEDTAVYYCVADRRYGGS | |
| | 2009/068627 A2 | LDPSAWDFWGQGTQVTVSS | |
| PMPIL12RB | SEQ ID NO: 2109 | EVQLVESGGGLVQPGGSLRLSCAVSGFTSDYYVIA | 154 |
| 2-135-1A | of PCT Publication | WFRQTPGHEREGVSSIRKGDGATYYADSVKGRFSI | |
| | No. WO | SRDNAKNMVYLQMNSLKPEDTAVYTCAARYDTLLG | |
| | 2009/068627 A2 | GGRPREFPYWGQGTQVTVSS | |
| PMPIL12RB | SEQ ID NO: 2110 | EVQLVESGGGLVQPGGSLRLSCAVSGFTSDYYVVA | 155 |
| 2-135-1C | of PCT Publication | WFRQTPGHEREGVSSIRIGDYATYYADSVKGRFSI | |
| | No. WO | SRDNAKNMVYLQMNSLKPEDTAVYTCAARYDSHLG | |
| | 2009/068627 A2 | GGRPREFPYWGQGTQVTVSS | |
| PMPIL12RB | SEQ ID NO: 2111 | EVQLVESGGGLVQPGGSLRLSCAVSGFTSDYYVIA | 156 |
| 2-135-1D | of PCT Publication | WFRQTPGHEREGVSSIRITDNATYYADSVKGRFSI | |
| | No. WO | SRDNAKNMVFLQMNSLKPEDTAVYTCAARYETLLG | |
| | 2009/068627 A2 | GGRPREFPYWGQGTQVTVSS | |
| PMPIL12RB | SEQ ID NO: 2112 | EVQLVESGGGLVQPGGSLILSCAVSGFTSDYYVIA | 157 |
| 2-135-1E | of PCT Publication | WFRQTPGHEREGVSSIRIGDGATYYADSVKGRFSI | |
| | No. WO | SRDNAKNMVYLQMNSLKPEDTAVYTCAARYDTLLG | |
| | 2009/068627 A2 | GGRPREFPYWGQGTQVTVSS | |
| PMPIL12RB | SEQ ID NO: 2113 | EVQLVESGGGLVQPGGSLRLSCAASGFSFSRNWMY | 158 |
| 2-135-1F | of PCT Publication | WVRQAPGKGLEWVGDISMEGTNTYYRDSVQGRFTI | |
| | No. WO | SRDNAKNILYLQMNSLKSEDTAVYYCARAKNEGFV | |
| | 2009/068627 A2 | PGGYDFDYRGQGTQVTVSS | |
| PMPIL12RB | SEQ ID NO: 2114 | EVQLVESGGGLVQPGGSLRLSCAVSGFTSDYYVIA | 159 |
| 2-135-1H | of PCT Publication | WFRQTPGHEREGLSSIRIGDSATFYADSVKGRFSI | |
| | No. WO | SRDNAKNMVYLQMNSLKPEDTAVYTCAGRYDTLLG | |
| | 2009/068627 A2 | GGRPREFPYWGQGTQVTVSS | |
| PMPIL12RB | SEQ ID NO: 2115 | EVQLVESGGGLVQPGGSLRLSCAVSGFTSDFYVIA | 160 |
| 2-135-3A | of PCT Publication | WFRQTPGHEREGVSSIRIGDNAAYYADSVKGRFSI | |
| | No. WO | SRDYAKNMVYLQMNSLKAEDTAVYTCAGRYDSNLG | |
| | 2009/068627 A2 | GGRPREFPYWGRGTQVTVSS | |
| PMPIL12RB | SEQ ID NO: 2116 | EVQLVESGGGLVQPGGSLRLSCAASGRTIAAYKMA | 161 |
| 2-135-3B | of PCT Publication | WFRQAPDKARERVALITSFGLTVYADSVKGRFTIS | |
| | No. WO | RDNAYNTVYLQMNHLKFEDTAVYYCAAGQQDSSNY | |
| | 2009/068627 A2 | NSWGQGTQVTVSS | |
| PMPIL12RB | SEQ ID NO: 2117 | EVQLVESRGGLVQPGGSLRLSCAVSGFTSDYYVIA | 162 |
| 2-135-3D | of PCT Publication | WFRQTPGHEREGVSSIRKGDGATYYADSVKGRFSI | |
| | No. WO | SRDNAKNMVYLQMNSLKPEDTAVYTCAARYDTLLG | |
| | 2009/068627 A2 | GGRPREFPYWGQGTQVTVSS | |
| PMPIL12RB | SEQ ID NO: 2118 | EVQLVESGGRLVQAGDSLRLSCAASGRTFTSYRMG | 163 |
| 2-135-7B | of PCT Publication | WFRQAPGKEREFVSALRWSSGNIDYTYYADSVKGR | |
| | No. WO | FSISGDYAKNTVYLQMNSLKAEDTAVYYCAASTRW | |
| | 2009/068627 A2 | GVMESDTEYTSWGQGTQVTVSS | |
| PMPIL12RB | SEQ ID NO: 2119 | EVQLVESGGGLVQAGGSLTLSCAASGRTFSSYHMG | 164 |
| 2-135-8A | of PCT Publication | WFRQAPGKEREYVAAISWSGHMTYYKDSAKGRFTI | |
| | No. WO | SRDNAKNTVYLQMNNLKPEDTAVYYCAARNRDYWS | |
| | 2009/068627 A2 | DFDVPGRYAYWGQGTQVTVSS | |
| PMPIL12RB | SEQ ID NO: 2120 | EVQLVESGGRLVQAGDSLRLSCAASGRTFTNYRVG | 165 |
| 2-135-8C | of PCT Publication | WFRQAPGKEREFVSALRWSSSNIDYTYYADSVKGR | |
| | No. WO | FSISGDYAKNTVYLQMNSLKAEDTAVYYCAASTSW | |
| | 2009/068627 A2 | GVLESDTEYTSWGQGTQVTVSS | |
| PMPIL12RB | SEQ ID NO: 2121 | EVQLVESGGGLVQPGGSLRLSCAVSGFTSEYYVIA | 166 |
| 2-135-1B | of PCT Publication | WFRQTPGHEREGVSSIRIRDNAAYYADSVKGRFSI | |
| | No. WO | SRDNHKNMVYLQMNSLKPEDTAVYTCAGRYDTLLG | |
| | 2009/068627 A2 | GGRPREFPYWGQGTQVTXSX | |
| PMPIL12RB | SEQ ID NO: 2122 | EVXLVESGGALVQDGGSLRLSCAASGLTLSNYVAA | 167 |
| 2-135-3C | of PCT Publication | WFRQAPGKEREYVGSIRWSSEQTYYANSVKGRFSI | |
| | No. WO | SRDNAKNAVYLEMNTLKPEDTAVYYCALGTSFSAL | |
| | 2009/068627 A2 | PKLYNYWGQGTQVTVSS | |
| PMPIL12RB | SEQ ID NO: 2123 | EVXLVESGGRLVQAGDSLRLSCAASGRTFISYRMG | 168 |
| 2-135-7A | of PCT Publication | WFRQAPGKEREFVAALRWSSSNIDYTYYADSVKGR | |
| | No. WO | FSISGDYAKNTVYLQMNSLKAEDTAVYYCAASTRW | |
| | 2009/068627 A2 | GVMESDTEYTSWGQGTQVTVSS | |
| PMPIL12RB | SEQ ID NO: 2124 | EVQLVESGGGLVQSGGSLRLSCAASEGTFTIYPLG | 169 |
| 2-136-4B | of PCT Publication | WFRQAPGKDRKFVAALPWSAGIPQYSDSVKGRFTI | |
| | No. WO | SRDNAKNTVYLQMNNLKPEDTAVYYCAXKGRDDSY | |
| | 2009/068627 A2 | QPXNYWGQGTQVTVSS | |

In some aspects, the IL12Rβ32 targeting moiety competes with an antibody set forth above in Table R4, for binding to the IL12Rβ32. In further aspects, the IL12Rβ32 targeting moiety comprises CDRs having CDR sequences of an antibody set forth in Table R4. In some embodiments, the IL12Rβ32 targeting moiety comprises all 3 CDR sequences of the antibody set forth in Table R4. In further aspects, an IL12Rβ32 targeting moiety comprises a VH (e.g., a VHH) comprising the amino acid sequence of the VH of an antibody set forth in Table R4.

In some embodiments, the IL12Rβ2 targeting moiety binds an epitope at similar proximity to cell membrane as the IL12Rβ1 targeting of the tumor-targeted split IL12 receptor agonist. In some embodiments, if the IL12Rβ1 targeting moiety binds to the D2 domain of IL12Rβ1, then the IL12Rβ2 targeting moiety binds to D1 domain of IL12Rβ2.

### 6.4.2.2. IL12Rβ2 Ligands

In some embodiments, the IL12Rβ2 binding domain of a tumor-targeted split IL12 receptor agonist is an IL12Rβ2 ligand. An IL12Rβ2 ligand refers to a variant IL12 moiety which has preferential binding to IL12Rβ2 vs. IL12Rβ1 as compared to wild-type IL12.

In some embodiments, the preferential binding to IL12Rβ2 is achieved through mutations in the p40 moiety that reduce its binding to IL12Rβ1, while maintaining or increasing the binding of the p35 moiety to IL12Rβ2.

In some embodiments, the IL12Rβ2 ligand comprises a p35 moiety whose amino acid sequence has at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ2 binding portion of a mammalian, e.g., human or murine, p35 (sometimes referred to as the alpha subunit of IL12 or IL12a). For example, the p35 moiety can comprise an amino acid having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO:3 or SEQ ID NO:5. In some embodiments, the p35 moiety comprises an amino acid having the amino acid sequence of SEQ ID NO:3 or SEQ ID NO:5 and/or a variant of SEQ ID NO:3 or SEQ ID NO:5 that does not have reduced IL12Rβ2 binding as compared to the amino acid sequence of SEQ ID NO:3 or SEQ ID NO:5.

In some embodiments, the IL12Rβ2 ligand comprises a p40 moiety whose amino acid sequence has at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL12Rβ1 binding portion of a mammalian, e.g., human or murine, p40 (sometimes referred to as the beta subunit of IL12 or IL12P). For example, the p40 moiety can comprise an amino acid having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of any one of SEQ ID NO:6, SEQ ID NO:7, SEQ ID NO:9 and SEQ ID NO:10. In some embodiments, the p40 moiety is a variant p40 moiety having an amino acid comprising one or more mutations that reduce IL12Rβ1 binding as compared to the amino acid sequence of SEQ ID NO:6, SEQ ID NO:7, SEQ ID NO:9 or SEQ ID NO:10. For example, in some embodiments, the variant p40 moiety can have up to 1,000-fold attenuated binding to human IL12Rβ1 as compared to wild type human p40. In some embodiments, the variant p40 moiety can have up to 100-fold, up to 50-fold, up to 25-fold, up to 20-fold, up to 15-fold, up to 10-fold, or up to 5-fold attenuated binding to human IL12Rβ1 as compared to wild type human IL12 p40.

Other characteristics of useful IL12 p40 variants may include the ability to destabilize dimerization with IL12 p35.

Exemplary amino acid substitutions include, but are not limited to substitutions at positions K6, W15, D18, E32, E33, D34, Q42, S43, E45, Q56, E59, F60, D62, E73, K84, D87, D93, K96, K99, E100, N103, K104, N113, Q144, R159, D161, K163, E187, N200, N218, Q229, E235, Y246, C252, Q256, K258, K260, E262, K264, N281, Y292, and E299, wherein amino acid positions, unless otherwise noted, are relative to the mature human IL12 p40 amino acid sequence, excluding the 22-amino acid signal sequence. Corresponding amino acid positions in the full-length human sequence, full-length murine sequence, and mature murine sequence are provided in Table B2. Table B2 also provides exemplary substitutions at each noted position.

TABLE B2
IL12 p40 moiety Amino Acid Substitutions

| | | Amino Acid | Amino | |
|---|---|---|---|---|
| Amino Acid | Amino Acid | (Murine - | Acid | |
| (Human - | (Human - | Full | (Murine - | Exemplary |
| Full Length) | Mature) | Length) | Mature) | substitutions |
| K28 | K6 | K28 | K6 | A |
| W37 | W15 | W37 | W15 | A |
| D40 | D18 | D40 | D18 | N, K, A |
| E54 | E32 | E54 | E32 | Q, A |
| E55 | E33 | E55 | E33 | Q, A |
| D56 | D34 | D56 | D34 | N, K, A |
| Q64 | Q42 | Q64 | Q42 | E |
| S65 | S43 | S65 | S43 | E, K |
| E67 | E45 | E67 | E45 | Q |
| Q78 | Q56 | T78 | T56 | E |
| E81 | E59 | E81 | E59 | K, Q, A |
| F82 | F60 | F82 | F60 | A |
| D84 | D62 | D84 | D62 | N |
| E95 | E73 | E95 | E73 | Q |
| K106 | K84 | K106 | K84 | A |
| D109 | D87 | N109 | N87 | A |
| D115 | D93 | E115 | E93 | A |
| K118 | K96 | K118 | K96 | A |
| K121 | K99 | K121 | K99 | E, Y, A |
| E122 | E100 | | | Q |
| N125 | N103 | N122 | N100 | D, Q |
| K126 | K104 | K123 | K101 | A |
| N135 | N113 | N132 | N110 | D, Q |
| Q166 | Q144 | R163 | R141 | E |
| R181 | R159 | T178 | T156 | E |
| D183 | D161 | D180 | D158 | N |
| K185 | K163 | R182 | R160 | E |
| C199 | C177 | C197 | C175 | S |
| E209 | E187 | E207 | E185 | Q |
| K219 | K197 | K217 | K195 | A |
| N222 | N200 | N220 | N198 | D, Q |
| N240 | N218 | N238 | N216 | Q |
| Q251 | Q229 | Q248 | Q226 | E |
| E257 | E235 | E254 | E232 | Q |
| Y268 | Y246 | Y265 | Y243 | V, F |
| C274 | C252 | F271 | F249 | S |
| Q278 | Q256 | Q275 | Q253 | N |
| K280 | K258 | K277 | K255 | E |
| K282 | K260 | E279 | K257 | E |
| E284 | E262 | N289 | N267 | Q |
| K286 | K264 | K291 | K269 | E |
| N303 | N281 | G307 | G285 | D, Q |
| Y314 | Y292 | Y318 | Y296 | F |
| E321 | E299 | K325 | K303 | Q |

An exemplary amino acid substitution at mature human K6 is K6A.

An exemplary amino acid substitution at mature human W15 is W15A.

Exemplary amino acid substitutions at mature human D18 include D18N, D18K, and D18A.

Exemplary amino acid substitutions at mature human E32 include E32Q and E32A.

Exemplary amino acid substitutions at mature human E33 include E33Q and E33A.

Exemplary amino acid substitutions at mature human D34 include D34N, D34K, and D34A.

An exemplary amino acid substitution at mature human Q42 is Q42E.

Exemplary amino acid substitutions at mature human S43 include S43E and S34K.

An exemplary amino acid substitution at mature human E45 is E45Q.

An exemplary amino acid substitution at mature human Q56 is Q56E.

Exemplary amino acid substitutions at mature human E59 include E59K, E59Q, and E59A.

An exemplary amino acid substitution at mature human F60 is F60A.

An exemplary amino acid substitution at mature human D62 is D62N.

An exemplary amino acid substitution at mature human E73 is E73Q.

An exemplary amino acid substitution at mature human K84 is K84A.

An exemplary amino acid substitution at mature human D87 is D87N.

An exemplary amino acid substitution at mature human D93 is D93A.

An exemplary amino acid substitution at mature human K96 is E93A.

Exemplary amino acid substitutions at mature human K99 include K99E, K99Y, and K99A.

An exemplary amino acid substitution at mature human E100 is E100 Q.

Exemplary amino acid substitutions at mature human N103 include N103D and N103Q.

An exemplary amino acid substitution at mature human K104 is K104A.

Exemplary amino acid substitutions at mature human N113 include N113D and N113Q.

An exemplary amino acid substitution at mature human Q144 is Q144E.

An exemplary amino acid substitution at mature human R159 is R159 E.

An exemplary amino acid substitution at mature human D161 is D161N.

An exemplary amino acid substitution at mature human K163 is K163E.

An exemplary amino acid substitution at mature human E187 is E187Q.

Exemplary amino acid substitutions at mature human N200 include N200D and N200Q.

An exemplary amino acid substitution at mature human N218 is N218Q.

An exemplary amino acid substitution at mature human Q229 is Q229E.

An exemplary amino acid substitution at mature human E235 is E235Q.

Exemplary amino acid substitutions at mature human Y246 include Y246V and Y246F.

An exemplary amino acid substitution at mature human C252 is C252S.

An exemplary amino acid substitution at mature human Q256 is Q256N.

An exemplary amino acid substitution at mature human K258 is K258E.

An exemplary amino acid substitution at mature human K260 is K260E.

An exemplary amino acid substitution at mature human E262 is E262Q.

An exemplary amino acid substitution at mature human K264 is K264E.

Exemplary amino acid substitutions at mature human N281 include N281D and N281Q.

An exemplary amino acid substitution at mature human Y292 is Y292F.

An exemplary amino acid substitution at mature human E299 is E299Q.

In certain embodiments, amino acid substitutions at mature human Y246 and/or Y292 destabilize the p40/p35 heterodimer by preventing formation of a disulfide bond between the two subunits. Exemplary amino acid substitutions at Y246 include Y246V and Y246F. An exemplary amino acid substitution at Y292 is Y292F.

### 6.5. Tumor-Associated Antigen Targeting Moieties

The tumor-targeted IL12Rβ1 agonist and the tumor-targeted IL12Rβ2 of the tumor-targeted split IL12 receptor agonists of the disclosure both comprise a tumor-associated antigen (“TAA”) targeting moiety. Typically, the TAA recognized by the TAA targeting moiety of the tumor-targeted IL12Rβ1 agonist and the TAA recognized by the TAA targeting moiety of the tumor-targeted IL12Rβ2 agonist are both expressed on the same cancer cell and may be the same TAA or different TAAs. If the TAA targeting moiety of the tumor-targeted IL12Rβ1 agonist and the TAA targeting moiety of the tumor-targeted IL12Rβ2 agonist are the same, in some embodiments binding the TAA targeting moiety of the tumor-targeted IL12Rβ1 agonist and the TAA targeting moiety of the tumor-targeted IL12Rβ2 agonist bind to the TAA in a non-competing fashion such that both the tumor-targeted IL12Rβ1 agonist and the tumor-targeted IL12Rβ2 agonist can bind to the same cell concurrently.

Without being bound by theory, the inventors believe that the incorporation of TAA targeting moieties that bind to the same tumor cell in both the tumor-targeted IL12Rβ1 agonist and the tumor-targeted IL12Rβ2 agonist permits the delivery of high concentrations of IL12 into the tumor microenvironment while engaging tumor reactive lymphocytes, resulting in enhancement of the cytotoxic response against tumor cells with a concomitant reduction of systemic exposure.

Suitable TAA targeting moiety formats are described in Section 6.7. The TAA targeting moiety is preferably an antigen binding moiety, for example an antibody or an antigen-binding portion of an antibody, e.g., a Fab, as described in Section 6.7.1, an scFv, as described in Section 6.7.2, or a single domain antibody, as described in Section 6.7.3.

Exemplary target molecules recognized by the TAA targeting moieties of the tumor-targeted IL12Rβ1 agonist and/or the tumor-targeted IL12Rβ2 agonist are Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, prostate-specific membrane antigen (PSMA), T-cell receptor/CD3-zeta chain, GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, pi20ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, Her3, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD20, melanotransferrin (MELTF; CD228), MCSP, PDGFPR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, PAP, LAGA-1a, prostein, survivin and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) and extra domain B (EDB) of fibronectin and the A1 domain of tenascin-C (TnC A1).

In some embodiments, the target molecule recognized by the TAA targeting moiety of the tumor-targeted IL12Rβ1 agonist and/or the tumor-targeted IL12Rβ2 agonist is BCMA. In some embodiments, the target molecule recognized by the TAA targeting moiety of the tumor-targeted IL12Rβ1 agonist and/or the tumor-targeted IL12Rβ2 agonist is CD20. In some embodiments, the target molecule recognized by the TAA targeting moiety of the tumor-targeted IL12Rβ1 agonist and/or the tumor-targeted IL12Rβ2 agonist is EGFR. In some embodiments, the target molecule recognized by the TAA targeting moiety of the tumor-targeted IL12Rβ1 agonist and/or the tumor-targeted IL12Rβ2 agonist is PSMA. In some embodiments, the target molecule recognized by the TAA targeting moiety of the tumor-targeted IL12Rβ1 agonist and/or the tumor-targeted IL12Rβ2 agonist is CA9. In some embodiments, the target molecule recognized by the TAA targeting moiety of the tumor-targeted IL12Rβ1 agonist and/or the tumor-targeted IL12Rβ2 agonist is MSLN. In some embodiments, the target molecule recognized by the TAA targeting moiety of the tumor-targeted IL12Rβ1 agonist and/or the tumor-targeted IL12Rβ2 agonist is EPCAM. In some embodiments, the target molecule recognized by the TAA targeting moiety of the tumor-targeted IL12Rβ1 agonist and/or the tumor-targeted IL12Rβ2 agonist is B7H3. In some embodiments, the target molecule recognized by the TAA targeting moiety of the tumor-targeted IL12Rβ1 agonist and/or the tumor-targeted IL12Rβ2 agonist is HER2/HER3. In some embodiments, the target molecule recognized by the TAA targeting moiety of the tumor-targeted IL12Rβ1 agonist and/or the tumor-targeted IL12Rβ2 agonist is STEAP1. In some embodiments, the target molecule recognized by the TAA targeting moiety of the tumor-targeted IL12Rβ1 agonist and/or the tumor-targeted IL12Rβ2 agonist is CEACAM5.

In some embodiments, the targeting moieties target the exemplary target molecules set forth in Table T1 below, which provides references to exemplary antibodies or antibody sequences upon which the targeting moiety can be based.

TABLE T1
Exemplary Target Molecules

| Target | Antibody Name and/or Binding Sequences |
|---|---|
| 5T4 | GEN1044 |
| Activin Receptor Type II | Bimagrumab |
| (ACVR2) | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID NO: 21, 26 or |
| | 31 of US 2021/0171641 A1. |
| | VL: the VL sequence of the light chain of SEQ ID NO: 20, 22 or |
| | 30 of US 2021/0171641 A1. |
| B7-H3 (CD276) | VH: the VH sequence of the heavy chain of SEQ ID NO: 21, 29 or |
| | 37 of US 2019/0002563 A1. |
| | VL: the VL sequence of the light chain of SEQ ID NO: 17, 25 or |
| | 33 of US 2019/0002563 A1. |
| B7-H3 (CD276) | VH: the VH sequence of the heavy chain of SEQ ID NO: 146, 147 |
| | or 148 of U.S. Pat. No. 10,640,563. |
| | VL: the VL sequence of the light chain of SEQ ID NO: 143, 144 or |
| | 145 of U.S. Pat. No. 10,640,563. |
| BCMA | VH: the VH sequence of the heavy chain of SEQ ID NO. 126 of |
| | US 2021/0206865 A1 |
| | VL: the VL sequence of the light chain of SEQ ID NO. 129 or |
| | SEQ ID NO. 132 of US 2021/0206865 A1 |
| CA125 (MUC16) | Igobumab |
| CA125 | OvaRex ™ (oregobumab) |
| Cadherin | The antibodies described in US Pub. No. US 2006/0039915. |
| N-cadherin | An antibody that binds to the amino acid sequence of SEQ ID |
| | NO: 10, 17 or 18 of US Pub. No. US 2010/0278821. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID NO: 124 of |
| | US Patent Pub. US 2017/0002060 A1 |
| | VL: the VL sequence of the light chain of SEQ ID NO: 125 of US |
| | Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular immunology |
| | 30:1361-1367 |
| CD33 | Lintuzumab |
| CD38 | Darzalex ™ (daratumumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | Flotetuzumab |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID NOs: 4-6 |
| | of U.S. Pat. No. 8,771,690 B2 |
| | CDR-L1, CDR-L2 and CDR-L3 sequences of SEQ ID NOs: 1-3 of |
| | U.S. Pat. No. 8,771,690 B2 |
| CEA | CEA binding portion of RO6958688/RG7802 from clinical trial |
| | NCT02324257 |
| CEA | Cibisatamab |
| CEA | CEA binding portion of MEDI-565/MT110/AMG211 from clinical |
| | trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. US 2020/0317806 A1 |
| | HL: SEQ ID NO: 3 of US Patent Pub. No. US 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™ (catumaxomab) |
| EpCAM | Vicineum ™ (oportuzumab monatox) |
| EpCAM | M701 |
| GD2 | 3F8 |
| | ReoPro ™ (abiciximab) |
| gpA33 | MGD007 |
| GPC3 | ERY974 |
| GUCY2C | PF-07062119 |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| Integrinα4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrinα4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrinα5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US Patent |
| | Pub. US 2022/0089744. |
| | VL:, SEQ ID NO: 4, 16, 18, 20, 22, 44-57 or 107-116 of US Patent |
| | Pub. US 2022/0089744. |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences having the |
| | amino acid sequences of any one of the following SEQ ID NO: |
| | pairs from US 2018/0118848A1: 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| Phosphatidylserine | (bavituximab) |
| PSCA | GEM3PSCA |
| PSMA | huJ591 |
| PSMA | Anti-PSMA antibodies having VH and VL sequences having the |
| | amino acid sequences of any one of the following SEQ ID NO: |
| | pairs from WO 2017/023761A1: 2/1642; 10/1642; 18/1642; |
| | 26/1642; 34/1642; 42/1642; 50/1642; 58/1642; 66/1642; 74/1642; |
| | 82/1642; 90/1642; 98/1642; 106/1642; 1 14/1642; 122/130; and |
| | 138/146. |
| PSMA | An antibody such as: PSMA 3.7, PSMA 3.8, PSMA 3.9, PSMA |
| | 3.11, PSMA 5.4, PSMA 7.1, PSMA 7.3, PSMA 10.3, PSMA 1.8.3, |
| | PSMA A3.1.3, PSMA A3.3.1, Abgenix 4.248.2, Abgenix 4.360.3, |
| | Abgenix 4.7.1, Abgenix 4.4.1, Abgenix 4.177.3, Abgenix 4.16.1, |
| | Abgenix 4.22.3, Abgenix 4.28.3, Abgenix 4.40.2, Abgenix 4.48.3, |
| | Abgenix 4.49.1, Abgenix 4.209.3, Abgenix 4.219.3, Abgenix |
| | 4.288.1, Abgenix 4.333.1, Abgenix 4.54.1, Abgenix 4.153.1, |
| | Abgenix 4.232.3, Abgenix 4.292.3, Abgenix 4.304.1, Abgenix |
| | 4.78.1 and Abgenix 4.152.1 described in WO2003034903A2 |
| | A hybridoma cell line such as: PSMA 3.7 (PTA-3257), PSMA 3.8, |
| | PSMA 3.9 (PTA-3258), PSMA 3.11 (PTA-3269), PSMA 5.4 |
| | (PTA-3268), PSMA 7.1 (PTA-3292), PSMA 7.3 (PTA-3293), |
| | PSMA 10.3 (PTA-3247) , PSMA 1.8.3 (PTA-3906), PSMA A3.1.3 |
| | (PTA-3904), PSMA A3.3.1 (PTA-3905), Abgenix 4.248.2 (PTA- |
| | 4427), Abgenix 4.360.3 (PTA- 4428), Abgenix 4.7.1 (PTA-4429), |
| | Abgenix 4.4.1 (PTA-4556), Abgenix 4.177.3 (PTA-4557), |
| | Abgenix 4.16.1 (PTA-4357), Abgenix 4.22.3 (PTA-4358), |
| | Abgenix 4.28.3 (PTA-4359), Abgenix 4.40.2 (PTA-4360), |
| | Abgenix 4.48.3 (PTA-4361), Abgenix 4.49.1 (PTA-4362), |
| | Abgenix 4.209.3 (PTA-4365), Abgenix 4.219.3 (PTA-4366), |
| | Abgenix 4.288.1 (PTA-4367), Abgenix 4.333.1 (PTA-4368), |
| | Abgenix 4.54.1 (PTA-4363), Abgenix 4.153.1 (PTA-4388), |
| | Abgenix 4.232.3 (PTA-4389), Abgenix 4.292.3 (PTA-4390), |
| | Abgenix 4.304.1 (PTA-4391), Abgenix 4.78.1 (PTA-4652), and |
| | Abgemx 4.152.1(PTA-4653) described in WO 2003/034903A2. |
| | VH of SEQ ID NOs: 2-7 described in WO 2003/034903A2 |
| | VL of SEQ ID NOs: 8-13 described in WO 2003/034903A2 |
| PSMA | VH: SEQ ID NOs: 225, 239, 253, 267, 281, 295, 309, 323, 337, |
| | 351, 365, 379, 393, 407, 421, 435, 449, 463, 477, 491, 505, 519, |
| | 533, 547, 561, 575, 589, 603 or 617 described in WO |
| | 2011/121110A1. |
| | VL SEQ ID NOs: 230, 244, 258, 272, 286, 300, 314, 328, 342, |
| | 356, 370, 384, 398, 412, 426, 440, 454, 468, 482, 496, 510, 524, |
| | 538, 552, 566, 580, 594, 608 or 622 described in WO |
| | 2011/121110A1. |
| | VH and VL SEQ ID Nos: 235, 249, 263, 277, 291, 305, 319, 333, |
| | 347, 361, 375, 389, 403, 417, 431, 445, 459, 473, 487, 501, 515, |
| | 529, 543, 557, 571, 585, 599, 613 or 627 described in WO |
| | 2011/121110A1. |
| PSMA | An anti-PMSA antibody having a VL amino acid sequence of any |
| | one of SEQ ID NOs: 229-312 of US 2022/0119525 A1 and a VH |
| | of SEQ ID NO: 217 of US 2022/0119525 A1. |
| PSMA | ES414 |
| PSMA | BAY2010112 (pasotuxizumab) |
| PSMA | CCW702 |
| PSMA | JNJ-63898081 |
| PSMA | CC-1 |
| PSMA | Acapatamab |
| PSMA | HPN424 |
| RAAG12 | RAV12 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 described in |
| | US20210179731A1. |
| | VHCDR2 SEQ ID NOs: 15, 21, 34, 182, 184 or 185 described in |
| | US20210179731A1. |
| | VHCDR3 SEQ ID NOs: 16 and 35 described in |
| | US20210179731A1. |
| | VH SEQ ID NOs: 182 or 184 described in US20210179731A1. |
| | VLCDR1 SEQ ID NOs: 11 or 30 described in |
| | US20210179731A1. |
| | VLCDR2 SEQ ID NOs: 12 or 31 described in |
| | US20210179731A1. |
| | VLCDR3 SEQ ID NOs: 13 or 32 described in |
| | US20210179731A1. |
| | VL SEQ ID NOs: 183 or 186 described in US20210179731A1. |
| STEAP1 | AMG509 |
| STEAP2 | Anti-STEAP 2 antibodies having CDR-H1, CDR-H2, CDR-H3, |
| | CDR-L1, CDR-L2 and CDR-L3 sequences selected from SEQ ID |
| | NOS: (1) 4-6-8-12-14-16; (2) 20-22-24-28-30-32; (3) 36-38-40- |
| | 44-46-48; (4) 52-54-56-60-62-64; (5) 68-70-72-60-62-64; (6) 76- |
| | 78-80-60-62-64; (7) 84-86-88-60-62-64; (8) 92-94-96-60-62-64; |
| | (9) 100-102-104-60-62-64; (10) 108-110-112-116-118-120; (11) |
| | 124-126-128-132-134-136; (12) 140-142-144-148-150-152; (13) |
| | 156-158-160-164-166-168; (14) 172-174-176-180-182-184; (15) |
| | 188-190-192-196-198-200; (16) 204-206-208-212-214-216; (17) |
| | 220-222-224-228-230-232; (18) 236-238-240-244-246-248; (19) |
| | 252-254-256-260-262-264; (20) 268-270-272-276-278-280; (21) |
| | 284-286-288-292-294-296; (22) 300-302-304-308-310-312; (23) |
| | 316-318-320-324-326-328; (24) 332-334-336-340-342-344; (25) |
| | 348-350-352-356-358-360; (26) 364-366-368-372-374-376; and |
| | (27) 380-382-384-388-390-392 of U.S. Pat. No. 10,772,972 B2. |
| | Anti-STEAP 2 antibodies having (a) a VH comprising the amino |
| | acid of any one of SEQ ID NOs: 2, 18, 34, 50, 66, 74, 82, 90, 98, |
| | 106, 122, 138, 154, 170, 186, 202, 218, 234, 250, 266, 282, 298, |
| | 314, 330, 346, 362, and 378 of U.S. Pat. No. 10,772,972 B2; |
| | and (b) a VL comprising the amino acid sequence of any one of |
| | SEQ ID NOs: 10; 26; 42; 58; 114; 130; 146; 162; 178; 194; 210; |
| | 226, 242; 258; 274; 290; 306; 322; 338; 354; 370; and 386 of |
| | U.S. Pat. No. 10,772,972 B2. |
| | Anti-STEAP 2 antibodies having a VH/VL pair comprising the |
| | amino acid sequences of any of the following pairs of SEQ ID |
| | NOs of U.S. Pat. No. 10,772,972 B2: 2/10; 18/26; 34/42; 50/58; |
| | 66/58; 74/58; 82/58; 90/58; 98/58; 106/114; 122/130; 138/146; |
| | 154/162; 170/178; 186/194; 202/210; 218/226; 234/242; 250/258; |
| | 266/274; 282/290; 298/306; 314/322; 330/338; 346/354; 362/370; |
| | and 378/386. |
| Syndecan-1 (CD 138) | The B-B4 antibody described in Wijdenes et al. (1996) Br. J. |
| | Haematol., 94: 318-323 |
| Syndecan-4 | The amino acid sequence of amino acids 93 and 121 of SEQ ID |
| | NO: 1 or the amino acid sequence of amino acids 92 and 122 of |
| | SEQ ID NO: 2 described in European Patent Pub. EP 2 603 236. |
| TNFR | Enbrel ™ (etanercept) |

In some aspects, the TAA targeting moiety competes with an antibody set forth in Table T1 for binding to the target molecule. In further aspects, the TAA targeting moiety comprises CDRs having CDR sequences of an antibody set forth in Table T1. In some embodiments, the targeting moiety comprises all 6 CDR sequences of the antibody set forth in Table T1. In other embodiments, the targeting moiety comprises at least the heavy chain CDR sequences (CDR-H1, CDR-H2, CDR-H3) of such antibody and the light chain CDR sequences of a universal light chain. In further aspects, a targeting moiety comprises a VH comprising the amino acid sequence of the VH of an antibody set forth in Table T1. In some embodiments, the targeting moiety further comprises a VL comprising the amino acid sequence of the VL of the antibody set forth in Table T1. In other embodiments, the targeting moiety further comprises a universal light chain VL sequence.

In some embodiments, the targeting moieties target the exemplary target molecules set forth in Table T2 below, which provides references to exemplary single domain antibodies or antibody sequences upon which the targeting moiety can be based.

TABLE T2
Exemplary Single Domain Antibody (sdAb) Amino Acid Sequences

| | | | SEQ |
|---|---|---|---|
| | | | ID |
| Target | Reference | Sequence | NO |
| B7H3 | SEQ ID NO: 1 of | HVQLVESGGGLVQPGRSLRLSCAASGFTFSSYWMYWV | 170 |
| | PCT Publication | RQTPGKGLEWVSTINRDGSATWYADSVKGRFTISRDN | |
| | No. WO | AKNTGYLQMNSLEPDDTAVYYCVSDPDNYSSDEMVPY | |
| | 2021/247794 A2 | WGQGTQVTVSS | |
| B7H3 | SEQ ID NO: 2 of | QVQLVESGGGLVQPGGSLRLSCAASGFTFSSYWMYWV | 171 |
| | PCT Publication | RQTPGKGLEWVSTINRDGSATWYADSVKGRFTISRDN | |
| | No. WO | AKNTGYLQMNSLKPDDTAVYYCVSDPDNYSSDEMVPY | |
| | 2021/247794 A2 | WGQGTQVTVSS | |
| B7H3 | SEQ ID NO: 3 of | XVQLVESGGGLVQPGXSLRLSCAASGFTFSSYWMYWV | 172 |
| | PCT Publication | RQTPGKGLEWVSTINRDGSATWYADSVKGRFTISRDN | |
| | No. WO | AKNTGYLQMNSLXPDDTAVYYCVSDPDNYSSDEMVPY | |
| | 2021/247794 A2 | WGQGTQVTVSS | |
| CA9 | SEQ ID NO: 1 of | QVQLVESGGGLVQAGGSLRLSCAASGFTFDDWAIGWF | 173 |
| | PCT Publication | RQAPGKEREGVSCISKRHGTTHYADSVKGRFTISSDN | |
| | No. WO | AKNTVYLRMNGLKPEDTAVYYCAASSWGSCTVATMRD | |
| | 2022/157714 A1 | VDRYDYDYWGQGTQVTVSS | |
| CEACAM | Jancewicz et al, | QVKLEESGGGLVQAGGSLRLSCRTSGRTNSVYTMGWF | 174 |
| | 2024, Cancer | RQAPGKEREFVAQIMWGAGTNTHYADSVKGRFTISRD | |
| | Immunol | SAESTVYLQMNSLKPEDTAVYYCAANRGIPIAGRQYD | |
| | Immunother. | YWGQGTQVTVSS | |
| | 73(2):30 | | |
| EpCAM | SEQ ID NO: 1 of | DVQLVESGGGSVQSGGSLRLSCAASGYTYRRYYMGWF | 175 |
| | PCT Publication | RQAPGEQREGVAVINNDGRTNYADSVKGRFRISRDNA | |
| | No. WO | ENTLHLEMNSLKPEDTAMYYCAATGNILPPMTAVPPL | |
| | 2023/044991 A1 | GROWYPYWGRGTLVTVSS | |
| EpCAM | SEQ ID NO: 2 of | HVQLVESGGGSVQSGGSLRLSCAASGYAVKNCMGWFR | 176 |
| | PCT Publication | QAPGKEREGVAVINRNGITTYADSVKGRFTISQDKDK | |
| | No. WO | NTLDLQMNSLKPEDTAMYYCAATPTLLTIPARFLCDV | |
| | 2023/044991 A1 | RNPSGFTDWGQGTLVTVSS | |
| EpCAM | SEQ ID NO: 3 of | QVQLVESGGGSVQAGGSLRLSCVVSAYSAYTYKTMCM | 177 |
| | PCT Publication | GWFRQAPGKEREGVAAIYRGGLNTYYADSVKGRFIIS | |
| | No. WO | RDNAESTMYLQMNSLKPEDTAMYYCAADWLRGDDCNI | |
| | 2023/044991 A1 | GANFDYWGQGTQVTVSS | |
| EpCAM | SEQ ID NO: 4 of | QVQLVESGGGSVQAGGSLRLSCVATGFTISRKCMGWF | 178 |
| | PCT Publication | REAPGKKREVIATINTGSSSPYYADGVKGRFTISQDN | |
| | No. WO | AKNTVYLQMNSLKPEDTAMYYCAATKGVVVGTGYCGG | |
| | 2023/044991 A1 | PYVERPNSAYWGQGTQVTVSS | |
| EpCAM | SEQ ID NO: 5 of | DVQLVESGGGSVQAGRSLRLSCELSDYTWSTVCMGWF | 179 |
| | PCT Publication | RQAPGKEREGVAVIYTRSGGTTYADSAKGRFTISRDN | |
| | No. WO | AKDTLYLQMDSLKPEDTAMYYCAAGPLYDGRCTYRSP | |
| | 2023/044991 A1 | AFHYWGQGTQVTVSS | |
| EpCAM | SEQ ID NO: 6 of | DVQLVESGGGSAQAGGSLRLSCAASGPTSSLRTMGWF | 180 |
| | PCT Publication | RQASGKERERVAVIWDGRTTDYDDSVQDRFTISQDNA | |
| | No. WO | KSTVYLQMNTLKPEDTAMYYCAASPRIVPFASTYFQH | |
| | 2023/044991 A1 | WGQGTQVTVSS | |
| EpCAM | SEQ ID NO: 7 of | HVQLVESGGGSVQAGGSLKLSCAASGSIFSGSIFSRC | 181 |
| | PCT Publication | GMRWYRQAPGKERELVSSTSKDGFTSYTDSVKGRFTI | |
| | No. WO | SQDNANNTLYLQMSSLKTEDTAVYSCAAICAVGGYSL | |
| | 2023/044991 A1 | STYTYWGQGTQVTVSS | |
| EpCAM | SEQ ID NO: 8 of | EVQLVESGGDSVQAGGSLRLSCAASGYSPGSYCMGWF | 182 |
| | PCT Publication | RQAPGKERERVAIIESRGTVTYVDSVKGRFTISKDNA | |
| | No. WO | KNTLYLQMNSLKPEDTAMYYCAASRPWSGVRCLHDKY | |
| | 2023/044991 A1 | DYWGQGTQVTVSS | |
| EpCAM | SEQ ID NO: 9 of | HVQLVESGGGSVQSGGSLRLSCAVSGYAYSSLAWFRQ | 183 |
| | PCT Publication | APGKEREGVAALLTAIGGPTRTTYADSVKGRLAISQD | |
| | No. WO | HAKNTLYLQMSSLKPEDTAMYYCAAGRPAGTPRWLLL | |
| | 2023/044991 A1 | APRDYNYWGQGTQVTVSS | |
| HER2 | SEQ ID NO: 7 of | QVQLQESGGGSVQAGGSLKLTCAASGYIFNSCGMGWY | 184 |
| | PCT Publication | RQSPGRERELVSRISGDGDTWHKESVKGRFTISQDNV | |
| | No. WO | KKTLYLQMNSLKPEDTAVYFCAVCYNLETYWGQGTQV | |
| | 2016/016021 A1 | TVSS | |
| HER2 | SEQ ID NO: 8 of | QVQLQESGGGLVQPGGSLRLSCAASGFIFSNDAMTWV | 185 |
| | PCT Publication | RQAPGKGLEWVSSINWSGTHTNYADSVKGRFTISRDN | |
| | No. WO | AKRTLYLQMNSLKDEDTALYYCVTGYGVTKTPTGQGT | |
| | 2016/016021 A1 | QVTVSS | |
| HER3 | SEQ ID NO: 265 of | QVQLVQSGGGLVQAGGSLSLSCAFSGRTFSMYTMGWF | 186 |
| | PCT Publication | RQAPGKEREFVAANRGRGLSPDIADSVNGRFTISRDN | |
| | No. WO | AKNTLYLQMDSLKPEDTAVYYCAADLQYGSSWPQRSS | |
| | 2021/188736 A1 | AEYDYWGQGTTVTVSS | |
| MSLN | SEQ ID NO: 1 of | QVQLVQSGGGLVHPGGSLRLSCAASGIDLSLYRMRWY | 187 |
| | U.S. Publication | RQAPGKERDLVALITDDGTSYYEDSVKGRFTITRDNP | |
| | No. US | SNKVFLQMNSLKPEDTAVYYCNAETPLSPVNYWGQGT | |
| | 2018/0002439 A1 | QVTVS | |
| MSLN | SEQ ID NO: 2 of | QVQLVQSGGGLVQAGGSLRLSCAPSGSIFGIRTMDWY | 188 |
| | U.S. Publication | RQAPGKERELVARITMDGRVFHADSVKGRFSGSRDGA | |
| | No. US | SNAVYLQMNSLKPDDTAVYYCRYSGLTSREDYWGPGT | |
| | 2018/0002439 A1 | QVTVSS | |
| MSLN | SEQ ID NO: 97 of | QVQLVQSGGGLVHPGGSLRLSCAASGIDLSLYRMRWY | 187 |
| | PCT Publication | RQAPGKERDLVALITDDGTSYYEDSVKGRFTITRDNP | |
| | No. WO | SNKVFLQMNSLKPEDTAVYYCNAETPLSPVNYWGQGT | |
| | 2020/023888A2 | QVTVS | |
| MSLN | SEQ ID NO: 98 of | QVQLVQSGGGLVQAGGSLRLSCAPSGSIFGIRTMDWY | 188 |
| | PCT Publication | RQAPGKERELVARITMDGRVFHADSVKGRFSGSRDGA | |
| | No. WO | SNAVYLQMNSLKPDDTAVYYCRYSGLTSREDYWGPGT | |
| | 2020/023888A2 | QVTVSS | |
| MUC16 | SEQ ID NO: 15 of | QVQLQESGGGLVQAGGSLRLSCAASGRTVSSLFMGWF | 189 |
| | PCT Publication | RQAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO: 20 of | QVQLQESGGGLVQAGDSLRLSCAASGRAVSSLFMGWF | 190 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO: 25 of | QVQLQESGGGLVQAGDSLRLSCAASGRTVSSLFMGWF | 191 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO: 30 of | QVQLQESGGGLVQPGDSMRLSCAAEGDSLDGYVVGWF | 192 |
| | PCT Publication | RQAPGKERQGVSSISGDGSMRYVADSVKGRFTISRDN | |
| | No. WO | AKNTVYLQMIDLKPEDTGVYYCAADPPTWDYWGQGTQ | |
| | 2020/023888 A2 | VTVSS | |
| MUC16 | SEQ ID NO: 35 of | QVQLQESGGGLVQPGGSLRLSCAASGRTVSSLFMGWF | 193 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| MUC16 | SEQ ID NO: 40 of | QVQLQESGGGLVQAGESLRLSCAASGRTVSSLFMGWF | 194 |
| | PCT Publication | RRAPGKERELVAAISRYSLYTYYADSVKGRFTISADN | |
| | No. WO | AKNAVYLQMNSLKPEDTAVYYCASKLEYTSNDYDSWG | |
| | 2020/023888 A2 | QGTQVTVSS | |
| PSMA | Xing et al., 2021 | EVQLVESGGGLVQPGGSLTLSCAASREMISEYSMHWV | 195 |
| | Int. J Mol Sci. | RQAPGKGLEWVSTINPAGTTDYAESVKGRFTISRDNA | |
| | 22(11):5501 | KNTLYLQMNSLKPEDTAVYYCDGYGYRGQGTQVTVSS | |
| PSMA | SEQ ID NO: 38 of | QLQLVESGGGLVHAGGSLRLSCAASGSTFSINAIGWY | 196 |
| | PCT Publication | RQAPGKQRELVAALSSGGSKNYADSVKGRFTISRDNA | |
| | No. WO | KNTVYLQMNRLKPEDTAVYYCNAEIYYSDGVDDGYRG | |
| | 2022/234473 A1 | MDYWGKGTQVTVSS | |
| PSMA | SEQ ID NO: 42 of | EVQVVESGGGLVQTGGSLRLSCAASGPPLSSYAVAWF | 197 |
| | PCT Publication | RQTPGKEREFVAAISWSGSNTYYADSVKGRFTISKDN | |
| | No. WO | AKNTVLVYLQMNSLKPEDTAVYYCAADRRGGPLSDYE | |
| | 2022/234473 A1 | WEDEYADWGQGTQVTVSS | |

In some aspects, the TAA targeting moiety competes with an antibody set forth above in Table T2, for binding to the target molecule. In further aspects, the TAA targeting moiety comprises CDRs having CDR sequences of an antibody set forth in Table T2. In some embodiments, the targeting moiety comprises all 3 CDR sequences of the antibody set forth in Table T2. In further aspects, a targeting moiety comprises a VH (e.g., a VHH) comprising the amino acid sequence of the VH of an antibody set forth in Table T2.

Additional target molecules that can be targeted by the IL12 receptor agonists are disclosed in Table I below and in, e.g., Hafeez et al., 2020, Molecules 25:4764, doi:10.3390/molecules25204764, particularly in Table 1. Table 1 of Hafeez et al. is incorporated by reference in its entirety herein.

### 6.6. Multispecific T-Cell Engagers

Aspects of the present disclosure are directed to combinations comprising (a) a tumor-targeted split IL12 receptor agonist (comprising a tumor-targeted IL12Rβ1 agonist and a tumor-targeted IL12Rβ2 agonist) and (b) a multispecific T-cell engager, as well as to methods comprising administration of a tumor-targeted split IL12 receptor agonist and a multispecific T-cell engager simultaneously, sequentially or separately. The tumor-targeted IL12Rβ1 agonist and the tumor-targeted IL12Rβ2 agonist of the tumor-targeted split IL12 receptor agonist can be in the same or separate compositions, and the multispecific T-cell engager can be in the same or separate composition as the tumor-targeted IL12Rβ1 agonist and the tumor-targeted IL12Rβ2 agonist.

As used herein, a “multispecific T-cell engager” describes a molecule comprising: (a) at least one TAA targeting moiety (e.g., as described in Section 6.5); and (b) at least one T-cell receptor (TCR) complex targeting moiety (e.g., as described below). In general, “multispecific T-cell engager,” as used herein, describes a molecule that does not comprise either an IL12Rβ1 targeting moiety or an IL12Rβ2 targeting moiety. Also disclosed are pharmaceutical compositions comprising such multispecific T-cell engagers, in some cases together also comprising a tumor-targeted IL12Rβ1 agonist and/or a tumor-targeted IL12Rβ2 agonist. Further disclosed are methods for use of such multispecific T-cell engagers in treatment of cancer in combination with a tumor-targeted split IL12 receptor agonist of the disclosure.

Suitable targeting moiety formats (useful for both the TAA targeting moiety and the TCR complex targeting moiety) are described in Section 6.7. The targeting moiety is preferably an antigen binding moiety, e.g., a Fab, as described in Section 6.7.1, an scFv, as described in Section 6.7.2, or a single domain antibody, as described in Section 6.7.3.

Example TAA targeting moieties which may be included in a multispecific T-cell engager disclosed herein are described in Section 6.5. In some embodiments, the TAA targeting moiety of a multispecific T-cell engager targets the same TAA as one or both TAA targeting moieties of the tumor-targeted split IL12 receptor agonist. In some embodiments, the TAA targeting moiety of a multispecific T-cell engager targets a different TAA from both TAA targeting moieties of the tumor-targeted split IL12 receptor agonist. In some embodiments, the TAA targeting moiety of a multispecific T-cell engager targets the same TAA as the TAA targeting moiety of the tumor-targeted IL12Rβ1 receptor agonist. In some embodiments, the TAA targeting moiety of a multispecific T-cell engager targets the same TAA as the TAA targeting moiety of the tumor-targeted IL12Rβ2 receptor agonist. In some embodiments, the TAA targeting moiety of a multispecific T-cell engager targets a different TAA as the TAA targeting moiety of the tumor-targeted IL12Rβ1 receptor agonist. In some embodiments, the TAA targeting moiety of a multispecific T-cell engager targets a different TAA as the TAA targeting moiety of the tumor-targeted IL12Rβ2 receptor agonist.

Certain example tumor-associated antigens and associated antibodies (or antibody sequences) are provided in Table T1. In some embodiments, a multispecific T-cell engager comprises a TAA targeting moiety that specifically binds to a TAA of Table T1. In some embodiments, the TAA is BCMA. In some embodiments, the TAA is CD19. In some embodiments, the TAA is CD20. In some embodiments, the TAA is CD22. In some embodiments, the TAA is EGFR. In some embodiments, the TAA is PSMA. In some embodiments, the TAA is MUC16. In some embodiments, the TAA is CA9. In some embodiments, the TAA is mesothelin (MSLN). In some embodiments, the TAA is EPCAM. In some embodiments, the TAA is B7H3. In some embodiments, the TAA is HER2/HER3. In some embodiments, the TAA is STEAP1. In some embodiments, the TAA is CEACAM5.

A multispecific T-cell engager comprises, in addition to a TAA targeting moiety, a T-cell receptor (TCR) complex targeting moiety. The TCR complex targeting moiety generally binds to any component of the TCR complex. Example targets for a TCR complex targeting moiety of the disclosure include, but are not limited to, CD3 and the T-cell receptor (e.g., TCRαβ or TCRγδ). In some embodiments, the target for the TCR complex targeting moiety is CD3. In some embodiments, the target for the TCR complex targeting moiety is the T-cell receptor (e.g., TCRαβ or TCRγδ). The epitope of the TCR complex targeting moiety can be an individual polypeptide (e.g., CD3 epsilon) or a multimeric component of a protein complex (e.g., the TCRαβ dimer or the TCRγδ dimer of the T-cell receptor complex).

In particular embodiments, a TCR complex targeting moiety of the present disclosure is a CD3 targeting moiety and/or a TCR targeting moiety. A CD3 targeting moiety may be or comprise an antigen-binding domain from an anti-CD3 antibody. A TCR targeting moiety may be or comprise an antigen-binding domain from an anti-TCR antibody.

Exemplary anti-CD3 and anti-TCR antibodies or antibody sequences are set forth in Table G below, upon which the TCR complex targeting moiety can be based.

TABLE G
Exemplary Anti-CD3 and Anti-TCR Antibodies

| Target | Antibody Name and/or Binding Sequences |
|---|---|
| CD3 | The CD3-binding portion of Catumaxomab |
| CD3 | The CD3-binding portion of ertumaxomab |
| CD3 | The CD3-binding portion of anti-PSMA/anti-CD3 antibodies described in |
| | WO2011121110A1 |
| CD3 | Anti-CD3 antibody sequences in U.S. Pat. No. 10,266,593 B2 |
| CD3 | Anti-CD3 antibody sequences in U.S. Pat. No. 8,846,042 B2 |
| CD3 | Anti-CD3 antibody sequences US 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in U.S. Pat. No. 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in U.S. Pat. No. 10,066,016 |
| CD3 | Anti-CD3 antibody sequences in US 2010/0150918 |
| CD3 | The CD3-binding portion of MT110 |
| CD3 | The CD3-binding portion of Acapatamab (AMG160) |
| CD3 | The CD3-binding portion of AMG199 |
| CD3 | The CD3-binding portion of AMG330 |
| CD3 | The CD3-binding portion of AMG427 (Emirodatamab) |
| CD3 | The CD3-binding portion of AMG562 |
| CD3 | The CD3-binding portion of AMG596 |
| CD3 | The CD3-binding portion of AMG673 |
| CD3 | The CD3-binding portion of AMG701 (Pavurutamab) |
| CD3 | The CD3-binding portion of Tarlatamab (AMG757) |
| CD3 | The CD3-binding portion of AMG910 (Gresonitamab) |
| CD3 | The CD3-binding portion of BAY2010112 (Pasotuxizumab) |
| CD3 | The CD3-binding portion of AMG420 |
| CD3 | The CD3-binding portion of AMG424 |
| CD3 | The CD3-binding portion of AMG509 |
| CD3 | The CD3-binding portion of AMV564 |
| CD3 | The CD3-binding portion of APVO436 |
| CD3 | The CD3-binding portion of Alnuctamab (CC-93269; BMS-986349) |
| CD3 | The CD3-binding portion of ERY974 |
| CD3 | The CD3-binding portion of A-319 |
| CD3 | The CD3-binding portion of GEM333 |
| CD3 | The CD3-binding portion of GEM3PSCA |
| CD3 | The CD3-binding portion of Cevostamab |
| CD3 | The CD3-binding portion of Runimotamab |
| CD3 | The CD3-binding portion of GEN1044 |
| CD3 | Epcoritamab (GEN3013) |
| CD3 | The CD3-binding portion of HPN424 |
| CD3 | The CD3-binding portion of ISB1302 |
| CD3 | The CD3-binding portion of ISB1342 |
| CD3 | The CD3-binding portion of IGM-2323 |
| CD3 | The CD3-binding portion of IMC-F106C |
| CD3 | The CD3-binding portion of IMC-C103C |
| CD3 | The CD3-binding portion of IMCnyeso |
| CD3 | The CD3-binding portion of JNJ-63709178 |
| CD3 | The CD3-binding portion of JNJ-63898081 (JNJ-081) |
| CD3 | The CD3-binding portion of Teclistamab |
| CD3 | The CD3-binding portion of Talquetamab (JNJ-64407564) |
| CD3 | The CD3-binding portion of JNJ-67571244 |
| CD3 | The CD3-binding portion of MGD007 |
| CD3 | The CD3-binding portion of Orlotamab (MGD009) |
| CD3 | The CD3-binding portion of Duvortuxizumab |
| CD3 | The CD3-binding portion of Flotetuzumab (MGD006) |
| CD3 | The CD3-binding portion of MCLA-117 |
| CD3 | The CD3-binding portion of PF-06671008 |
| CD3 | The CD3-binding portion of Elranatamab |
| CD3 | The CD3-binding portion of Odronextamab |
| CD3 | The CD3-binding portion of REGN5458 |
| CD3 | The CD3-binding portion of REGN5459 |
| CD3 | The CD3-binding portion of REGN4018 |
| CD3 | The CD3-binding portion of Glofitamab (RO7082859) |
| CD3 | The CD3-binding portion of RO6958688 (RG7802) |
| CD3 | The CD3-binding portion of SAR440234 |
| CD3 | The CD3-binding portion of TNB-383B |
| CD3 | The CD3-binding portion of M802 |
| CD3 | The CD3-binding portion of Xmab 13676 |
| CD3 | The CD3-binding portion of Xmab18087 |
| CD3 | The CD3-binding portion of Vibecotamab (XmAb14045) |
| CD3 | The CD3-binding portion of Nivatrotamab (Hu3F8-BsAb) |
| CD3 | Anti-CD3 antibody sequences in US 2019/0211100 |
| CD3 | Anti-CD3 antibody sequences in EP1629011B |
| CD3 | VH of SEQ ID NOS. 90 and 98 disclosed in US 2021/0206865 A1 |
| | CDR-H1 of SEQ ID NO: 92 and 100 disclosed in US 2021/0206865 A1 |
| | CDR-H2 of SEQ ID NO: 94 and 102 disclosed in US 2021/0206865 A1 |
| | CDR-H3 of SEQ ID NO: 96 and 104 disclosed in US 2021/0206865 A1 |
| | HC of SEQ ID NO: 127 or SEQ ID NO: 128 disclosed in US 2021/0206865 A1 |
| | LC of SEQ ID NO: 129 or SEQ ID NO: 132 disclosed in US 2021/0206865 A1 |
| CD3 | Anti-CD3 Heavy chain of SEQ ID NO: 2 disclosed in US 2021/0206870 A1 |
| | Anti-CD3 VH SEQ ID NO: 5 disclosed in US 2021/0206870 A1 |
| | Anti-CD3 VL of SEQ ID NO: 6 disclosed in US 2021/0206870 A1 |
| | Anti-CD3 CDR-H1 of SEQ ID NO: 10 disclosed in US 2021/0206870 A1 |
| | Anti-CD3 CDR-H2 of SEQ ID NO: 11 disclosed in US 2021/0206870 A1 |
| | Anti-CD3 CDR-H3 of SEQ ID NO: 12 disclosed in US 2021/0206870 A1 |
| CD3 | Anti-CD3 VH of SEQ ID NO: 92, 102, 112, 122, 132, 142, 156, 166, 176, 186, 196 |
| | or 206 disclosed in US 2022/0119525 A1 |
| | Anti-CD3 CDR-H1 of SEQ ID NO: 93, 103, 113, 123, 133, 143, 157, 167, 177, |
| | 187, 197 or 207 disclosed in US 2022/0119525 A1 |
| | Anti-CD3 CDR-H2 of SEQ ID NO: 94, 104, 114, 124, 134, 144, 158, 168, 178, |
| | 188, 198 or 208 disclosed in US 2022/0119525 A1 |
| | Anti-CD3 CDR-H3 of SEQ ID NO: 95, 105, 115, 125, 135, 145, 159, 169, 179, |
| | 189, 199 or 209 disclosed in US 2022/0119525 A1 |
| | Anti-CD3 VL of SEQ ID NO: 96, 106, 116, 126, 136, 146, 152, 162, 172, 182, 192 |
| | or 202 disclosed in US 2022/0119525 A1 |
| | Anti-CD3 CDR-L1 of SEQ ID NO: 97, 107, 117, 127, 137, 147, 153, 163, 173, |
| | 183, 193 or 203 disclosed in US 2022/0119525 A1 |
| | Anti-CD3 CDR-L2 of SEQ ID NO. 98, 108, 118, 128, 138, 148, 154, 164, 174, |
| | 184, 194 or 204 disclosed in US 2022/0119525 A1 |
| | Anti-CD3 CDR-L3 of SEQ ID NO. 99, 109, 119, 129, 139, 149, 155, 165, 175, |
| | 185, 195 or 205 disclosed in US 2022/0119525 A1 |
| CD3 | L2K |
| CD3 | A2J |
| CD3 | 6G12 |
| CD3 | 1A4 |
| CD3 | OKT3 (Ortho Kung T3; Muromonab-CD3) |
| CD3 | Teplizumab (PRV-031; MGA03) |
| CD3 | Otelixizumab (TRX4) |
| CD3 | Anti-CD3 VH of SEQ ID NO: 2, 18, 34, 50, 66, 82, 98, 114, 130, 146, 162, 178, |
| | 194, 210, 226, 242, 258, 274, 290, 306, 322, 338, 354, 370, 386, 402, 418, 434, |
| | 450, 466, 482, 498, 514, 530, 546, 562, 578, 594, 610, 626, 642, 658, 674, 690, |
| | 706, 722, 738, 754, 770, 786, 802, 818, 834, 850, 866, 882, 898, 914, 930, 946, |
| | 962, 978, 994, 1010, 1026, 1042, 1050, 1058, 1066, 1074, 1082, 1090, 1098, |
| | 1106, 1114, 1122, 1130, 1138, 1146, 1154, 1162, 1170, 1178, 1186, 1194, 1202, |
| | 1210, 1218, or 1226 disclosed in U.S. Pat. No. 9,657,102 B2 |
| | Anti-CD3 CDR-H1 of SEQ ID NO: 4, 20, 36, 52, 68, 84, 100, 116, 132, 148, 164, |
| | 180, 196, 212, 228, 244, 260, 276, 292, 308, 324, 340, 356, 372, 388, 404, 420, |
| | 436, 452, 468, 484, 500, 516, 532, 548, 564, 580, 596, 612, 628, 644, 660, 676, |
| | 692, 708, 724, 740, 756, 772, 788, 804, 820, 836, 852, 868, 884, 900, 916, 932, |
| | 948, 964, 980, 996, 1012, 1028, 1044, 1052, 1060, 1068, 1076, 1084, 1092, |
| | 1100, 1108, 1116, 1124, 1132, 1140, 1148, 1156, 1164, 1172, 1180, 1188, 1196, |
| | 1204, 1212, or 1220, 1228 disclosed in U.S. Pat. No. 9,657,102 B2 |
| | Anti-CD3 CDR-H2 of SEQ ID NO: 6, 22, 38, 54, 70, 86, 102, 118, 134, 150, 166, |
| | 182, 198, 214, 230, 246, 262, 278, 294, 310, 326, 342, 358, 374, 390, 406, 422, |
| | 438, 454, 470, 486, 502, 518, 534, 550, 566, 582, 598, 614, 630, 646, 662, 678, |
| | 694, 710, 726, 742, 758, 774, 790, 806, 822, 838, 854, 870, 886, 902, 918, 934, |
| | 950, 966, 982, 998, 1014, 1030, 1046, 1054, 1062, 1070, 1078, 1086, 1094, |
| | 1102, 1110, 1118, 1126, 1134, 1142, 1150, 1158, 1166, 1174, 1182, 1190, 1198, |
| | 1206, 1214, or 1222, 1230 disclosed in U.S. Pat. No. 9,657,102 B2 |
| | Anti-CD3 CDR-H3 of SEQ ID NO: 8, 24, 40, 56, 72, 88, 104, 120, 136, 152, 168, |
| | 184, 200, 216, 232, 248, 264, 280, 296, 312, 328, 344, 360, 376, 392, 408, 424, |
| | 440, 456, 472, 488, 504, 520, 536, 552, 568, 584, 600, 616, 632, 648, 664, 680, |
| | 696, 712, 728, 744, 460, 776, 792, 808, 824, 840, 856, 872, 888, 904, 920, 936, |
| | 952, 968, 984, 1000, 1016, 1032, 1048, 1056, 1064, 1072, 1080, 1088, 1096, |
| | 1104, 1112, 1120, 1128, 1136, 1144, 1152, 1160, 1168, 1176, 1184, 1192, 1200, |
| | 1208, 1216, or 1224, 1232 disclosed in U.S. Pat. No. 9,657,102 B2 |
| | Anti-CD3 VL of SEQ ID NO: 10, 26, 42, 58, 74, 90, 106, 122, 138, 154, 170, 186, |
| | 202, 218, 234, 250, 266, 282, 298, 314, 330, 346, 362, 378, 394, 410, 426, 442, |
| | 458, 474, 490, 506, 522, 538, 554, 570, 586, 602, 618, 634, 650, 666, 682, 698, |
| | 714, 730, 746, 762, 778, 794, 810, 826, 842, 858, 874, 890, 906, 922, 938, 954, |
| | 970, 986, 1002, 1018, 1034, 1234, 1234, 1234, 1234, 1234, 1234, 1234, 1234, |
| | 1234, 1234, 1234, 1234, 1234, 1234, 1234, 1234, 1234, 1234, 1234, 1234, 1234, |
| | 1234, or 1234, 1234 disclosed in U.S. Pat. No. 9,657,102 B2 |
| | Anti-CD3 CDR-L1 of SEQ ID NO: 12, 28, 44, 60, 76, 92, 108, 124, 140, 156, 172, |
| | 188, 204, 220, 236, 252, 268, 284, 300, 316, 332, 348, 364, 380, 396, 412, 428, |
| | 444, 460, 476, 492, 508, 524, 540, 556, 572, 588, 604, 620, 636, 652, 668, 684, |
| | 700, 716, 732, 748, 764, 780, 796, 812, 828, 844, 860, 876, 892, 908, 924, 940, |
| | 956, 972, 988, 1004, 1020, 1036, 1236, 1236, 1236, 1236, 1236, 1236, 1236, |
| | 1236, 1236, 1236, 1236, 1236, 1236, 1236, 1236, 1236, 1236, 1236, 1236, 1236, |
| | 1236, 1236, or 1236, 1236 disclosed in U.S. Pat. No. 9,657,102 B2 |
| | Anti-CD3 CDR-L2 of SEQ ID NO. 14, 30, 46, 62, 78, 94, 110, 126, 142, 158, 174, |
| | 190, 206, 222, 238, 254, 270, 286, 302, 318, 334, 350, 366, 382, 398, 414, 430, |
| | 446, 462, 478, 494, 510, 526, 542, 558, 574, 590, 606, 622, 638, 654, 670, 686, |
| | 702, 718, 734, 750, 766, 782, 798, 814, 830, 846, 862, 878, 894, 910, 926, 942, |
| | 958, 974, 990, 1006, 1022, 1038, 1238, 1238, 1238, 1238, 1238, 1238, 1238, |
| | 1238, 1238, 1238, 1238, 1238, 1238, 1238, 1238, 1238, 1238, 1238, 1238, 1238, |
| | 1238, 1238, or 1238, 1238 disclosed in U.S. Pat. No. 9,657,102 B2 |
| | Anti-CD3 CDR-L3 of SEQ ID NO. 16, 32, 48, 64, 80, 96, 112, 128, 144, 160, 176, |
| | 192, 208, 224, 240, 256, 272, 288, 304, 320, 336, 352, 368, 384, 400, 416, 432, |
| | 448, 464, 480, 496, 512, 528, 544, 560, 576, 592, 608, 624, 640, 656, 672, 688, |
| | 704, 720, 736, 752, 768, 784, 800, 816, 832, 848, 864, 880, 896, 912, 928, 944, |
| | 960, 976, 992, 1008, 1024, 1040, 1240, 1240, 1240, 1240, 1240, 1240, 1240, |
| | 1240, 1240, 1240, 1240, 1240, 1240, 1240, 1240, 1240, 1240, 1240, 1240, 1240, |
| | 1240, 1240, or 1240, 1240 disclosed in U.S. Pat. No. 9,657,102 B2 |
| | (see also U.S. Pat. No. 9,657,102 B2 at Table 1, incorporated herein by |
| | reference) |
| CD3 | VH: |
| | EVQLVESGGGLVQPGRSLRLSCAASGFTFADYTMHWVRQAPGKGLEWVSDIS |
| | WNSGSIAYADSVKGRFTISRDNAKNSLYLQMNSLRTEDTAFYYCAKDSRGYG |
| | HYKYLGLDVWGQGTTVTVSS (SEQ ID NO: 46) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 47) |
| CD3 | VH: |
| | EVQLVESGGGLVQPGRSLRLSCAASGFTFDDYSMHWVRQAPGKGLEWVSGIS |
| | WNSGSKGYADSVKGRFTISRDNAKNSLYLQMNSLRAEDTALYYCAKYGSGYG |
| | KFYHYGLDVWGQGTTVTVSS (SEQ ID NO: 48) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 47) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKDGSGYG |
| | KFYYYGMDVWGQGTTVTVSS (SEQ ID NO: 49) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 47) |
| CD3 | VH: |
| | EVQLVESGGGLVQPGRSLRLSCAASGFTFDDYSMHWVRQAPGKGLEWVSGIS |
| | WNSGSIGYADSVKGRFTISRDNAKNSLYLQMNSLRAEDTALYYCAKYGSGYG |
| | KFYYYGMDVWGQGTTVTVSS (SEQ ID NO: 50) |
| | VL: |
| | DIQMTQSPSSLSASVGDRVTITCRASQSISSYLNWYQQKPGKAPKLLIYAAS |
| | SLQSGVPSRFSGSGSGTDFTLTISSLQPEDFATYYCQQSYSTPPITFGQGTR |
| | LEIK (SEQ ID NO: 47) |
| TCRαβ | BMA031 sequences disclosed in US 2012/0034221 |
| TCRγδ | 6TCS1 antibody disclosed in U.S. Pat. No. 5,980,892 |

In some aspects, the TCR complex targeting moiety competes with an antibody set forth in Table G for binding to the target (e.g., CD3 or a T-cell receptor). In further aspects, the TCR complex targeting moiety comprises CDRs having CDR sequences of an antibody set forth in Table G. In some embodiments, the TCR complex targeting moiety comprises all 6 CDR sequences of an antibody set forth in Table G. In other embodiments, the TCR complex targeting moiety comprises at least the heavy chain CDR sequences (CDR-H1, CDR-H2, CDR-H-3) or an antibody set forth in Table G and the light chain CDR sequences of a universal light chain. In further aspects, a TCR complex targeting moiety comprises a VH comprising the amino acid sequence of the VH of an antibody set forth in Table G. In some embodiments, the TCR complex targeting moiety further comprises a VL comprising the amino acid sequence of the VL of an antibody set forth in Table G. In other embodiments, the TCR complex targeting moiety further comprises a universal light chain VL sequence.

In some embodiments, the multispecific T-cell engager is a bispecific T-cell engager. Certain example bispecific T-cell engagers are provided in Table K. In some embodiments, a bispecific T-cell engager useful in combination with a tumor-targeted split IL12 agonist of the disclosure is a bispecific T-cell engager of Table K. In some embodiments, a bispecific T-cell engager comprises one or more CDR, VH, and/or VL sequences from a bispecific T-cell engager of Table K.

TABLE K
Exemplary Bispecific T-Cell Engagers

| Targets | Name and/or Binding Sequences |
|---|---|
| CD3 × BCMA | Bispecific antibodies bsAb25441D and bsAb25442D described in US |
| | 2022/0306758 A1 and US 2021/0206865 A1 |
| CD3 × CD20 | Bispecific antibodies BS3/20-001, BS3/20-002, BS3/20-003, BS3/20-004, |
| | BS3/20-005, BS3/20-007, and BS3/20-009 described in US 2018/0215823 |
| | A1 |
| CD3 × CD20 | Bispecific antibodies Antibody 1 and Antibody 2 described in US |
| | 20180194841 A1 |
| CD3 × MUC16 | Bispecific antibody BSMUC16/CD3-001 described in US 2020/0399371 A1 |
| CD3 × PSMA | Bispecific antibody PSMA/CD3-005 described in US 2020/0399372 A1 |
| CD3 × CD33 | Bispecific antibodies mAb2 G1 C-LC DANAPA IgG1, mAb2 D5 N-LC |
| | DANAPA IgG1 and mAb2 D5 C-LC DANAPA IgG1 described in US |
| | 2019/0153096A1 |
| CD3 × CLEC12A | Bispecific antibody 5196x4327 DM-Fc bsAb described in WO 2017/010874 |
| | A1 |
| CD3 × PSMA | Bispecific antibodies BSPSMA/CD3-001, BSPSMA/CD3-002, BSPSMA/CD3- |
| | 003, BSPSMA/CD3-200, BSPSMA/CD3-300, BSPSMA/CD3-400, |
| | BSPSMA/CD3-004, BSPSMA/CD3-800, BSPSMA/CD3-900, BSPSMA/CD3- |
| | 1000, BSPSMA/CD3-1100, BSPSMA/CD3-1200, BSPSMA/CD3-1300, |
| | BSPSMA/CD3-1400, BSPSMA/CD3-1500, BSPSMA/CD3-1600, |
| | BSPSMA/CD3-1700, BSPSMA/CD3-1800, BSPSMA/CD3-1900, |
| | BSPSMA/CD3-005, BSPSMA/CD3-2100 described in US 2021/0403595 A1 |
| CD3 × BCMA | Bispecific antibodies BCMB72, BC3B7, BC3B8, BC3B9, BC3B10, BC3B11, |
| | BC3B12 described in WO 2017/031104 A1 |
| CD3 × EpCAM | Catumaxomab, MT110 |
| CD3 × EpCAM | MT110 |
| CD3 × HER2/neu | Ertumaxomab |
| CD3 × HER2 | ISB1302 |
| CD3 × HER2 | Runimotamab |
| CD3 × HER2 | M802 |
| CD3 × PSMA | Acapatamab |
| CD3 × PSMA | BAY2010112 (Pasotuxizumab) |
| CD3 × PSMA | JNJ-63898081 (JNJ-081) |
| CD3 × MUC17 | AMG199 |
| CD3 × CD33 | AMG330 |
| CD3 × CD33 | AMG673 |
| CD3 × CD33 | AMV564 |
| CD3 × CD33 | GEM333 |
| CD3 × CD33 | JNJ-67571244 |
| CD3 × FLT3 | AMG427 (Emirodatamab) |
| CD3 × CD19 | AMG562 |
| CD3 × CD19 | A-319 |
| CD3 × CD19 | Duvortuxizumab |
| CD3 × EGFRvIII | AMG596 |
| CD3 × BCMA | Alnuctamab (CC-93269, BMS-986349) |
| CD3 × BCMA | AMG701 (Pavurutamab) |
| CD3 × BCMA | AMG420 |
| CD3 × BCMA | Teclistamab |
| CD3 × BCMA | Elranatamab |
| CD3 × BCMA | REGN5458 |
| CD3 × BCMA | REGN5459 |
| CD3 × BCMA | TNB-383B |
| CD3 × NY-ESO-1 | IMCnyeso |
| CD3 × MAGE-A4 | IMC-C103C |
| CD3 × PRAME | IMC-F106C |
| CD3 × 5T4 | GEN1044 |
| CD3 × DLL3 | AMG757 |
| CD3 × CLDN18.2 | AMG910 (Gresonitamab) |
| CD3 × GPC3 | ERY974 |
| CD3 × gpA33 | MGD007 |
| CD3 × B7-H3 | Orlotamab (MGD007) |
| CD3 × SSTR2 | XmAb-18087 |
| CD3 × PSCA | GEM3PSCA |
| CD3 × CD38 | AMG424 |
| CD3 × CD38 | ISB1342 |
| CD3 × STEAP1 | AMG509 |
| CD3 × FCRL5 | Cevostamab |
| CD3 × CD123 | APVO436 |
| CD3 × CD123 | JNJ-63709178 |
| CD3 × CD123 | Flotetuzumab (MGD006) |
| CD3 × CD123 | SAR440234 |
| CD3 × CD123 | Vibecotamab (XmAb14045) |
| CD3 × CD20 | Epcoritamab (GEN3013) |
| CD3 × CD20 | IGM-2323 |
| CD3 × CD20 | Odronextamab |
| CD3 × CD20 | Glofitamab (RO7082859) |
| CD3 × CD20 | XmAb13676 |
| CD3 × GPRC5D | Talquetamab (JNJ-64407564) |
| CD3 × CLEC12A | MCLA-117 |
| CD3 × MUC 16 | REGN4018 |
| CD3 × CEA | RO6958688 (RG7802) |
| CD3 × GD2 | Nivatrotamab (Hu3F8-BsAb) |

### 6.7. Targeting Moiety Formats

In certain aspects, a targeting moiety (e.g., a TAA targeting moiety, an IL12Rβ1 targeting moiety, an IL12Rβ2 targeting moiety, or a TCR complex targeting moiety) can be any type of antibody or fragment thereof that retains specific binding to an antigenic determinant. In one embodiment the antigen binding moiety is a full-length antibody. In one embodiment the antigen binding moiety is an immunoglobulin molecule, particularly an IgG class immunoglobulin molecule, more particularly an IgG1 or IgG4 immunoglobulin molecule. In another embodiment, the antigen binding moiety is single domain antibody. Antibody fragments include, but are not limited to, VH (or VH) fragments, VL (or VL) fragments, Fab fragments, F(ab′)2 fragments, scFv fragments, Fv fragments, VHH domains, minibodies, diabodies, triabodies, and tetrabodies.

In some embodiments, the TAA targeting moiety of the tumor-targeted IL12Rβ1 agonist and the TAA targeting moiety of the tumor-targeted IL12Rβ2 agonist share the same format (e.g., Fab, scFv or sdAb). In another embodiment, the TAA targeting moiety of the tumor-targeted IL12Rβ1 agonist and the TAA targeting moiety of the tumor-targeted IL12Rβ2 agonist do not share the same format.

In some embodiments the TAA targeting moieties of the tumor-targeted IL12Rβ1 agonist and the tumor-targeted IL12Rβ2 agonist are Fabs. In other embodiments, the TAA targeting moieties of the tumor-targeted IL12Rβ1 agonist and the tumor-targeted IL12Rβ2 agonist are scFvs. In yet other embodiments, and the TAA targeting moieties of the tumor-targeted IL12Rβ1 agonist and the tumor-targeted IL12Rβ2 agonist are sdAbs.

In some embodiments, where the IL12Rβ1 and IL12Rβ2 binding moieties of a tumor-targeted split IL12 receptor agonist are IL12Rβ1 and IL12Rβ2 targeting moieties, the IL12Rβ1 and IL12Rβ2 targeting moieties share the same format (e.g., Fab, scFv or sdAb). In other embodiments, where the IL12Rβ1 and IL12Rβ2 binding moieties of a tumor-targeted split IL12 receptor agonist are IL12Rβ1 and IL12Rβ2 targeting moieties, the IL12Rβ1 and IL12Rβ2 targeting moieties do not share the same format.

In some embodiments the IL12Rβ1 and IL12Rβ2 targeting moieties are Fabs. In other embodiments, the IL12Rβ1 and IL12Rβ2 targeting moieties are scFvs. In yet other embodiments, and the IL12Rβ1 and IL12Rβ2 targeting moieties are sdAbs.

In some embodiments, the TAA targeting moieties and the IL12Rβ1 and IL12Rβ2 targeting moieties share the same format (e.g., Fab, scFv or sdAb). In other embodiments, the the TAA targeting moieties and the IL12Rβ1 and IL12Rβ2 targeting moieties do not share the same format (e.g., the TAA targeting moieties are Fabs and the IL12Rβ1 and IL12Rβ2 targeting moieties are sdAbs or vice versa).

### 6.7.1. Fabs

Fab domains were traditionally produced by proteolytic cleavage of immunoglobulin molecules using enzymes such as papain. In the tumor-targeted split IL12 receptor agonists of the disclosure, the Fab domains can be recombinantly expressed as part of the tumor-targeted IL12Rβ1 agonist and/or the tumor-targeted IL12Rβ2 agonist.

The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the tumor-target split IL12 receptor agonists of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table F below can be used:

TABLE F
Fab Heterodimerization Strategies

| STRATEGY | VH | CH1 | VL | CL | REFERENCE |
|---|---|---|---|---|---|
| CrossMabC | WT | CL domain | WT | CH1 domain | Schaefer et al., |
| H1-CL | | | | | 2011, Cancer Cell |
| | | | | | 2011; 20:472-86; |
| | | | | | PMID:22014573. |
| orthogonal | 39K, 62E | H172A, | 1R, 38D, | L135Y, | Lewis et al., 2014, |
| Fab | | F174G | (36F) | S176W | Nat Biotechnol |
| VHVRD1CH | | | | | 32:191-8 |
| 1CRD2 - | | | | | |
| VLVRD1Cλ | | | | | |
| CRD2 | | | | | |
| orthogonal | 39Y | WT | 38R | WT | Lewis et al., 2014, |
| Fab | | | | | Nat Biotechnol |
| VHVRD2CH | | | | | 32:191-8 |
| 1wt - | | | | | |
| VLVRD2Cλ | | | | | |
| wt | | | | | |
| TCR CαCβ | 39K | TCR Cα | 38D | TCR Cβ | Wu et al., 2015, |
| | | | | | MAbs 7:364-76 |
| CR3 | WT | T192E | WT | N137K, | Golay at al., 2016, J |
| | | | | S114A | Immunol 196:3199- |
| | | | | | 211. |
| MUT4 | WT | L143Q, | WT | V133T, | Golay at al., 2016, J |
| | | S188V | | S176V | Immunol 196:3199- |
| | | | | | 211. |
| DuetMab | WT | F126C | WT | S121C | Mazor et al., 2015, |
| | | | | | MAbs 7:377-89; |
| | | | | | Mazor et al., 2015, |
| | | | | | MAbs 7:461-669. |
| Domain | WT | CH3 + knob | WT | CH3 + hole | Wozniak-Knopp et |
| exchanged | | or hole | | or knob | al., 2018, |
| | | mutation | | mutation | PLoSONE13(4):e01 |
| | | | | | 95442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T-cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of an IL12 receptor agonist of the disclosure. In various embodiments, employing a common light chain as described herein reduces the number of inappropriate species of IL12 receptor agonists as compared to employing original cognate VLs. In various embodiments, the VL domains of the IL12 receptor agonists are identified from monospecific antibodies comprising a common light chain. In various embodiments, the VH regions of the IL12 receptor agonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

6.7.2. scFvs

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.9.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5.3 (typically a repeat of a sequence containing the amino acids glycine and serine, such as the amino acid sequence (Gly4-Ser)₃ (SEQ ID NO:51), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.7.3. Single Domain Antibodies

In some embodiments, a targeting moiety e.g., a TAA targeting moiety, an IL12Rβ1 targeting moiety, or an IL12Rβ2 targeting moiety) is a single-domain antibody. A single-domain antibody (sdAb) describes a single antigen-binding domain capable of binding to a cognate antigen. sdAbs are often derived from heavy-chain only antibodies, however they also include single VH domains capable of binding to their cognate antigen in the absence of an associated light chain. In some embodiments, sdAbs also include single VL domains capable of binding to their cognate antigen in the absence of an associated light chain. Single VH or VL domains may have amino acid changes relative to native VH or VL sequences that stabilize the domains and/or reduce or eliminate aggregation.

Heavy-chain only antibodies lack both light chains and a functional CH1 domain and thus rely exclusively on a heavy chain variable domain for antigen binding. Heavy-chain only antibodies are produced naturally in the Camelidae family (e.g., camels, dromedaries, llamas, vicunas, guanaco, and alpacas) as well as in cartilaginous fish (e.g., sharks). In addition to natural sources, transgenic mammals (e.g., mice) have been engineered to express heavy-chain only antibodies. Such transgenic mammals include, for example, transgenic animals described in U.S. Patent Publications 2015/0289489 A1, 2023/0270086 A1, and 2023/0062964 A1, and 2020/0267951 A1, each of which is incorporated herein by reference.

In some embodiments, an sdAb is generated by immunizing an animal that produces heavy-chain only antibodies, including a natural producer (e.g., camelids, sharks) or an engineered non-human mammal (e.g., a transgenic mouse), to obtain heavy-chain only antibodies. Such antibodies may be screened to identify those having desirable properties (e.g., target affinity). Once produced and identified, the variable region of the antibody heavy chain is cloned to construct a single domain antibody consisting of only one heavy chain variable region.

sdAbs can also be obtained by immunizing animals that generate traditional antibodies (e.g., rabbits) followed by screening for VHs having high binding affinity in the absence of their cognate light chain (see e.g., Shinozaki et al., 2017, Scientific Reports, 7(1):5794).

sdAbs can be humanized by replacing natural (e.g., camelid) framework sequences with human sequences (see, e.g., Vincke, 2009, The Journal of Biological Chemistry, 285(5):3273-3284; Murakami et al., 2022, Antibodies, 11(1):10; and U.S. Patent Publication No. 2016/0237142 A1, incorporated herein by reference).

Fully human sdAbs can also be obtained using human VH single domains (see, e.g., Rouet et al., 2015, The Journal of Biological Chemistry, 290(19):11905-11917).

Additional methods for producing heavy-chain only antibodies and/or sdAbs are recognized in the art and include, for example, those described in Muyldermans, 2021, The FEBS journal, 288(7):2084-2102.

In some cases, an sdAb is engineered to enhance certain properties. For example, in some embodiments, a disulfide bond is introduced within a VHH to increase stability (see e.g., Hagihara et al., 2007, The Journal of Biological Chemistry, 282(50):36489-36495).

### 6.8. Fc Domains

The tumor-targeted IL12Rβ1 agonist and the tumor-targeted IL12Rβ2 agonist include an Fc domain of the tumor-targeted split IL12 receptor agonists of the disclosure each comprises a pair of Fc domains to which the TAA targeting moiety and the IL12Rβ1 binding moiety (in the case of the tumor-targeted IL12Rβ1 agonist) or the TAA targeting moiety and the IL12Rβ2 binding moiety (in the case of the tumor-targeted IL12Rβ2 agonist) are operably linked.

In some embodiments, the tumor-targeted IL12Rβ1 agonist comprises an Fc region formed by the association of an Fc pair, one comprising a TAA targeting moiety at its N-terminus and the other comprising an IL12Rβ1 binding moiety (e.g., an IL12Rβ1 targeting moiety or an IL12Rβ1 ligand) at its N-terminus.

In some embodiments, the tumor-targeted IL12Rβ2 agonist comprises an Fc region formed by the association of an Fc pair, one comprising a TAA targeting moiety at its N-terminus and the other comprising an IL12Rβ2 binding moiety (e.g., an IL12Rβ2 targeting moiety or an IL12Rβ2 ligand) at its N-terminus.

In one embodiment the Fc domains of the tumor-targeted IL12Rβ1 agonist and the tumor-targeted IL12Rβ2 agonist are derived from a human Fc domain.

The Fc domains that can be incorporated into a tumor-targeted IL12Rβ1 agonist and/or a tumor-targeted IL12Rβ2 agonist can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domains of both the tumor-targeted IL12Rβ1 agonist and the tumor-targeted IL12Rβ2 agonist are derived from IgG1, IgG2, IgG3 or IgG4. In one embodiment, one or both pairs of Fc domains are derived from IgG1. In one embodiment, one or both pairs of Fc domains are derived from IgG4.

The two Fc domains within the Fc region of the tumor-targeted IL12Rβ1 agonist and/or a tumor-targeted IL12Rβ2 agonist can be the same or different from one another. In a native antibody the Fc domains are typically identical, but for the purpose of producing molecules with different binding domains (e.g., a TAA targeting moiety and an IL12Rβ1 binding moiety or an IL12Rβ2 binding moiety, the Fc domains might advantageously be different to allow for heterodimerization, as described in Section 6.8.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In the tumor-targeted IL12Rβ1 agonists and/or tumor-targeted IL12Rβ2 agonists of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the tumor-targeted IL12Rβ1 agonists and/or tumor-targeted IL12Rβ2 agonists of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the tumor-targeted IL12Rβ1 agonists and/or tumor-targeted IL12Rβ2 agonists of the present disclosure do not comprise a tailpiece.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:11. In some embodiments, the a tumor-targeted IL12Rβ1 agonist and/or tumor-targeted IL12Rβ2 agonist comprises an Fc domain that has the amino acid sequence of SEQ ID NO: 11, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:12. In some embodiments, the a tumor-targeted IL12Rβ1 agonist and/or tumor-targeted IL12Rβ2 agonist comprises an Fc domain that has the amino acid sequence of SEQ ID NO:12, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:13. In some embodiments, the a tumor-targeted IL12Rβ1 agonist and/or tumor-targeted IL12Rβ2 agonist comprises an Fc domain that has the amino acid sequence of SEQ ID NO:13, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:14. In some embodiments, the a tumor-targeted IL12Rβ1 agonist and/or tumor-targeted IL12Rβ2 agonist comprises an Fc domain that has the amino acid sequence of SEQ ID NO:14, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:15. In some embodiments, the a tumor-targeted IL12Rβ1 agonist and/or tumor-targeted IL12Rβ2 agonist comprises an Fc domain that has the amino acid sequence of SEQ ID NO:15, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:41. In some embodiments, the a tumor-targeted IL12Rβ1 agonist and/or tumor-targeted IL12Rβ2 agonist comprises an Fc domain that has the amino acid sequence of SEQ ID NO:41, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:42. In some embodiments, the a tumor-targeted IL12Rβ1 agonist and/or tumor-targeted IL12Rβ2 agonist comprises an Fc domain that has the amino acid sequence of SEQ ID NO:42, optionally with one more mutations that facilitate heterodimerization or purification, e.g., (a) knob or hole mutations and/or (b) star mutations.

In some embodiments, a tumor-targeted IL12Rβ1 agonist and/or tumor-targeted IL12Rβ2 agonist comprises an Fc domain that has at least 95%, at least 96%, at least 97%, at least 98% or at least 99% sequence identity to the amino acid sequence of SEQ ID NO:43. In some embodiments, the a tumor-targeted IL12Rβ1 agonist and/or tumor-targeted IL12Rβ2 agonist comprises an Fc domain that has the amino acid sequence of SEQ ID NO:43, optionally with one more mutations that facilitate heterodimerization or purification, e.g., (a) knob or hole mutations and/or (b) star mutations.

The Fc domains that are incorporated into the tumor-targeted IL12Rβ1 agonists and/or tumor-targeted IL12Rβ2 agonists of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.8.1.

The Fc domains can also be altered to include modifications that improve manufacturability of asymmetric tumor-targeted IL12Rβ1 agonists and/or tumor-targeted IL12Rβ2 agonists, for example by allowing heterodimerization, which is the preferential pairing of non-identical Fc domains over identical Fc domains. Heterodimerization permits the production of tumor-targeted IL12Rβ1 agonists and/or tumor-targeted IL12Rβ2 agonists in which different polypeptide components are connected to one another by an Fc region containing Fc domains that differ in sequence. Examples of heterodimerization strategies are exemplified in Section 6.8.2.

It will be appreciated that any of the modifications mentioned above can be combined in any suitable manner to achieve the desired functional properties and/or combined with other modifications to alter the properties of the tumor-targeted IL12Rβ1 agonists and/or tumor-targeted IL12Rβ2 agonists.

6.8.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IL12 monomer) or the Fc region (e.g., one or both Fc domains of a tumor-targeted IL12Rβ1 agonists and/or tumor-targeted IL12Rβ2 agonist that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table C below: In some embodiments, the Fc domain includes only the bolded portion of the sequences shown below:

TABLE C

| | | SEQ |
|---|---|---|
| Fc Domain | Sequence | ID NO |
| SEQ ID NO: 1 of | DKRVESKYGP PCPPCPAPPV AGPSVFLFPP KPKDTLMISR | 16 |
| WO2014/121087 | TPEVTCVVVD VSQEDPEVQF NWYVDGVEVH NAKTKPREEQ | |
| | FNSTYRVVSV LTVLHQDWLN GKEYKCKVSN KGLPSSIEKT | |
| | ISKAKGQPRE PQVYTLPPSQ EEMTKNQVSL TCLVKGFYPS | |
| | DIAVEWESNG QPENNYKTTP PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL FPPKPKDTLM | 17 |
| WO2014/121087 | ISRTPEVTCV VVDVSQEDPE VQFNWYVDGV EVHNAKTKPR | |
| | EEQFNSTYRV VSVLTVLHQD WLNGKEYKCK VSNKGLPSSI | |
| | EKTISKAKGQ PREPQVYTLP PSRDELTKNQ VSLTCLVKGF | |
| | YPSDIAVEWE SNGQPENNYK TTPPVLDSDG SFFLYSKLTV | |
| | DKSRWQQGNV FSCSVMHEAL HNHYTQKSLS LSPGK | |
| SEQ ID NO: 30 of | ASTKGPSVFP LAPSSKSTSG GTAALGCLVK DYFPEPVTVS | 18 |
| WO2014/121087 | WNSGALTSGV HTFPAVLQSS GLYSLSSVVT VPSSSLGTQT | |
| | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| SEQ ID NO: 31 of | ASTKGPSVFP LAPCSRSTSE STAALGCLVK DYFPEPVTVS | 19 |
| WO2014/121087 | WNSGALTSGV HTFPAVLQSS GLYSLSSVVT VPSSSLGTKT | |
| | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNHYTQKSL | |
| | SLSLGK | |
| SEQ ID NO: 37 of | ASTKGPSVFP LAPSSKSTSG GTAALGCLVK DYFPEPVTVS | 20 |
| WO2014/121087 | WNSGALTSGV HTFPAVLQSS GLYSLSSVVT VPSSSLGTQT | |
| | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNRFTQ | |
| | KSLSLSPGK | |
| SEQ ID NO: 38 of | ASTKGPSVFP LAPCSRSTSE STAALGCLVK DYFPEPVTVS | 21 |
| WO2014/121087 | WNSGALTSGV HTFPAVLQSS GLYSLSSVVT VPSSSLGTKT | |
| | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNRFTQKSL | |
| | SLSLGK | |

In a particular embodiment, the IgG4 with reduced effector function comprises the bolded portion of the amino acid sequence of SEQ ID NO: 19 (SEQ ID NO:31 of WO2014/121087), sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:18 (SEQ ID NO:30 of WO2014/121087) (or the bolded portion thereof) and an Fc domain comprising the amino acid sequence of SEQ ID NO:20 (SEQ ID NO:37 of WO2014/121087) (or the bolded portion thereof) or an Fc region comprising an Fc domain comprising the amino acid sequence of SEQ ID NO:19 (SEQ ID NO:31 of WO2014/121087) (or the bolded portion thereof) and an Fc domain comprising the amino acid sequence of SEQ ID NO:21 (SEQ ID NO:38 of WO2014/121087) (or the bolded portion thereof).

### 6.8.2. Fc Heterodimerization Variants

Certain tumor-targeted IL12Rβ1 agonists and/or tumor-targeted IL12Rβ2 agonists entail dimerization between two Fc domains that, unlike a native immunoglobulin, are operably linked to non-identical N-terminal regions, e.g., one Fc domain connected to a TAA targeting moiety and the other Fc domain connected to an IL12Rβ1 or IL12Rβ2 binding moiety. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the tumor-targeted IL12Rβ1 agonists and/or tumor-targeted IL12Rβ2 agonists of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides tumor-targeted IL12Rβ1 agonists and/or tumor-targeted IL12Rβ2 agonists comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired tumor-targeted IL12Rβ1 agonist and/or tumor-targeted IL12Rβ2 agonist, while homodimerization of identical heavy chains will reduce yield of the desired tumor-targeted IL12Rβ1 agonist and/or tumor-targeted IL12Rβ2 agonist. Thus, in a preferred embodiment, the polypeptides that associate to form a tumor-targeted IL12Rβ1 agonist and/or tumor-targeted IL12Rβ2 agonist of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (WA). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL12 receptor agonists comprise a first CH3 domain and a second Ig CH3 domain, wherein the first and second Ig CH3 domains differ from one another by at least one amino acid, and wherein at least one amino acid difference reduces binding of the tumor-targeted IL12Rβ1 agonist and/or tumor-targeted IL12Rβ2 agonist to Protein A as compared to a corresponding tumor-targeted IL12Rβ1 agonist and/or tumor-targeted IL12Rβ2 agonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.9. Linkers

In certain aspects, the present disclosure provides tumor-targeted split IL12 receptor agonists in which two or more components of a tumor-targeted IL12Rβ1 agonist and/or a tumor-targeted IL12Rβ2 agonist are connected to one another by a peptide linker. By way of example and not limitation, linkers can be used to connect (a) an IL12Rβ1 binding moiety or a IL12Rβ2 binding moiety (e.g., an IL12 moiety or an anti-IL12Rβ1 or anti-IL12Rβ2 Fab, scFv, or sdAb) and an Fc domain; (b) a tumor targeting moiety and an Fc domain; (c) different domains within an IL12Rβ1 binding moiety or a IL12Rβ2 binding moiety (e.g., different domains within an IL12 moiety, such as a p35 moiety and a p40 moiety); or (d) different domains within a tumor targeting moiety (e.g., the VH and VL domains in a scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the tumor-targeted split IL12 receptor agonists of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS (SEQ ID NO: 23) or SGn, where n is an integer from 1 to 10, e.g., 1 2, 3, 4, 5, 6, 7, 8, 9 or 10 (SEQ ID NO: 24). In one embodiment, the linker is or comprises a monomer or multimer of repeat of G4S (SEQ ID NO: 25) e.g., (GGGGS)n (SEQ ID NO: 25).

Polyglycine linkers can suitably be used in the tumor-targeted split IL12 receptor agonists of the disclosure. In some embodiments, a peptide linker comprises two consecutive glycines (2Gly), three consecutive glycines (3Gly), four consecutive glycines (4Gly) (SEQ ID NO: 27), five consecutive glycines (5Gly) (SEQ ID NO: 28), six consecutive glycines (6Gly) (SEQ ID NO: 29), seven consecutive glycines (7Gly) (SEQ ID NO: 30), eight consecutive glycines (8Gly) (SEQ ID NO: 31) or nine consecutive glycines (9Gly) (SEQ ID NO: 22).

### 6.9.1. Hinge Sequences

In other embodiments, the tumor-targeted split IL12 receptor agonists of the disclosure comprise a linker that is a hinge region. In particular, where a tumor-targeted IL12Rβ1 agonist and/or a tumor-targeted IL12Rβ2 of a tumor-targeted split IL12 receptor agonist contains an immunoglobulin-based targeting moiety, the hinge can be used to connect the targeting moiety, e.g., a Fab domain, to a multimerization domain, e.g., an Fc domain. The hinge region can be a native or a modified hinge region. Hinge regions are typically found at the N-termini of Fc regions. The term “hinge region”, unless the context dictates otherwise, refers to a naturally or non-naturally occurring hinge sequence that in the context of a single or monomeric polypeptide chain is a monomeric hinge domain and in the context of a dimeric polypeptide (e.g., a heterodimeric tumor-targeted IL12Rβ1 agonist or a tumor-targeted IL12Rβ2 agonist formed by the association of two Fc domains) can comprise two associated hinge sequences on separate polypeptide chains.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, a tumor-targeted IL12Rβ1 agonist of the disclosure comprises an Fc region in which one or both Fc domains possesses an intact hinge region at its N-terminus.

In one embodiment, a tumor-targeted IL12Rβ2 agonist of the disclosure comprises an Fc region in which one or both Fc domains possesses an intact hinge region at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the tumor-targeted IL12Rβ1 agonists and/or the tumor-targeted IL12Rβ2 agonists of the disclosure comprise a modified hinge region that reduces binding affinity for an Fcγ receptor relative to a wild-type hinge region of the same isotype (e.g., human IgG1 or human IgG4).

In one embodiment, the tumor-targeted IL12Rβ1 agonists and/or the tumor-targeted IL12Rβ2 agonists of the disclosure comprise an Fc region in which each Fc domain possesses an intact hinge region at its N-terminus, where each Fc domain and hinge region is derived from IgG4 and each hinge region comprise the modified sequence CPPC (SEQ ID NO: 32). The core hinge region of human IgG4 contains the sequence CPSC (SEQ ID NO: 33) compared to IgG1 that contains the sequence CPPC (SEQ ID NO: 32). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.9.1.1. Chimeric Hinge Sequences

The hinge region can be a chimeric hinge region.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 34) (previously disclosed as SEQ ID NO:8 of WO2014/121087, which is incorporated by reference in its entirety herein) or ESKYGPPCPPCPAPPVA (SEQ ID NO: 35) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

6.9.1.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 36) (previously disclosed as SEQ ID NO:1 of WO2016161010A2), CPPCPAPGG--GPSVF (SEQ ID NO: 37) (previously disclosed as SEQ ID NO:2 of WO2016161010A2), CPPCPAPG---GPSVF (SEQ ID NO: 38) (previously disclosed as SEQ ID NO:3 of WO2016161010A2), or CPPCPAP----GPSVF (SEQ ID NO: 39) (previously disclosed as SEQ ID NO:4 of WO2016161010A2).

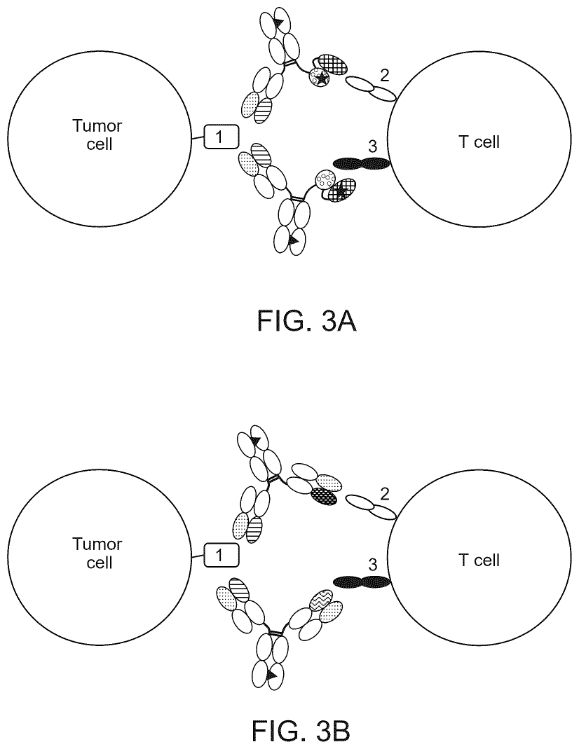

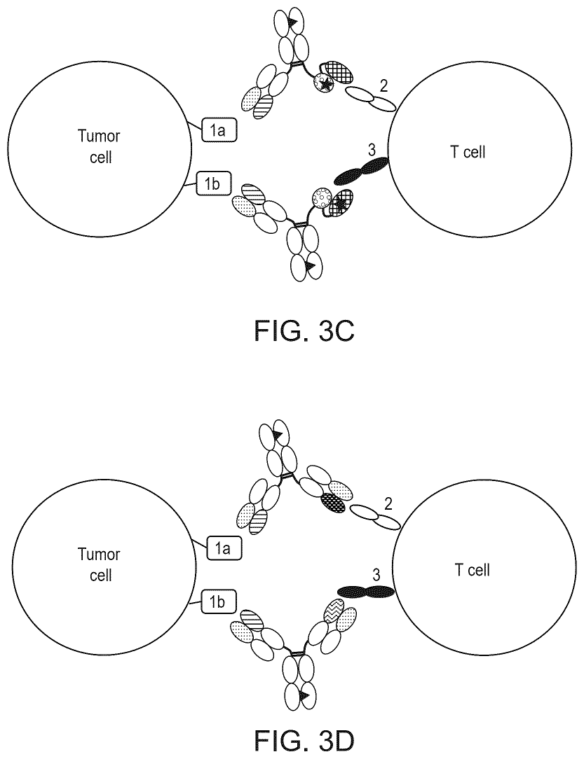

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1.1).

The linkers useful in the tumor-targeted split IL12 receptor agonists of the disclosure are typically non-cleavable linkers. A non-cleavable linker is one whose amino acid sequences lacks a (canonical) substrate sequence a for a protease, for example a substrate as set forth in Table B on pages 45-49 of international patent publication no. WO2024040249A1 and/or a protease as set forth in Table A on pages 43-44 of international application publication no. WO2024040249A1. The contents of Tables A and B of WO2024040249A1 are incorporated by reference herein.

### 6.10. Nucleic Acids and Host Cells

In another aspect, the disclosure provides nucleic acids encoding the tumor-targeted split IL12 receptor agonists of the disclosure and/or their individual components (the tumor-targeted IL12Rβ1 agonists and the tumor-targeted IL12Rβ2 agonists). In some embodiments, the tumor-targeted split IL12 receptor agonists, the tumor-targeted IL12Rβ1 agonists and/or the tumor-targeted IL12Rβ2 agonists are encoded by a single nucleic acid. In other embodiments, the tumor-targeted IL12Rβ1 agonists and the tumor-targeted IL12Rβ2 agonists are encoded by separate nucleic acids. In other embodiments, for example in the case of a heterodimeric tumor-targeted IL12Rβ1 agonists and/or tumor-targeted IL12Rβ2 agonist, one or both of the tumor-targeted IL12Rβ1 agonists and/or the tumor-targeted IL12Rβ2 agonists, e.g., when comprising an Fc heterodimer or a targeting moiety composed of more than one polypeptide chain, the tumor-targeted IL12Rβ1 agonists and/or the tumor-targeted IL12Rβ2 agonist are encoded by a plurality of (e.g., two, three, four or more) nucleic acids.

A single nucleic acid can encode a tumor-targeted IL12Rβ1 agonist and/or a tumor-targeted IL12Rβ2 agonist that comprises a single polypeptide chain, a tumor-targeted IL12Rβ1 agonists and/or a tumor-targeted IL12Rβ2 agonist that comprises two or more polypeptide chains, or a portion of a tumor-targeted IL12Rβ1 agonist and/or a tumor-targeted IL12Rβ2 agonist that comprises more than two polypeptide chains (for example, a single nucleic acid can encode two polypeptide chains of a tumor-targeted IL12Rβ1 agonists and/or a tumor-targeted IL12Rβ2 agonist comprising three, four or more polypeptide chains, or three polypeptide chains of a tumor-targeted IL12Rβ1 agonist and/or a tumor-targeted IL12Rβ2 agonist comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a tumor-targeted IL12Rβ1 agonist and/or a tumor-targeted IL12Rβ2 agonist comprising two or more polypeptide chains is encoded by two or more nucleic acids. The number of nucleic acids encoding an IL12 receptor agonist can be equal to or less than the number of polypeptide chains in the IL12 receptor agonist (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.10.1. Vectors

The disclosure provides vectors comprising nucleotide sequences encoding a tumor-targeted IL12Rβ1 agonist and/or a tumor-targeted IL12Rβ2 agonist or component thereof described herein, for example one or two of the polypeptide chains of a tumor-targeted IL12Rβ1 agonists and/or a tumor-targeted IL12Rβ2 agonist. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.10.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.11. Pharmaceutical Compositions

The tumor-targeted split IL12 receptor agonists of the disclosure may be in the form of compositions comprising the tumor-targeted IL12Rβ1 agonists and/or the tumor-targeted IL12Rβ2 agonists and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the tumor-targeted split IL12 receptor agonist and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a tumor-targeted IL12Rβ1 receptor agonist and/or tumor-targeted IL12Rβ2 receptor agonist per dose. The quantity of the tumor-targeted IL12Rβ1 receptor agonist and/or tumor-targeted IL12Rβ2 receptor agonist included in a unit dose will depend on the disease being treated, as well as other factors as are well known in the art. Such unit dosages may be in the form of a lyophilized dry powder containing an amount of the tumor-targeted IL12Rβ1 receptor agonist and/or tumor-targeted IL12Rβ2 receptor agonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of the tumor-targeted IL12Rβ1 receptor agonist and/or tumor-targeted IL12Rβ2 receptor agonist suitable for a single administration.

The pharmaceutical compositions may also be supplied in bulk from containing quantities of the tumor-targeted IL12Rβ1 agonist and/or the tumor-targeted IL12Rβ2 agonist suitable for multiple administrations.

When formulated into a single formulation, the tumor-targeted IL12Rβ1 receptor agonist and tumor-targeted IL12Rβ2 receptor agonist can be used in approximately equimolar quantities.

Pharmaceutical compositions may be prepared for storage as lyophilized formulations or aqueous solutions by mixing a tumor-targeted IL12Rβ1 agonist and/or a tumor-targeted IL12Rβ2 agonist having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IL12 receptor agonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.12. Therapeutic Indications and Methods of Treatment

The present disclosure provides methods for using and applications for the tumor-targeted split IL12 receptor agonists of the disclosure.

Tumor-targeted split IL12 receptor agonists of the disclosure are useful in treating disease states where stimulation of the immune system of the host is beneficial, in particular conditions where an enhanced cellular immune response is desirable. These may include disease states where the host immune response is insufficient or deficient.

Disease states for which the tumor-targeted split IL12 receptor agonists of the disclosure can be administered comprise, for example, a tumor or infection where a cellular immune response would be a critical mechanism for specific immunity. Specific disease states for which tumor-targeted split IL12 receptor agonists of the present disclosure can be employed include cancer, including breast cancer, prostate cancer, and colorectal cancer. The tumor-targeted split IL12 receptor agonists of the disclosure may be administered per se or in any suitable pharmaceutical composition.

In various embodiments, the tumor-targeted split IL12 receptor agonists of the disclosure are useful for the treatment of cancer, for the prevention or treatment of metastasis, for stimulating the formation, stability and/or activity of a cytotoxic immune synapse, for inducing tumor cytolysis, for inducing anti-tumor cytotoxicity, for stimulating an immune response against a tumor, or any combination of two or more of the foregoing uses.

In one aspect, tumor-targeted split IL12 receptor agonists of the disclosure for use as a medicament are provided. In further aspects, tumor-targeted split IL12 receptor agonists of the disclosure for use in treating a disease are provided. In certain embodiments, tumor-targeted split IL12 receptor agonists of the disclosure for use in a method of treatment are provided. In one embodiment, the disclosure provides a tumor-targeted split IL12 receptor agonist as described herein for use in the treatment of a disease in a subject in need thereof. In certain embodiments, the disclosure provides a tumor-targeted split IL12 receptor agonist for use in a method of treating a subject having a disease comprising administering to the individual a therapeutically effective amount of the tumor-targeted split IL12 receptor agonist. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer. In further embodiments, the disclosure provides a tumor-targeted split IL12 receptor agonist for use in stimulating the immune system. In certain embodiments, the disclosure provides a tumor-targeted split IL12 receptor agonist for use in a method of stimulating the immune system in a subject comprising administering to the individual an effective amount of the tumor-targeted split IL12 receptor agonist to stimulate the immune system. An “individual” according to any of the above embodiments is a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL12 receptors, an increase in T-cell responsiveness, an increase in natural killer cell activity or lymphokine-activated killer (LAK) cell activity, and the like.

In a further aspect, the disclosure provides for the use of a tumor-targeted split IL12 receptor agonist of the disclosure in the manufacture or preparation of a medicament for the treatment of a disease in a subject in need thereof. In one embodiment, the medicament is for use in a method of treating a disease comprising administering to a subject having the disease a therapeutically effective amount of the medicament. In certain embodiments the disease to be treated is a proliferative disorder. In a preferred embodiment the disease is cancer. In one such embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL12 receptors, an increase in T-cell responsiveness, an increase in natural killer cell activity or lymphokine-activated killer (LAK) cell activity, and the like.

In a further aspect, the disclosure provides a method for treating a disease in a subject, comprising administering to said individual a therapeutically effective amount of a tumor-targeted split IL12 receptor agonist of the disclosure (with the tumor-targeted IL12Rβ1 agonist and the tumor-targeted IL12Rβ2 agonist in separate pharmaceutical preparations or the same pharmaceutical preparation. In one embodiment one or two compositions comprising a tumor-targeted IL12Rβ1 agonist and a tumor-targeted IL12Rβ2 agonist in a pharmaceutically acceptable form, e.g., in equimolar amounts, are administered to said individual. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer. In a further aspect, the disclosure provides a method for stimulating the immune system in a subject, comprising administering to the individual an effective amount of a tumor-targeted split IL12 receptor agonist to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL12 receptors, an increase in T-cell responsiveness, an increase in natural killer cell activity or lymphokine-activated killer (LAK) cell activity, and the like.

In certain aspects, the disclosure provides a method of treating cancer, comprising administering to a subject in need thereof a tumor-targeted split IL12 receptor agonist or (a) pharmaceutical composition(s) comprising the tumor-targeted IL12Rβ1 agonist and the tumor-targeted IL12Rβ2 agonist as described herein.

In some embodiments, the disclosure provides a method of treating cancer with a tumor-targeted split IL12 receptor agonist that is targeted to cancer tissue, comprising administering to a subject in need thereof a tumor-targeted split IL12 receptor agonist or (a) pharmaceutical composition(s) comprising the tumor-targeted IL12Rβ1 agonist and the tumor-targeted IL12Rβ2 agonist as described herein, with the tumor-targeted IL12Rβ1 agonist and the tumor-targeted IL12Rβ2 agonist each comprising a targeting moiety that recognizes a target molecule that is expressed on the cancer cells.

The present disclosure further provides a method of localized delivery of a tumor-targeted split IL12 agonist, comprising administering to a subject a tumor-targeted split IL12 receptor agonist or (a) pharmaceutical composition(s) comprising the tumor-targeted IL12Rβ1 agonist and the tumor-targeted IL12Rβ2 agonist as described herein, where the tumor-targeted IL12Rβ1 agonist and the tumor-targeted IL12Rβ2 agonist comprise a targeting moiety that recognizes a target molecule that is expressed by a tissue to which the tumor-targeted split IL12 receptor agonist is to be locally delivered. As used herein, the term “locally delivered” does not require local administration but rather indicates that the tumor-targeted IL12 receptor agonist be selectively localized to a tissue of interest following administration.

The present disclosure further provides a method of administering to the subject IL12 therapy with reduced systemic exposure and/or reduced systemic toxicity and/or an improved therapeutic index, comprising administering to a subject the IL12 therapy in the form of a tumor-targeted split IL12 receptor agonist or (a) pharmaceutical composition(s) comprising the tumor-targeted IL12Rβ1 agonist and the tumor-targeted IL12Rβ2 agonist as described herein. Accordingly, the foregoing methods permit IL12 therapy with reduced off-target side effects by virtue of preferential targeting of an IL12 receptor agonist to a particular target tissue and/or improved anti-tumor cytotoxicity at the site of intended activity.

The present disclosure further provides method of locally inducing an immune response in a target tissue, comprising administering to a subject a tumor-targeted split IL12 receptor agonist or (a) pharmaceutical composition(s) comprising the tumor-targeted IL12Rβ1 agonist and the tumor-targeted IL12Rβ2 agonist as described herein, where the tumor-targeted IL12Rβ1 agonist and the tumor-targeted IL12Rβ2 agonist each comprise a targeting moiety capable of binding a target molecule expressed in the target tissue. The tumor-targeted split IL12 receptor agonist can then induce the immune response against at least one cell type in the target tissue. In some embodiments, the target tissue is cancer tissue.

In some embodiments, the administration is not local to the tissue. For example, when the target tissue is cancer tissue, the administration can be systemic or subcutaneous.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a tumor-targeted split IL12 receptor agonist of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. Similarly, other cell proliferation disorders can also be treated by the IL12 receptor agonists of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

Table I below shows exemplary indications for which tumor-targeted split IL12 receptor agonists targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| MELTF | melanoma |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional target molecules and corresponding indications are disclosed in, e.g., Hafeez et al., 2020, Molecules 25:4764, doi:10.3390/molecules25204764, particularly in Table 1. Table 1 is incorporated by reference in its entirety here.

A skilled artisan readily recognizes that in many cases the tumor-targeted split IL12 receptor agonists may not provide a cure but may only provide partial benefit. In some embodiments, a physiological change having some benefit is also considered therapeutically beneficial. Thus, in some embodiments, an amount of tumor-targeted split IL12 receptor agonist that provides a physiological change is considered an “effective amount” or a “therapeutically effective amount”.

The subject, patient, or individual in need of treatment is typically a mammal, more specifically a human.

The appropriate dosage of a tumor-targeted split IL12 receptor agonist of the disclosure (when used alone or in combination with one or more other additional therapeutic agents, e.g., a multispecific T-cell engager) will depend on the type of disease to be treated, the route of administration, the body weight of the patient, the particular tumor-targeted IL12Rβ1 agonist and tumor-targeted IL12Rβ2 agonist in the tumor-targeted split IL12 receptor agonist, the severity and course of the disease, whether the antibody is administered for preventive or therapeutic purposes, previous or concurrent therapeutic interventions, the patient's clinical history and response to the tumor-targeted split IL12 receptor agonist, and the discretion of the attending physician. In some embodiments, the tumor-targeted IL12Rβ1 agonist and tumor-targeted IL12Rβ2 are administered concurrently and/or in equimolar amounts. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A single administration of unconjugated IL12 can range from about 50,000 IU/kg to about 1,000,000 IU/kg or more, more typically about 600,000 IU/kg of IL12. This may be repeated several times a day (e.g., 2-3 times), for several days (e.g., about 3-5 consecutive days) and then may be repeated one or more times following a period of rest (e.g., about 7-14 days). Thus, a therapeutically effective amount may comprise only a single administration or many administrations over a period of time (e.g., about 20-30 individual administrations of about 600,000 IU/kg of IL12 each given over about a 10-20 day period).

Similarly, the tumor-targeted split IL12 receptor agonist is suitably administered to the patient at one time or over a series of treatments, each comprising administration of both a tumor-targeted IL12Rβ1 agonist and a tumor-targeted IL12Rβ2 agonist. Depending on the type and severity of the disease, about 1 μg/kg to 15 mg/kg (e.g., 0.1 mg/kg-10 mg/kg) of each of the tumor-targeted IL12Rβ1 agonist and tumor-targeted IL12Rβ2 agonist can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the IL12 receptor agonist would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the tumor-targeted split IL12 receptor agonist). An initial higher loading dose, followed by one or more lower doses may be administered. However, other dosage regimens may be useful. The progress of this therapy is easily monitored by conventional techniques and assays.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the tumor-targeted IL12Rβ1 agonist and tumor-targeted IL12Rβ2 agonist which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the tumor-targeted IL12Rβ1 agonist and tumor-targeted IL12Rβ2 agonist may not be related to plasma concentration. One having skill in the art will be able to optimize therapeutically effective local dosages without undue experimentation.

Due to lower toxicity, the tumor-targeted split IL12 receptor agonists of the disclosure can have higher maximum therapeutic doses than wild type IL12, although, the tumor-targeted split IL12 receptor agonists are typically administered at lower doses than wild type IL12 due to the prolonged half-lives.

### 6.13. Combination Therapy

The tumor-targeted split IL12 receptor agonists disclosed herein may be administered in combination with one or more other agents in therapy. For instance, a tumor-targeted split IL12 receptor agonist of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In particular embodiments, the additional therapeutic is a multispecific T-cell engager as described in Section 6.6, including but not limited to the multispecific T-cell engagers set forth in Table K.

Such other agents are suitably present in combination in amounts that are effective for the purpose intended. The effective amount of such other agents depends on the amount of tumor-targeted split IL12 receptor agonists used, the type of disorder or treatment, and other factors discussed above. The tumor-targeted split IL12 receptor agonists are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the tumor-targeted split IL12 receptor agonists can occur prior to, simultaneously, and/or following, administration of the additional therapeutic agent and/or adjuvant. Tumor-targeted split IL12 receptor agonists of the disclosure can also be used in combination with radiation therapy.

### 7. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.
1. A combination comprising:
   (a) a tumor-targeted IL12Rβ1 agonist comprising:
      (i) a first tumor-targeting moiety (e.g., an antibody or antibody fragment comprising means for binding a tumor-associated antigen); and
      (ii) an IL12Rβ1 binding moiety (e.g., an antibody or antibody fragment comprising means for binding IL12Rβ1); and
   (b) a tumor-targeted IL12Rβ2 agonist comprising:
      (i) a second tumor-targeting moiety (e.g., an antibody or antibody fragment comprising means for binding a tumor-associated antigen); and
      (ii) a IL12Rβ2 binding moiety (e.g., an antibody or antibody fragment comprising means for binding IL12Rβ2);
   for use as a combination therapy, optionally for use as a combination therapy for the treatment of cancer, for use as a combination therapy for the prevention or treatment of metastasis, for use as combination therapy for stimulating the formation, stability and/or activity of a cytotoxic immune synapse, for use as combination therapy for clustering of IL12Rβ1 and IL12Rβ2 receptor subunits in a lymphocyte, for eliciting signaling through the IL12 receptor in a lymphocyte, for use as combination therapy for inducing tumor cytolysis, for use combination therapy for inducing anti-tumor cytotoxicity, for use combination therapy for stimulating an immune response against a tumor, or any combination of two or more of the foregoing uses.
2. The combination of embodiment 1, for use as a combination therapy for the treatment of cancer.
3. The combination of embodiment 1 or embodiment 2, for use as a combination therapy for the prevention or treatment of metastasis.
4. The combination of any one of embodiments 1 to 3, for use as combination therapy for stimulating the formation, stability and/or activity of a cytotoxic immune synapse.
5. The combination of any one of embodiments 1 to 4, for use as combination therapy for clustering of IL12Rβ1 and IL12Rβ2 receptor subunits in a lymphocyte.
6. The combination of any one of embodiments 1 to 5, for use as combination therapy for eliciting signaling through the IL12 receptor in a lymphocyte.
7. The combination of any one of embodiments 1 to 6, for use as a combination therapy for inducing tumor cytolysis.
8. The combination of any one of embodiments 1 to 7, for use as a combination therapy for inducing anti-tumor cytotoxicity.
9. The combination of any one of embodiments 1 to 8, for use as a combination therapy for stimulating an immune response against a tumor.
10. A method comprising administering to a subject in need thereof a combination comprising:
   (a) a tumor-targeted IL12Rβ1 agonist (“R1 agonist”) comprising:
      (i) a first tumor-targeting moiety (e.g., an antibody or antibody fragment comprising means for binding a tumor-associated antigen); and
      (ii) an IL12Rβ1 binding moiety (e.g., an antibody or antibody fragment comprising means for binding IL12Rβ1); and
   (b) a tumor-targeted IL12Rβ2 agonist (“R2 agonist”) comprising:
      (i) a second tumor-targeting moiety (e.g., an antibody or antibody fragment comprising means for binding a tumor-associated antigen); and
      (ii) a IL12Rβ2 binding moiety (e.g., an antibody or antibody fragment comprising means for binding IL12Rβ2),
   optionally wherein the method is a method of combination therapy for the treatment of cancer, a method of combination therapy for the prevention or treatment of metastasis, a method of combination therapy for stimulating the formation, stability and/or activity of a cytotoxic immune synapse, a method for clustering of IL12Rβ1 and IL12Rβ2 receptor subunits in a lymphocyte, a method for eliciting signaling through the IL12 receptor in a lymphocyte, a method of combination therapy for inducing tumor cytolysis, a method of combination therapy for inducing anti-tumor cytotoxicity, a method of combination therapy for stimulating an immune response against a tumor, or a combination of any two or more of the foregoing methods.
11. The method of embodiment 10, which is a method for the treatment of cancer.
12. The method of embodiment 10 or embodiment 11, which is a method for the prevention or treatment of metastasis.
13. The method of any one of embodiments 10 to 12, which is a method for stimulating the formation, stability and/or activity of a cytotoxic immune synapse.
14. The method of any one of embodiments 10 to 13, which is a method for clustering of IL12Rβ1 and IL12Rβ2 receptor subunits in a lymphocyte.
15. The method of any one of embodiments 10 to 14, which is a method for eliciting signaling through the IL12 receptor in a lymphocyte.
16. The method of any one of embodiments 10 to 15, which is a method for inducing tumor cytolysis.
17. The method of any one of embodiments 10 to 16, which is a method for inducing anti-tumor cytotoxicity.
18. The method of any one of embodiments 10 to 17, which is a method for stimulating an immune response against a tumor.
19. The combination of any one of embodiments 1 to 9 or the method of any one of embodiments 10 to 18, wherein the IL12Rβ1 binding moiety and the IL12Rβ2 binding moiety each comprises or consists of an antigen binding domain of an antibody.
20. The combination or method of embodiment 19, wherein the IL12Rβ1 binding moiety and the IL12Rβ2 binding moiety are Fabs.
21. The combination or method of embodiment 19, wherein the IL12Rβ1 binding moiety and the IL12Rβ2 binding moiety are scFvs.
22. The combination or method of embodiment 19, wherein the IL12Rβ1 binding moiety and the IL12Rβ2 binding moiety are sdAbs.
23. The combination or method of embodiment 22, wherein the IL12Rβ1 binding moiety and the IL12Rβ2 binding moiety are sdVHs.
24. The combination or method of any one of embodiments 19 to 22, wherein the antigen binding domain of an antibody of the IL12Rβ1 binding moiety comprises means for binding human IL12Rβ1.
25. The combination or method of any one of embodiments 19 to 22, wherein the antigen binding domain of an antibody of the IL12Rβ2 binding moiety comprises means for binding human IL12Rβ2.
26. The combination or method of any one of embodiments 19 to 22, wherein the IL12Rβ1 binding moiety binds to the D2 domain of IL12Rβ1 and the IL12Rβ2 binding moiety binds to the D1 domain of IL12Rβ2.
27. The combination or method of any one of embodiments 19 to 22, wherein the IL12Rβ1 binding moiety comprises means for binding to the D2 domain of IL12Rβ1 and the IL12Rβ2 binding moiety comprises means for binding to the D1 domain of IL12Rβ2.
28. The combination of any one of embodiments 1 to 9 or the method of any one of embodiments 10 to 18, wherein:
   (a) the IL12Rβ1 binding moiety is a first IL12 moiety comprising a first p40 moiety optionally associated with a first p35 moiety; and
   (b) the IL12Rβ2 binding moiety is a second IL12 moiety comprising a second p35 moiety optionally associated with a second p40 moiety.
29. The combination or method of embodiment 28, wherein:
   (a) the first IL12 moiety has greater selectivity to IL12Rβ1 than IL12Rβ2 as compared to wild-type IL12 (e.g., wild-type human IL12); and/or
   (b) the second IL12 moiety has greater selectivity to IL12Rβ2 than IL12Rβ1 as compared to wild-type IL12 (e.g., wild-type human IL12).
30. The combination or method of embodiment 28 or embodiment 29, wherein the first IL12 moiety comprises a first p35 moiety and a first p40 moiety.
31. The combination or method of any one of embodiments 28 to 30, wherein the first p35 moiety comprises an amino acid sequence having at least 95% sequence identity to the amino acid sequence of SEQ ID NO:2.
32. The combination or method of any one of embodiments 28 to 30, wherein the first p35 moiety comprises an amino acid sequence having at least 96% sequence identity to the amino acid sequence of SEQ ID NO:2.
33. The combination or method of any one of embodiments 28 to 30, wherein the first p35 moiety comprises an amino acid sequence having at least 97% sequence identity to the amino acid sequence of SEQ ID NO:2.
34. The combination or method of any one of embodiments 28 to 30, wherein the first p35 moiety comprises an amino acid sequence having at least 98% sequence identity to the amino acid sequence of SEQ ID NO:2.
35. The combination or method of any one of embodiments 28 to 30, wherein the first p35 moiety comprises an amino acid sequence having at least 99% sequence identity to the amino acid sequence of SEQ ID NO:2.
36. The combination or method of any one of embodiments 28 to 35, wherein the first p35 moiety is a variant p35 moiety having reduced binding to IL12Rβ2 as compared to a wild-type p35 moiety (e.g., a p35 moiety having the amino acid sequence of SEQ ID NO:2).
37. The combination or method of embodiment 36, wherein the variant p35 moiety comprises one or more of the mutations set forth in Table 1, optionally wherein the one or more mutations comprise or consist of Y189E as compared to wild-type p35 (e.g., wherein the variant p35 moiety comprises the amino acid sequence of SEQ ID NO:40).
38. The combination or method of any one of embodiments 28 to 30, wherein the first p35 moiety comprises the amino acid sequence of SEQ ID NO:2.
39. The combination or method of any one of embodiments 28 to 38, wherein the first p40 moiety comprises an amino acid sequence having at least 95% sequence identity to the amino acid sequence of SEQ ID NO:6 or SEQ ID NO:7.
40. The combination or method of any one of embodiments 28 to 38, wherein the first p40 moiety comprises an amino acid sequence having at least 96% sequence identity to the amino acid sequence of SEQ ID NO:6 or SEQ ID NO:7.
41. The combination or method of any one of embodiments 28 to 38, wherein the first p40 moiety comprises an amino acid sequence having at least 97% sequence identity to the amino acid sequence of SEQ ID NO:6 or SEQ ID NO:7.
42. The combination or method of any one of embodiments 28 to 38, wherein the first p40 moiety comprises an amino acid sequence having at least 98% sequence identity to the amino acid sequence of SEQ ID NO:6 or SEQ ID NO:7.
43. The combination or method of any one of embodiments 28 to 38, wherein the first p40 moiety comprises an amino acid sequence having at least 99% sequence identity to the amino acid sequence of SEQ ID NO:6 or SEQ ID NO:7.
44. The combination or method of any one of embodiments 28 to 38, wherein the first p40 moiety comprises the amino acid sequence of SEQ ID NO:6 or SEQ ID NO:7.
45. The combination or method of any one of embodiments 28 to 44, wherein the second IL12 moiety comprises a second p35 moiety and a second p40 moiety.
46. The combination or method of embodiment 45, wherein the second p35 moiety comprises an amino acid sequence having at least 95% sequence identity to the amino acid sequence of SEQ ID NO:2.
47. The combination or method of embodiment 45, wherein the second p35 moiety comprises an amino acid sequence having at least 96% sequence identity to the amino acid sequence of SEQ ID NO:2.
48. The combination or method of embodiment 45, wherein the second p35 moiety comprises an amino acid sequence having at least 97% sequence identity to the amino acid sequence of SEQ ID NO:2.
49. The combination or method of embodiment 45, wherein the second p35 moiety comprises an amino acid sequence having at least 98% sequence identity to the amino acid sequence of SEQ ID NO:2.
50. The combination or method of embodiment 45, wherein the second p35 moiety comprises an amino acid sequence having at least 99% sequence identity to the amino acid sequence of SEQ ID NO:2.
51. The combination or method of embodiment 45, wherein the second p35 moiety comprises the amino acid sequence of SEQ ID NO:2.
52. The combination or method of any one of embodiments 45 to 51, wherein the second p40 moiety comprises an amino acid sequence having at least 95% sequence identity to the amino acid sequence of SEQ ID NO:6 or SEQ ID NO:7.
53. The combination or method of any one of embodiments 45 to 51, wherein the second p40 moiety comprises an amino acid sequence having at least 96% sequence identity to the amino acid sequence of SEQ ID NO:6 or SEQ ID NO:7.
54. The combination or method of any one of embodiments 45 to 51, wherein the second p40 moiety comprises an amino acid sequence having at least 97% sequence identity to the amino acid sequence of SEQ ID NO:6 or SEQ ID NO:7.
55. The combination or method of any one of embodiments 45 to 51, wherein the second p40 moiety comprises an amino acid sequence having at least 98% sequence identity to the amino acid sequence of SEQ ID NO:6 or SEQ ID NO:7.
56. The combination or method of any one of embodiments 45 to 51, wherein the second p40 moiety comprises an amino acid sequence having at least 99% sequence identity to the amino acid sequence of SEQ ID NO:6 or SEQ ID NO:7.
57. The combination or method of embodiment 56, wherein the second p40 moiety is a variant p40 moiety having reduced binding to IL12Rβ1 as compared to a wild-type p40 moiety.
58. The combination or method of embodiment 57, wherein the variant p40 moiety comprises a D1 domain mutation or a D1 domain deletion.
59. The combination or method of any one of embodiments 45 to 58, wherein the second p40 moiety comprises the amino acid sequence of SEQ ID NO:6.
60. The combination or method of any one of embodiments 28 to 59, wherein the first p35 moiety and the first p40 moiety are separated by a linker (a first “IL12 moiety linker”) and/or the second p35 moiety and the second p40 moiety are separated by a linker (a second “IL12 moiety linker”).
61. The combination or method of embodiment 60, wherein the first IL12 moiety linker and/or the second IL12 moiety linker are each at least 5 amino acids in length.
62. The combination or method of embodiment 60, wherein the first IL12 moiety linker and/or the second IL12 moiety linker are each least 10 amino acids in length.
63. The combination or method of embodiment 60, wherein the first IL12 moiety linker and/or the second IL12 moiety linker are each least 15 amino acids in length.
64. The combination or method of any one of embodiments 60 to 63, wherein the first IL12 moiety linker and/or the second IL12 moiety linker are a glycine-serine linker.
65. The combination or method of any one of embodiments 60 to 64, wherein the first IL12 moiety linker and/or the second IL12 moiety linker comprises the amino acid sequence G4S.
66. The combination or method of embodiment 64, wherein the first IL12 moiety linker and/or the second IL12 moiety linker comprises a multimer of the amino acid sequence G4S.
67. The combination or method of embodiment 66, wherein the multimer comprises 2, 3, 4, 5, 6 or more repeats of the amino acid sequence G4S.
68. The combination or method of any one of embodiments 60 to 67, wherein the first IL12 moiety linker and/or the second IL12 moiety linker is a non-cleavable linker.
69. The combination of any one of embodiments 1 to 9 and 19 to 68 or the method of any one of embodiments 10 to 68, wherein the first tumor-targeting moiety binds to a first tumor-associated antigen and the second tumor-targeting moiety binds to a second tumor-associated antigen.
70. The combination or method of embodiment 69, wherein the first tumor-associated antigen and the second tumor-associated antigen are expressed on the same tumor cell.
71. The combination or method of embodiment 69 or embodiment 70, wherein the first tumor-associated antigen and the second tumor-associated antigen are different.
72. The combination or method of embodiment 69 or embodiment 70, wherein the first tumor-associated antigen and the second tumor-associated antigen are the same.
73. The combination or method of embodiment 72, wherein the first tumor-targeting moiety and the second tumor-targeting moiety are the same.
74. The combination or method of embodiment 72, wherein the first tumor-targeting moiety and the second tumor-targeting moiety are different, e.g., bind to different epitopes.
75. The combination or method of embodiment 72 or embodiment 74, wherein the first tumor-targeting moiety and the second tumor-targeting moiety do not compete for binding to the tumor-associated antigen.
76. The combination or method of any one of embodiments 69 to 75, wherein the first tumor-targeting moiety and/or the second tumor-targeting moiety are Fabs.
77. The combination or method of any one of embodiments 69 to 75, wherein the first tumor-targeting moiety and/or the second tumor-targeting moiety are scFvs.
78. The combination or method of any one of embodiments 69 to 75, wherein the first tumor-targeting moiety and/or the second tumor-targeting moiety are sdAbs.
79. The combination or method of embodiment 78, wherein the first tumor-targeting moiety and/or the second tumor-targeting moiety are sdVHs.
80. The combination or method of any one of embodiments 69 to 79, wherein the first tumor-targeting moiety and/or second tumor-targeting moiety bind(s) to Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, prostate-specific membrane antigen (PSMA), T-cell receptor/CD3-zeta chain, GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, pi20ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, Her3, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD20, MCSP, PDGFPR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, PAP, LAGA-1a, prostein, survivin and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) of fibronectin or the A1 domain of tenascin-C (TnC A1).
81. The combination or method of any one of embodiments 69 to 79, wherein the first tumor-targeting moiety and/or second tumor-targeting moiety bind(s) to human PSMA.
82. The combination or method of any one of embodiments 69 to 79, wherein the first tumor-targeting moiety and/or second tumor-targeting moiety bind(s) to human MSLN.
83. The combination or method of any one of embodiments 69 to 79, wherein the first tumor-targeting moiety and/or second tumor-targeting moiety bind(s) to human MUC16.
84. The combination or method of any one of embodiments 69 to 79, first tumor-targeting moiety and/or second tumor-targeting moiety comprise means for binding to a tumor antigen.
85. The combination or method of any one of embodiments 69 to 79, first tumor-targeting moiety and/or second tumor-targeting moiety comprise means for binding human PSMA.
86. The combination or method of any one of embodiments 69 to 79, first tumor-targeting moiety and/or second tumor-targeting moiety comprise means for binding human MSLN.
87. The combination or method of any one of embodiments 69 to 79, first tumor-targeting moiety and/or second tumor-targeting moiety comprise means for binding human MUC16.
88. The combination of any one of embodiments 1 to 9 and 19 to 87 or the method of any one of embodiments 10 to 87, wherein the tumor-targeted IL12Rβ1 agonist comprises
   (a) a first polypeptide chain comprising, in N- to C-terminal orientation:
      (i) the first tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
      (ii) optionally, a linker (a “TAA-Fc linker”); and
      (iii) a first Fc domain; and
   (b) a second polypeptide chain comprising, in N- to C-terminal orientation:
      (i) the IL12Rβ1 binding moiety or component thereof (or a component thereof, e.g., a VH-CH1 or VL-CL), optionally associated with another component thereof on a separate polypeptide chain (or a component thereof, e.g., a VL-CL or VH-CH1);
      (ii) optionally, a linker (e.g., where the IL12Rβ1 binding moiety is an IL12 moiety as defined in any one of embodiments 28 to 68) (“an IL12-Fc linker”), optionally wherein the IL12-Fc linker is as defined in any one of embodiments 61 to 67); and
      (iii) a second Fc domain associated with the first Fc domain.
89. The combination or method of embodiment 88, wherein the first and second Fc domains are IgG1, IgG2, IgG3 or IgG4 Fc domains.
90. The combination or method of embodiment 88 or embodiment 89, wherein the first Fc domain and second Fc domain each comprises an amino acid sequence having at least 95% sequence identity to the amino acid sequence of SEQ ID NO:11.
91. The combination or method of embodiment 88 or embodiment 89, wherein the first Fc domain and second Fc domain each comprises an amino acid sequence having at least 95% sequence identity to the amino acid sequence of SEQ ID NO:12.
92. The combination or method of embodiment 88 or embodiment 89, wherein the first Fc domain and second Fc domain each comprises an amino acid sequence having at least 95% sequence identity to the amino acid sequence of SEQ ID NO:13.
93. The combination or method of any one of embodiments 88 to 92, wherein the first Fc domain and second Fc domain each comprises a chimeric hinge domain.
94. The combination or method of embodiment 93, wherein the first Fc domain and second Fc domain each comprises an amino acid sequence having at least 98% or at least 99% sequence identity to the amino acid sequence of SEQ ID NO:14.
95. The combination or method of embodiment 93, wherein the first Fc domain and second Fc domain each comprises an amino acid sequence having at least 98% or at least 99% sequence identity to the amino acid sequence of SEQ ID NO:15.
96. The combination or method of any one of embodiments 88 to 95, wherein the first Fc domain and second Fc domain each has reduced effector function.
97. The combination or method of any one of embodiments 88 to 96, wherein the first Fc domain and second Fc domain form an Fc heterodimer.
98. The combination of any one of embodiments 1 to 9 and 19 to 97 or the method of any one of embodiments 10 to 97, wherein the tumor-targeted IL12Rβ2 agonist comprises
   (a) a third polypeptide chain comprising, in N- to C-terminal orientation:
      (i) the second tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
      (ii) optionally, a linker (a “TAA-Fc linker”); and
      (iii) a third Fc domain; and
   (b) a third polypeptide chain comprising, in N- to C-terminal orientation:
      (i) the IL12Rβ2 binding moiety or component thereof (or a component thereof, e.g., a VH-CH1 or VL-CL), optionally associated with another component thereof on a separate polypeptide chain (or a component thereof, e.g., a VL-CL or VH-CH1);
      (ii) optionally, a linker (e.g., where the IL12Rβ2 binding moiety is an IL12 moiety as defined in any one of embodiments 28 to 68) (“an IL12-Fc linker”), optionally wherein the IL12-Fc linker is as defined in any one of embodiments 61 to 67); and
      (iii) a fourth Fc domain associated with the third Fc domain.
99. The combination or method of embodiment 98, wherein the third and fourth Fc domains are IgG1, IgG2, IgG3 or IgG4 Fc domains.
100. The combination or method of embodiment 98 or embodiment 99, wherein the third Fc domain and fourth Fc domain each comprises an amino acid sequence having at least 95% sequence identity to the amino acid sequence of SEQ ID NO:11.
101. The combination or method of embodiment 98 or embodiment 100, wherein the third Fc domain and fourth Fc domain each comprises an amino acid sequence having at least 95% sequence identity to the amino acid sequence of SEQ ID NO:12.
102. The combination or method of embodiment 98 or embodiment 100, wherein the third Fc domain and fourth Fc domain each comprises an amino acid sequence having at least 95% sequence identity to the amino acid sequence of SEQ ID NO:13.
103. The combination or method of any one of embodiments 98 to 102, wherein the third Fc domain and fourth Fc domain each comprises a chimeric hinge domain.
104. The combination or method of any one of embodiments 98 to 103, wherein the third Fc domain and fourth Fc domain each has reduced effector function.
105. The combination or method of any one of embodiments 98 to 104, wherein the third Fc domain and fourth Fc domain form an Fc heterodimer.
106. The combination of any one of embodiments 1 to 9 and 19 to 105 or the method of any one of embodiments 10 to 105, wherein the tumor-targeted IL12Rβ1 agonist is monovalent for the first tumor-targeting moiety.
107. The combination of any one of embodiments 1 to 9 and 19 to 106 or the method of any one of embodiments 10 to 106, wherein the tumor-targeted IL12Rβ1 agonist is monovalent for the IL12Rβ1 binding moiety.
108. The combination of any one of embodiments 1 to 9 and 19 to 107 or the method of any one of embodiments 10 to 107, wherein the tumor-targeted IL12Rβ2 agonist is monovalent for the second tumor-targeting moiety.
109. The combination of any one of embodiments 1 to 9 and 19 to 108 or the method of any one of embodiments 10 to 108, wherein the tumor-targeted IL12Rβ2 agonist is monovalent for the IL12Rβ2 binding moiety.
110. The combination of any one of embodiments 1 to 9 and 19 to 109 or the method of any one of embodiments 10 to 109, wherein the tumor-targeted IL12Rβ1 agonist and the tumor-targeted IL12Rβ2 agonist are both in the form of a pharmaceutical composition comprising the agonist and an excipient.
111. The combination or method of embodiment 110, wherein the tumor-targeted IL12Rβ1 agonist and the tumor-targeted IL12Rβ2 agonist are in the same pharmaceutical composition.
112. The combination or method of embodiment 110, wherein the tumor-targeted IL12Rβ1 agonist and the tumor-targeted IL12Rβ2 agonist are in different pharmaceutical compositions.
113. The combination of any one of embodiments 1 to 9 and 19 to 112 or the method of any one of embodiments 10 to 112, wherein tumor-targeted IL12Rβ1 agonist is configured as illustrated in FIG. 1A.
114. The combination of any one of embodiments 1 to 9 and 19 to 112 or the method of any one of embodiments 10 to 112, wherein tumor-targeted IL12Rβ1 agonist is configured as illustrated in FIG. 1B.
115. The combination of any one of embodiments 1 to 9 and 19 to 112 or the method of any one of embodiments 10 to 112, wherein tumor-targeted IL12Rβ1 agonist is configured as illustrated in FIG. 1C.
116. The combination of any one of embodiments 1 to 9 and 19 to 112 or the method of any one of embodiments 10 to 112, wherein tumor-targeted IL12Rβ1 agonist is configured as illustrated in FIG. 1D.
117. The combination of any one of embodiments 1 to 9 and 19 to 112 or the method of any one of embodiments 10 to 112, wherein tumor-targeted IL12Rβ1 agonist is configured as illustrated in FIG. 1E.
118. The combination of any one of embodiments 1 to 9 and 19 to 112 or the method of any one of embodiments 10 to 112, wherein tumor-targeted IL12Rβ1 agonist is configured as illustrated in FIG. 1F.
119. The combination of any one of embodiments 1 to 9 and 19 to 112 or the method of any one of embodiments 10 to 112, wherein tumor-targeted IL12Rβ1 agonist is configured as illustrated in FIG. 1G.
120. The combination of any one of embodiments 1 to 9 and 19 to 112 or the method of any one of embodiments 10 to 112, wherein tumor-targeted IL12Rβ1 agonist is configured as illustrated in FIG. 1H.
121. The combination of any one of embodiments 1 to 9 and 19 to 112 or the method of any one of embodiments 10 to 112, wherein tumor-targeted IL12Rβ1 agonist is configured as illustrated in FIG. 1I.
122. The combination of any one of embodiments 1 to 9 and 19 to 112 or the method of any one of embodiments 10 to 112, wherein tumor-targeted IL12Rβ1 agonist is configured as illustrated in FIG. 1J.
123. The combination of any one of embodiments 1 to 9 and 19 to 112 or the method of any one of embodiments 10 to 112, wherein tumor-targeted IL12Rβ1 agonist is configured as illustrated in FIG. 1K.
124. The combination of any one of embodiments 1 to 9 and 19 to 112 or the method of any one of embodiments 10 to 112, wherein tumor-targeted IL12Rβ1 agonist is configured as illustrated in FIG. 1L.
125. The combination of any one of embodiments 1 to 9 and 19 to 124 or the method of any one of embodiments 10 to 124, wherein tumor-targeted IL12Rβ2 agonist is configured as illustrated in FIG. 2A.
126. The combination of any one of embodiments 1 to 9 and 19 to 124 or the method of any one of embodiments 10 to 124, wherein tumor-targeted IL12Rβ2 agonist is configured as illustrated in FIG. 2B.
127. The combination of any one of embodiments 1 to 9 and 19 to 124 or the method of any one of embodiments 10 to 124, wherein tumor-targeted IL12Rβ2 agonist is configured as illustrated in FIG. 2C.
128. The combination of any one of embodiments 1 to 9 and 19 to 124 or the method of any one of embodiments 10 to 124, wherein tumor-targeted IL12Rβ2 agonist is configured as illustrated in FIG. 2D.
129. The combination of any one of embodiments 1 to 9 and 19 to 124 or the method of any one of embodiments 10 to 124, wherein tumor-targeted IL12Rβ2 agonist is configured as illustrated in FIG. 2E.
130. The combination of any one of embodiments 1 to 9 and 19 to 124 or the method of any one of embodiments 10 to 124, wherein tumor-targeted IL12Rβ2 agonist is configured as illustrated in FIG. 2F.
131. The combination of any one of embodiments 1 to 9 and 19 to 124 or the method of any one of embodiments 10 to 124, wherein tumor-targeted IL12Rβ2 agonist is configured as illustrated in FIG. 2G.
132. The combination of any one of embodiments 1 to 9 and 19 to 124 or the method of any one of embodiments 10 to 124, wherein tumor-targeted IL12Rβ2 agonist is configured as illustrated in FIG. 2H.
133. The combination of any one of embodiments 1 to 9 and 19 to 124 or the method of any one of embodiments 10 to 124, wherein tumor-targeted IL12Rβ2 agonist is configured as illustrated in FIG. 2I.
134. The combination of any one of embodiments 1 to 9 and 19 to 124 or the method of any one of embodiments 10 to 124, wherein tumor-targeted IL12Rβ2 agonist is configured as illustrated in FIG. 2J.
135. The combination of any one of embodiments 1 to 9 and 19 to 124 or the method of any one of embodiments 10 to 124, wherein tumor-targeted IL12Rβ2 agonist is configured as illustrated in FIG. 2K.
136. The combination of any one of embodiments 1 to 9 and 19 to 124 or the method of any one of embodiments 10 to 124, wherein tumor-targeted IL12Rβ2 agonist is configured as illustrated in FIG. 2L.
137. The combination of any one of embodiments 1 to 9 and 19 to 135 or the method of any one of embodiments 10 to 135, wherein the combination further comprises or the method further comprises administering a multispecific T-cell engager (e.g., simultaneously, sequentially or separately).
138. The combination or method of embodiment 137, wherein the multispecific T-cell engager is a bispecific T-cell engager.
139. The combination or method of embodiment 137 or 138, wherein the multispecific T-cell engager comprises means for binding a TAA and means for binding CD3.
140. The combination or method of embodiment 137 or 138, wherein the multispecific T-cell engager comprises a TAA targeting moiety (e.g., an antibody or antigen-binding fragment comprising means for binding a TAA) and a CD3 targeting moiety (e.g., an antibody or antigen-binding fragment comprising means for binding CD3).
141. The combination or method of embodiment 140, wherein the TAA targeting moiety of the multispecific T-cell engager targets the same TAA as the TAA targeted by the tumor-targeted IL12Rβ1 agonist and/or the tumor-targeted IL12Rβ2 agonist.
142. The combination or method of embodiment 140, wherein the TAA targeting moiety of the multispecific T-cell engager targets a TAA that is different from the TAA targeted by the tumor-targeted IL12Rβ1 agonist and/or the tumor-targeted IL12Rβ2 agonist.
143. The combination of any one of embodiments 1 to 9 and 19 to 142 for use as a medicament.
144. The combination for use of embodiment 143 for use in a method for the treatment of cancer.
145. The combination for use of embodiment 143 or 144 for use in a method for the prevention or treatment of metastasis.
146. The combination for use of any one of embodiments 143 to 145, for use in a method of stimulating the formation, stability and/or activity of a cytotoxic immune synapse.
147. The combination for use of any one of embodiments 143 to 146, for use in a method of clustering of IL12Rβ1 and IL12Rβ2 receptor subunits in a lymphocyte.
148. The combination for use of any one of embodiments 143 to 147, for use in a method of eliciting signaling through the IL12 receptor in a lymphocyte.
149. The combination for use of any one of embodiments 143 to 148, for use in a method of inducing tumor cytolysis.
150. The combination for use of any one of embodiments 143 to 149, for use in a method of inducing anti-tumor cytotoxicity.
151. The combination for use of any one of embodiments 143 to 150, for use in a method of stimulating an immune response against a tumor.
152. The combination for use of any one of embodiments 143 to 151, for use in a method for the treatment of cancer.
153. A tumor-targeted IL12Rβ1 agonist for use in a method for the treatment of cancer, wherein
   (a) the IL12Rβ1 agonist is defined as in any one of embodiments 1 to 136, and
   (b) the method comprises administering to a subject in need thereof the tumor-targeted IL12Rβ1 agonist and a tumor-targeted IL12Rβ2 agonist as defined in any one of embodiments 1 to 136.
154. The tumor-targeted IL12Rβ1 agonist for use of embodiment 153, wherein the method is a method of combination therapy for the prevention or treatment of metastasis.
155. The tumor-targeted IL12Rβ1 agonist for use of embodiment 153 or 154, wherein the method is a method of combination therapy for stimulating the formation, stability and/or activity of a cytotoxic immune synapse.
156. The tumor-targeted IL12Rβ1 agonist for use of any one of embodiments 153 to 155, wherein the method is a method of clustering of IL12Rβ1 and IL12Rβ2 receptor subunits in a lymphocyte.
157. The tumor-targeted IL12Rβ1 agonist for use of any one of embodiments 153 to 156, wherein the method is a method of eliciting signaling through the IL12 receptor in a lymphocyte.
158. The tumor-targeted IL12Rβ1 agonist for use of any one of embodiments 153 to 157, wherein the method is a method of inducing tumor cytolysis.
159. The tumor-targeted IL12Rβ1 agonist for use of any one of embodiments 153 to 158, wherein the method is a method of inducing anti-tumor cytotoxicity.
160. The tumor-targeted IL12Rβ1 agonist for use of any one of embodiments 153 to 159, wherein the method is a method of stimulating an immune response against a tumor.
161. A tumor-targeted IL12Rβ2 agonist for use in a method for the treatment of cancer, wherein
   (a) the IL12Rβ2 agonist is defined as in any one of embodiments 1 to 136, and
   (b) the method comprises administering to a subject in need thereof the tumor-targeted IL12Rβ2 agonist and a tumor-targeted IL12Rβ1 agonist as defined in any one of embodiments 1 to 136.
162. The tumor-targeted IL12Rβ2 agonist for use of embodiment 161, wherein the method is a method of combination therapy for the prevention or treatment of metastasis.
163. The tumor-targeted IL12Rβ2 agonist for use of embodiment 161 or 162, wherein the method is a method of combination therapy for stimulating the formation, stability and/or activity of a cytotoxic immune synapse.
164. The tumor-targeted IL12Rβ2 agonist for use of any one of embodiments 161 to 163, wherein the method is a method of clustering of IL12Rβ1 and IL12Rβ2 receptor subunits in a lymphocyte.
165. The tumor-targeted IL12Rβ2 agonist for use of any one of embodiments 161 to 164, wherein the method is a method of eliciting signaling through the IL12 receptor in a lymphocyte.
166. The tumor-targeted IL12Rβ2 agonist for use of any one of embodiments 161 to 165, wherein the method is a method of inducing tumor cytolysis.
167. The tumor-targeted IL12Rβ2 agonist for use of any one of embodiments 161 to 166, wherein the method is a method of inducing anti-tumor cytotoxicity.
168. The tumor-targeted IL12Rβ2 agonist for use of any one of embodiments 161 to 167, wherein the method is a method of stimulating an immune response against a tumor.

### 8. EXAMPLES

### 8.1. Materials and Methods

### 8.1.1. Design and Production of Tumor-Targeted IL12Rβ1 and IL12Rβ2 Agonist Constructs

Exemplary tumor-targeted IL12Rβ1 agonists as depicted in FIG. 1A were designed to comprise two polypeptides herein referred to as the first and second polypeptides, the first polypeptide comprising from N- to C-terminal, a first tumor targeting moiety in Fab format, a linker (“TAA linker”), and a first Fc domain which enables dimerization; and the second polypeptide comprising from N- to C-terminal, an IL12Rβ1 binding moiety comprising an IL12 moiety in the format of a p40-p35 fusion peptide comprising a wild-type p40 moiety linked to a mutated p35 moiety with an IL12 moiety linker, a linker (“IL12-Fc linker”), and a second Fc domain that forms a dimer with the first Fc domain. Exemplary tumor-targeted IL12Rβ1 agonists as depicted in FIG. 1B were designed similarly, this time the IL12Rβ1 binding moiety comprising an anti-IL12Rβ1 Fab moiety.

Similarly, exemplary tumor-targeted IL12Rβ2 agonists as depicted in FIG. 2A were designed to comprise two polypeptides herein referred to as the third and fourth polypeptides, the third polypeptide comprising from N- to C-terminal, a second tumor targeting moiety in Fab format, a linker (“TAA linker”), and a third Fc domain which enables dimerization; and the fourth polypeptide comprising from N- to C-terminal, an IL12 moiety in the format of a p40-p35 fusion peptide comprising a mutated p40 moiety linked to a wild-type p35 moiety with an IL12 moiety linker, a linker (“IL12-Fc linker”), and a fourth Fc domain that forms a dimer with the third Fc domain. Exemplary tumor-targeted IL12Rβ2 agonists as depicted in FIG. 21B were designed similarly, this time the IL12Rβ2 binding moiety comprising an anti-IL12Rβ2 Fab moiety.

The details of exemplary tumor-targeted IL12Rβ1 and IL12Rβ2 agonist constructs produced are provided in Table E1 below.

TABLE E1

| Construct Name/ | |
|---|---|
| Components | Sequence |
| IL12Rβ1-Agonist 1 | Chain 1_PSMA11838_HC (Knob) |
| (IL12Rβ1-Ag1): | QVQLVESGGGVVQPGRSLRLSCAASGFTFSSYGMHWVRQAPGKGLEWVAFMSY |
| Anti-PSMA Fab 1 | DGSNKFYSDSVKGRFTISRDNSRKMLFLQMNNLRAEDTAVYYCARDQYYDFLT |
| (PSMA11838) | DHGVFDYWGQGTLVTVSSASTKGPSVFPLAPCSRSTSESTAALGCLVKDYFPE |
| (Knob) x hIL 12 | PVTVSWNSGALTSGVHTFPAVLQSSGLYSLSSVVTVPSSSLGTKTYTCNVDHK |
| [p40wt- | PSNTKVDKRVESKYGPPCPPCPAPPVAGPSVFLFPPKPKDTLMISRTPEVTCV |
| p35(Y189E)] | VVDVSQEDPEVQFNWYVDGVEVHNAKTKPREEQFNSTYRVVSVLTVLHQDWLN |
| (Hole*) | GKEYKCKVSNKGLPSSIEKTISKAKGQPREPQVYTLPPSQEEMTKNQVSLWCL |
| | VKGFYPSDIAVEWESNGQPENNYKTTPPVLDSDGSFFLYSRLTVDKSRWQEGN |
| | VFSCSVMHEALHNHYTQKSLSLSLGK (SEQ ID NO: 198) |
| | Chain 2_1-39 ULC |
| | DIQMTQSPSSLSASVGDRVTITCRASQSISSYLNWYQQKPGKAPKLLIYAASS |
| | LQSGVPSRFSGSGSGTDFTLTISSLOPEDFATYYCQQSYSTPPITFGQGTRLE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 199) |
| | Chain 3 hIL 12 [p40wt-p35(Y189E)] Hole*) |
| | IWELKKDVYVVELDWYPDAPGEMVVLTCDTPEEDGITWTLDQSSEVLGSGKTL |
| | TIQVKEFGDAGQYTCHKGGEVLSHSLLLLHKKEDGIWSTDILKDQKEPKNKTF |
| | LRCEAKNYSGRFTCWWLTTISTDLTFSVKSSRGSSDPQGVTCGAATLSAERVR |
| | GDNKEYEYSVECQEDSACPAAEESLPIEVMVDAVHKLKYENYTSSFFIRDIIK |
| | PDPPKNLQLKPLKNSRQVEVSWEYPDTWSTPHSYFSLTFCVQVQGKSKREKKD |
| | RVFTDKTSATVICRKNASISVRAQDRYYSSSWSEWASVPCSGGGGSGGGGSGG |
| | GGSRNLPVATPDPGMFPCLHHSQNLLRAVSNMLQKARQTLEFYPCTSEEIDHE |
| | DITKDKTSTVEACLPLELTKNESCLNSRETSFITNGSCLASRKTSFMMALCLS |
| | SIYEDLKMYQVEFKTMNAKLLMDPKRQIFLDQNMLAVIDELMQALNENSETVP |
| | QKSSLEEPDFEKTKIKLCILLHAFRIRAVTIDRVMSYLNASGGGGSGGGGSGG |
| | GGSESKYGPPCPPCPAPPVAGPSVFLFPPKPKDTLMISRTPEVTCVVVDVSQE |
| | DPEVQFNWYVDGVEVHNAKTKPREEQFNSTYRVVSVLTVLHQDWLNGKEYKCK |
| | VSNKGLPSSIEKTISKAKGQPREPQVYTLPPSQEEMTKNQVSLSCAVKGFYPS |
| | DIAVEWESNGQPENNYKTTPPVLDSDGSFFLVSRLTVDKSRWQEGNVFSCSVM |
| | HEALHNRFTQKSLSLSPGK (SEQ ID NO: 200) |
| IL 12Rβ2-Agonist 1 | Chain 1_PSMA11835_HC (Knob) |
| (IL12Rβ2-Ag 1): | EVQLVESGGGLVQSGGSLRLSCEASGFTFSNYWMTWIRQGPGKGLEWVANIKP |
| Anti-PSMA Fab 2 | DGTENYYVDSVKGRFTISRDNARNSLYLQMTSLKAEDTAVYYCGRMIQFVINI |
| (PSMA11835) | WGQGTLVTVSSASTKGPSVFPLAPCSRSTSESTAALGCLVKDYFPEPVTVSWN |
| (Knob) x hIL 12 | SGALTSGVHTFPAVLQSSGLYSLSSVVTVPSSSLGTKTYTCNVDHKPSNTKVD |
| [p40(D2-D3)- | KRVESKYGPPCPPCPAPPVAGPSVFLFPPKPKDTLMISRTPEVTCVVVDVSQE |
| p35wt] (Hole*) | DPEVQFNWYVDGVEVHNAKTKPREEQFNSTYRVVSVLTVLHQDWLNGKEYKCK |
| | VSNKGLPSSIEKTISKAKGQPREPQVYTLPPSQEEMTKNQVSLWCLVKGFYPS |
| | DIAVEWESNGQPENNYKTTPPVLDSDGSFFLYSRLTVDKSRWQEGNVFSCSVM |
| | HEALHNHYTQKSLSLSLGK (SEQ ID NO: 201) |
| | Chain 2_1-39 ULC |
| | DIQMTQSPSSLSASVGDRVTITCRASQSISSYLNWYQQKPGKAPKLLIYAASS |
| | LQSGVPSRFSGSGSGTDFTLTISSLQPEDFATYYCQQSYSTPPITFGQGTRLE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 199) |
| | Chain 3 hIL12 [p40(D2-D3)-p35wt] (Hole*) |
| | DGIWSTDILKDQKEPKNKTFLRCEAKNYSGRFTCWWLTTISTDLTFSVKSSRG |
| | SSDPQGVTCGAATLSAERVRGDNKEYEYSVECQEDSACPAAEESLPIEVMVDA |
| | VHKLKYENYTSSFFIRDIIKPDPPKNLQLKPLKNSRQVEVSWEYPDTWSTPHS |
| | YFSLTFCVQVQGKSKREKKDRVFTDKTSATVICRKNASISVRAQDRYYSSSWS |
| | EWASVPCSGGGGSGGGGSGGGGSRNLPVATPDPGMFPCLHHSQNLLRAVSNML |
| | QKARQTLEFYPCTSEEIDHEDITKDKTSTVEACLPLELTKNESCLNSRETSFI |
| | TNGSCLASRKTSFMMALCLSSIYEDLKMYQVEFKTMNAKLLMDPKRQIFLDQN |
| | MLAVIDELMQALNFNSETVPQKSSLEEPDFYKTKIKLCILLHAFRIRAVTIDR |
| | VMSYLNASGGGGSGGGGSGGGGSESKYGPPCPPCPAPPVAGPSVFLFPPKPKD |
| | TLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQ |
| | EEMTKNQVSLSCAVKGFYPSDIAVEWESNGQPENNYKTTPPVLDSDGSFFLVS |
| | RLTVDKSRWQEGNVFSCSVMHEALHNRFTQKSLSLSPGK (SEQ ID NO: |
| | 202) |
| IL12Rβ2-Agonist 2 | Chain 1_PSMA11453_HC (Knob) |
| (IL12Rβ2-Ag2): | EFQVVESGGGLVKPGGSLRLSCVVSGFTFSNYNMNWVRQAPAKGLEWVSSIST |
| Anti-PSMA Fab 3 | GSSDIYYADSVKGRFTISRDNAKNSLYLQMNSLRVEDTAVYYCARDIIGTTRD |
| (PSMA11453) | WGQGTLVTVSSASTKGPSVFPLAPCSRSTSESTAALGCLVKDYFPEPVTVSWN |
| (Knob) x hIL12 | SGALTSGVHTFPAVLQSSGLYSLSSVVTVPSSSLGTKTYTCNVDHKPSNTKVD |
| [p40(D2-D3)- | KRVESKYGPPCPPCPAPPVAGPSVFLFPPKPKDTLMISRTPEVTCVVVDVSQE |
| p35wt] (Hole*) | DPEVQFNWYVDGVEVHNAKTKPREEQFNSTYRVVSVLTVLHQDWLNGKEYKCK |
| | VSNKGLPSSIEKTISKAKGQPREPQVYTLPPSQEEMTKNQVSLWCLVKGFYPS |
| | DIAVEWESNGQPENNYKTTPPVLDSDGSFFLYSRLTVDKSRWQEGNVFSCSVM |
| | HEALHNHYTQKSLSLSLGK (SEQ ID NO: 203) |
| | Chain 2_1-39 ULC |
| | DIQMTQSPSSLSASVGDRVTITCRASQSISSYLNWYQQKPGKAPKLLIYAASS |
| | LQSGVPSRFSGSGSGTDFTLTISSLQPEDFATYYCQQSYSTPPITFGQGTRLE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 199) |
| | Chain 3 hIL12 [p40(D2-D3)-p35wt] (Hole*) |
| | DGIWSTDILKDQKEPKNKTFLRCEAKNYSGRFTCWWLTTISTDLTFSVKSSRG |
| | SSDPQGVTCGAATLSAERVRGDNKEYEYSVECQEDSACPAAEESLPIEVMVDA |
| | VHKLKYENYTSSFFIRDIIKPDPPKNLQLKPLKNSRQVEVSWEYPDTWSTPHS |
| | YFSLTFCVQVQGKSKREKKDRVFTDKTSATVICRKNASISVRAQDRYYSSSWS |
| | EWASVPCSGGGGSGGGGSGGGGSRNLPVATPDPGMFPCLHHSQNLLRAVSNML |
| | QKARQTLEFYPCTSEEIDHEDITKDKTSTVEACLPLELTKNESCLNSRETSFI |
| | TNGSCLASRKTSFMMALCLSSIYEDLKMYQVEFKTMNAKLLMDPKRQIFLDQN |
| | MLAVIDELMQALNFNSETVPQKSSLEEPDFYKTKIKLCILLHAFRIRAVTIDR |
| | VMSYLNASGGGGSGGGGSGGGGSESKYGPPCPPCPAPPVAGPSVFLFPPKPKD |
| | TLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQ |
| | EEMTKNQVSLSCAVKGFYPSDIAVEWESNGQPENNYKTTPPVLDSDGSFFLVS |
| | RLTVDKSRWQEGNVFSCSVMHEALHNRFTQKSLSLSPGK (SEQ ID NO: |
| | 202) |

The constructs were expressed in Expi293F™ cells by transient transfection following the manufacturer's protocol (Thermo Fisher Scientific). Proteins in Expi293F™ supernatant were purified using the ProteinMaker system (Protein BioSolutions, Gaithersburg, MD) with either HiTrap™ Protein or MabSelect SuRe columns (Cytiva). After single step elution, the constructs were neutralized, dialyzed into a final buffer of phosphate buffered saline (PBS) with 5% glycerol, aliquoted and stored at −80° C. until use.

### 8.1.2. STAT3 Reporter Assay

A Signal Transducer and Activator of Transcription 3 (STAT3)-driven luciferase-based reporter assay was used to evaluate the ability of IL12 polypeptides to activate STAT3-mediated transcription in the human natural killer cell line, NK92. Briefly, NK92 cells were transduced with a STAT3 response element driven luciferase reporter construct and maintained in alpha minimum essential medium without nucleosides+2 mM L-glutamine/Pen/Strep+1.5 g/L sodium bicarbonate+12.5% horse serum+12.5% FBS+0.2 mM inositol+0.1 mM 2-mercaptoethanol+0.02 mM folic acid+200 U/mL recombinant hIL-2+1 mg/mL puromycin.

RPM11640 supplemented with 10% FBS and P/S/G was used as assay medium to prepare cell suspensions and fusion protein dilutions. A day prior to the assay, cells were spun down and resuspended at 5×10⁵ cells/mL in alpha minimum essential medium without nucleosides+2 mM L-glutamine/Pen/Strep+1.5 g/L sodium bicarbonate+12.5% horse serum+12.5% FBS+0.2 mM inositol+0.1 mM 2-mercaptoethanol+0.02 mM folic acid. On the day of the assay, NK92/STAT3-Luc cells were spun down, resuspended in assay medium and added to plates at 2.5×10⁴ cells/well. Target cells (Raji Raji/hPSMA, Jurkat, or Jurkat/PSMA) were spun down, resuspended in assay medium and added to plates at 2.5×10⁴ cells/well. Tumor-targeted IL12Rβ1 and IL12Rβ2 agonists were serially diluted (range: 50 nM to 29.8 fM) alone or in equal molar combination and added to cells for 4 hours at 37° C. and 5% CO₂ prior to addition of One-Glo Luciferase Substrate to lyse cells and detect luciferase activity. The emitted light was captured in relative light units (RLU) on a multilabel plate reader Envision (PerkinElmer). All serial dilutions were tested in duplicates. EC50 values of the antibodies were determined using GraphPad Prism™ software from a four-parameter logistic equation over a 10-point dose-response curve.

### 8.1.3. IFNγ Release Assay

X-Vivo 15 supplemented with 10% FBS, HEPES, NEAA, Sodium Pyruvate and 10 uM BME was used as assay medium to prepare cell suspensions and protein dilutions. Human peripheral blood mononuclear cells (PBMCs) were isolated from healthy donor leukocyte packs using the EasySep™ Direct Human PBMC Isolation Kit and following the manufacturers recommended protocol. Subsequently, CD3+ T-cells were isolated using an EasySep™ Human CD3+ T Cell Isolation Kit from StemCell Technologies and following the manufacturer's recommended instructions. T-cells were spun down, resuspended in assay medium and added to plates at 3×10⁴ cells/well. Target cells (Raji, Raji/hPSMA, or LNCaP) were spun down, resuspended in assay medium and added to plates at 5×10³ cells/well. A CD3xCD20 antibody (for Raji and Raji/PSMA cells) or CD3xSTEAP1 antibody (for LNCaP cells) was diluted in assay medium and added to plates at a constant amount of 30 nM or 750 pM, respectively. Tumor-targeted IL12Rβ1 and IL12Rβ2 agonists were serially diluted (range: 50 nM to 29.8 fM) alone or in equal molar combination and added to cells, and plates were incubated for 72 hours at 37° C. and 5% CO₂. On Day 3, 5 μL of supernatant was removed from each well and IFNγ levels were detected using IFNγ alphaLISA (Perkin Elmer). All serial dilutions were tested in duplicates. EC50 values of the antibodies were determined using GraphPad Prism™ software from a four-parameter logistic equation over a 10-point dose-response curve.

8.1.4. pSTAT4 Assay

Human peripheral blood mononuclear cells (PBMCs) were pre-activated by culturing for 72 hours in assay medium (RPMI+10% FBS+2 mM L-glutamine/Pen/Strep) in the presence of CD3/CD28 Dynabeads™ (Thermo/11132D) at a 1:2 (beads:PBMC) ratio and in the presence of 30 U/mL human IL-2 (proleukin). Beads were removed and activated PBMCs were rested in assay medium for 1 hour at 37° C. C4-2 or OVCAR3 tumor cells were spun down, resuspended in assay medium and added to plates at 5×10⁴ cells/well. Tumor-targeted IL12Rβ1 and IL12Rβ2 agonists were serially diluted (range: 100 nM to 47.7 fM) alone or in equal molar combination and added to the tumor cells, followed by addition of activated PBMCs at 5×10⁴ cells/well. Plates were incubated for 1 hour at 37° C. before fixation with Cytofix buffer (BD/554655) for 12 minutes at 37° C. Cells were then permeabilization with pre-chilled Perm Buffer III (BD/558050) for 20 mins on ice and washed 2 times. Cells were then stained with aCD3 (BD/563918), aCD8 (BD/563795), aCD4 (BD/612936), aCD25 (BD/562442), and pSTAT4 (BD/558137) for 1 hour. Cells were washed twice before data was acquired using a Cytek Aurora flow cytometer. % pSTAT4 positive or pSTAT4 gMFI (geometric mean fluorescence intensity) is plotted.

### 8.1.5. In Vitro Target Cell Killing Assay

Human peripheral blood mononuclear cells (PBMCs) were isolated from healthy donor leukocyte packs using the EasySep™ Direct Human PBMC Isolation Kit and following the manufacturers recommended protocol. Subsequently, CD3+ T-cells were isolated using an EasySep™ Human CD3+ T Cell Isolation Kit from StemCell Technologies and following the manufacturer's recommended instructions. Media used for maintaining CD3+ T-cells and conducting experiments consisted of X-VIVO 15 cell culture media supplemented with 10% FBS, HEPES, NaPyr, NEAA, and 0.01 mM BME. CD3+ T-cells were pre-activated with CD3/CD28 Dynabeads™ (Thermo/11132D) and human IL-2 (proleukin) for 3 days. Target cells for this assay were HEK293 cells engineered to co-express hPSMA and hMUC16, HEK293 cells which only express MUC16, LNCaP tumor cells, or OVCAR3 tumor cells. All target cells also express a luminescent tag containing a caspase cleavable domain, such that when caspases are active luminescence is lost. Thus, as target cells die the RLU signal will be reduced. Activated T-cells and target cells were incubated together with 6 pM constant dose of MUC16xCD3 (for HEK293 cells), 750 pM constant dose of STEAP1xCD3 (for LNCaP cells), a constant dose of MSLNxCD3 (for OVCAR3 cells treated with MUC16-targeted IL12Rβ1 and IL12Rβ2 agonists), or a constant dose of MUC16xCD3 (for OVCAR3 cells treated with MSLN-targeted IL12Rβ1 and IL12Rβ2 agonists), along with a serial dilution (either 9 point, 5-fold titration starting at 20 nM or a 10-point, 5-fold titration starting at 100 nM) of hPSMA, hMUC16, or hMSLN-targeted IL12Rβ1 and IL12Rβ2 agonists alone or in equal molar combination. The lowest point on the curve contains no titrated IL12R or Isotype control molecules. Assay was read out after 3 days at 37 C 5% CO₂. Prior to addition of One-Glo to sample wells for detecting luminescence, supernatant was collected for assessing IFNγ release. For the luminescent readout emitted light was measured in RLU on a multilabel plate reader Envision (PerkinElmer). EC50 values were determined from a 4-parameter logistic equation over either a 9 point or 10-point dose response curve using GraphPad Prism software. For assessing IFNγ release, commercially available immunoassays were used.

### 8.2. Example 1: Activation of STAT3-Signaling by the Combination of IL12 Moiety-Comprising Tumor-Targeted IL12Rβ1 and IL12Rβ2 Agonists

IL12 moiety comprising tumor-targeted IL12Rβ1 and IL12Rβ2 agonists IL12Rβ1-Agonist 1 (IL12Rβ1-Ag1), IL12Rβ2-Agonist 1 (IL12Rβ2-Ag1), and IL12Rβ2-Agonist 2 (IL12Rβ2-Ag2) were designed and produced as described in Section 8.1.1. The tumor targeting moieties and IL12R binding moieties of these tumor-targeted IL12Rβ1 and IL12Rβ2 agonist constructs used in this example are set forth in Table E2. The ability of IL12 moiety-comprising tumor-targeted IL12Rβ1 and IL12Rβ2 agonists to activate STAT3-signaling was assessed in a STAT3-reporter cell-based assay as described in Section 8.1.2.

TABLE E2

| Construct | Tumor targeting moiety | IL12R targeting moiety |
|---|---|---|
| IL12Rβ1-Agonist 1 (IL12Rβ1- | Anti-PSMA Fab-1 | IL12 moiety comprising wt |
| Ag1) | | p40 and p35-null |
| IL12Rβ2-Agonist 1 (IL12Rβ2- | Anti-PSMA Fab-2 | IL12 moiety comprising wt |
| Ag1) | | p35 and p40-null |
| IL12Rβ2-Agonist 2 (IL12Rβ2- | Anti-PSMA Fab-3 | IL12 moiety comprising wt |
| Ag2) | | p35 and p40-null |

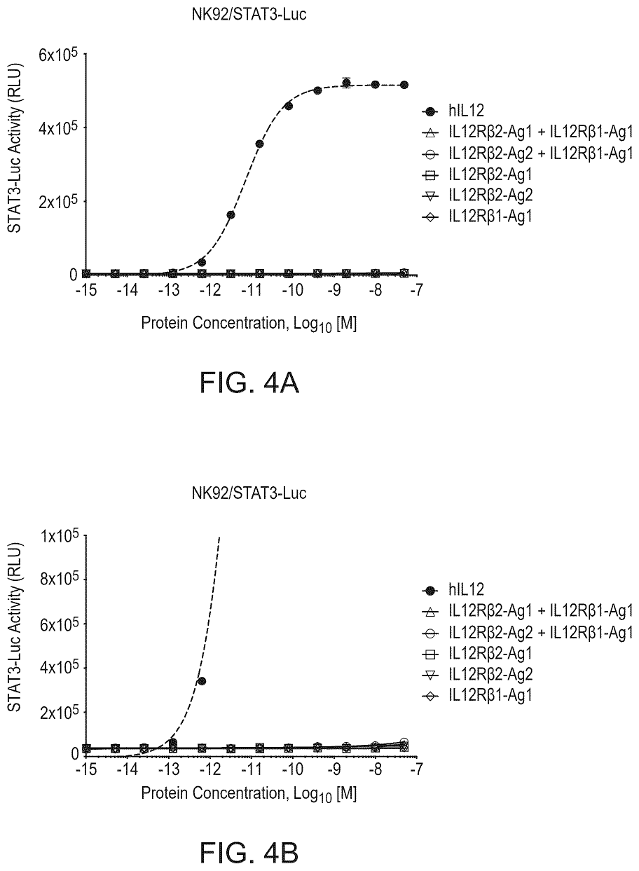

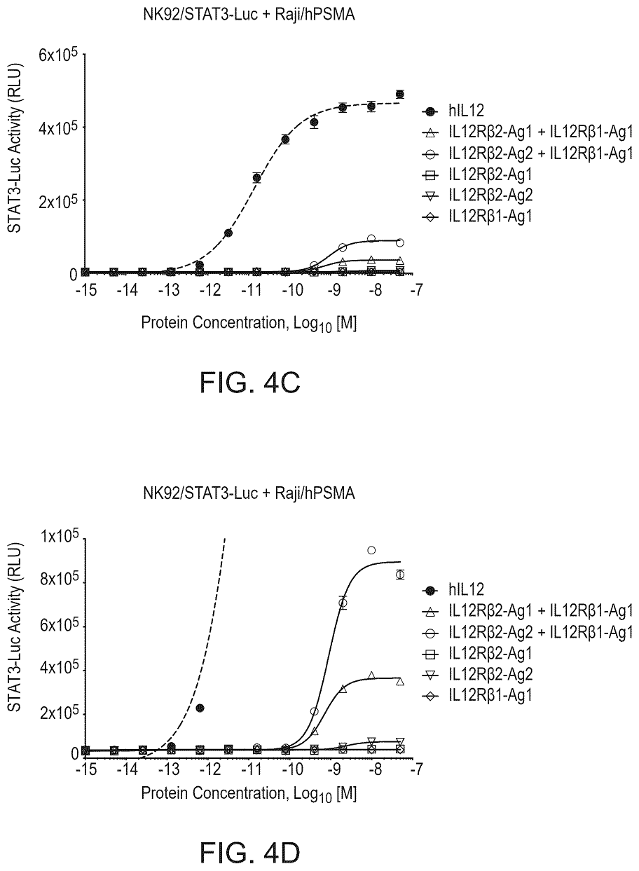

The constructs IL12Rβ1-Ag1, IL12Rβ2-Ag1, and IL12Rβ2-Ag2 alone or combinations comprising IL12Rβ1-Ag1+IL12Rβ2-Ag1 or IL12Rβ1-Ag1+IL12Rβ2-Ag2 did not trigger STAT signaling activation in the absence of PSMA-expressing cells (FIGS. 4A-4B).

When NK92/STAT3-Luc cells were co-cultured with hPSMA-expressing Raji cells, both combinations were associated with STAT3-Luc activity, in which combination of IL12Rβ1-Ag1+IL12Rβ2-Ag2 produced a stronger activation that that produced by the combination of IL12Rβ1-Ag1+IL12Rβ2-Ag1 (FIGS. 4C-4D).

### 8.3. Example 2: Activation of STAT3-Signaling and IFNγ Release by the Combination of Tumor-Targeted IL12Rβ1 and IL12Rβ2 Agonists

Tumor-targeted IL12Rβ1 and IL12Rβ2 agonists comprising Fab arms were designed and produced as described in Section 8.1.1. The tumor targeting moieties and IL12R binding moieties of these tumor-targeted IL12Rβ1 and IL12Rβ2 agonist constructs used in this example are set forth in Table E3. The ability of tumor-targeted IL12Rβ1 and IL12Rβ2 agonists to activate STAT3-signaling was assessed in a STAT3-reporter cell-based assay as described in Section 8.1.2. The ability of tumor-targeted IL12Rβ1 and IL12Rβ2 agonists to induce the release of IFNγ from T-cells was assessed as described in Section 8.1.3.

TABLE E3

| Construct | Tumor targeting moiety | IL12R targeting moiety |
|---|---|---|
| IL12Rβ1-Agonist 2 (IL12Rβ1-Ag2) | Anti-PSMA Fab-2 | Anti-IL12β1 Fab-1 |
| IL12Rβ1-Agonist 3 (IL12Rβ1-Ag3) | Anti-PSMA Fab-2 | Anti-IL12β1 Fab-2 |
| IL12Rβ1-Agonist 4 (IL12Rβ1-Ag4) | Anti-PSMA Fab-1 | Anti-IL12β1 Fab-1 |
| IL12Rβ1-Agonist 5 (IL12Rβ1-Ag5) | Anti-PSMA Fab-1 | Anti-IL12β1 Fab-2 |
| IL12Rβ2-Agonist 3 (IL12Rβ2-Ag3) | Anti-PSMA Fab-2 | Anti-IL12β2 Fab-1 |
| IL12Rβ2-Agonist 4 (IL12Rβ2-Ag4) | Anti-PSMA Fab-2 | Anti-IL12β2 Fab-2 |
| IL12Rβ2-Agonist 5 (IL12Rβ2-Ag5) | Anti-PSMA Fab-1 | Anti-IL12β2 Fab-1 |
| IL12Rβ2-Agonist 6 (IL12Rβ2-Ag6) | Anti-PSMA Fab-1 | Anti-IL12β2 Fab-2 |

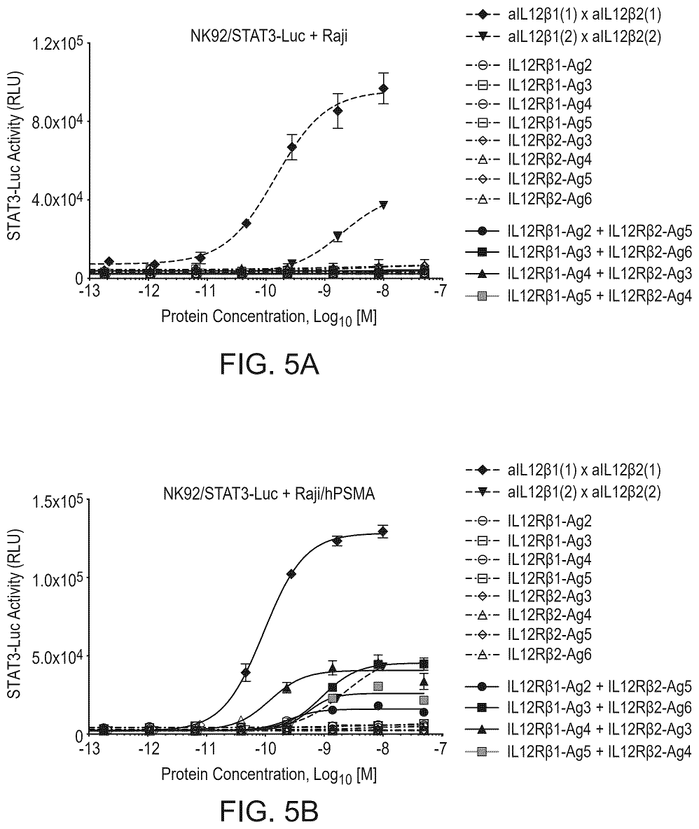

First, the tumor-targeted IL12Rβ1 and IL12Rβ2 agonists were evaluated alone or in combinations using NK92/STAT3-Luc cells that were co-cultured with Raji cells that do not express PSMA. Only control bispecific antibodies comprising an anti-IL12p1 Fab arm and an anti-IL12P2 Fab arm were associated with STAT3-Luc activity (FIG. 5A). Next, the tumor-targeted IL12Rβ1 and IL12Rβ2 agonists were evaluated alone or in combinations using NK92/STAT3-Luc cells that were co-cultured with hPSMA-expressing Raji cells. In this case, the four tumor-targeted IL12Rβ1 and IL12Rβ2 agonist combinations resulted in STAT3-Luc activity, and the strongest STAT3-Luc activation among the four combinations was observed with IL12Rβ1-Ag4+IL12Rβ2-Ag3. These results suggest that the tumor-targeted IL12Rβ1 and IL12Rβ2 agonist combinations induce STAT signaling activation only in the presence of PSMA-expressing tumor cells.

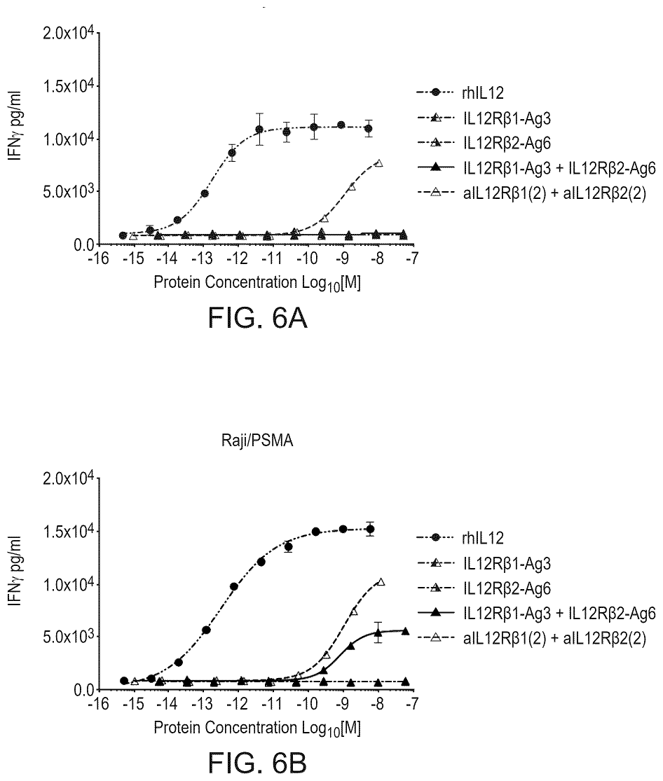

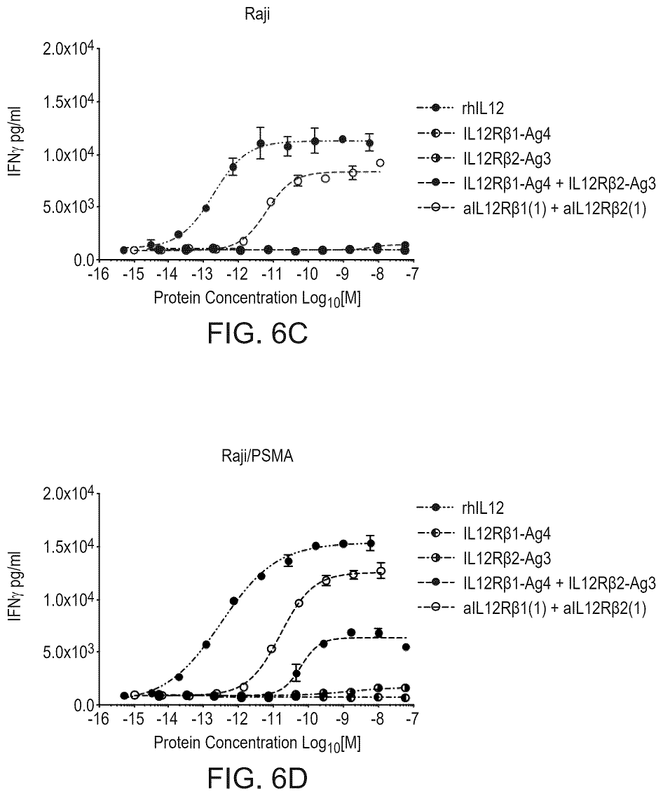

Next, the combinations of tumor-targeted IL12Rβ1 and IL12Rβ2 agonists to induce the release of IFNγ from T-cells was evaluated in the presence of either Raji cells that do not express PSMA or in the presence of PSMA-expressing Raji cells. The combination of IL12Rβ1-Ag3+IL12Rβ2-Ag6 was not associated with IFNγ release in the presence of Raji cells that do not express PSMA (FIG. 6A), but induced IFNγ release in the presence of hPSMA-expressing Raji cells (FIG. 6B). Similarly, the combination of IL12Rβ1-Ag4+IL12Rβ2-Ag3 was not associated with IFNγ release in the presence of Raji cells that do not express PSMA (FIG. 6C), but induced IFNγ release in the presence of hPSMA-expressing Raji cells (FIG. 6D). These results suggest that the combinations of tumor-targeted IL12Rβ1 and IL12Rβ2 agonists induce the release of IFNγ from T-cells only in the presence of PSMA-expressing cells.

### 8.4. Example 3: Activation of pSTAT4 Signaling, IFNγ Release and Target Cell Killing by Tumor-Targeted IL12Rβ1 and IL12Rβ2 Agonist Combinations

The tumor-targeted IL12Rβ1 and IL12Rβ2 agonists generated and used in Section 8.3 were evaluated for their ability to activate pSTAT4 signaling as described in Section 8.1.4, for their ability to induce IFNγ release as described in Section 8.1.3, and for their ability to induce target cell death as described in Section 8.1.5.

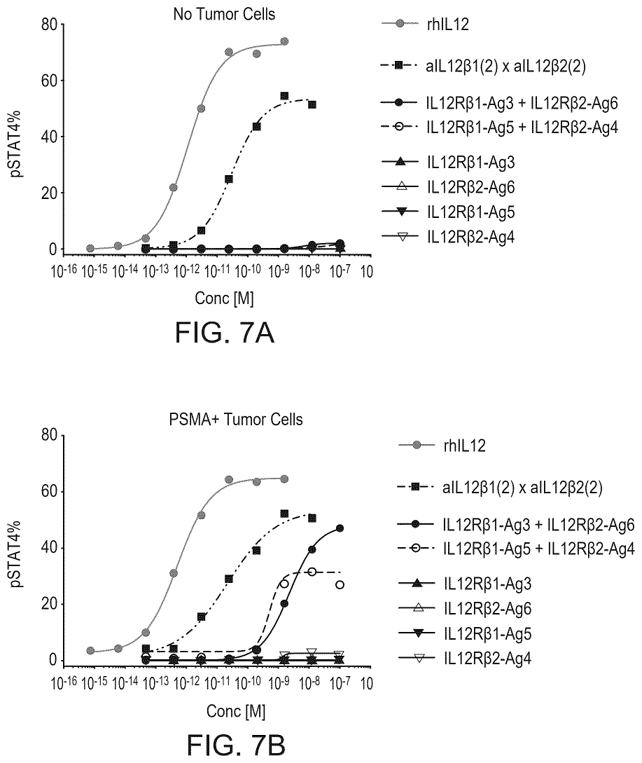

The combinations of tumor-targeted IL12Rβ1 and IL12Rβ2 agonists to activate pSTAT4 signaling in activated T-cells was evaluated in the presence and absence of PSMA-expressing C4-2 tumor cells. In the absence of tumor cells, tumor-targeted IL12Rβ1 and IL12Rβ2 agonists, alone or in combination, did not activate pSTAT4 signaling (FIG. 7A). In the presence of PSMA-expressing cells, both combinations that were evaluated (IL12Rβ1-Ag3+IL12Rβ2-Ag6 and IL12Rβ1-Ag5+IL12Rβ2-Ag4) activated pSTAT4 signaling in CD25+ T-cells (FIG. 7B). None of the tumor-targeted IL12Rβ1 and IL12Rβ2 agonist constructs were associated with pSTAT4 activations when tested alone (FIG. 7B).

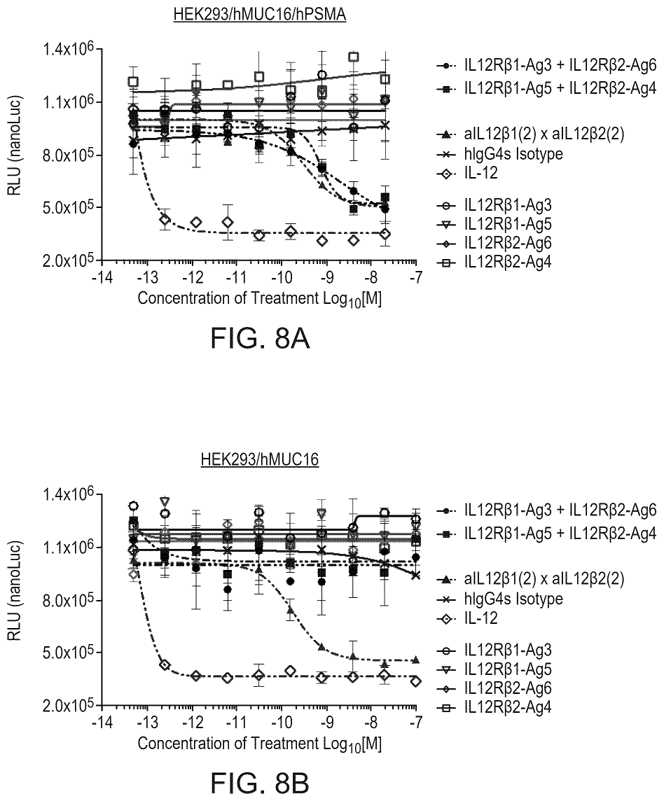

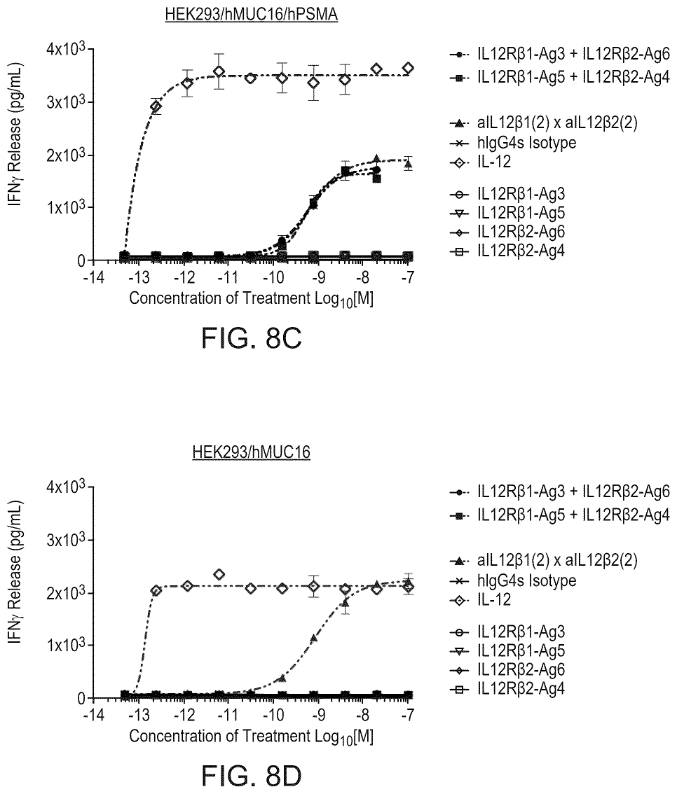

The combinations of tumor-targeted IL12Rβ1 and IL12Rβ2 agonists to promote target cell killing and IFNγ release by human T-cells was evaluated with target HEK293 cells that express only hMUC16 or both hMUC16 and hPSMA. In the presence of a MUC16xCD3 antibody, tumor-targeted IL12Rβ1 and IL12Rβ2 agonist combinations IL12Rβ1-Ag3+IL12Rβ2-Ag6 and IL12Rβ1-Ag5+IL12Rβ2-Ag4 promoted cell killing only if the target cells expressed PSMA (FIGS. 8A-8B). Similarly, these combinations were associated with IFNγ release only when HEK293 cells expressed hPSMA (FIGS. 8C-8D).

### 8.5. Example 4: Activation of STAT3-Signaling, pSTAT4 Signaling, IFNγ Release and Target Cell Killing by PSMA-Targeted IL12Rβ1 and IL12Rβ2 Agonist Combinations

The tumor-targeted IL12Rβ1 and IL12Rβ2 agonists generated and used in Section 8.3 were evaluated for their ability to activate STAT3-signaling as described in Section 8.1.2, to activate pSTAT4 signaling as described in Section 8.1.4, to induce IFNγ release as described in Section 8.1.3, and to induce target cell death as described in Section 8.1.5.

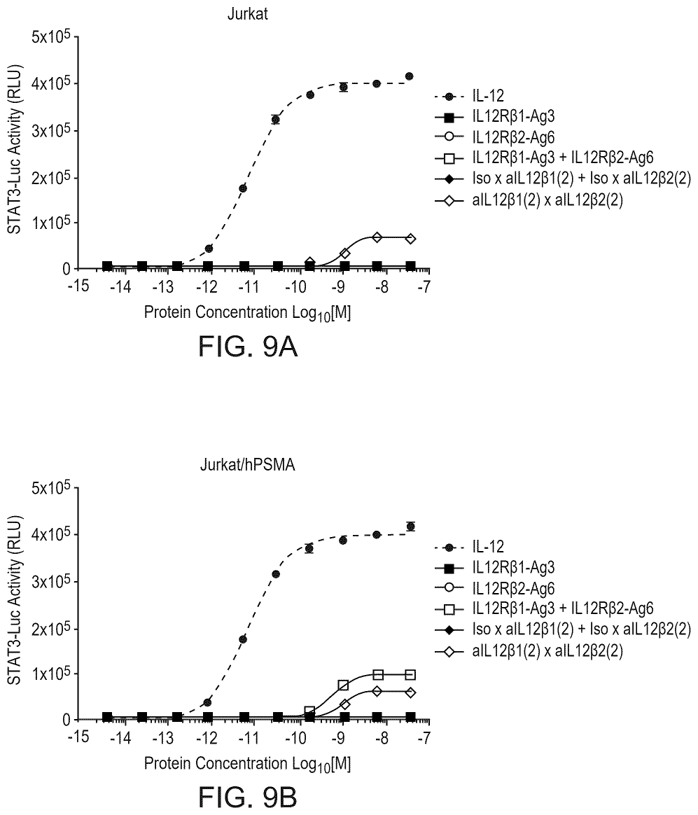

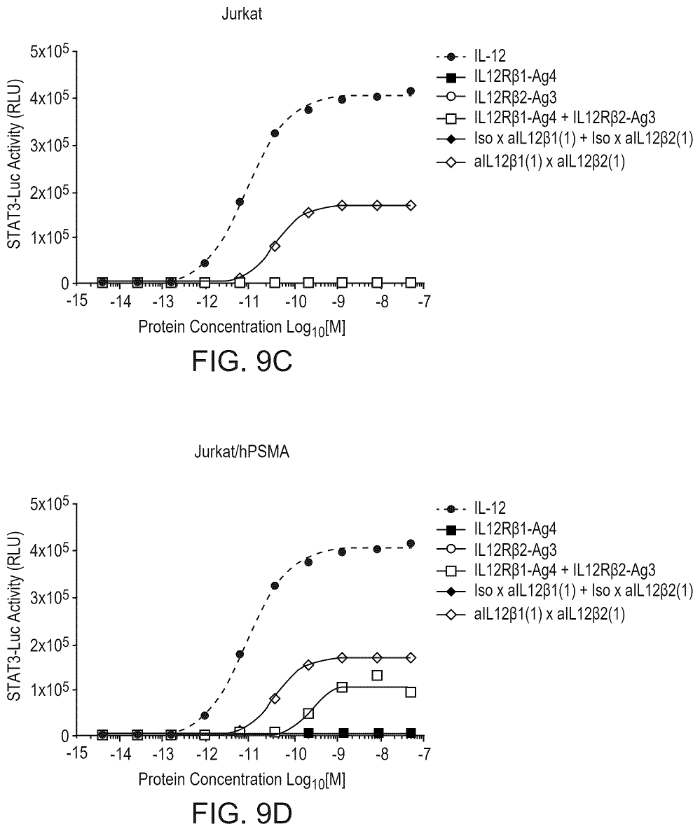

The PSMA-targeted IL12Rβ1 and IL12Rβ2 agonists did not trigger STAT3 signaling, either alone or in combination, in Jurkat cells which did not express PSMA, either for the first combination of IL12Rβ1-Ag3+IL12Rβ1-Ag6 (FIG. 9A) or for the second combination of IL12Rβ1-Ag4+IL12Rβ1-Ag3 (FIG. 9C). IL12Rβ1-Ag3+IL12Rβ1-Ag6 in combination triggered STAT3 activation in Jurkat/PSMA cells, but not when administered alone (FIG. 9B). Similarly, IL12Rβ1-Ag4+IL12Rβ1-Ag3 in combination triggered STAT3 activation in Jurkat/PSMA cells, but not when administered alone (FIG. 9D).

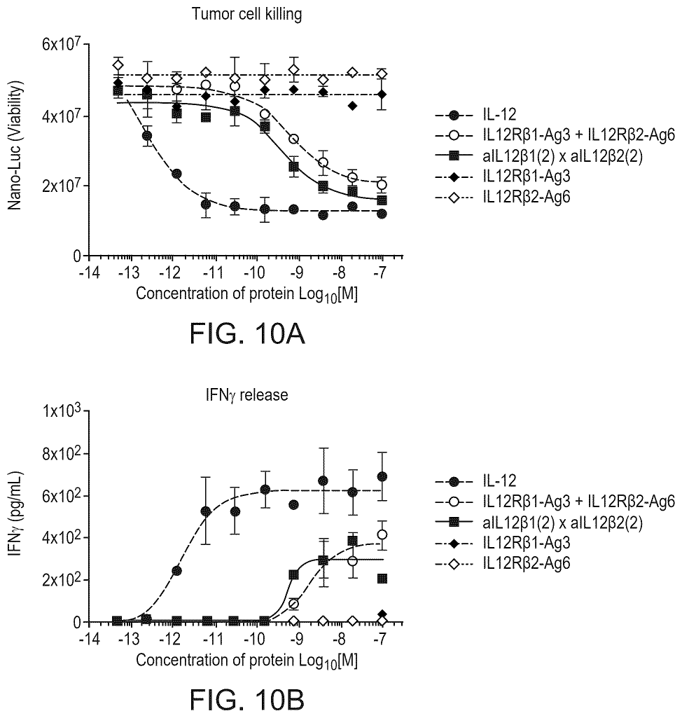

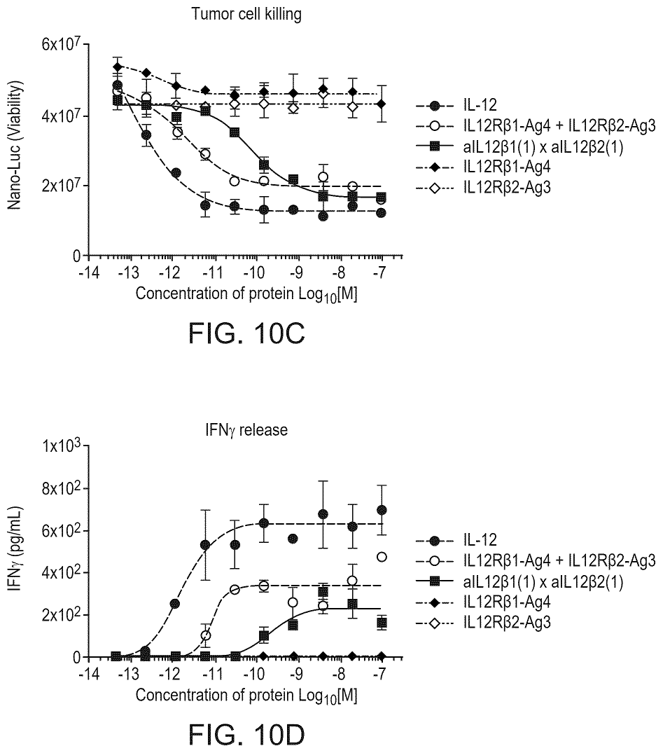

The PSMA-targeted IL12Rβ1 and IL12Rβ2 agonists were evaluated for their ability to induce cell killing and IFNγ release in LNCaP tumor cells, which express PSMA at high levels. The combination of IL12Rβ1-Ag3+IL12Rβ2-Ag6 (together with STEAP1xCD3 antibody) promoted cell killing (FIG. 10A) and IFNγ release (FIG. 10B). Similarly, the combination of IL12Rβ1-Ag4+IL12Rβ2-Ag3 (together with STEAP1xCD3 antibody) promoted cell killing (FIG. 10C) and IFNγ release (FIG. 10D). No cell killing or IFNγ release was observed with administration of IL12Rβ1-Ag3, IL12Rβ2-Ag6, IL12Rβ2-Ag3 or IL12Rβ1-Ag4 alone.

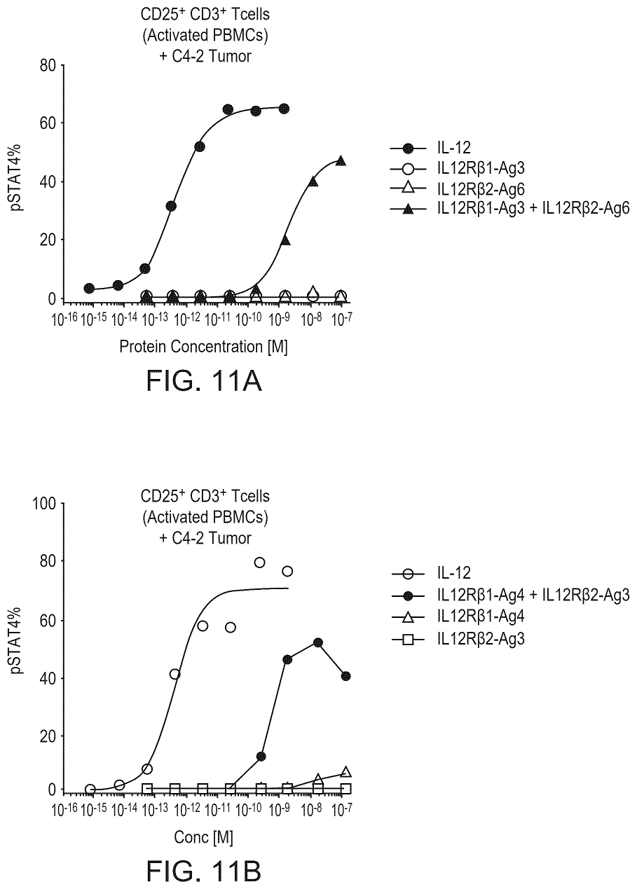

Finally, the PSMA-targeted IL12Rβ1 and IL12Rβ2 agonists were evaluated for their ability to activate pSTAT4 signaling in activated T-cells in the presence of PSMA-positive C4-2 tumor cells. Both combinations, IL12Rβ1-Ag3+IL12Rβ2-Ag6 (FIG. 11A) and IL12Rβ1-Ag4+IL12Rβ2-Ag3 (FIG. 11B) activated STAT5 signaling in CD25+ T-cells.

### 8.6. Example 5: Activation of pSTAT4 Signaling and Target Cell Killing by MUC16- and MSLN-Targeted IL12Rβ1 and IL12Rβ2 Agonist Combinations

Tumor-targeted IL12Rβ1 and IL12Rβ2 agonists comprising Fab arms were designed and produced as described in Section 8.1.1. The tumor targeting moieties and IL12R binding moieties of these tumor-targeted IL12Rβ1 and IL12Rβ2 agonist constructs used in this example are set forth in Table E4. The ability of tumor-targeted IL12Rβ1 and IL12Rβ2 agonists to activate pSTAT4 signaling as described in Section 8.1.4 and to induce target cell death as described in Section 8.1.5 was evaluated in OVCAR3 cells, which express MUC16 and MSLN at high levels.

TABLE E4

| | Tumor targeting | IL 12R targeting |
|---|---|---|
| Construct | moiety | moiety |
| IL12Rβ1-Agonist 11 (IL12Rβ1-Ag11) | Anti-MUC16 Fab-1 | Anti-IL12β1 Fab-1 |
| IL12Rβ2-Agonist 11 (IL12Rβ2-Ag11) | Anti-MUC16 Fab-2 | Anti-IL12β2 Fab-2 |
| IL12Rβ1-Agonist 21 (IL12Rβ1-Ag21) | Anti-MSLN Fab-1 | Anti-IL12β1 Fab-1 |
| IL12Rβ2-Agonist 21 (IL12Rβ2-Ag21) | Anti-MSLN Fab-2 | Anti-IL12β2 Fab-2 |

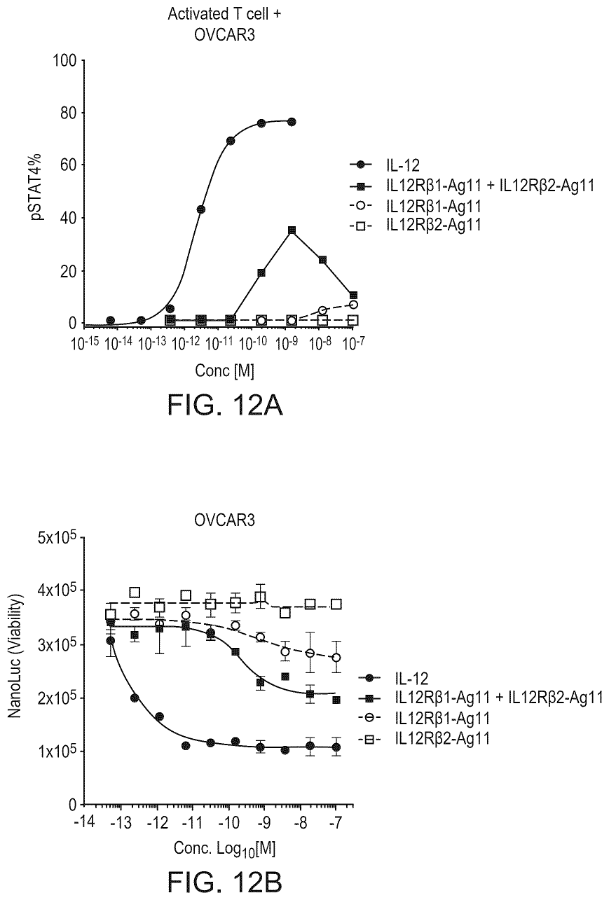

The pair of MUC16-targeted constructs (IL12Rβ1-Ag11+IL12Rβ2-Ag11), when administered in combination, induced pSTAT4 signaling in activated T-cells in the presence of OVCAR3 tumor cells (FIG. 12A). The pair of MUC16-targeted constructs also induced cell killing of OVCAR3 tumor cells when administered in combination to a significantly higher degree than either construct alone (FIG. 12B).

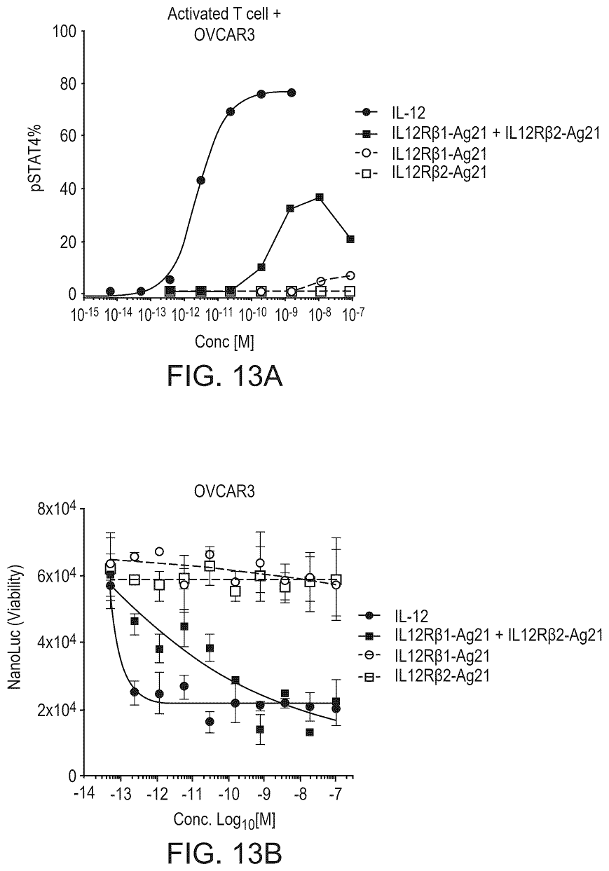

The pair of MSLN-targeted constructs (IL12Rβ1-Ag21+IL12Rβ2-Ag21), when administered in combination, induced pSTAT4 signaling in activated T-cells in the presence of OVCAR3 tumor cells (FIG. 13A). The pair of MSLN-targeted constructs also induced cell killing of OVCAR3 tumor cells when administered in combination, while no cell killing was observed with administration of either construct alone (FIG. 13B).

### 8.7. Example 6: Activation of STAT3 Signaling by Additional PSMA-Targeted IL12Rβ1 and IL12Rβ2 Agonist Combinations

PSMA-targeted IL12Rβ1 and IL12Rβ2 agonists comprising Fab arms were designed and produced as described in Section 8.1.1. The tumor targeting moieties and IL12R binding moieties of these PSMA-targeted IL12Rβ1 and IL12Rβ2 agonist constructs used in this example are set forth in Table E5. The ability of PSMA-targeted IL12Rβ1 and IL12Rβ2 agonists to activate STAT3-signaling was assessed in a STAT3-reporter cell-based assay as described in Section 6.1.2.

TABLE E5

| | Tumor targeting | IL12R targeting |
|---|---|---|
| Construct | moiety | moiety |
| IL12Rβ1-Agonist 4 (IL12Rβ1-Ag4) | Anti-PMSA Fab-1 | Anti-IL12β1 Fab-1 |
| IL12Rβ1-Agonist 3 (IL12Rβ1-Ag3) | Anti-PSMA Fab-2 | Anti-IL12β1 Fab-2 |
| IL12Rβ1-Agonist 6 (IL12Rβ1-Ag6) | Anti-PSMA Fab-2 | Anti-IL12β1 Fab-3 |
| IL12Rβ1-Agonist 7 (IL12Rβ1-Ag7) | Anti-PMSA Fab-2 | Anti-IL12β1 Fab-4 |
| IL12Rβ2-Agonist 3 (IL12Rβ2-Ag3) | Anti-PSMA Fab-2 | Anti-IL12β2 Fab-1 |
| IL12Rβ2-Agonist 6 (IL12Rβ2-Ag6) | Anti-PSMA Fab-1 | Anti-IL12β2 Fab-2 |
| IL12Rβ2-Agonist 7 (IL12Rβ2-Ag7) | Anti-PMSA Fab-4 | Anti-IL12β2 Fab-3 |
| IL12Rβ2-Agonist 8 (IL12Rβ2-Ag8) | Anti-PMSA Fab-4 | Anti-IL12β2 Fab-4 |

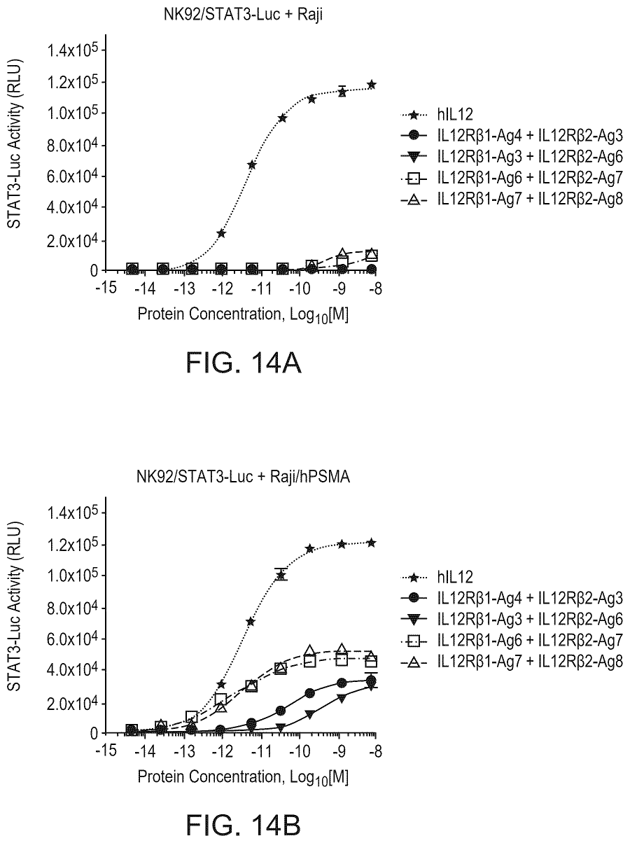

The combinations of PSMA-targeted IL12Rβ1 and IL12Rβ2 agonist constructs triggered minimal STAT3 signaling activation in NK92/STAT3-Luc cells in the absence of PSMA-expressing cells (FIG. 14A).

When NK92/STAT3-Luc cells were co-cultured with hPSMA-expressing Raji cells, all combinations were associated with STAT3-Luc activity, in which combinations of IL12Rβ1-Ag6+IL12Rβ2-Ag7 and IL12Rβ1-Ag7+IL12Rβ2-Ag8 produced a stronger activation than that produced by the combinations of IL12Rβ1-Ag4+IL12Rβ2-Ag3 and IL12Rβ1-Ag3+IL12Rβ2-Ag6 (FIG. 14B).

### 8.8. Example 7: Activation of pSTAT4 Signaling by Combinations of PSMA-Targeted IL12Rβ1 and IL12Rβ2 Agonists with Different Formats

PSMA-targeted IL12Rβ1 and IL12Rβ2 agonists comprising scFv arms were designed and produced similarly to the protocol described in Section 8.1.1. The PSMA-targeting moieties and IL12R binding moieties of the PSMA-targeted IL12Rβ1 and IL12Rβ2 agonist constructs used in this example are set forth in Table E6. The ability of PSMA-targeted IL12Rβ1 and IL12Rβ2 agonists to activate pSTAT4 signaling was evaluated as described in Section 8.1.4 in the absence and presence of C4-2 tumor cells.

TABLE E6

| | Tumor targeting | |
|---|---|---|
| Construct | moiety | IL12R targeting moiety |
| IL12Rβ1-Agonist 4 (IL12Rβ1-Ag4) | Anti-PSMA Fab-1 | Anti-IL12β1 Fab-1 |
| IL12Rβ1-Agonist 4s (IL12Rβ1-Ag4s) | Anti-PSMA scFv-1 | Anti-IL12β1 scFv-1 |
| IL12Rβ2-Agonist 3 (IL12Rβ2-Ag3) | Anti-PSMA Fab-2 | Anti-IL12β2 Fab-1 |
| IL12Rβ2-Agonist 3s (IL12Rβ2-Ag3s) | Anti-PSMA scFv-2 | Anti-IL12β2 scFv-1 |

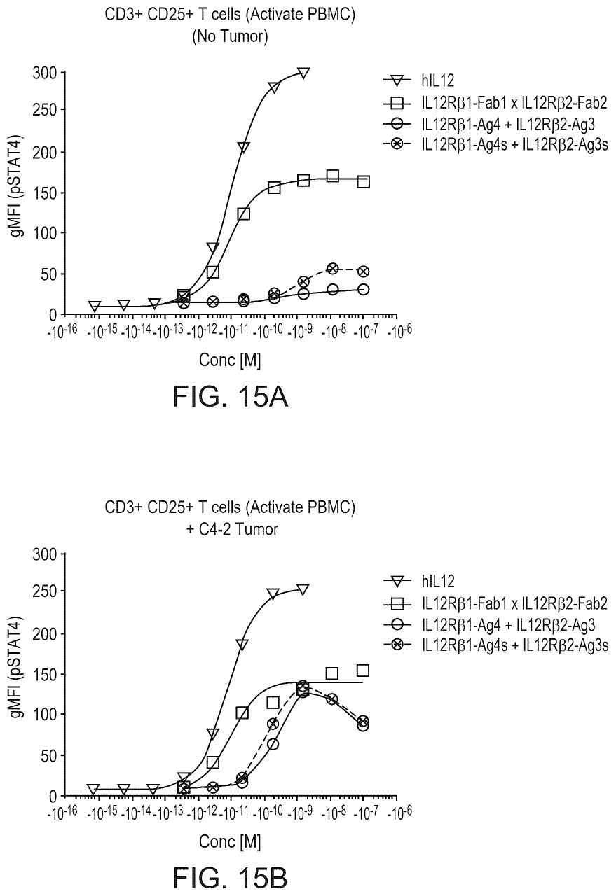

STAT4 signaling of combinations PSMA-targeted IL12Rβ1 and IL12Rβ2 agonists with Fab or scFv arms was evaluated. The results of this assessment showed that both combinations were associated with minimal STAT4 signaling in the absence of C4-2 tumor cells (FIG. 15A) but displayed increased levels of STAT4 phosphorylation in the presence of C4-2 tumor cells (FIG. 15B).

### 9. SEQUENCE LISTING

Exemplary sequences of the present disclosure are provided in Table S below (with the column “SEQ” indicating the SEQ ID NO:).

TABLE S

| SEQ | DESCRIPTION | SEQUENCE |
|---|---|---|
| 1 | WT full length | MCPARSLLLVATLVLLDHLSLARNLPVATPDPGMFPCLHHSQNLL |
| | human p35 | RAVSNMLQKARQTLEFYPCTSEEIDHEDITKDKTSTVEACLPLEL |
| | | TKNESCLNSRETSFITNGSCLASRKTSFMMALCLSSIYEDLKMYQ |
| | | VEFKTMNAKLLMDPKRQIFLDQNMLAVIDELMQALNFNSETVPQK |
| | | SSLEEPDFYKTKIKLCILLHAFRIRAVTIDRVMSYLNAS |
| 2 | WT mature human | RNLPVATPDPGMFPCLHHSQNLLRAVSNMLQKARQTLEFYPCTSE |
| | p35 | EIDHEDITKDKTSTVEACLPLELTKNESCLNSRETSFITNGSCLA |
| | | SRKTSFMMALCLSSIYEDLKMYQVEFKTMNAKLLMDPKRQIFLDQ |
| | | NMLAVIDELMQALNFNSETVPQKSSLEEPDFYKTKIKLCILLHAF |
| | | RIRAVTIDRVMSYLNAS |
| 3 | WT full length | MCQSRYLLFLATLALLNHLSLARVIPVSGPARCLSQSRNLLKTTD |
| | murine p35 | DMVKTAREKLKHYSCTAEDIDHEDITRDQTSTLKTCLPLELHKNE |
| | | SCLATRETSSTTRGSCLPPQKTSLMMTLCLGSIYEDLKMYQTEFQ |
| | | AINAALQNHNHQQIILDKGMLVAIDELMQSLNHNGETLRQKPPVG |
| | | EADPYRVKMKLCILLHAFSTRVVTINRVMGYLSSA |
| 4 | WT mature murine | RVIPVSGPARCLSQSRNLLKTTDDMVKTAREKLKHYSCTAEDIDH |
| | p35 | EDITRDQTSTLKTCLPLELHKNESCLATRETSSTTRGSCLPPQKT |
| | | SLMMTLCLGSIYEDLKMYQTEFQAINAALQNHNHQQIILDKGMLV |
| | | AIDELMQSLNHNGETLRQKPPVGEADPYRVKMKLCILLHAFSTRV |
| | | VTINRVMGYLSSA |
| 5 | WT full length | MCHQQLVISWFSLVFLASPLVAIWELKKDVYVVELDWYPDAPGEM |
| | human p40 | VVLTCDTPEEDGITWTLDQSSEVLGSGKTLTIQVKEFGDAGQYTC |
| | | HKGGEVLSHSLLLLHKKEDGIWSTDILKDQKEPKNKTFLRCEAKN |
| | | YSGRFTCWWLTTISTDLTFSVKSSRGSSDPQGVTCGAATLSAERV |
| | | RGDNKEYEYSVECQEDSACPAAEESLPIEVMVDAVHKLKYENYTS |
| | | SFFIRDIIKPDPPKNLQLKPLKNSRQVEVSWEYPDTWSTPHSYFS |
| | | LTFCVQVQGKSKREKKDRVFTDKTSATVICRKNASISVRAQDRYY |
| | | SSSWSEWASVPCS |
| 6 | WT mature human | DGIWSTDILKDQKEPKNKTFLRCEAKNYSGRFTCWWLTTISTDLT |
| | p40, D2-D3 | FSVKSSRGSSDPQGVTCGAATLSAERVRGDNKEYEYSVECQEDSA |
| | domains | CPAAEESLPIEVMVDAVHKLKYENYTSSFFIRDIIKPDPPKNLQL |
| | | KPLKNSRQVEVSWEYPDTWSTPHSYFSLTFCVQVQGKSKREKKDR |
| | | VFTDKTSATVICRKNASISVRAQDRYYSSSWSEWASVPCS |
| 7 | WT mature human | IWELKKDVYVVELDWYPDAPGEMVVLTCDTPEEDGITWTLDQSSE |
| | p40 | VLGSGKTLTIQVKEFGDAGQYTCHKGGEVLSHSLLLLHKKEDGIW |
| | | STDILKDQKEPKNKTFLRCEAKNYSGRFTCWWLTTISTDLTFSVK |
| | | SSRGSSDPQGVTCGAATLSAERVRGDNKEYEYSVECQEDSACPAA |
| | | EESLPIEVMVDAVHKLKYENYTSSFFIRDIIKPDPPKNLQLKPLK |
| | | NSRQVEVSWEYPDTWSTPHSYFSLTFCVQVQGKSKREKKDRVFTD |
| | | KTSATVICRKNASISVRAQDRYYSSSWSEWASVPCS |
| 8 | WT full length | MCPQKLTISWFAIVLLVSPLMAMWELEKDVYVVEVDWTPDAPGET |
| | murine p40 | VNLTCDTPEEDDITWTSDQRHGVIGSGKTLTITVKEFLDAGQYTC |
| | | HKGGETLSHSHLLLHKKENGIWSTEILKNFKNKTFLKCEAPNYSG |
| | | RFTCSWLVQRNMDLKFNIKSSSSSPDSRAVTCGMASLSAEKVTLD |
| | | QRDYEKYSVSCQEDVTCPTAEETLPIELALEARQQNKYENYSTSF |
| | | FIRDIIKPDPPKNLQMKPLKNSQVEVSWEYPDSWSTPHSYFSLKF |
| | | FVRIQRKKEKMKETEEGCNQKGAFLVEKTSTEVQCKGGNVCVQAQ |
| | | DRYYNSSCSKWACVPCRVRS |
| 9 | WT mature murine | NGIWSTEILKNFKNKTFLKCEAPNYSGRFTCSWLVQRNMDLKFNI |
| | p40, D2-D3 | KSSSSSPDSRAVTCGMASLSAEKVTLDQRDYEKYSVSCQEDVTCP |
| | domains | TAEETLPIELALEARQQNKYENYSTSFFIRDIIKPDPPKNLQMKP |
| | | LKNSQVEVSWEYPDSWSTPHSYFSLKFFVRIQRKKEKMKETEEGC |
| | | NQKGAFLVEKTSTEVQCKGGNVCVQAQDRYYNSSCSKWACVPCRV |
| | | RS |
| 10 | WT mature murine | MWELEKDVYVVEVDWTPDAPGETVNLTCDTPEEDDITWTSDQRHG |
| | p40 | VIGSGKTLTITVKEFLDAGQYTCHKGGETLSHSHLLLHKKENGIW |
| | | STEILKNFKNKTFLKCEAPNYSGRFTCSWLVQRNMDLKFNIKSSS |
| | | SSPDSRAVTCGMASLSAEKVTLDQRDYEKYSVSCQEDVTCPTAEE |
| | | TLPIELALEARQQNKYENYSTSFFIRDIIKPDPPKNLQMKPLKNS |
| | | QVEVSWEYPDSWSTPHSYFSLKFFVRIQRKKEKMKETEEGCNQKG |
| | | AFLVEKTSTEVQCKGGNVCVQAQDRYYNSSCSKWACVPCRVRS |
| 11 | hIgG1 Fc | EPKSCDKTHTCPPCPAPELLGGPSVFLFPPKPKDTLMISRTPEVT |
| | (amino acids | CVVVDVSHEDPEVKFNWYVDGVEVHNAKTKPREEQYNSTYRVVSV |
| | 99-330 of | LTVLHQDWLNGKEYKCKVSNKALPAPIEKTISKAKGQPREPQVYT |
| | UniprotKB | LPPSRDELTKNQVSLTCLVKGFYPSDIAVEWESNGQPENNYKTTP |
| | P01857-1) | PVLDSDGSFFLYSKLTVDKSRWQQGNVFSCSVMHEALHNHYTQKS |
| | | LSLSPGK |
| 12 | hIgG2 Fc | ERKCCVECPPCPAPPVAGPSVFLFPPKPKDTLMISRTPEVTCVVV |
| | (amino acids | DVSHEDPEVQFNWYVDGVEVHNAKTKPREEQFNSTFRVVSVLTVV |
| | 99-326 of | HQDWLNGKEYKCKVSNKGLPAPIEKTISKTKGQPREPQVYTLPPS |
| | UniprotKB | REEMTKNQVSLTCLVKGFYPSDISVEWESNGQPENNYKTTPPMLD |
| | P01859-1) | SDGSFFLYSKLTVDKSRWQQGNVFSCSVMHEALHNHYTQKSLSLS |
| | | PGK |
| 13 | hIgG4 Fc | ESKYGPPCPSCPAPEFLGGPSVFLFPPKPKDTLMISRTPEVTCVV |
| | (amino acids | VDVSQEDPEVQFNWYVDGVEVHNAKTKPREEQFNSTYRVVSVLTV |
| | 99-327 of | LHQDWLNGKEYKCKVSNKGLPSSIEKTISKAKGQPREPQVYTLPP |
| | UniprotKB | SQEEMTKNQVSLTCLVKGFYPSDIAVEWESNGQPENNYKTTPPVL |
| | P01861-1) | DSDGSFFLYSRLTVDKSRWQEGNVFSCSVMHEALHNHYTQKSLSL |
| | | SLGK |
| 14 | Human IgG4s Fc | ESKYGPPCPPCPAPPVAGPSVFLFPPKPKDTLMISRTPEVTCVVV |
| | Variant hIgG4 | DVSQEDPEVQFNWYVDGVEVHNAKTKPREEQFNSTYRVVSVLTVL |
| | hinge and Fc, with | HQDWLNGKEYKCKVSNKGLPSSIEKTISKAKGQPREPQVYTLPPS |
| | IgG2-based hinge | QEEMTKNQVSLTCLVKGFYPSDIAVEWESNGQPENNYKTTPPVLD |
| | region with S108P | SDGSFFLYSRLTVDKSRWQEGNVFSCSVMHEALHNHYTQKSLSLS |
| | mutation (S228P by | LGK |
| | EU numbering), | |
| | and IgG1 CH2 and | |
| | CH3 | |
| 15 | human IgG1 PVA | EPKSCDKTHTCPPCPAPPVAGPSVFLFPPKPKDTLMISRTPEVTC |
| | Variant hIgG1 | VVVDVSHEDPEVKFNWYVDGVEVHNAKTKPREEQYNSTYRVVSVL |
| | hinge and Fc, with | TVLHQDWLNGKEYKCKVSNKALPAPIEKTISKAKGQPREPQVYTL |
| | IgG2-based hinge | PPSRDELTKNQVSLTCLVKGFYPSDIAVEWESNGQPENNYKTTPP |
| | region and IgG1 | VLDSDGSFFLYSKLTVDKSRWQQGNVFSCSVMHEALHNHYTQKSL |
| | CH2 and CH3 | SLSPGK |
| 16 | Fc domain (SEQ ID | DKRVESKYGP PCPPCPAPPV AGPSVFLFPP KPKDTLMISR |
| | NO: 1 of | TPEVTCVVVD VSQEDPEVQF NWYVDGVEVH NAKTKPREEQ |
| | WO2014/121087) | FNSTYRVVSV LTVLHQDWLN GKEYKCKVSN KGLPSSIEKT |
| | | ISKAKGQPRE PQVYTLPPSQ EEMTKNQVSL TCLVKGFYPS |
| | | DIAVEWESNG QPENNYKTTP PVLDSDGSFF LYSRLTVDKS |
| | | RWQEGNVFSC SVMHEALHNH YTQKSLSLSL GK |
| 17 | Fc domain (SEQ ID | DKKVEPKSCD KTHTCPPCPA PPVAGPSVFL FPPKPKDTLM |
| | NO: 2 of | ISRTPEVTCV VVDVSQEDPE VQFNWYVDGV EVHNAKTKPR |
| | WO2014/121087) | EEQFNSTYRV VSVLTVLHQD WLNGKEYKCK VSNKGLPSSI |
| | | EKTISKAKGQ PREPQVYTLP PSRDELTKNQ VSLTCLVKGF |
| | | YPSDIAVEWE SNGQPENNYK TTPPVLDSDG SFFLYSKLTV |
| | | DKSRWQQGNV FSCSVMHEAL HNHYTQKSLS LSPGK |
| 18 | Fc domain (SEQ ID | ASTKGPSVFP LAPSSKSTSG GTAALGCLVK DYFPEPVTVS |
| | NO: 30 of | WNSGALTSGV HTFPAVLQSS GLYSLSSVVT VPSSSLGTQT |
| | WO2014/121087) | YICNVNHKPS NTKVDKKVEP KSCDKTHTCP PCPAPPVAGP |
| | | SVFLFPPKPK DTLMISRTPE VTCVVVDVSQ EDPEVQFNWY |
| | | VDGVEVHNAK TKPREEQFNS TYRVVSVLTV LHQDWLNGKE |
| | | YKCKVSNKGL PSSIEKTISK AKGQPREPQV YTLPPSRDEL |
| | | TKNQVSLTCL VKGFYPSDIA VEWESNGQPE NNYKTTPPVL |
| | | DSDGSFFLYS KLTVDKSRWQ QGNVFSCSVM HEALHNHYTQ |
| | | KSLSLSPGK |
| 19 | Fc domain (SEQ ID | ASTKGPSVFP LAPCSRSTSE STAALGCLVK DYFPEPVTVS |
| | NO: 31 of | WNSGALTSGV HTFPAVLQSS GLYSLSSVVT VPSSSLGTKT |
| | WO2014/121087) | YTCNVDHKPS NTKVDKRVES KYGPPCPPCP APPVAGPSVF |
| | | LFPPKPKDTL MISRTPEVTC VVVDVSQEDP EVQFNWYVDG |
| | | VEVHNAKTKP REEQFNSTYR VVSVLTVLHQ DWLNGKEYKC |
| | | KVSNKGLPSS IEKTISKAKG QPREPQVYTL PPSQEEMTKN |
| | | QVSLTCLVKG FYPSDIAVEW ESNGQPENNY KTTPPVLDSD |
| | | GSFFLYSRLT VDKSRWQEGN VFSCSVMHEA LHNHYTQKSL |
| | | SLSLGK |
| 20 | Fc domain (SEQ ID | ASTKGPSVFP LAPSSKSTSG GTAALGCLVK DYFPEPVTVS |
| | NO: 37 of | WNSGALTSGV HTFPAVLQSS GLYSLSSVVT VPSSSLGTQT |
| | WO2014/121087) | YICNVNHKPS NTKVDKKVEP KSCDKTHTCP PCPAPPVAGP |
| | | SVFLFPPKPK DTLMISRTPE VTCVVVDVSQ EDPEVQFNWY |
| | | VDGVEVHNAK TKPREEQFNS TYRVVSVLTV LHQDWLNGKE |
| | | YKCKVSNKGL PSSIEKTISK AKGQPREPQV YTLPPSRDEL |
| | | TKNQVSLTCL VKGFYPSDIA VEWESNGQPE NNYKTTPPVL |
| | | DSDGSFFLYS KLTVDKSRWQ QGNVFSCSVM HEALHNRFTQ |
| | | KSLSLSPGK |
| 21 | Fc domain (SEQ ID | ASTKGPSVFP LAPCSRSTSE STAALGCLVK DYFPEPVTVS |
| | NO: 38 of | WNSGALTSGV HTFPAVLQSS GLYSLSSVVT VPSSSLGTKT |
| | WO2014/121087) | YTCNVDHKPS NTKVDKRVES KYGPPCPPCP APPVAGPSVF |
| | | LFPPKPKDTL MISRTPEVTC VVVDVSQEDP EVQFNWYVDG |
| | | VEVHNAKTKP REEQFNSTYR VVSVLTVLHQ DWLNGKEYKC |
| | | KVSNKGLPSS IEKTISKAKG QPREPQVYTL PPSQEEMTKN |
| | | QVSLTCLVKG FYPSDIAVEW ESNGQPENNY KTTPPVLDSD |
| | | GSFFLYSRLT VDKSRWQEGN VFSCSVMHEA LHNRFTQKSL |
| | | SLSLGK |
| 22 | Linker | GGGGGGGGG |
| 23 | Linker | GnS |
| 24 | Linker | SGn |
| 25 | Linker | G4S |
| 26 | Linker | (GGGGS)n |
| 27 | Linker | GGGG |
| 28 | Linker | GGGGG |
| 29 | Linker | GGGGGG |
| 30 | Linker | GGGGGGG |
| 31 | Linker | GGGGGGGG |
| 32 | Hinge core | CPPC |
| 33 | Hinge core | CPSC |
| 34 | Chimeric hinge | EPKSCDKTHTCPPCPAPPVA |
| | region (SEQ ID | |
| | NO: 8 of | |
| | WO2014/121087) | |
| 35 | Chimeric hinge | ESKYGPPCPPCPAPPVA |
| | region (SEQ ID | |
| | NO: 9 of | |
| | WO2014/121087) | |
| 36 | Modified hinge | CPPCPAPGGG-GPSVF |
| 37 | Modified hinge | CPPCPAPGG--GPSVF |
| 38 | Modified hinge | CPPCPAPG---GPSVF |
| 39 | Modified hinge | CPPCPAP----GPSVF |
| 40 | Mature human | RNLPVATPDPGMFPCLHHSQNLLRAVSNMLQKARQTLEFYPCTSE |
| | p35(Y189E) | EIDHEDITKDKTSTVEACLPLELTKNESCLNSRETSFITNGSCLA |
| | | SRKTSFMMALCLSSIYEDLKMYQVEFKTMNAKLLMDPKRQIFLDQ |
| | | NMLAVIDELMQALNFNSETVPQKSSLEEPDFEKTKIKLCILLHAF |
| | | RIRAVTIDRVMSYLNAS |
| 41 | hIgG4us Fc | ESKYGPPCPPCPAPGGGGPSVFLFPPKPKDTLMISRTPEVTCVVV |
| | | DVSQEDPEVQFNWYVDGVEVHNAKTKPREEQFNSTYRVVSVLTVL |
| | | HQDWLNGKEYKCKVSNKGLPSSIEKTISKAKGQPREPQVYTLPPS |
| | | QEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLS |
| | | LGK |
| 42 | hIgG1s Fc | DKKVEPKSCDKTHTCPPCPAPPVAGPSVFLFPPKPKDTLMISRTP |
| | | EVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHYT |
| | | QKSLSLSPGK |
| 43 | hIgG1us Fc | DKKVEPKSCDKTHTCPPCPAPGGGGPSVFLFPPKPKDTLMISRTP |
| | | EVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHYT |
| | | QKSLSLSPGK |

What is claimed is:

1. A method comprising administering to a subject:
(a) a tumor-targeted IL12Rβ1 agonist (“R1 agonist”) comprising:
(i) a first tumor-targeting moiety; and
(ii) an IL12Rβ1 binding moiety; and
(b) a tumor-targeted IL12Rβ2 agonist (“R2 agonist”) comprising:
(i) a second tumor-targeting moiety; and
(ii) a IL12Rβ2 binding moiety.

2. The method of claim 1, wherein the IL12Rβ1 binding moiety and the IL12Rβ2 binding moiety each comprises or consists of an antigen binding domain of an antibody.

3. The method of claim 2, wherein the IL12Rβ1 binding moiety and the IL12Rβ2 binding moiety are Fabs.

4. The method of claim 2, wherein the IL12Rβ1 binding moiety and the IL12Rβ2 binding moiety are scFvs.

5. The method of claim 2, wherein the IL12Rβ1 binding moiety and the IL12Rβ2 binding moiety are sdAbs.

6. The method of any one of claims 2 to 5, wherein the IL12Rβ1 binding moiety binds to the D2 domain of IL12Rβ1 and the IL12Rβ2 binding moiety binds to the D1 domain of IL12Rβ2.

7. The method of any one of claims 1 to 6, wherein:
(a) the IL12Rβ1 binding moiety is a first IL12 moiety comprising a first p40 moiety associated with a first p35 moiety; and
(b) the IL12Rβ2 binding moiety is a second IL12 moiety comprising a second p35 moiety associated with a second p40 moiety.

8. The method of claim 7, wherein:
(a) the first IL12 moiety has greater selectivity to IL12Rβ1 than IL12Rβ2 as compared to wild-type human IL12; and/or
(b) the second IL12 moiety has greater selectivity to IL12Rβ2 than IL12Rβ1 as compared to wild-type human IL12.

9. The method of claim 7 or 8, wherein the first p35 moiety comprises an amino acid sequence having at least 95%, at least 96%, at least 97%, at least 98%, or at least 99% sequence identity to the amino acid sequence of SEQ ID NO:2.

10. The method of any one of claims 7 to 9, wherein the first p35 moiety is a variant p35 moiety having reduced binding to IL12Rβ2 as compared to a p35 moiety having the amino acid sequence of SEQ ID NO:2.

11. The method of claim 10, wherein the variant p35 moiety comprises one or more of the mutations set forth in Table 1, optionally wherein the variant p35 moiety comprises the amino acid sequence of SEQ ID NO:40.

12. The method of claim 7 or 8, wherein the first p35 moiety comprises the amino acid sequence of SEQ ID NO:2.

13. The method of any one of claims 7 to 12, wherein the first p40 moiety comprises an amino acid sequence having at least 95%, at least 96%, at least 97%, at least 98%, or at least 99% sequence identity to the amino acid sequence of SEQ ID NO:6 or SEQ ID NO:7.

14. The method of any one of claims 7 to 13, wherein the first p40 moiety comprises the amino acid sequence of SEQ ID NO:6 or SEQ ID NO:7.

15. The method of any one of claims 7 to 14, wherein the second p35 moiety comprises an amino acid sequence having at least 95%, at least 96%, at least 97%, at least 98%, or at least 99% sequence identity to the amino acid sequence of SEQ ID NO:2.

16. The method of any one of claims 7 to 14, wherein the second p35 moiety comprises the amino acid sequence of SEQ ID NO:2.

17. The method of any one of claims 7 to 16, wherein the second p40 moiety comprises an amino acid sequence having at least 95%, at least 96%, at least 97%, at least 98%, or at least 99% sequence identity to the amino acid sequence of SEQ ID NO:6 or SEQ ID NO:7.

18. The method of claim 17, wherein the second p40 moiety is a variant p40 moiety having reduced binding to IL12Rβ1 as compared to a wild-type p40 moiety.

19. The method of claim 18, wherein the variant p40 moiety comprises a D1 domain mutation or a D1 domain deletion.

20. The method of any one of claims 7 to 19, wherein the second p40 moiety comprises the amino acid sequence of SEQ ID NO:6.

21. The method of any one of claims 7 to 20, wherein the first p35 moiety and the first p40 moiety are separated by a linker (a first “IL12 moiety linker”) and/or the second p35 moiety and the second p40 moiety are separated by a linker (a second “IL12 moiety linker”).

22. The method of claim 21, wherein the first IL12 moiety linker and/or the second IL12 moiety linker are each at least 5, at least 10, or at least 15 amino acids in length.

23. The method of claim 21 or 22, wherein the first IL12 moiety linker and/or the second IL12 moiety linker is a non-cleavable linker.

24. The method of any one of claims 1 to 23, wherein the first tumor-targeting moiety binds to a first tumor-associated antigen and the second tumor-targeting moiety binds to a second tumor-associated antigen.

25. The method of claim 24, wherein the first tumor-associated antigen and the second tumor-associated antigen are expressed on the same tumor cell.

26. The method of claim 24 or claim 25, wherein the first tumor-associated antigen and the second tumor-associated antigen are different.

27. The method of claim 24 or claim 25, wherein the first tumor-associated antigen and the second tumor-associated antigen are the same.

28. The method of claim 27, wherein the first tumor-targeting moiety and the second tumor-targeting moiety are the same.

29. The method of claim 27, wherein the first tumor-targeting moiety and the second tumor-targeting moiety are different.

30. The method of claim 27 or claim 29, wherein the first tumor-targeting moiety and the second tumor-targeting moiety do not compete for binding to the tumor-associated antigen.

31. The method of any one of claims 24 to 30, wherein the first tumor-targeting moiety and/or the second tumor-targeting moiety are Fabs.

32. The method of any one of claims 24 to 30, wherein the first tumor-targeting moiety and/or the second tumor-targeting moiety are scFvs.

33. The method of any one of claims 24 to 30, wherein the first tumor-targeting moiety and/or the second tumor-targeting moiety are sdAbs.

34. The method of any one of claims 24 to 33, wherein the first tumor-targeting moiety and/or second tumor-targeting moiety bind(s) to PSMA, MSLN, or MUC16.

35. The method of any one of claims 1 to 34, wherein the tumor-targeted IL12Rβ1 agonist comprises
(a) a first polypeptide chain comprising, in N- to C-terminal orientation:
(i) the first tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
(ii) optionally, a linker (a “TAA-Fc linker”); and
(iii) a first Fc domain; and
(b) a second polypeptide chain comprising, in N- to C-terminal orientation:
(i) the IL12Rβ1 binding moiety or component thereof (or a component thereof, e.g., a VH-CH1 or VL-CL), optionally associated with another component thereof on a separate polypeptide chain (or a component thereof, e.g., a VL-CL or VH-CH1);
(ii) optionally, a linker (“an IL12-Fc linker”); and
(iii) a second Fc domain associated with the first Fc domain.

36. The method of claim 35, wherein the first Fc domain and second Fc domain each comprises an amino acid sequence having at least 95% sequence identity to the amino acid sequence of SEQ ID NO:11, SEQ ID NO:12, or SEQ ID NO:13.

37. The method of claim 35 or 36, wherein the first Fc domain and second Fc domain each comprises a chimeric hinge domain.

38. The method of claim 37, wherein the first Fc domain and second Fc domain each comprises an amino acid sequence having at least 98% or at least 99% sequence identity to the amino acid sequence of SEQ ID NO:14 or SEQ ID NO:15.

39. The method of any one of claims 35 to 38, wherein the first Fc domain and second Fc domain each has reduced effector function.

40. The method of any one of claims 35 to 38, wherein the first Fc domain and second Fc domain form an Fc heterodimer.

41. The method of any one of claims 1 to 40, wherein the tumor-targeted IL12Rβ2 agonist comprises
(a) a third polypeptide chain comprising, in N- to C-terminal orientation:
(i) the second tumor-targeting moiety or component thereof (or a component thereof, e.g., a VH-CH1 or VL-CL), optionally associated with another component thereof on a separate polypeptide chain (or a component thereof, e.g., a VL-CL or VH-CH1);
(ii) optionally, a linker (a “TAA-Fc linker”); and
(iii) a third Fc domain; and
(b) a third polypeptide chain comprising, in N- to C-terminal orientation:
(i) the IL12Rβ2 binding moiety or component thereof (or a component thereof, e.g., a VH-CH1 or VL-CL), optionally associated with another component thereof on a separate polypeptide chain (or a component thereof, e.g., a VL-CL or VH-CH1);
(ii) optionally, a linker (“an IL12-Fc linker”); and
(iii) a fourth Fc domain associated with the third Fc domain.

42. The method of claim 41, wherein the third Fc domain and fourth Fc domain each comprises an amino acid sequence having at least 95% sequence identity to the amino acid sequence of SEQ ID NO:11, SEQ ID NO:12, or SEQ ID NO:13.

43. The method of claim 41 or 42, wherein the third Fc domain and fourth Fc domain each comprises a chimeric hinge domain.

44. The method of any one of claims 41 to 43, wherein the third Fc domain and fourth Fc domain each has reduced effector function.

45. The method of any one of claims 41 to 44, wherein the third Fc domain and fourth Fc domain form an Fc heterodimer.

46. The method of any one of claims 1 to 45, wherein the tumor-targeted IL12Rβ1 agonist is monovalent for the first tumor-targeting moiety.

47. The method of any one of claims 1 to 46, wherein the tumor-targeted IL12Rβ1 agonist is monovalent for the IL12Rβ1 binding moiety.

48. The method of any one of claims 1 to 47, wherein the tumor-targeted IL12Rβ2 agonist is monovalent for the second tumor-targeting moiety.

49. The method of any one of claims 1 to 48, wherein the tumor-targeted IL12Rβ2 agonist is monovalent for the IL12Rβ2 binding moiety.

50. The method of any one of claims 1 to 49, wherein the tumor-targeted IL12Rβ1 agonist and the tumor-targeted IL12Rβ2 agonist are both in the form of a pharmaceutical composition comprising the agonist and an excipient.

51. The method of claim 50, wherein the tumor-targeted IL12Rβ1 agonist and the tumor-targeted IL12Rβ2 agonist are in the same pharmaceutical composition.

52. The method of claim 50, wherein the tumor-targeted IL12Rβ1 agonist and the tumor-targeted IL12Rβ2 agonist are in different pharmaceutical compositions.

53. The method of any one of claims 1 to 52, wherein tumor-targeted IL12Rβ1 agonist is configured as illustrated in FIG. 1A, 1B, 1C, 1D, 1E, 1F, 1G, 1H, 1I, 1J, 1K, or 1L.

54. The method of any one of claims 1 to 52, wherein tumor-targeted IL12Rβ2 agonist is configured as illustrated in FIG. 2A, 2B, 2C, 2D, 2E, 2F, 2G, 2H, 2I, 2J, 2K, or 2L.

55. The method of any one of claims 1 to 54, wherein the method further comprises administering a multispecific T-cell engager.

56. The method of claim 55, wherein the multispecific T-cell engager is a bispecific T-cell engager.

57. The method of claim 55 or 56, wherein the multispecific T-cell engager comprises a TAA targeting moiety and a CD3 targeting moiety.
